(19) United States
(12) Patent Application Publication
(10) Pub. No.: US 2022/0008473 A1
(43) Pub. Date: Jan. 13, 2022

(54) USE OF CHIMERIC ANTIGEN RECEPTOR T CELLS AND NK CELL INHIBITORS FOR TREATING CANCER

(71) Applicant: CRISPR Therapeutics AG, Zug (CH)

(72) Inventors: Jonathan Alexander Terrett, Cambridge, MA (US); Lawrence Klein, Cambridge, MA (US); Ewelina Morawa, Cambridge, MA (US)

(21) Appl. No.: 17/488,215

(22) Filed: Sep. 28, 2021

Related U.S. Application Data
(63) Continuation of application No. PCT/IB2020/056085, filed on Jun. 26, 2020.
(60) Provisional application No. 62/951,732, filed on Dec. 20, 2019.
(60) Provisional application No. 62/867,764, filed on Jun. 27, 2019.

(51) Int. Cl. A61K 35/17; A61P 35/02; A61K 39/00; C07K 14/725

(52) U.S. Cl. CPC A61K 35/17; A61P 35/02; A61K 39/001112; A61K 38/00; C07K 14/7051; A61K 2039/5156; A61K 39/001117

(57) ABSTRACT

Methods for improving a clinical outcome in a subject comprising administering to a subject in need of the treatment a population of genetically engineered immune cells (e.g., T cells), which express a chimeric antigen receptor (CAR) and a natural killer (NK) cell inhibitor (e.g., daratumumab). The genetically engineered immune cells may comprise a disrupted TRAC gene, a disrupted B2M gene, or both. The disclosure also features compositions for use in the methods.

### CROSS REFERENCE TO RELATED APPLICATIONS

The present application is a bypass continuation of International Patent Application No. PCT/IB2020/056085, filed Jun. 26, 2020, which claims the benefit of the filing dates of U.S. Provisional Application No. 62/867,764, filed Jun. 27, 2019, and U.S. Provisional Application No. 62/951,732, filed Dec. 20, 2019. The entire contents of each of the prior applications are incorporated by reference herein.

### SEQUENCE LISTING

The application contains a Sequence Listing that has been filed electronically in the form of a text file, created Sep. 28, 2021, and named “095136-0372_Seq-Listing.txt” (133,130 bytes), the contents of which are incorporated by reference herein in their entirety.

### BACKGROUND OF THE INVENTION

Chimeric antigen receptor (CAR) T cell therapies are adoptive T cell therapeutics used to treat human malignancies. Although CAR T cell therapy has led to tremendous clinical success, including durable remission in relapsed/refractory non-Hodgkin lymphoma (NHL) and pediatric acute lymphoblastic leukemia (ALL), the approved products are autologous and require patient-specific cell collection and manufacturing. Because of this, some patients have experienced disease progression or death while awaiting treatment. Allogeneic CAR T cell therapy, comprising disrupted MHC-1 complexes, presents an attractive off-the-shelf option to autologous CAR T cell therapy. The disrupted MHC class I in the allogeneic T cells, however, renders the CAR T cells susceptible to elimination by the host immune system, for example, by natural killer (NK) cell-mediated immune response. Accordingly, there remains a need for improved CAR T cell therapy

### SUMMARY OF THE INVENTION

The present disclosure is based, at least in part, on the unexpected discoveries that an anti-CD38 antibody (daratumumab), which is an exemplary NK cell inhibitor, successfully depleted NK cells both in vitro and in vivo but did not affect T cell numbers, including numbers of genetically engineered T cells expressing a chimeric antigen receptor (CAR), and did not activate CAR T cells. Further, it was found that, unexpectedly, daratumumab pre-treatment significantly reduced NK cell-mediated CAR T cell lysis (e.g., by approximately 50%) and preserves the viability and number of allogeneic CAR T cells. Moreover, combined therapy of daratumumab and CAR-T cells exhibited synergistic effect in reducing tumor burden and extending survival rates in a xenograft mouse model, even in the presence of NK cells.

Accordingly, one aspect of the present disclosure features a combined therapy to improve a clinical outcome, wherein the combined therapy involves genetically engineered T cells expressing a chimeric antigen receptor (“CAR” and “CAR T cells”) and an NK cell inhibitor (e.g., an anti-CD38 antibody such as daratumumab or a functional variant thereof).

In some embodiments, provided herein is a method for improving a clinical outcome in a subject receiving a chimeric antigen receptor (CAR) T cell therapy, the method comprising: administering to the subject an effective amount of a population of engineered human CAR T cells, wherein the engineered human CAR T cells comprise disrupted MHC class I, and wherein the subject has received or is receiving an effective amount of an NK cell inhibitor, thereby improving a clinical outcome in the subject.

In some examples, a method for improving a clinical outcome in a subject, comprises: administering to the subject a chimeric antigen receptor (CAR) T cell therapy comprising a population of engineered human CAR T cells, wherein the engineered human CAR T cells comprise (i) a disrupted B2M gene; (ii) a disrupted TRAC gene; and (iii) a nucleic acid encoding a CAR, and wherein the subject has received or is receiving an effective amount of an anti-CD38 antibody, thereby improving a clinical outcome in the subject.

In some embodiments, provided herein is a method for improving a clinical outcome in a subject receiving a chimeric antigen receptor (CAR) T cell therapy, the method comprising: administering to the subject an effective amount of an NK cell inhibitor, wherein the subject has received or is receiving an effective amount of a population of engineered human CAR T cells, and wherein the engineered human CAR T cells comprise disrupted MHC class I, thereby reducing NK cell activity in the subject, thereby improving a clinical outcome in the subject.

In some examples, a method for improving a clinical outcome in a subject comprises: administering to the subject an effective amount of an anti-CD38 antibody, wherein the subject has received or is receiving a chimeric antigen receptor (CAR) T cell therapy comprising a population of engineered human CAR T cells, wherein the engineered human CAR T cells comprise (i) a disrupted B2M gene; (ii) a disrupted TRAC gene; and (iii) a nucleic acid encoding a CAR, thereby improving a clinical outcome in the subject.

In some embodiments, provided herein is a method for improving a clinical outcome in a subject receiving a chimeric antigen receptor (CAR) T cell therapy, the method comprising administering to the subject: an effective amount of: (a) an effective amount of an NK cell inhibitor; and (b) an effective amount of a population of engineered human CAR T cells, wherein the engineered human CAR T cells comprise disrupted MHC class I, thereby improving a clinical outcome in the subject.

In some examples, a method for improving a clinical outcome in a subject, the method comprising administering to the subject: (a) an effective amount of a population of engineered human CAR T cells, wherein the engineered human CAR T cells comprise (i) a disrupted B2M gene; (ii) a disrupted TRAC gene; and (iii) a nucleic acid encoding a CAR; and (b) an effective amount of an anti-CD38 monoclonal antibody, thereby improving a clinical outcome in the subject.

In any of the methods disclosed herein, the improved clinical outcome may comprise one or more of the following: (i) reducing natural killer (NK) cell activity in the subject; (ii) increasing a clinical response to the CAR-T therapy in the subject; (iii) increasing persistence of the engineered human CAR-T cells in the subject; and (iv) decreasing cell lysis of the engineered human CAR-T cells in the subject. In some embodiments, the subject is a cancer patient, and the improved clinical outcome comprises one or more of the following: (i) reducing tumor size or tumor cell numbers in the subject; and (ii) increasing an anti-tumor response in the subject.

In some examples, a clinical response to the CAR-T therapy (e.g., anti-tumor response) in the subject may be increased relative to the CAR-T therapy alone. Alternatively, the clinical response to the CAR-T therapy in the subject may be increased relative to the therapy comprising the NK cell inhibitor alone. In yet other examples, the clinical response increase may be additive. Alternatively, the clinical response increase may be synergistic.

In some examples, cell lysis of the engineered human CAR-T cells can be reduced by 10%, 20%, 30%, 40%, 50%, 60%, 70%, 80%, 90%, or 100% in the subject relative to a subject receiving the engineered human CAR T cells without the NK cell inhibitor.

In any of the methods disclosed herein, expression of the MHC class I in the engineered human CAR-T cells is inhibited. In some instances, the engineered human CAR T cells may comprise a disrupted beta-2-microglobulin (B2M) gene. Alternatively or in addition, the engineered human CAR T cells may comprise a disrupted HLA-A, HLA-B or HLA-C gene.

In some embodiments, the engineered human CAR T cells comprise: (i) a disrupted T cell receptor alpha chain constant region (TRAC) gene; (ii) a disrupted B2M gene (e.g., as those described herein); and (iii) a nucleic acid encoding a CAR. For example, the disrupted B2M gene may comprise an insertion, deletion and/or substitution of at least one nucleotide base pair. In specific examples, the disrupted B2M gene of the engineered human CAR-T cells may comprise at least one nucleotide sequence of SEQ ID NO: 9; SEQ ID NO: 10; SEQ ID NO: 11; SEQ ID NO: 12; SEQ ID NO: 13; and SEQ ID NO: 14. Alternatively or in addition, the engineered human T cells may comprise a deletion of the nucleotide sequence of SEQ ID NO: 29 in the TRAC gene relative to unmodified T cells. In some instances, the nucleic acid encoding the CAR may be located inside the disrupted TRAC gene.

In some embodiments, at least 50% of the engineered human T cells may express a detectable level of the CAR. Alternatively or in addition, less than 0.5% of the population of cells expresses a detectable level of TCR. Further, at least 50% of the engineered human CAR T cells may not express a detectable level of B2M surface protein.

In some examples, the engineered human CAR T cells express a CAR comprising an ectodomain that comprises an antigen-binding fragment, which binds a tumor antigen. Exemplary tumor antigens include, but are not limited to, CD19, CD33, CD70, and BCMA. In some examples, the tumor antigen is CD19 and the anti-CD19 antigen-binding fragment in a CAR is an anti-CD19 scFv. In some instances, the anti-CD19 antigen-binding fragment is a human or humanized anti-CD19 antigen-binding fragment. In some examples, the tumor antigen is BCMA and the anti-BCMA antigen-binding fragment in a CAR is an anti-BCMA scFv. In some instances, the anti-CD19 antigen-binding fragment is a human or humanized anti-BCMA antigen-binding fragment. In some examples, the tumor antigen is CD70 and the anti-CD70 antigen-binding fragment in a CAR is an anti-CD70 scFv. In some instances, the anti-CD70 antigen-binding fragment is a human or humanized anti-CD70 antigen-binding fragment. In some examples, the tumor antigen is CD33 and the anti-CD33 antigen-binding fragment in a CAR is an anti-CD33 scFv. In some instances, the anti-CD33 antigen-binding fragment is a human or humanized anti-CD33 antigen-binding fragment.

In any of the methods disclosed herein, the NK cell inhibitor may reduce the number of NK cells, inhibits an activity of the NK cells, or both. In some examples, the NK cells inhibitor may reduce the number of NK cells by at least 10%, 20%, 30%, 40%, 50%, 60%, 70%, 80%, or 90%. Without being bound by theory, the NK cell inhibitor reduces the number of NK cells by antibody-dependent cell-mediated cytotoxicity (ADCC), antibody-dependent cellular phagocytosis (ADCP), complement dependent cytotoxicity (CDC), apoptosis, or any combinations thereof.

In some embodiments, the NK cell inhibitor does not significantly reduce endogenous T cell numbers. Alternatively or in addition, the NK inhibitor does not significantly activate the engineered human CAR T cells.

In some embodiments, the NK cell inhibitor may be a small molecule, a monoclonal antibody or an antigen-binding fragment thereof, a polypeptide, a polynucleotide, or combinations thereof. In some examples, the NK cell inhibitor can be an antibody that specifically binds CD38. For example, the NK cell inhibitor can be daratumumab, SAR650984, or MOR202, or an antigen-binding fragment thereof. In some instances, the NK cell inhibitor is an antibody that binds to the same epitope as daratumumab and/or competes with daratumumab for binding to CD38. In specific examples, the antibody may comprise the same heavy chain and light chain complementary determining regions as daratumumab. For example, the antibody may comprise the same heavy chain variable region and the same light chain variable region as daratumumab.

In any of the methods disclosed herein, the NK cell inhibitor can be administered concurrently with administration of the population of engineered human CAR T cells. Alternatively, the population of engineered human CAR T cells is administered prior to administration of the NK cell inhibitor. In other examples, the NK cell inhibitor is administered prior to administration of the population of engineered human CAR T cells.

Any of the methods disclosed herein may further comprise a pre-conditioning regimen prior to administration of the population of engineered human CAR T cells. Such a pre-conditioning regimen comprises a lymphodepletion regimen. In some examples, a lymphodepletion regimen may comprise administering at least one chemotherapeutic agent. Examples include cyclophosphamide, fludarabine, or a combination thereof. In specific examples, a lymphodepletion regimen may comprise a combination of fludarabine and cyclophosphamide administered via intravenous infusion.

In some instances, the population of engineered human CAR T cells can be administered at least 48 hours after the lymphodepletion regimen. In some examples, the population of engineered human CAR T cells can be administered at least two days, at least three days (e.g., at least four days, at least five days, at least six days, or at least seven days) after the lymphodepletion regimen. In some examples, the population of engineered human CAR T cells can be administered no more than seven days after the lymphodepletion regimen.

In some instances, the lymphodepletion regimen can be administered for at least one day (e.g., at least two days, at least three days, or at least four days). In specific examples, the population of engineered human CAR T cells can be administered between 48 hours and seven days after the lymphodepletion regimen, and the lymphodepletion regimen can be administered for two to three days.

In some embodiments, the population of engineered human CAR T cells can be administered by one or more intravenous infusions. In some examples, the population of engineered human CAR T cells is administered by a single intravenous infusion. Alternatively, the population of engineered human CAR T cells is administered by more than one intravenous infusion. In some examples, a single container comprises a dose of the population of engineered human CAR T cells. In other examples, more than one container comprises the dose of the population of engineered human CAR T cells.

In some embodiments, the NK cell inhibitor can be administered by one or more intravenous infusions. In some examples, the NK cell inhibitor is daratumumab, which can be administered at a dose of 1 to 24 mg/kg. In specific examples, the NK cell inhibitor is daratumumab, which is administered as a single dose infusion at 16 mg/kg. In further examples, the NK cell inhibitor is daratumumab, which is administered as a split dose infusion at 8 mg/kg. The split dose is administered on consecutive days.

In some instances, any of the method disclosed herein may comprise administering to the subject a subsequent dose of the NK cell inhibitor. In some examples, the subsequent dose of the NK cell inhibitor is administered to the subject when NK cell numbers in the subject recover to about 10%, 20%, 30%, 40%, 50%, 60%, 70%, 80%, 90%, or 100% of NK cell numbers prior to administration of the NK cell inhibitor.

In one example, the subject is administered a dose of about 1×10⁷-3×10⁸ engineered human CAR T cells expressing a detectable level of the CAR at least 48 hours but no more than seven days after the lymphodepletion therapy.

Further, the present disclosure provides a composition comprising a population of engineered human chimeric antigen receptor (CAR) T cells as those disclosed herein for use in a CAR T cell therapy in combination with an effective amount of an NK cell inhibitor as also disclosed herein, for the treatment of a cancer. The present disclosure also provides uses of the composition comprising a population of engineered human chimeric antigen receptor (CAR) T cells as those disclosed herein for manufacturing a first medicament for use in a CAR T cell therapy for treating a cancer in a subject in need thereof. The first medicament comprises the population of cells comprising engineered human CAR T cells and the first medicament is administered in combination with a second medicament comprising an NK cell inhibitor and optionally a pharmaceutically acceptable carrier.

Also within the scope of the present disclosure are:

(a) a kit comprising a first medicament comprising the composition comprising the population of CAR-T cells as disclosed herein, and a package insert comprising instructions for administration of the composition in combination with a second medicament comprising a composition that comprises an NK cell inhibitor as also disclosed herein, and an optional pharmaceutically acceptable carrier, to a subject in need thereof;

(b) a kit comprising a first composition comprising the population of engineered human CAR T cells as disclosed herein, a second composition comprising the NK cell inhibitor as also disclosed herein, and a package insert comprising instructions for administration of the first composition in combination with the second composition to a subject in need thereof; and

(c) a kit comprising a first composition comprising a population of engineered human CAR T cells as disclosed herein, for use in a CAR T cell therapy, a second composition comprising an NK cell inhibitor as also disclosed herein, and a package insert comprising instructions for administration of the first composition in combination with the second composition, to a subject for the treatment of cancer.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1C provide flow cytometry plots showing CD38 expression on CAR T cells. FIG. 1A shows CD38 expression on anti-BCMA CAR T cells measured by flow cytometry. FIG. 1B shows CD38 expression on anti-CD19 CAR T cells as measured by flow cytometry. FIG. 1C provides flow cytometry plots showing fluorescent minus one (FMO) control stained cells were used to set the gate for measuring CD38+ cells.

FIGS. 2A-2D are flow cytometry plots showing CD38 expression on normal immune cells (PBMCs) collected from a healthy donor (Donor 3469) and cultured in media alone or media supplemented with 10% complement. FIG. 2A shows the percentage of CD38 expressing T cells from PBMCs cultured in media alone. FIG. 2B shows the percentage of CD38 expressing T cells from PBMCs cultured in media supplemented with 10% complement. FIG. 2C shows the percentage of CD38 expressing NK cells from PBMCs cultured in media alone. FIG. 2D shows the percentage of CD38 expressing NK cells from PBMCs cultured in media supplemented with 10% complement.

FIGS. 3A-3D are flow cytometry plots showing CD38 expression on normal immune cells (PBMCs) collected from a healthy donor (Donor 3383) and cultured in media alone or media supplemented with 10% complement. FIG. 3A shows the percentage of CD38 expressing T cells from PBMCs cultured in media alone. FIG. 3B shows the percentage of CD38 expressing T cells from PBMCs cultured in media supplemented with 10% complement. FIG. 3C shows the percentage of CD38 expressing NK cells from PBMCs cultured in media alone. FIG. 3D shows the percentage of CD38 expressing NK cells from PBMCs cultured in media supplemented with 10% complement.

FIGS. 4A-4D are flow cytometry plots showing CD38 expression on normal immune cells (PBMCs) collected from a healthy donor (Donor 3469) after in vitro culture for 72 hours in either media alone or media supplemented with 10% complement. FIG. 4A shows the percentage of CD38 expressing T cells from PBMCs cultured in media alone for 72 hours. FIG. 4B shows the percentage of CD38 expressing T cells from PBMCs cultured in media supplemented with 10% complement for 72 hours. FIG. 4C shows the percentage of CD38 expressing NK cells from PBMCs cultured in media alone for 72 hours. FIG. 4D shows the percentage of CD38 expressing NK cells from PBMCs cultured in media supplemented with 10% complement for 72 hours.

FIGS. 5A-5D are flow cytometry plots showing CD38 expression on normal immune cells (PBMCs) collected from a healthy donor (Donor 3383) after in vitro culture for 72 hours in either media alone or media supplemented with 10% complement. FIG. 5A shows the percentage of CD38 expressing T cells from PBMCs cultured in media alone for 72 hours. FIG. 5B shows the percentage of CD38 expressing T cells from PBMCs cultured in media supplemented with 10% complement for 72 hours. FIG. 5C shows the percentage of CD38 expressing NK cells from PBMCs cultured in media alone for 72 hours. FIG. 5D shows the percentage of CD38 expressing NK cells from PBMCs cultured in media supplemented with 10% complement for 72 hours.

FIGS. 6A-6D are graphs showing the effect of daratumumab (Dara) on normal immune cells (PBMCs) collected from a healthy donor 96 hours after culture in either media alone or media supplemented with 10% complement. Daratumumab was used at doses of 0.01, 0.1, or 1 μg/mL. Some cells were treated with control isotype mAb (Hu IgG1k). FIG. 6A shows the frequency of NK cells after these treatments. FIG. 6B shows the number of NK cells after these treatments. FIG. 6C shows the frequency of T cells after these treatments. FIG. 6D shows the number of T cells after these treatments.

FIGS. 7A-7B are graphs showing the frequency and number of anti-BCMA CAR T cells after 72 hours culture with daratumumab (Dara) or control isotype mAb (Hu IgG1k), with or without 10% complement. Daratumumab was used at doses of 0.01, 0.1, or 1 μg/mL. FIG. 7A shows the frequency of anti-BCMA CAR T cells after these treatments. FIG. 7B shows the number of anti-BCMA CAR T cells after these treatments.

FIG. 8A-8N are flow cytometry plots measuring the early activation marker CD69 expression in anti-CD19 CAR T cells after a 24 hour co-culture with daratumumab at 0.01, 0.1 or 1 μg/mL and with addition of 2 μg/mL goat anti-human antibody. Expression of CD69 markers after treatment with control isotype mAb (IgG1k) and with addition of 2 μg/mL goat anti-human antibody were also measured. FIG. 8A shows CD69 expression in anti-CD19 CAR T cells after a 24 hour co-culture with no treatment. FIG. 8B shows CD69 expression in anti-CD19 CAR T cells after a 24 hour co-culture with daratumumab at 0.01 μg/mL. FIG. 8C shows CD69 expression in anti-CD19 CAR T cells after a 24 hour co-culture with daratumumab at 0.1 μg/mL.

FIG. 8D shows CD69 expression in anti-CD19 CAR T cells after a 24 hour co-culture with daratumumab at 1 μg/mL. FIG. 8E shows CD69 expression in anti-CD19 CAR T cells after a 24 hour co-culture with 2 μg/mL goat anti-human antibody. FIG. 8F shows CD69 expression in anti-CD19 CAR T cells after a 24 hour co-culture with daratumumab at 0.01 μg/mL and 2 μg/mL goat anti-human antibody. FIG. 8G shows CD69 expression in anti-CD19 CAR T cells after a 24 hour co-culture with daratumumab at 0.1 μg/mL and 2 μg/mL goat anti-human antibody. FIG. 8H shows CD69 expression in anti-CD19 CAR T cells after a 24 hour co-culture with daratumumab at 1 μg/mL and 2 μg/mL goat anti-human antibody. FIG. 8I shows CD69 expression in anti-CD19 CAR T cells after a 24 hour co-culture with IgG1k at 0.01 μg/mL. FIG. 8J shows CD69 expression in anti-CD19 CAR T cells after a 24 hour co-culture with IgG1k at 0.1 μg/mL. FIG. 8K shows CD69 expression in anti-CD19 CAR T cells after a 24 hour co-culture with IgG1k at 1 μg/mL. FIG. 8L shows CD69 expression in anti-CD19 CAR T cells after a 24 hour co-culture with IgG1k at 0.01 μg/mL and 2 μg/mL goat anti-human antibody. FIG. 8M shows CD69 expression in anti-CD19 CAR T cells after a 24 hour co-culture with IgG1k at 0.1 μg/mL and 2 μg/mL goat anti-human antibody. FIG. 8N shows CD69 expression in anti-CD19 CAR T cells after a 24 hour co-culture with IgG1k at 1 μg/mL and 2 μg/mL goat anti-human antibody.

FIGS. 9A-9B provide diagrams showing CAR T cell lysis in the presence of NK cells. FIG. 9A shows the frequency anti-BCMA CAR T cell lysis in a co-culture of anti-BCMA CAR T cells and purified NK cells from a normal donor that were pre-treated with daratumumab or isotype control mAb at 0.01, 0.1 or 1 μg/mL. Error bars represent standard error of mean (SEM) where n=3. FIG. 9B is a flow cytometry plot showing the levels of TCRa/b and β2M expression in the anti-BCMA CAR T cells prior to co-culture with NK cells as described in FIG. 9A.

FIGS. 10A-10C provide diagrams NK-cell mediated CAR T cell lysis in the presence or absence of daratumumab. FIG. 10A shows anti-BCMA CAR T cell frequency of CAR T cells after a 24 hour co-culture with daratumumab at 0.1, 1, or 10 μg/mL. Error bars represent standard error of mean (SEM) where n=3. FIG. 10B shows the protection from NK mediated cell lysis in co-cultures of anti-BCMA CAR T cells deficient in B2M and daratumumab-treated NK cells at a 1:1 ratio. The NK cells were from a normal donor and were pre-treated for 60 hours with daratumumab or isotype control mAb at 0.1, 1, or 10 μg/mL. FIG. 10C shows the protection from NK mediated cell lysis in co-cultures with a 3:1 ratio of daratumumab-treated NK cells to anti-BCMA CAR T cells. Error bars represent standard error of mean (SEM) where n=3.

FIGS. 11A-11B are graphs showing NK and CAR T cells numbers after CAR T cells anti-BCMA CAR T cells deficient in B2M were co-cultured with purified NK cells that were pre-treated for 60 hours with either daratumumab at concentrations of 0.01, 0.1, 1, 10, 100 or 300 μg/mL. Error bars represent standard error of mean (SEM) where n=3. FIG. 11A shows NK cell numbers after co-culturing for 72 hours. FIG. 11B shows T cell numbers after co-culturing for 72 hours.

FIG. 12 is a graph depicting NK cell numbers as measured by flow cytometry of blood collected from mice that were injected with 100 μg per mouse of daratumumab and/or 0.4×10⁶ NK cells per mouse. Error bars represent standard error of mean (SEM) where n=3.

FIG. 13 is a graph depicting weekly bioluminescence measurements of immune-deficient mice intravenously injected with 0.5×10⁶ Nalm6 cells/mouse, the measurements collected after another intravenous injection of the mice with NK cells, PBS, daratumumab (DARA), IgG1, and/or anti-CD19 CAR T cells (4×10⁶ cells/mouse). Error bars represent standard error of mean (SEM) where n=3.

FIG. 14A-14C are graphs showing tumor volume and survival of immune-deficient mice intravenously injected with 5×10⁶ MM.1S cells/mouse, and treated with daratumumab, anti-BCMA CAR-T cells, or a combination thereof. FIG. 14A are graphs showing tumor volume (top) and survival (bottom) of mice treated with a low dose of anti-BCMA CAR-T cells (0.8×10⁶ CAR⁺ T cells) alone or in combination with daratumumab (15 mg/kg). FIG. 14B are graphs showing tumor volume (top) and survival (bottom) of mice treated with a high dose of anti-BCMA CAR-T cells (2.4×10⁶ CAR⁺ T cells) alone or in combination with daratumumab (15 mg/kg). FIG. 14C is a graph showing tumor volume at day 26 of mice treated with a high dose of anti-BCMA CAR-T cells alone or in combination with daratumumab.

### DETAILED DESCRIPTION OF THE INVENTION

Without wishing to be bound by theory, it is believed that CAR T cells with disrupted MHC Class I are not able to provide the required MHC Class I-NK KIR receptor binding that prevents NK-cells from eliminating MHC-Class I sufficient cells, i.e., self-cells. Thus, allogeneic CAR T cells with disrupted MHC Class I are susceptible to elimination by NK-mediated immune surveillance. It was discovered that the administration of an NK cell inhibitor, using an anti-CD38 monoclonal antibody as an example, resulted in a reduction of NK cell numbers. The depletion of NK cells, in turn, protects the allogeneic CAR T cell from host NK-mediated cell lysis. The combination of CAR T cell therapy and NK cell inhibitors thus presents an improvement over the existing CAR T cell therapy.

It was demonstrated that T cells isolated from PBMCs also express CD38 protein on the cell surface. Surprisingly, the addition of an anti-CD38 monoclonal antibody at doses that depleted NK cells did not affect T cell numbers, even after multi-day culture with an anti-CD38 monoclonal antibody. Nor does the addition of anti-CD38 monoclonal antibody at doses that depleted NK cell numbers induce CAR T cell activation. Accordingly, without wishing to be bound by theory, it is believed that anti-CD38 monoclonal antibody treatment is NK cell-specific, and induces reduction of NK cells without causing undesirable non-specific CAR T cell activation or elimination. The addition of an NK cell inhibitor, such as an anti-CD38 monoclonal antibody, represents an improvement to existing CAR T cell therapy.

It was further demonstrated that the effect of the anti-CD38 antibody on NK cells was not complement-dependent, as the addition of complement to co-culture of anti-CD38 antibody and PBMC did not affect the magnitude of NK cell depletion. More importantly, the addition of complement did not result in the depletion of T cells or affected CAR T cell activation status. Accordingly, without wishing to be bound by theory, it is believed that administration of an NK cell inhibitor, such as an anti-CD38 antibody, in combination with a CAR T cell therapy improves CAR T cell persistence and efficacy. Moreover, it was observed in an animal model that an anti-CD38 antibody successfully enhanced the anti-tumor effect of CAR-T cells targeting a tumor antigen (e.g., CD19 or BCMA). Without wishing to be bound by theory, it is believed that the combination therapy improves clinical response in the subject, for example, by increasing anti-tumor activity of the CAR T cell therapy.

The present disclosure provides methods and/or compositions of engineered T cells comprising disrupted major histocompatibility complex (MHC) and expressing a chimeric antigen receptor (CAR) for use in combination with an NK cell inhibitor for treating cancer. In some embodiments, these engineered T cells lack a functional T cell receptor (TCR) and comprise a disrupted MHC Class I to reduce the risk of rejection by a subject.

Accordingly, one aspect of the present disclosure features a combined therapy to improve a clinical outcome, wherein the combined therapy involves genetically engineered T cells expressing a chimeric antigen receptor (“CAR” and “CAR T cells”) and an NK cell inhibitor (e.g., an anti-CD38 antibody such as daratumumab or a functional variant thereof).

### I. Genetically Engineered Chimeric Antigen Receptor (CAR) T Cells

In some embodiments, the present disclosure provides engineered human T cells expressing a chimeric antigen receptor (CAR) (i.e., engineered human CAR T cells). Methods of making CAR T cells are known to those of ordinary skill in the art and are described in greater detail infra. CAR T cell therapy refers to compositions and/or methods comprising a population of engineered human CAR T cells (i.e., engineered human CAR T cells) for use in treating a target disease such as a cancer in a subject in need of the treatment. In some embodiments, the CAR T cell therapy may further comprise a pre-conditioning regimen, which may comprise a lymphodepletion regimen. An exemplary lymphodepletion regimen may comprise administering chemotherapy drugs as part of the lymphodepletion regimen. In some embodiments, the CAR T cell therapy may include one dose of the engineered human CAR T cells. In other embodiments, the CAR T cell therapy disclosed herein may comprise multiple doses (for example, 2 doses or 3 doses) of the engineered human CAR T cells.

The genetically engineered CAR T cells (e.g., human CAR-T cells disclosed herein) express a chimeric antigen receptor having specificity to an antigen of interest (e.g., a cancer or tumor antigen). In some embodiments, the genetically engineered CAR T cells may comprise further genetic edits in one or more target genes, for example, a MHC Class I gene (e.g., B2M) and/or a T cell receptor gene (e.g., TRAC).

A. Chimeric Antigen Receptor (CAR)

A chimeric antigen receptor refers to an artificial immune cell receptor that is engineered to recognize and bind to an antigen expressed by tumor cells. Generally, a CAR is designed for a T cell and is a chimera of a signaling domain of the T-cell receptor (TCR) complex and an antigen-recognizing domain (e.g., an antibody single chain variable fragment (scFv) or other antigen binding fragment) (Enblad et al., Human Gene Therapy. 2015; 26(8):498-505). A T cell that expresses a CAR is referred to as a CAR T cell. CARs have the ability to redirect T-cell specificity and reactivity toward a selected target in a non-MHC-restricted manner. The non-MHC-restricted antigen recognition gives T-cells expressing CARs the ability to recognize an antigen independent of antigen processing, thus bypassing a major mechanism of tumor escape. Moreover, when expressed in T-cells, CARs advantageously do not dimerize with endogenous T-cell receptor (TCR) alpha and beta chains.

There are four generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional domain, e.g., CD28, 4-1BB (41BB), or ICOS, to supply a costimulatory signal. Third-generation CARs contain two costimulatory domains fused with the TCR CD3ζ chain. Third-generation costimulatory domains may include, e.g., a combination of CD3ζ, CD27, CD28, 4-1BB, ICOS, or OX40. CARs, in some embodiments, contain an ectodomain (e.g., CD3ζ), commonly derived from a single chain variable fragment (scFv), a hinge, a transmembrane domain, and an endodomain with one (first generation), two (second generation), or three (third generation) signaling domains derived from CD3z and/or co-stimulatory molecules (Maude et al., Blood. 2015; 125(26):4017-4023; Kakarla and Gottschalk, Cancer J. 2014; 20(2):151-155).

CARs typically differ in their functional properties. The CD3ζ signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs improved replicative capacity and persistence of modified T-cells. Similar antitumor effects are observed in vitro with CD28 or 4-1BB CARs. Clinical trials suggest that both of these second-generation CARs are capable of inducing substantial T-cell proliferation in vivo. Third generation CARs combine multiple signaling domains (costimulatory) to augment potency.

In some embodiments, a chimeric antigen receptor is a first generation CAR. In other embodiments, a chimeric antigen receptor is a second generation CAR. In yet other embodiments, a chimeric antigen receptor is a third generation CAR.

A CAR, in some embodiments, comprises an extracellular (ecto) domain comprising an antigen binding domain (e.g., an antibody, such as an scFv), a transmembrane domain, and a cytoplasmic (endo) domain.

(i) Ectodomain

The ectodomain is the region of the CAR that is exposed to the extracellular fluid and, in some embodiments, includes an antigen binding domain, and optionally a signal peptide, a spacer domain, and/or a hinge domain. In some embodiments, the antigen binding domain is a single-chain variable fragment (scFv) that includes the VL and VH of immunoglobins connected with a short linker peptide. The linker, in some embodiments, includes hydrophilic residues with stretches of glycine and serine for flexibility as well as stretches of glutamate and lysine for added solubility. A single-chain variable fragment (scFv) is a fusion protein of the variable regions of the heavy (VH) and light chains (VL) of immunoglobulins, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker.

In some embodiments, the scFv of the present disclosure is humanized. In other embodiments, the scFv is fully human. In yet other embodiments, the scFv is a chimera (e.g., of mouse and human sequence).

In some examples, the ectodomain of the CAR disclosed herein is an anti-CD19 scFv, e.g., an anti-CD19 scFv including the amino acid sequence of SEQ ID NO: 54 (VH) and SEQ ID NO: 55 (VL). In one example, the anti-CD19 scFv may comprise the amino acid sequence of SEQ ID NO:50.

In some examples, the ectodomain of the CAR disclosed herein is an anti-CD70 scFv, e.g., an anti-CD70 scFv including the amino acid sequence of SEQ ID NO: 81 (VH) and SEQ ID NO: 82 (VL). In one example, the anti-CD70 scFv may comprise the amino acid sequence of SEQ ID NO: 78 or SEQ ID NO: 80.

In other examples, the ectodomain of the CAR disclosed herein is an anti-BCMA scFv, e.g., an anti-BCMA scFv including the amino acid sequence of SEQ ID NO: 89 (VH) and SEQ ID NO: 90 (VL). In one example, the anti-BCMA scFv may comprise the amino acid sequence of SEQ ID NO: 88.

In yet other examples, the ectodomain of the CAR disclosed herein can be an anti-CD33 scFv, e.g., an anti-CD33 scFv including the amino acid sequence of SEQ ID NO: 116 (VH) and SEQ ID NO: 117 (VL). In one example, the anti-CD33 scFv may comprise the amino acid sequence of SEQ ID NO: 113.

Further information regarding scFv sequences for use in constructing a CAR as disclosed herein, e.g., a CAR targeting CD19, BCMA, CD70, or CD33, may be found in WO2019/097305, WO2019/215500, and WO2020/095107, the relevant disclosures of each of which are incorporated by reference for the purpose and subject matter referenced herein.

The signal peptide can enhance the antigen specificity of CAR binding. Signal peptides can be derived from antibodies, such as, but not limited to, CD8, as well as epitope tags such as, but not limited to, GST or FLAG. Examples of signal peptides include MLLLVTSLLLCELPHPAFLLIP (SEQ ID NO: 30) and MALPVTALLLPLALLLHAARP (SEQ ID NO: 31). Other signal peptides may be used.

In some embodiments, a spacer domain or hinge domain is located between an extracellular domain (comprising the antigen binding domain) and a transmembrane domain of a CAR, or between a cytoplasmic domain and a transmembrane domain of the CAR. A spacer domain is any oligopeptide or polypeptide that functions to link the transmembrane domain to the extracellular domain and/or the cytoplasmic domain in the polypeptide chain. A hinge domain is any oligopeptide or polypeptide that functions to provide flexibility to the CAR, or domains thereof, or to prevent steric hindrance of the CAR, or domains thereof. In some embodiments, a spacer domain or a hinge domain may comprise up to 300 amino acids (e.g., 10 to 100 amino acids, or 5 to 20 amino acids). In some embodiments, one or more spacer domain(s) may be included in other regions of a CAR. In some embodiments, the hinge domain is a CD8 hinge domain. Other hinge domains may be used.

In some embodiments, the ectodomain of any of the CAR constructs disclosed herein targets (e.g., specifically binds to) a cancer or tumor antigen. The terms “cancer antigen” and “tumor antigen” are used interchangeably herein. In some embodiments, a tumor or cancer antigen is a “tumor associated antigen,” referring an immunogenic molecule, such as a protein, that is generally expressed at a higher level in tumor cells than in non-tumor cells, in which it may not be expressed at all, or only at low levels. In some embodiments, tumor-associated structures which are recognized by the immune system of the tumor-harboring host are referred to as tumor-associated antigens. In some embodiments, a tumor-associated antigen is a universal tumor antigen that is broadly expressed by most tumors. In some embodiments, tumor-associated antigens are differentiation antigens, mutational antigens, overexpressed cellular antigens or viral antigens. In some embodiments, a tumor or cancer antigen is a “tumor specific antigen” or “TSA,” referring to an immunogenic molecule, such as a protein, that is unique to a tumor cell. Tumor specific antigens are exclusively expressed in tumor cells. Exemplary cancer antigens are provided below.

CD19

In some embodiments, the T cells of the present disclosure are engineered with a chimeric antigen receptor (CAR) designed to target CD19. Cluster of Differentiation 19 (CD19) is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM-001178098. CD19 is expressed on most B lineage cancers, including, e.g., acute lymphoblastic leukemia, chronic lymphocyte leukemia and non-Hodgkin's lymphoma. It is also an early marker of B cell progenitors. See, e.g., Nicholson et al. Mol. Immun. 34 (16-17): 1157-1165 (1997).

Thus, in some embodiments, T cells of the present disclosure are engineered to express a CAR comprising an anti-CD19 antibody (e.g., anti-CD19 scFv). In some embodiments, the anti-CD19 antibody is an anti-CD19 scFv encoded by the nucleotide sequence set forth in SEQ ID NO: 49. In some embodiments, the anti-CD19 antibody is an anti-CD19 scFv comprising the amino acid sequence set forth in SEQ ID NO: 50. In some embodiments, the anti-CD19 antibody is an anti-CD19 scFv comprising a variable heavy chain (VH) comprising the amino acid sequence set forth in SEQ ID NO: 54. In some embodiments, the anti-CD19 antibody is an anti-CD19 scFv comprising a variable light chain (VL) comprising the amino acid sequence set forth in SEQ ID NO: 55. In some embodiments, a CAR comprising an anti-CD19 antibody is encoded by the nucleotide sequence set forth in SEQ ID NO: 39. In some embodiments, a CAR comprising an anti-CD19 antibody comprises the amino acid sequence set forth in SEQ ID NO: 40.

In some embodiments, the anti-CD19 antibody is an anti-CD19 scFv encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 49 and optionally encoding the amino acid sequence of SEQ ID NO:50. In some embodiments, the anti-CD19 antibody is an anti-CD19 scFv comprising an amino acid sequence having at least 80%, 81%, 82%, 83%, 84%, 85%, 86%, 87%, 88%, 89%, 90%, 91%, 92%, 93%, 94%, 95%, 96%, 97% 98% or 99% identity to SEQ ID NO: 50. In some embodiments, the anti-CD19 antibody is an anti-CD19 scFv comprising a VH comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 54. In some embodiments, the anti-CD19 antibody is an anti-CD19 scFv comprising a VL comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 55. In some embodiments, a CAR comprising an anti-CD19 antibody is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 39 and optionally encoding the amino acid sequence of SEQ ID NO:40. In some embodiments, a CAR comprising an anti-CD19 antibody comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 40.

BCMA

In some embodiments, the T cells of the present disclosure are engineered with a CAR designed to target BCMA. B-cell maturation antigen (BCMA, CD269) is a member of the tumor necrosis factor receptor (TNF) superfamily. BCMA binds B-cell activating factor (BAFF) and a proliferation inducing ligand (APRIL). Among nonmalignant cells, BCMA is expressed primarily by plasma cells and subsets of mature B cells. BCMA is selectively expressed by B-lineage cells including multiple myeloma cells and non-Hodgkin's lymphoma, thus BCMA is also an attractive therapeutic target.

Thus, in some embodiments, T cells of the present disclosure are engineered to express a CAR comprising an anti-BCMA antibody (e.g., anti-BCMA scFv). In some embodiments, the anti-BCMA antibody is an anti-BCMA scFv encoded by the nucleotide sequence of SEQ ID NO: 87. In some embodiments, the anti-BCMA antibody is an anti-BCMA scFv comprising the amino acid sequence of SEQ ID NO: 88. In some embodiments, the anti-BCMA antibody is an anti-BCMA scFv comprising a VH comprising the amino acid sequence of SEQ ID NO: 89. Alternatively or in addition, the anti-BCMA antibody is an anti-BCMA scFv comprising a VL comprising the amino acid sequence of SEQ ID NO: 90. In some embodiments, a CAR comprising an anti-BCMA antibody is encoded by the sequence of SEQ ID NO: 85. In some embodiments, a CAR comprising an anti-BCMA antibody comprises the sequence of SEQ ID NO: 86.

In some embodiments, the anti-BCMA antibody is an anti-BCMA scFv encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 87 and optionally encoding the amino acid sequence of SEQ ID NO:88. In some embodiments, the anti-BCMA antibody is an anti-BCMA scFv comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 88. In some embodiments, the anti-BCMA antibody is an anti-BCMA scFv comprising a VH comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 89. Alternatively or in addition, the anti-BCMA antibody is an anti-BCMA scFv comprising a VL comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 90. In some embodiments, a CAR comprising an anti-BCMA antibody is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 85 and optionally encoding the amino acid sequence of SEQ ID NO:86. In some embodiments, a CAR comprising an anti-BCMA antibody comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 86.

CD33

In some embodiments, the T cells of the present disclosure are engineered with a CAR designed to target CD33. CD33, also known as Siglec3, is a transmembrane receptor expressed on cells of myeloid lineage that is known to bind sialic acids. As CD33 is expressed in cancer cells (e.g., acute myeloid leukemia), it is thought that CD33 represents a cell surface marker for targeting these malignancies.

Thus, in some embodiments, T cells of the present disclosure are engineered to express a CAR comprising an anti-CD33 antibody (e.g., anti-CD33 scFv). In some embodiments, the anti-CD33 antibody is an anti-CD33 scFv encoded by the nucleotide sequence of SEQ ID NO: 114. In some embodiments, the anti-CD33 antibody is an anti-CD33 scFv comprising the amino acid sequence of SEQ ID NO: 113. In some embodiments, the anti-CD33 antibody is an anti-CD33 scFv comprising a VH comprising the amino acid sequence of SEQ ID NO: 116. Alternatively or in addition, the anti-CD33 antibody is an anti-CD33 scFv comprising a VL comprising the amino acid sequence of SEQ ID NO: 117. In some embodiments, a CAR comprising an anti-CD33 antibody is encoded by the nucleotide sequence of SEQ ID NO: 112. In some embodiments, a CAR comprising an anti-CD33 antibody comprises the amino acid sequence of SEQ ID NO: 115.

In some embodiments, the anti-CD33 antibody is an anti-CD33 scFv encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 114 and optionally encoding the amino acid sequence of SEQ ID NO:113. In some embodiments, the anti-CD33 antibody is an anti-CD33 scFv comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 113. In some embodiments, the anti-CD33 antibody is an anti-CD19 scFv comprising a VH comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 116. Alternatively or in addition, the anti-CD33 antibody is an anti-CD33 scFv comprising a VL comprising an amino acid sequence having at least 80%, 81%, 82%, 83%, 84%, 85%, 86%, 87%, 88%, 89%, 90%, 91%, 92%, 93%, 94%, 95%, 96%, 97% 98% or 99% identity to SEQ ID NO: 117. In some embodiments, a CAR comprising an anti-CD33 antibody is encoded by a nucleotide sequence having at least 80%, 81%, 82%, 83%, 84%, 85%, 86%, 87%, 88%, 89%, 90%, 91%, 92%, 93%, 94%, 95%, 96%, 97% 98% or 99% identity to SEQ ID NO: 112 and optionally encoding the amino acid sequence of SEQ ID NO:115. In some embodiments, a CAR comprising an anti-CD33 antibody comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 115.

CD70

In some embodiments, the T cells of the present disclosure are engineered with a chimeric antigen receptor (CAR) designed to target CD70. CD70 was initially identified as the ligand for CD27, a co-stimulatory receptor involved in T cell proliferation and survival. CD70 is only found on a small percentage of activated T cells and antigen presenting cells in draining lymph nodes during viral infection. Many human tumors also express CD70 including, but not limited to, solid cancers such as clear cell renal cancer, breast cancer, gastric cancer, ovarian cancer, glioblastoma, and hematological malignancies. Due to its restricted expression pattern on normal tissues and overexpression in numerous cancers, CD70 is an attractive therapeutic target.

Thus, in some embodiments, T cells of the present disclosure are engineered to express a CAR comprising an anti-CD70 antibody (e.g., anti-CD70 scFv). In some embodiments, the anti-CD70 antibody is an anti-CD70 scFv encoded by the nucleotide sequence of SEQ ID NO: 77 or 79. In some embodiments, the anti-CD70 antibody is an anti-CD70 scFv comprising the amino acid sequence of SEQ ID NO: 78 or 80. In some embodiments, the anti-CD70 antibody is an anti-CD70 scFv comprising a VH comprising the amino acid sequence of SEQ ID NO: 81. Alternatively or in addition, the anti-CD70 antibody is an anti-CD70 scFv comprising a VL comprising the amino acid sequence of SEQ ID NO: 82. In some embodiments, a CAR comprising an anti-CD70 antibody is encoded by the nucleotide sequence of SEQ ID NO: 75. In some embodiments, a CAR comprising an anti-CD70 antibody comprises the amino acid sequence of SEQ ID NO: 76.

In some embodiments, the anti-CD70 antibody is an anti-CD70 scFv encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 77 or 79, and optionally encoding the amino acid sequences of SEQ ID NOs:78 and 80, respectively. In some embodiments, the anti-CD70 antibody is an anti-CD70 scFv comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 78 or 80. In some embodiments, the anti-CD70 antibody is an anti-CD70 scFv comprising a VH comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 81. Alternatively or in addition, the anti-CD70 antibody is an anti-CD70 scFv comprising a VL comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 82. In some embodiments, a CAR comprising an anti-CD70 antibody is encoded by a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 75 and optionally encoding the amino acid sequence of SEQ ID NO:76. In some embodiments, a CAR comprising an anti-CD70 antibody comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO: 76.

(ii) Transmembrane Domain

The transmembrane domain is a hydrophobic alpha helix that spans the membrane. The transmembrane domain provides stability of the CAR. In some embodiments, the transmembrane domain of a CAR as provided herein is a CD8 transmembrane domain. In other embodiments, the transmembrane domain is a CD28 transmembrane domain. In yet other embodiments, the transmembrane domain is a chimera of a CD8 and CD28 transmembrane domain. Other transmembrane domains may be used. In some embodiments, the transmembrane domain is a CD8a transmembrane domain comprising the amino acid sequence set forth in SEQ ID NO: 32.

(iii) Endodomain

The endodomain is the functional end of the receptor. Following antigen recognition, receptors cluster and a signal is transmitted to the cell. The most commonly used endodomain component is CD3-zeta, which contains three (3) immunoreceptor tyrosine-based activation motif (ITAM)s. This transmits an activation signal to the T cell after the antigen is bound. An exemplary CD3-zeta comprises the amino acid sequence of SEQ ID NO: 38, which may be encoded by the nucleotide sequence of SEQ ID NO: 37. In many cases, CD3-zeta may not provide a fully competent activation signal and, thus, a co-stimulatory signaling is used. For example, CD28 and/or 4-1BB may be used with CD3-zeta (CD3ζ) to transmit a proliferative/survival signal. Thus, in some embodiments, the co-stimulatory domain of a CAR as provided herein is from a CD28 co-stimulatory molecule (e.g., a co-stimulatory domain comprising the amino acid sequence of SEQ ID NO: 36, which may be encoded by the nucleotide sequence of SEQ ID NO: 35). In other embodiments, the co-stimulatory domain can be from a 4-1BB co-stimulatory molecule (e.g., a co-stimulatory domain comprising the amino acid sequence of SEQ ID NO: 34, which may be encoded in the nucleotide sequence of SEQ ID NO: 33). In some embodiments, a CAR includes CD3ζ and a CD28 co-stimulatory domain. In other embodiments, a CAR includes CD3-zeta and a 4-1BB co-stimulatory domain. In still other embodiments, a CAR includes CD3ζ, a CD28 co-stimulatory domain, and a 4-1BB co-stimulatory domain.

(iv) Exemplary CAR Constructs

Provided below are a number of exemplary CAR constructs that can be expressed in the genetically engineered CAR-T cells for use in the combined therapy disclosed herein. Further information regarding CAR constructs for use in the present disclosure, e.g., a CAR targeting CD19, BCMA, CD70, or CD33, may be found in WO2019/097305, WO2019/215500, and WO2020/095107, the relevant disclosures of each of which are incorporated by reference for the purpose and subject matter referenced herein.

Anti-CD19 CAR

In some embodiments, the engineered T cells described herein comprise a CD19 targeting chimeric antigen receptor (CAR), also referred to herein as an anti-CD19 CAR or anti-CD19 CAR T cells. In some embodiments, the anti-CD19 CAR comprises (i) an ectodomain that comprises an anti-CD19 antigen-binding fragment, (ii) a transmembrane domain, and (iii) an endodomain comprising at least one co-stimulatory domain.

In some embodiments, the anti-CD19 CAR comprises (i) an ectodomain that comprises an anti-CD19 antigen-binding fragment, (ii) a CD8 transmembrane domain, and (iii) an endodomain that comprises a CD28 or 41BB co-stimulatory domain, and a CD3-zeta co-stimulatory domain. In some embodiments, the anti-CD19 CAR comprises (i) an ectodomain that comprises an anti-CD19 antigen-binding fragment, (ii) a CD8 transmembrane domain, and (iii) an endodomain that comprises a CD28 co-stimulatory domain and a CD3-zeta co-stimulatory domain. In some embodiments, the anti-CD19 CAR comprises (i) an ectodomain that comprises an anti-CD19 antigen-binding fragment, (ii) a CD8 transmembrane domain, and (iii) an endodomain that comprises a 41BB co-stimulatory domain and a CD3-zeta co-stimulatory domain.

In some embodiments, the anti-CD19 CAR comprises an anti-CD19 antibody (e.g., anti-CD19 scFv) that comprises a VH and a VL comprising the amino acid sequences set forth in SEQ ID NOs: 54 and 55, respectively. In some embodiments, the anti-CD19 antibody (e.g., anti-CD19 scFv) comprises three CDRs (CDR1, CDR2 and CDR2) of the VH set forth in SEQ ID NO:54, and three CDRs (CDR1, CDR2 and CDR3) of the VL set forth in SEQ ID NO: 55. In some embodiments, the anti-CD19 antibody (e.g., anti-CD19 scFv) comprises three CDRs (CDR1, CDR2 and CDR2) of the VH set forth in SEQ ID NO: 54, and three CDRs (CDR1, CDR2 and CDR3) of the VL set forth in SEQ ID NO: 55, wherein the CDRs are determined according to Kabat. In some embodiments, the anti-CD19 antibody (e.g., anti-CD19 scFv) comprises three CDRs (CDR1, CDR2 and CDR2) of the VH set forth in SEQ ID NO: 54, and three CDRs (CDR1, CDR2 and CDR3) of the VL set forth in SEQ ID NO: 55, wherein the CDRs are determined according to Chothia. In some embodiments, the anti-CD19 antibody (e.g., anti-CD19 scFv) comprises three CDRs (CDR1, CDR2 and CDR2) of the VH set forth in SEQ ID NO: 54, and three CDRs (CDR1, CDR2 and CDR3) of the VL set forth in SEQ ID NO: 55, wherein the CDRs are determined according to AbM. In some embodiments, the anti-CD19 antibody (e.g., anti-CD19 scFv) comprises heavy chain CDR1, CDR2 and CDR3 sequences set forth in SEQ ID NOs: 127, 128, and 129, respectively, and light chain CDR1, CDR2 and CDR3 sequences set forth in SEQ ID NOs: 124, 125, and 126, respectively. In some embodiments, the anti-CD19 antibody (e.g., anti-CD19 scFv) comprises heavy chain CDR1, CDR2 and CDR3 sequences set forth in SEQ ID NOs: 130, 131, and 129, respectively, and light chain CDR1, CDR2 and CDR3 sequences set forth in SEQ ID NOs: 124, 125, 126, respectively. In some embodiments, the anti-CD19 antibody is an anti-CD19 scFv comprising the amino acid sequence set forth in SEQ ID NO: 50. In some embodiments, the anti-CD19 antibody is an anti-CD19 scFv encoded by the nucleotide sequence set forth in SEQ ID NO: 49.

In some embodiments, the anti-CD19 CAR comprises (i) an ectodomain that comprises an anti-CD19 antigen-binding fragment, (ii) a CD8 transmembrane domain comprising the amino acid sequence set forth in SEQ ID NO: 32, and (iii) an endodomain that comprises a CD28 co-stimulatory domain comprising the amino acid sequence set forth in SEQ ID NO: 36 and a CD3-zeta co-stimulatory domain comprising the amino acid sequence set forth in SEQ ID NO: 38.

In some embodiments, the anti-CD19 CAR comprises (i) an ectodomain that comprises an anti-CD19 antigen-binding fragment comprising the amino acid sequence set forth in SEQ ID NO: 50, (ii) a CD8 transmembrane domain comprising the amino acid sequence set forth in SEQ ID NO: 32, and (iii) an endodomain that comprises a CD28 co-stimulatory domain comprising the amino acid sequence set forth in SEQ ID NO: 36 and a CD3-zeta co-stimulatory domain comprising the amino acid sequence set forth in SEQ ID NO: 38.

In some embodiments, the anti-CD19 CAR comprises (i) an ectodomain that comprises an anti-CD19 antigen-binding fragment comprising variable heavy and light chain regions comprising the amino acid sequences set forth in SEQ ID NOs: 54 and 55, respectively, (ii) a CD8 transmembrane domain comprising the amino acid sequence set forth in SEQ ID NO: 32, and (iii) an endodomain that comprises a CD28 co-stimulatory domain comprising the amino acid sequence set forth in SEQ ID NO: 36 and a CD3-zeta co-stimulatory domain comprising the amino acid sequence set forth in SEQ ID NO: 38. In some embodiments, the anti-CD19 CAR comprises (i) an ectodomain that comprises an anti-CD19 antigen-binding fragment, (ii) a CD8 transmembrane domain comprising the amino acid sequence set forth in SEQ ID NO: 32, and (iii) an endodomain that comprises a CD28 co-stimulatory domain encoded by the nucleotide sequence set forth in SEQ ID NO: 35, and a CD3-zeta co-stimulatory domain encoded by the nucleotide sequence set forth in SEQ ID NO: 37.

In some embodiments, the anti-CD19 CAR comprises (i) an ectodomain that comprises an anti-CD19 antigen-binding fragment encoded by the nucleotide sequence set forth in SEQ ID NO: 49, (ii) a CD8 transmembrane domain comprising the amino acid sequence set forth in SEQ ID NO: 32, and (iii) an endodomain that comprises a CD28 co-stimulatory domain encoded by the nucleotide sequence set forth in SEQ ID NO: 35, and a CD3-zeta co-stimulatory domain encoded by the nucleotide sequence set forth in SEQ ID NO: 37.

In some embodiments, the anti-CD19 CAR comprises (i) an ectodomain that comprises an anti-CD19 antigen-binding fragment comprising variable heavy and light chain regions comprising the amino acid sequences set forth in SEQ ID NOs: 54 and 55, respectively, (ii) a CD8 transmembrane domain comprising the amino acid sequence set forth in SEQ ID NO: 32, and (iii) an endodomain that comprises a CD28 co-stimulatory domain encoded by the nucleotide sequence set forth in SEQ ID NO: 35, and a CD3-zeta co-stimulatory domain encoded by the nucleotide sequence set forth in SEQ ID NO: 37.

In some embodiments, the anti-CD19 CAR comprises the amino acid sequence set forth in SEQ ID NO: 40. In some embodiments, the anti-CD19 CAR is encoded by the nucleotide sequence set forth in SEQ ID NO: 39. In some embodiments, the anti-CD19 CAR is encoded by a nucleotide sequence having at least 80%, 85%, 90%, 95%, 96%, 97%, 98%, 99% or 100% identity to the nucleotide sequence set forth in SEQ ID NO: 39 and optionally encoding the amino acid sequence of SEQ ID NO:40.

Anti-CD33 CAR

In some embodiments, the engineered T cells described herein comprise a CD33 targeting CAR, also referred to herein as CD33 CAR, anti-CD33 CAR or anti-CD33 CAR T cells. In some embodiments, the anti-CD33 CAR comprises (i) an ectodomain that comprises an anti-CD33 antigen-binding domain, (ii) a transmembrane domain, and (iii) an endodomain comprising at least one co-stimulatory domain.

In some embodiments, the anti-CD33 CAR comprises (i) an ectodomain that comprises an anti-CD33 antigen-binding domain, (ii) a CD8 transmembrane domain, and (iii) an endodomain that comprises a CD28 or 41BB co-stimulatory domain, and a CD3-zeta signaling domain. In some embodiments, the anti-CD33 CAR comprises (i) an ectodomain that comprises an anti-CD33 antigen-binding domain, (ii) a CD8 transmembrane domain, and (iii) an endodomain that comprises a CD28 co-stimulatory domain and a CD3-zeta signaling domain. In some embodiments, the anti-CD33 CAR comprises (i) an ectodomain that comprises an anti-CD33 antigen-binding domain, (ii) a CD8 transmembrane domain, and (iii) an endodomain that comprises a 41BB co-stimulatory domain and a CD3-zeta signaling domain.

In some embodiments, the anti-CD33 antibody (e.g., anti-CD33 scFv) in the anti-CD33 CAR disclosed herein comprises a VH and a VL comprising the amino acid sequences set forth in SEQ ID NOs: 116 and 117, respectively. In some embodiments, the anti-CD33 antibody (e.g., anti-CD33 scFv) comprises three CDRs (CDR1, CDR2 and CDR2) of the VH set forth in SEQ ID NO: 116, and three CDRs (CDR1, CDR2 and CDR3) of the VL set forth in SEQ ID NO: 117. In some embodiments, the anti-CD33 antibody (e.g., anti-CD33 scFv) comprises three CDRs (CDR1, CDR2 and CDR2) of the VH set forth in SEQ ID NO:116, and three CDRs (CDR1, CDR2 and CDR3) of the VL set forth in SEQ ID NO: 117, wherein the CDRs are determined according to Kabat. In some embodiments, the anti-CD33 antibody (e.g., anti-CD33 scFv) comprises three CDRs (CDR1, CDR2 and CDR2) of the VH set forth in SEQ ID NO: 116, and three CDRs (CDR1, CDR2 and CDR3) of the VL set forth in SEQ ID NO: 117, wherein the CDRs are determined according to Chothia. In some embodiments, the anti-CD33 antibody (e.g., anti-CD33 scFv) comprises three CDRs (CDR1, CDR2 and CDR2) of the VH set forth in SEQ ID NO: 116, and three CDRs (CDR1, CDR2 and CDR3) of the VL set forth in SEQ ID NO: 117, wherein the CDRs are determined according to AbM. In some embodiments, the anti-CD33 antibody (e.g., anti-CD33 scFv) comprises heavy chain CDR1, CDR2 and CDR3 sequences set forth in SEQ ID NOs: 118, 119, and 120, respectively, and light chain CDR1, CDR2 and CDR3 sequences set forth in SEQ ID NOs: 121, 122, and 123. In some embodiments, the anti-CD33 antibody (e.g., anti-CD33 scFv) comprises heavy chain CDR1 sequence set forth in SEQ ID NO: 132, heavy chain CDR2 sequence set forth in SEQ ID NO: 94, and heavy chain CDR3 sequence set forth in SEQ ID NO: 120, and light chain CDR1, CDR2 and CDR3 sequences set forth in SEQ ID NOs: 121, 122, and 123, respectively, (see Sequence Table). In some embodiments, the anti-CD33 antibody is an anti-CD33 scFv comprising the amino acid sequence set forth in SEQ ID NO: 113. In some embodiments, the anti-CD33 antibody is an anti-CD33 scFv encoded by the nucleotide sequence set forth in SEQ ID NO: 114.

In some embodiments, the anti-CD33 CAR comprises (i) an ectodomain that comprises an anti-CD33 antigen-binding domain, (ii) a CD8 transmembrane domain comprising the amino acid sequence set forth in SEQ ID NO: 32, and (iii) an endodomain that comprises a 41BB co-stimulatory domain comprising the amino acid sequence set forth in SEQ ID NO: 34 and a CD3-zeta signaling domain comprising the amino acid sequence set forth in SEQ ID NO: 38.

In some embodiments, the anti-CD33 CAR comprises (i) an ectodomain that comprises an anti-CD33 scFv comprising the amino acid sequence set forth in SEQ ID NO:113, (ii) a CD8 transmembrane domain comprising the amino acid sequence set forth in SEQ ID NO: 32, and (iii) an endodomain that comprises a 41BB co-stimulatory domain comprising the amino acid sequence set forth in SEQ ID NO: 34 and a CD3-zeta signaling domain comprising the amino acid sequence set forth in SEQ ID NO:38.

In some embodiments, the anti-CD33 CAR comprises (i) an ectodomain that comprises an anti-CD33 scFv comprising variable heavy and light chain regions comprising the amino acid sequences set forth in SEQ ID NOs: 116 and 117, respectively, (ii) a CD8 transmembrane domain comprising the amino acid sequence set forth in SEQ ID NO: 32, and (iii) an endodomain that comprises a 41BB co-stimulatory domain comprising the amino acid sequence set forth in SEQ ID NO: 34 and a CD3-zeta signaling domain comprising the amino acid sequence set forth in SEQ ID NO: 38.

In some embodiments, the anti-CD33 CAR comprises the amino acid sequence set forth in SEQ ID NO: 115. In some embodiments, the anti-CD33 CAR is encoded by the nucleotide sequence set forth in SEQ ID NO: 112. In some embodiments, the anti-CD33 CAR is encoded by a nucleotide sequence having at least 80%, 85%, 90%, 95%, 96%, 97%, 98%, 99% or 100% identity to the nucleotide sequence set forth in SEQ ID NO: 112 and optionally encoding the amino acid sequence of SEQ ID NO:115.

Anti-BCMA CAR

In some embodiments, the engineered T cells described herein comprise a BCMA targeting CAR, also referred to herein as BCMA CAR, anti-BCMA CAR or anti-BCMA CAR T cells. In some embodiments, the anti-BCMA CAR comprises (i) an ectodomain that comprises an anti-BCMA antigen-binding domain, (ii) a transmembrane domain, and (iii) an endodomain comprising at least one co-stimulatory domain.

In some embodiments, the anti-BCMA CAR comprises (i) an ectodomain that comprises an anti-BCMA antigen-binding domain, (ii) a CD8 transmembrane domain, and (iii) an endodomain that comprises a CD28 or 41BB co-stimulatory domain, and a CD3-zeta signaling domain. In some embodiments, the anti-BCMA CAR comprises (i) an ectodomain that comprises an anti-BCMA antigen-binding domain, (ii) a CD8 transmembrane domain, and (iii) an endodomain that comprises a CD28 co-stimulatory domain and a CD3-zeta signaling domain. In some embodiments, the anti-BCMA CAR comprises (i) an ectodomain that comprises an anti-BCMA antigen-binding domain, (ii) a CD8 transmembrane domain, and (iii) an endodomain that comprises a 41BB co-stimulatory domain and a CD3-zeta signaling domain.

In some embodiments, the anti-BCMA antibody (e.g., anti-BCMA scFv) in the anti-BCMA CAR disclosed herein comprises a VH and a VL comprising the amino acid sequences set forth in SEQ ID NOs: 89 and 90, respectively. In some embodiments, the anti-BCMA antibody (e.g., anti-BCMA scFv) comprises three CDRs (CDR1, CDR2 and CDR2) of the VH set forth in SEQ ID NO: 89, and three CDRs (CDR1, CDR2 and CDR3) of the VL set forth in SEQ ID NO:90. In some embodiments, the anti-BCMA antibody (e.g., anti-BCMA scFv) comprises three CDRs (CDR1, CDR2 and CDR2) of the VH set forth in SEQ ID NO: 89, and three CDRs (CDR1, CDR2 and CDR3) of the VL set forth in SEQ ID NO: 90, wherein the CDRs are determined according to Kabat. In some embodiments, the anti-BCMA antibody (e.g., anti-BCMA scFv) comprises three CDRs (CDR1, CDR2 and CDR2) of the VH set forth in SEQ ID NO: 89, and three CDRs (CDR1, CDR2 and CDR3) of the VL set forth in SEQ ID NO: 90, wherein the CDRs are determined according to Chothia. In some embodiments, the anti-BCMA antibody (e.g., anti-BCMA scFv) comprises three CDRs (CDR1, CDR2 and CDR2) of the VH set forth in SEQ ID NO: 89, and three CDRs (CDR1, CDR2 and CDR3) of the VL set forth in SEQ ID NO: 90, wherein the CDRs are determined according to AbM. In some embodiments, the anti-BCMA antibody (e.g., anti-BCMA scFv) comprises heavy chain CDR1, CDR2 and CDR3 sequences set forth in SEQ ID NOs: 106, 108, and 110, respectively, and light chain CDR1, CDR2 and CDR3 sequences set forth in SEQ ID NOs: 103, 104, and 105. In some embodiments, the anti-BCMA antibody (e.g., anti-BCMA scFv) comprises heavy chain CDR1, CDR2 and CDR3 sequences set forth in SEQ ID NOs: 107, 109, and 110, respectively, and light chain CDR1, CDR2 and CDR3 sequences set forth in SEQ ID NOs: 103, 104, and 105. In some embodiments, the anti-BCMA antibody is an anti-BCMA scFv comprising the amino acid sequence set forth in SEQ ID NO: 88. In some embodiments, the anti-BCMA antibody is an anti-BCMA scFv encoded by the nucleotide sequence set forth in SEQ ID NO: 87.

In some embodiments, the anti-BCMA CAR comprises (i) an ectodomain that comprises an anti-BCMA antigen-binding domain, (ii) a CD8 transmembrane domain comprising the amino acid sequence set forth in SEQ ID NO: 32, and (iii) an endodomain that comprises a 41BB co-stimulatory domain comprising the amino acid sequence set forth in SEQ ID NO: 34 and a CD3-zeta signaling domain comprising the amino acid sequence set forth in SEQ ID NO: 38.

In some embodiments, the anti-BCMA CAR comprises (i) an ectodomain that comprises an anti-BCMA scFv comprising the amino acid sequence set forth in SEQ ID NO: 88, (ii) a CD8 transmembrane domain comprising the amino acid sequence set forth in SEQ ID NO: 32, and (iii) an endodomain that comprises a 41BB co-stimulatory domain comprising the amino acid sequence set forth in SEQ ID NO: 34 and a CD3-zeta signaling domain comprising the amino acid sequence set forth in SEQ ID NO:38.

In some embodiments, the anti-BCMA CAR comprises (i) an ectodomain that comprises an anti-BCMA scFv comprising variable heavy and light chain regions comprising the amino acid sequences set forth in SEQ ID NOs: 89 and 90, respectively, (ii) a CD8 transmembrane domain comprising the amino acid sequence set forth in SEQ ID NO: 32, and (iii) an endodomain that comprises a 41BB co-stimulatory domain comprising the amino acid sequence set forth in SEQ ID NO: 34 and a CD3-zeta signaling domain comprising the amino acid sequence set forth in SEQ ID NO: 38.

In some embodiments, the anti-BCMA CAR comprises the amino acid sequence set forth in SEQ ID NO: 86. In some embodiments, the anti-BCMA CAR is encoded by the nucleotide sequence set forth in SEQ ID NO: 85. In some embodiments, the anti-BCMA CAR is encoded by a nucleotide sequence having at least 80%, 85%, 90%, 95%, 96%, 97%, 98%, 99%, or 100% identity to the nucleotide sequence set forth in SEQ ID NO: 85 and optionally encoding the amino acid sequence of SEQ ID NO:86.

Anti-CD70 CAR

In some embodiments, the engineered T cells described herein comprise a CD70 targeting CAR, also referred to herein as CD70 CAR, anti-CD70 CAR or anti-CD70 CAR T cells. In some embodiments, the anti-CD70 CAR comprises (i) an ectodomain that comprises an anti-CD70 antigen-binding domain, (ii) a transmembrane domain, and (iii) an endodomain comprising at least one co-stimulatory domain.

In some embodiments, the anti-CD70 CAR comprises (i) an ectodomain that comprises an anti-CD70 antigen-binding domain, (ii) a CD8 transmembrane domain, and (iii) an endodomain that comprises a CD28 or 41BB co-stimulatory domain, and a CD3-zeta signaling domain. In some embodiments, the anti-CD70 CAR comprises (i) an ectodomain that comprises an anti-CD70 antigen-binding domain, (ii) a CD8 transmembrane domain, and (iii) an endodomain that comprises a CD28 co-stimulatory domain and a CD3-zeta signaling domain. In some embodiments, the anti-CD70 CAR comprises (i) an ectodomain that comprises an anti-CD70 antigen-binding domain, (ii) a CD8 transmembrane domain, and (iii) an endodomain that comprises a 41BB co-stimulatory domain and a CD3-zeta signaling domain.

In some embodiments, the anti-CD70 antibody (e.g., anti-CD70 scFv) in the anti-CD70 CAR disclosed herein comprises a VH and a VL comprising the amino acid sequences set forth in SEQ ID NOs: 81 and 82, respectively. In some embodiments, the anti-CD70 antibody (e.g., anti-CD70 scFv) comprises three CDRs (CDR1, CDR2 and CDR2) of the VH set forth in SEQ ID NO: 81, and three CDRs (CDR1, CDR2 and CDR3) of the VL set forth in SEQ ID NO: 82. In some embodiments, the anti-CD70 antibody (e.g., anti-CD70 scFv) comprises three CDRs (CDR1, CDR2 and CDR2) of the VH set forth in SEQ ID NO: 81, and three CDRs (CDR1, CDR2 and CDR3) of the VL set forth in SEQ ID NO: 82, wherein the CDRs are determined according to Kabat. In some embodiments, the anti-CD70 antibody (e.g., anti-CD70 scFv) comprises three CDRs (CDR1, CDR2 and CDR2) of the VH set forth in SEQ ID NO: 81, and three CDRs (CDR1, CDR2 and CDR3) of the VL set forth in SEQ ID NO: 82, wherein the CDRs are determined according to Chothia. In some embodiments, the anti-CD70 antibody (e.g., anti-CD70 scFv) comprises three CDRs (CDR1, CDR2 and CDR2) of the VH set forth in SEQ ID NO: 81, and three CDRs (CDR1, CDR2 and CDR3) of the VL set forth in SEQ ID NO: 82, wherein the CDRs are determined according to AbM. In some embodiments, the anti-CD70 antibody (e.g., anti-CD70 scFv) comprises heavy chain CDR1, CDR2 and CDR3 sequences set forth in SEQ ID NOs: 97, 99, and 101, respectively, and light chain CDR1, CDR2 and CDR3 sequences set forth in SEQ ID NOs: 91, 93, and 95. In some embodiments, the anti-CD70 antibody (e.g., anti-CD70 scFv) comprises heavy chain CDR1, CDR2 and CDR3 sequences set forth in SEQ ID NOs: 98, 100, and 102, respectively, and light chain CDR1 sequence set forth in SEQ ID NO: 92, light chain CDR2 sequence set forth as LAS, and light chain CDR3 sequence set forth in SEQ ID NO: 96 (see Sequence Table below). In some embodiments, the anti-CD70 antibody is an anti-CD70 scFv comprising the amino acid sequence set forth in SEQ ID NO: 80. In some embodiments, an anti-CD70 antibody is an anti-CD70 scFv encoded by the nucleotide sequence set forth in SEQ ID NO: 79. In some embodiments, the anti-CD70 antibody is an anti-CD70 scFv comprising the amino acid sequence set forth in SEQ ID NO: 78. In some embodiments, the anti-CD70 antibody is an anti-CD70 scFv encoded by the nucleotide sequence set forth in SEQ ID NO: 77.

In some embodiments, the anti-CD70 CAR comprises (i) an ectodomain that comprises an anti-CD70 antigen-binding domain, (ii) a CD8 transmembrane domain comprising the amino acid sequence set forth in SEQ ID NO: 32, and (iii) an endodomain that comprises a 41BB co-stimulatory domain comprising the amino acid sequence set forth in SEQ ID NO: 34 and a CD3-zeta signaling domain comprising the amino acid sequence set forth in SEQ ID NO: 38.

In some embodiments, the anti-CD70 CAR comprises (i) an ectodomain that comprises an anti-CD70 scFv comprising the amino acid sequence set forth in SEQ ID NO: 80, (ii) a CD8 transmembrane domain comprising the amino acid sequence set forth in SEQ ID NO: 32, and (iii) an endodomain that comprises a 41BB co-stimulatory domain comprising the amino acid sequence set forth in SEQ ID NO: 74 and a CD3-zeta signaling domain comprising the amino acid sequence set forth in SEQ ID NO: 38.

In some embodiments, the anti-CD70 CAR comprises (i) an ectodomain that comprises an anti-CD70 scFv comprising variable heavy and light chain regions comprising the amino acid sequences set forth in SEQ ID NOs: 81 and 82, respectively, (ii) a CD8 transmembrane domain comprising the amino acid sequence set forth in SEQ ID NO: 32, and (iii) an endodomain that comprises a 41BB co-stimulatory domain comprising the amino acid sequence set forth in SEQ ID NO: 34 and a CD3-zeta signaling domain comprising the amino acid sequence set forth in SEQ ID NO: 38.

In some embodiments, the anti-CD70 CAR comprises the amino acid sequence set forth in SEQ ID NO: 76. In some embodiments, the anti-CD70 CAR is encoded by the nucleotide sequence set forth in SEQ ID NO: 75. In some embodiments, the anti-CD70 CAR is encoded by a nucleotide sequence having at least 80%, 85%, 90%, 95%, 96%, 97%, 98%, 99%, or 100% identity to the nucleotide sequence set forth in SEQ ID NO: 75 and optionally encoding the amino acid sequence of SEQ ID NO:76.

(v) Expression of Chimeric Antigen Receptor in T Cells

In some embodiments, a nucleic acid encoding a CAR is introduced into an engineered cell by methods known to those of skill in the art. For example, a CAR may be introduced into an engineered cell by a vector. A variety of different methods known in the art can be used to introduce any of the nucleic acids or expression vectors disclosed herein into an immune effector cell. Non-limiting examples of methods for introducing nucleic acid into a cell include: lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes), microinjection, electroporation, cell squeezing, sonoporation, protoplast fusion, impalefection, hydrodynamic delivery, gene gun, magnetofection, viral transfection, and nucleofection.

In some embodiments, a nucleic acid encoding a CAR is introduced into an engineered cell as a donor template as described infra.

B. Gene Editing of T Cells

In some embodiments, the engineered T cells of the present disclosure include at least one gene edit, e.g., a disrupted MHC Class I. For example, an engineered T cell may comprise a disrupted beta-2-microglobulin (β2M) gene. In some embodiments, the engineered T cells of the present disclosure include more than one gene edit, for example, in more than one gene. For example, an engineered T cell may comprise a disrupted T cell receptor alpha chain constant region (TRAC) gene, a disrupted β2M gene, or a combination thereof. In some embodiments, an engineered T cell comprises a disrupted TRAC gene and a disrupted β2M gene.

It should be understood that gene disruption encompasses gene modification through gene editing (e.g., using CRISPR/Cas gene editing to insert or delete one or more nucleotides). In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

Provided herein, in some embodiments, are populations of cells in which a certain percentage of the cells has been edited (e.g., β2M gene edited), resulting in a certain percentage of cells not expressing a particular gene and/or protein. In some embodiments, at least 50% (e.g., 50%, 55%, 60%, 65%, 70%, 75%, 80%, 85%, 90%, or 85%) of the cells of a gene-edited population of cells are β2M knockout cells. In some embodiments, at least 50% of the cells (e.g. T cells) of the population do not express detectable levels of β2M protein. In some embodiments, at least 55%, at least 60%, at least 65%, at least 70%, at least 75%, at least 80%, at least 85%, at least 90%, or at least 95% of the cells of a gene-edited population of cells may be β2M knockout cells.

Methods of using CRISPR-Cas gene editing technology to create a genomic deletion in a cell (e.g., to knock out a gene in a cell) are known (Bauer D E et al. Vis. Exp. 2015; 95; e52118).

(i) Disrupted MHC Class I

In some embodiments, the present disclosure provides engineered human T cells (e.g., engineered human CAR T cells) with disrupted Major Histocompatibility Complex (MHC) for use in the methods described herein. The terms “MCH Class I” and “MHC class I complex” are used interchangeably herein. HLA genes, of which there are more than 40 in humans, encode three classes of MHC proteins (I, II, III) that are expressed on cell surfaces or are components of the complement system. HLA genes are highly diverse, with hundreds of known allelic variations. This genetic diversity results in the expression of highly variable MHC molecules. The MHC molecules play an essential role in immune regulation, as their primary function is to bind peptide antigens for presentation to immune cells. The structurally diverse MHC molecules bind to protein antigens uniquely, and the subsequent antigen presentation by the MHC Class I is a critical step in balancing the immune response to self-antigens (maintain tolerance to avoid autoimmune reaction) versus foreign-antigens (mount immune attack against infected or malignant cells). See e.g., Skelton T S et al., Open J. Immunol. (2011), 1(2):15-26; Petersdorf E W, Blood (2013), 122(11):1862-1872.

MHC class I are present in all nucleated cells and comprise two components (e.g.: MHC Class I subunits): a first a heavy chain comprising amino acid sequences (i.e., cl, a2, and a3) encoded by the HLA-A, B, and C genes, non-covalently associated with a second 02-microglobulin (β2M) peptide. These two components form a unique peptide-binding groove, upon which a self- or foreign-peptide antigen is bound, forming MHC class I. In the context of transplantation, MHC class I from an allogeneic (non-HLA-matched) donor display different, non-self MHC class I, which trigger an immune reaction to repel the “foreign invaders.”

To prevent recognition of allogeneic (non-self) MHC class I and subsequent elimination of cells comprising allogeneic (non-self) MHC class I by recipient or host T cells, allogeneic CAR T cells are engineered to disrupt or eliminate one or more MHC-class I subunit(s). Disruption of an MHC-class I subunit on allogeneic engineered CAR T cells averts immune activation caused by engagement of foreign MHC-class I with host immune T cells. In addition, engineered CAR T cells with disrupted MHC class I fail to engage host NK cell inhibitor receptors (e.g., Killer Ig-like receptors (KIRs)) required to maintain self-tolerance. Absent the MHC Class I:NK cell receptor interaction, the engineered CAR T cells are susceptible to host/recipient NK-mediated elimination.

Accordingly, in some aspects, this disclosure provides methods for reducing NK-mediated elimination of the engineered CAR T cells. In some aspects, the disclosure also provides methods for reducing NK cell activity in a subject receiving engineered human CAR T cells. In some embodiments, the engineered human CAR T cells comprise disrupted MHC class I. In some embodiments, the expression of MHC class I in the engineered human CAR T cells is inhibited. In some embodiments, inhibition of MHC class I is by genetic inactivation or mutation of the HLA genes. In some embodiments, inhibition of MHC class I is by genetic inactivation or mutation of the β2M gene.

In some embodiments, cells comprising disrupted MHC class I result in loss of function of the MHC class I. In some embodiments, the disrupted MHC class I complex renders the engineered CAR T cell non-alloreactive and suitable for allogeneic transplantation. In some embodiments, the disrupted MHC class I complex minimizes the risk of host vs. graft diseases.

In some embodiments, gene disruption encompasses gene modification through gene editing (e.g., using CRISPR/Cas gene editing). Methods of gene editing are known in the art and are provided in this disclosure. In some embodiments, a disrupted gene is a gene that does not encode a functional protein. In some embodiments, a cell that comprises a disrupted gene does not express (e.g., at the cell surface) a detectable level (e.g., by antibody, e.g., by flow cytometry) of the protein encoded by the gene. A cell that does not express a detectable level of the protein may be referred to as a knockout cell. For example, a cell having an MHC class I gene edit may be considered an MHC class I knockout cell if the MHC class I complex or subunit cannot be detected at the cell surface using an antibody that specifically binds one or more proteins of the MHC Class I.

In some embodiments, a disrupted gene is a gene that encodes fewer copies of the encoded protein. A cell that expresses a reduced level of the protein may be referred to as a knockdown cell. For example, a cell having an MHC class I gene edit may be considered an MHC class I knockdown cell if the MHC class I molecule detected at the cell surface using an antibody that specifically binds one or more proteins on the MHC Class I molecule has lower expression compared to an unmodified cell.

In some embodiments, the disrupted MHC class I subunit is the α chain (e.g.: HLA-A, HLA-B, HLA-C). In some embodiments, the disrupted MHC class I subunit is the β2M peptide. In some embodiments, the disrupted MHC class I is the α chain associated with the β2M peptide. In some embodiments, the disrupted MHC class I is the α chain associated with the β2M peptide, additionally comprising a peptide antigen bound within the peptide binding groove. In some embodiments, the disrupted MHC class I is caused by a mutation (e.g., insertion, deletion, and/or substitution) in the β2M gene. In some embodiments, the disrupted MHC class I is caused by a mutation (e.g., insertion, deletion, and/or substitution) in the HLA-A, HLA-B, and/or HLA-C gene.

In some embodiments, less than about 10% of the engineered human CAR T cells express disrupted MHC class I. In some embodiments, less than about 50% of the engineered human CAR T cells express disrupted MHC class I. In some embodiments, less than about 90%, 80%, 70%, 60%, 40%, 30%, or 20% of the engineered human CAR T cells express disrupted MHC class I.

(ii) TRAC Gene Edit

In some embodiments, an engineered T cell comprises a disrupted TRAC gene. This disruption leads to loss of function of the TCR and renders the engineered T cell non-alloreactive and suitable for allogeneic transplantation, minimizing the risk of graft versus host disease. In some embodiments, expression of the endogenous TRAC gene is eliminated to prevent a graft-versus-host response. In some embodiments, gRNAs targeting the TRAC genomic region create Indels in the TRAC gene disrupting expression of the mRNA or protein. In some embodiments, a disruption in the TRAC gene expression is created by gRNAs targeting the TRAC genomic region. In some embodiments, a disruption in the TRAC gene expression is created by knocking an exogenous sequence (e.g., a nucleic acid encoding a chimeric antigen receptor) into the TRAC gene (e.g., using an adeno-associated viral (AAV) vector and donor template). In some embodiments, a genomic deletion in the TRAC gene is created by a gRNA and/or knocking an exogenous sequence (e.g., a nucleic acid encoding a chimeric antigen receptor) into the TRAC gene (e.g., using an AAV vector and donor template). In some embodiments, a disruption in the TRAC gene expression is created by gRNAs targeting the TRAC genomic region and knocking a chimeric antigen receptor (CAR) into the TRAC gene.

Non-limiting examples of modified and unmodified TRAC gRNA sequences that may be used as provided herein to create a genomic disruption in the TRAC gene include those comprising the nucleotide sequence of SEQ ID NO: 18 or SEQ ID NO: 19. See also International Application No. PCT/US2018/032334, filed May 11, 2018, incorporated herein by reference. Other gRNA sequences may be designed using the TRAC gene sequence located on chromosome 14 (GRCh38: chromosome 14: 22,547,506-22,552,154; Ensembl; ENSG00000277734).

In some embodiments, at least 50% of a population of engineered T cells do not express a detectable level of T cell receptor (TCR) surface protein. For example, at least 55%, at least 60%, at least 70%, at least 75%, at least 80%, at least 85%, at least 90%, or at least 95% of a population may not express a detectable level of TCR surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the population of engineered T cells do not express a detectable level of TCR surface protein. In some embodiments, less than 0.5% of a population of engineered T cells express a detectable level of TCR surface protein.

In some embodiments, gRNAs targeting the TRAC genomic region create Indels in the TRAC gene comprising at least one nucleotide sequence selected from SEQ ID NOs: 1-8 (“−” indicates deletions and nucleotide in boldface indicates insertions). See Table 1 below. In some embodiments, the disrupted TRAC gene comprises a deletion of the TRAC gene target sequence, e.g., the disrupted TRAC gene comprises a deletion of the nucleotide sequence of SEQ ID NO: 28.

TABLE 1
Exemplary Edited TRAC Gene Sequences.

| | SEQ |
|---|---|
| | ID |
| Sequence | NO: |
| AA---------------------GAGCAACAAATCTGACT | 1 |
| AAGAGCAACAGTGCTGT-GCCTGGAGCAACAAATCTGACT | 2 |
| AAGAGCAACAGTG-------CTGGAGCAACAAATCTGACT | 3 |
| AAGAGCAACAGT------GCCTGGAGCAACAAATCTGACT | 4 |
| AAGAGCAACAGTG---------------------CTGACT | 5 |
| AAGAGCAACAGTGCTGTGGGCCTGGAGCAACAAATCTGACT | 6 |
| AAGAGCAACAGTGC--TGGCCTGGAGCAACAAATCTGACT | 7 |
| AAGAGCAACAGTGCTGTGTGCCTGGAGCAACAAATCTGACT | 8 |

(iii) 2M Gene Edit

In some embodiments, an engineered T cell comprises a disrupted β2M gene. β2M is a common (invariant) component of MHC I complexes. Disrupting its expression by gene editing will prevent host versus therapeutic allogeneic T cells responses leading to increased allogeneic T cell persistence. In some embodiments, expression of the endogenous β2M gene is eliminated to prevent a host-versus-graft response.

Non-limiting examples of modified and unmodified β2M gRNA sequences that may be used as provided herein to create a genomic disruption in the β2M gene include those comprising the nucleotide sequence of SEQ ID NO:20 or SEQ ID NO:21. See also International Application No. PCT/US2018/032334, filed May 11, 2018, incorporated herein by reference. Other gRNA sequences may be designed using the β2M gene sequence located on Chromosome 15 (GRCh38 coordinates: Chromosome 15: 44,711,477-44,718,877; Ensembl: ENSG00000166710).

In some embodiments, gRNAs targeting the β2M genomic region create Indels in the β2M gene disrupting expression of the mRNA or protein.

In some embodiments, at least 50% of the engineered T cells of a population of cells does not express a detectable level of β2M surface protein. For example, at least 55%, at least 60%, at least 70%, at least 75%, at least 80%, at least 85%, at least 90%, or at least 95% of the engineered T cells of a population may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

In some embodiments, less than 50% of the engineered T cells of a population of cells express a detectable level of β2M surface protein. In some embodiments, less than 30% of the engineered T cells of a population of cells express a detectable level of β2M surface protein. For example, less than 50%, less than 30%, less than 25%, less than 20%, less than 15%, less than 10%, or less than 5% of the engineered T cells of a population of cells express a detectable level of β2M surface protein. In some embodiments, 40%-30%, 40%-20%, 40%-10%, 40%-5%, 30%-20%, 30%-10%, 30%-5%, 20%-10%, 20%-5%, or 10%-5% of the engineered T cells of a population of cells express a detectable level of β2M surface protein.

In some embodiments, gRNAs targeting the β2M genomic region create Indels in the β2M gene. In some embodiments, a disrupted β2M gene may comprise at least one sequence selected from SEQ ID NOs: 9-14 (“−” indicates deletions and nucleotide in boldface indicates insertions.). See Table 2 below.

TABLE 2
Exemplary Edited 32M Gene Sequences.

| Sequence (Deletions indicated | SEQ |
|---|---|
| by dashes (-); | ID |
| insertions indicated by bold) | NO: |
| CGTGGCCTTAGCTGTGCTCGCGCTACTCTCTCTTTCT-GC | 9 |
| CTGGAGGCTATCCAGCGTGAGTCTCTCCTACCCTCCCGCT | |
| CGTGGCCTTAGCTGTGCTCGCGCTACTCTCTCTTTC--GC | 10 |
| CTGGAGGCTATCCAGCGTGAGTCTCTCCTACCCTCCCGCT | |
| CGTGGCCTTAGCTGTGCTCGCGCTACTCTCTCTTT----- | 11 |
| CTGGAGGCTATCCAGCGTGAGTCTCTCCTACCCTCCCGCT | |
| CGTGGCCTTAGCTGTGCTCGCGCTACTCTCTCTTTCTGGA | 12 |
| TAGCCTGGAGGCTATCCAGCGTGAGTCTCTCCTACCCTCC | |
| CGCT | |
| CGTGGCCTTAGCTGTGCTCGC------------------- | 13 |
| ------GCTATCCAGCGTGAGTCTCTCCTACCCTCCCGCT | |
| CGTGGCCTTAGCTGTGCTCGCGCTACTCTCTCTTTCTGTG | 14 |
| GCCTGGAGGCTATCCAGCGTGAGTCTCTCCTACCCTCCCG | |
| CT | |

(iv) Gene Editing Methods

Gene editing (including genomic editing) is a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out or knocked-down due to the sequence alteration. Therefore, targeted editing may be used to disrupt endogenous gene expression. “Targeted integration” refers to a process involving insertion of one or more exogenous sequences, with or without deletion of an endogenous sequence at the insertion site. Targeted integration can result from targeted gene editing when a donor template containing an exogenous sequence is present.

Targeted editing can be achieved either through a nuclease-independent approach, or through a nuclease-dependent approach. In the nuclease-independent targeted editing approach, homologous recombination is guided by homologous sequences flanking an exogenous polynucleotide to be introduced into an endogenous sequence through the enzymatic machinery of the host cell. The exogenous polynucleotide may introduce deletions, insertions or replacement of nucleotides in the endogenous sequence.

Alternatively, the nuclease-dependent approach can achieve targeted editing with higher frequency through the specific introduction of double strand breaks (DSBs) by specific rare-cutting nucleases (e.g., endonucleases). Such nuclease-dependent targeted editing also utilizes DNA repair mechanisms, for example, non-homologous end joining (NHEJ), which occurs in response to DSBs. DNA repair by NHEJ often leads to random insertions or deletions (indels) of a small number of endogenous nucleotides. In contrast to NHEJ mediated repair, repair can also occur by a homology directed repair (HDR). When a donor template containing exogenous genetic material flanked by a pair of homology arms is present, the exogenous genetic material can be introduced into the genome by HDR, which results in targeted integration of the exogenous genetic material.

Available endonucleases capable of introducing specific and targeted DSBs include, but not limited to, zinc-finger nucleases (ZFN), transcription activator-like effector nucleases (TALEN), and RNA-guided CRISPR-Cas9 nuclease (CRISPR/Cas9; Clustered Regular Interspaced Short Palindromic Repeats Associated 9). Additionally, DICE (dual integrase cassette exchange) system utilizing phiC31 and Bxb1 integrases may also be used for targeted integration.

ZFNs are targeted nucleases comprising a nuclease fused to a zinc finger DNA binding domain (ZFBD), which is a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A designed zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A selected zinc finger domain is a domain not found in nature whose production results primarily from an empirical process such as phage display, interaction trap or hybrid selection. ZFNs are described in greater detail in U.S. Pat. Nos. 7,888,121 and 7,972,854. The most recognized example of a ZFN is a fusion of the FokI nuclease with a zinc finger DNA binding domain.

A TALEN is a targeted nuclease comprising a nuclease fused to a TAL effector DNA binding domain. A “transcription activator-like effector DNA binding domain”, “TAL effector DNA binding domain”, or “TALE DNA binding domain” is a polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940. The most recognized example of a TALEN in the art is a fusion polypeptide of the FokI nuclease to a TAL effector DNA binding domain.

Additional examples of targeted nucleases suitable for use as provided herein include, but are not limited to, Bxb1, phiC31, R4, PhiBT1, and Wp/SPBc/TP901-1, whether used individually or in combination.

Other non-limiting examples of targeted nucleases include naturally-occurring and recombinant nucleases, e.g., CRISPR/Cas9, restriction endonucleases, meganucleases homing endonucleases, and the like.

(v) CRISPR-Cas9 Gene Editing

The CRISPR-Cas9 system is a naturally-occurring defense mechanism in prokaryotes that has been repurposed as a RNA-guided DNA-targeting platform used for gene editing. It relies on the DNA nuclease Cas9, and two noncoding RNAs-crisprRNA (crRNA) and trans-activating RNA (tracrRNA)—to target the cleavage of DNA.

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, single-guide RNA (sgRNA), if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end-joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells and occurs at a relatively low frequency in most cell types. In many embodiments of the present disclosure, NHEJ is utilized as the repair operant.

In some embodiments, the Cas9 (CRISPR associated protein 9) endonuclease is from Streptococcus pyogenes, although other Cas9 homologs may be used. It should be understood, that wild-type Cas9 may be used or modified versions of Cas9 may be used (e.g., evolved versions of Cas9, or Cas9 orthologues or variants), as provided herein. In some embodiments, Cas9 may be substituted with another RNA-guided endonuclease, such as Cpf1 (of a class II CRISPR/Cas system).

Guide RNAs

The present disclosure provides a genome-targeting nucleic acid that can direct the activities of an associated polypeptide (e.g., a site-directed polypeptide) to a specific target sequence within a target nucleic acid. The genome-targeting nucleic acid can be an RNA. A genome-targeting RNA is referred to as a “guide RNA” or “gRNA” herein. A guide RNA comprises at least a spacer sequence that hybridizes to a target nucleic acid sequence of interest, and a CRISPR repeat sequence. In Type II systems, the gRNA also comprises a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid is a double-molecule guide RNA. In some embodiments, the genome-targeting nucleic acid is a single-molecule guide RNA.

A double-molecule guide RNA comprises two strands of RNA. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (sgRNA) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins.

A single-molecule guide RNA (referred to as a “sgRNA” or “gRNA”) in a Type V system comprises, in the 5′ to 3′ direction, a minimum CRISPR repeat sequence and a spacer sequence.

The sgRNA can comprise a 20 nucleotide spacer sequence at the 5′ end of the sgRNA sequence. The sgRNA can comprise a less than 20 nucleotide spacer sequence at the 5′ end of the sgRNA sequence. The sgRNA can comprise a more than 20 nucleotide spacer sequence at the 5′ end of the sgRNA sequence. The sgRNA can comprise a variable length spacer sequence with 17-30 nucleotides at the 5′ end of the sgRNA sequence (see, e.g., SEQ ID NOs: 15-17).

The sgRNA can comprise no uracil at the 3′ end of the sgRNA sequence. The sgRNA can comprise one or more uracil at the 3′ end of the sgRNA sequence. For example, the sgRNA can comprise 1 uracil (U) at the 3′ end of the sgRNA sequence. The sgRNA can comprise 2 uracil (UU) at the 3′ end of the sgRNA sequence. The sgRNA can comprise 3 uracil (UUU) at the 3′ end of the sgRNA sequence. The sgRNA can comprise 4 uracil (UUUU) at the 3′ end of the sgRNA sequence. The sgRNA can comprise 5 uracil (UUUUU) at the 3′ end of the sgRNA sequence. The sgRNA can comprise 6 uracil (UUUUUU) at the 3′ end of the sgRNA sequence. The sgRNA can comprise 7 uracil (UUUUUUU) at the 3′ end of the sgRNA sequence. The sgRNA can comprise 8 uracil (UUUUUUUU) at the 3′ end of the sgRNA sequence.

The sgRNA can be unmodified or modified. For example, modified sgRNAs can comprise one or more 2′-O-methyl phosphorothioate nucleotides.

By way of illustration, guide RNAs used in the CRISPR/Cas/Cpf1 system, or other smaller RNAs can be readily synthesized by chemical means, as illustrated below and described in the art. While chemical synthetic procedures are continually expanding, purifications of such RNAs by procedures such as high performance liquid chromatography (HPLC, which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs, such as those encoding a Cas9 or Cpf1 endonuclease, are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

Spacer Sequence

A gRNA comprises a spacer sequence. A spacer sequence is a sequence (e.g., a 20 nucleotide sequence) that defines the target sequence (e.g., a DNA target sequences, such as a genomic target sequence) of a target nucleic acid of interest. In some embodiments, the spacer sequence is 15 to 30 nucleotides. In some embodiments, the spacer sequence is 15, 16, 17, 18, 19, 20, 21, 22, 23, 24, 25, 26, 27, 28, 29, or 30 nucleotides. In some embodiments, a spacer sequence is 20 nucleotides.

The “target sequence” is adjacent to a PAM sequence and is the sequence modified by an RNA-guided nuclease (e.g., Cas9). The “target nucleic acid” is a double-stranded molecule: one strand comprises the target sequence and is referred to as the “PAM strand,” and the other complementary strand is referred to as the “non-PAM strand.” One of skill in the art recognizes that the gRNA spacer sequence hybridizes to the reverse complement of the target sequence, which is located in the non-PAM strand of the target nucleic acid of interest. Thus, the gRNA spacer sequence is the RNA equivalent of the target sequence. For example, if the target sequence is 5′-AGAGCAACAGTGCTGTGGCC-3′ (SEQ ID NO: 28), then the gRNA spacer sequence is 5′-AGAGCAACAGUGCUGUGGCC-3′ (SEQ ID NO: 19). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM of the Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence comprises 20 nucleotides. In some embodiments, the target nucleic acid comprises less than 20 nucleotides. In some embodiments, the target nucleic acid comprises more than 20 nucleotides. In some embodiments, the target nucleic acid comprises at least: 5, 10, 15, 16, 17, 18, 19, 20, 21, 22, 23, 24, 25, 30 or more nucleotides. In some embodiments, the target nucleic acid comprises at most: 5, 10, 15, 16, 17, 18, 19, 20, 21, 22, 23, 24, 25, 30 or more nucleotides. In some embodiments, the target nucleic acid sequence comprises 20 bases immediately 5′ of the first nucleotide of the PAM. For example, in a sequence comprising 5′-NNNNNNNNNNNNNNNNNNNNNRG-3′, the target nucleic acid comprises the sequence that corresponds to the Ns, wherein N is any nucleotide, and the underlined NRG sequence is the S. pyogenes PAM.

Non-limiting examples of TRAC gRNAs that may be used as provided herein may comprise any one of SEQ ID NOs: 18-19 and 22-23, which target SEQ ID NO: 26 in the TRAC gene. Non-limiting examples of β2M gRNAs that may be used as provided herein may comprise any one of SEQ ID NOs: 20-21 and 24-25, which target SEQ ID NO: 27 in the β2M gene. See Table 3 below. See also US Publication No. US 2018-0325955 (incorporated herein by reference for the purpose and subject matter referenced herein).

TABLE 3
gRNA Sequences/Target Sequences
gRNA Sequences

| | Unmodified | Modified |
|---|---|---|
| Name | Sequence | Sequence |
| TRAC sgRNA | AGAGCAACAG | a*g*a*gcaa |
| | UGCUGUGGCC | cagugcugug |
| | guuuuagagc | GCCguuuuag |
| | uagaaauagc | agcuagaaau |
| | aaguuaaaau | agcaaguuaa |
| | aaggcuaguc | aauaaggcua |
| | cguuaucaac | guccguuauc |
| | uugaaaaagu | aacuugaaaa |
| | ggcaccgagu | aguggcaccg |
| | cggugcUUUU | agucggugcU |
| | (SEQ ID | *U*U*U |
| | NO: 18) | (SEQ ID |
| | | NO: 22 |
| TRAC sgRNA | AGAGCAACAG | A*G*A*GCAA |
| spacer | UGCUGUGGCC | CAGUGCUGUG |
| | (SEQ ID | GCC |
| | NO: 19) | (SEQ ID |
| | | NO: 23) |
| β2M sgRNA | GCUACUCUCU | G*C*U*ACUC |
| | CUUUCUGGCC | UCUCUUUCUG |
| | guuuuagagc | GCCguuuuag |
| | uagaaauagc | agcuagaaau |
| | aaguuaaaau | agcaaguuaa |
| | aaggcuaguc | aauaaggcua |
| | cguuaucaac | guccguuauc |
| | uugaaaaagu | aacuugaaaa |
| | ggcaccgagu | aguggcaccg |
| | cggugcUUUU | agucggugcU |
| | (SEQ ID | *U*U*U |
| | NO: 20) | (SEQ ID |
| | | NO: 24) |
| β2M sgRNA | GCUACUCUCU | G*C*U*ACUC |
| spacer | CUUUCUGGCC | UCUCUUUCUG |
| | (SEQ ID | GCC |
| | NO: 21) | (SEQ ID |
| | | NO: 25) |

Target Sequences

| Guide Name | Target Sequence (PAM) |
|---|---|
| TRAC sgRNA | AGAGCAACAGTGCTGTGGCC (TGG) |
| | (SEQ ID NO: 26) |
| β2M sgRNA | GCTACTCTCTCTTTCTGGCC (TGG) |
| | (SEQ ID NO: 27) |

*2′-O-methyl phosphorothioate residue

### Donor Template

The nucleic acid encoding a CAR may be delivered to a T cell using a vector (e.g., an AAV vector) that comprises what is referred to herein as a donor template (also referred to as a donor polynucleotide). A donor template can contain a non-homologous sequence, such as the nucleic acid encoding a CAR, flanked by two regions of homology to allow for efficient HDR at a genomic location of interest. Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, is inserted so that its expression is driven by the endogenous promoter at the integration site, namely the promoter that drives expression of the endogenous gene into which the donor is inserted. However, in some embodiments, the donor template comprises an exogenous promoter and/or enhancer, for example a constitutive promoter, an inducible promoter, or tissue-specific promoter. In some embodiments, the exogenous promoter is an EF1α promoter comprising a sequence of SEQ ID NO: 47. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

In some embodiments, the donor template comprises a nucleotide sequence having at least 80%, 85%, 90%, 95%, 96%, 97%, 98% or 98% identity to SEQ ID NO: 74 and optionally encoding an anti-CD70 CAR comprising the amino acid sequence of SEQ ID NO:76. In some embodiments, the donor template comprises the nucleotide sequence of SEQ ID NO: 74.

In some embodiments, the donor template comprises a nucleotide sequence having at least 80%, 85%, 90%, 95%, 96%, 97%, 98% or 98% identity to SEQ ID NO: 84 and optionally encoding an anti-BCMA CAR comprising the amino acid sequence of SEQ ID NO:86. In some embodiments, the donor template comprises the nucleotide sequence of SEQ ID NO: 84.

In some embodiments, the donor template comprises a nucleotide sequence having at least 80%, 85%, 90%, 95%, 96%, 97%, 98% or 98% identity to SEQ ID NO: 111 and optionally encoding an anti-CD33 CAR comprising the amino acid sequence of SEQ ID NO:115. In some embodiments, the donor template comprises the nucleotide sequence of SEQ ID NO: 111.

In some embodiments, the donor template comprises a nucleotide sequence having at least 80%, 85%, 90%, 95%, 96%, 97%, 98% or 98% identity to SEQ ID NO: 58, and optionally encoding an anti-CD19 CAR comprising the amino acid sequence of SEQ ID NO:40. In some embodiments, the donor template comprises the nucleotide sequence of SEQ ID NO: 58.

(vi) Delivery Methods and Constructs

Nucleases and/or donor templates may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and donor templates in cells (e.g., T cells). Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

Adeno-Associated Viral Delivery

The donor nucleic acid encoding a CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

(vii) Homology-Directed Repair (HDR)

The donor nucleic acid encoding a CAR is inserted by homology directed repair (HDR) into the target gene locus. Both strands of the DNA at the target locus are cut by a CRISPR Cas9 enzyme. HDR then occurs to repair the double-strand break (DSB) and insert the donor DNA. For this to occur correctly, the donor sequence is designed with flanking residues which are complementary to the sequence surrounding the DSB site in the target gene (hereinafter “homology arms”). These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

The target gene can be associated with an immune response in a subject, wherein permanently deleting at least a portion of the target gene will modulate the immune response. For example, to generate a CAR T cell, the target gene can be the TCRu constant region (TRAC). Disruption of TRAC leads to loss of function of the endogenous TCR.

In some embodiments, the target gene is in a safe harbor locus.

C. Generating Genetically Engineered CAR-T Cells

Any of the genetically engineered CAR-T cells as disclosed herein may be generated using conventional methods and/or methods disclosed herein.

(i) Production of Genetically Engineered CAR-T Cells

In some embodiments, the engineered T cells described herein are generated by modifying the genome of the cells. In some embodiments, a double stranded break (DSB) at a site in a target gene is induced. In some embodiments, the DSB is repaired using one or more endogenous DNA repair pathways. In some embodiments, a DNA repair pathway does not require a homologous sequence (e.g., the non-homologous end joining pathway or NHEJ pathway). In some embodiments, a repair pathway requires a homologous sequence (e.g., the homology-directed pathway or HDR pathway).

In some embodiments, the engineered T cells described herein are generated by inducing a DSB with CRISPR-Cas9 as an endonuclease, and one or more non-coding RNAs, and repairing the DSB using HDR and a donor polynucleotide template described herein.

In some embodiments, the engineered T cells described herein are generated using a gRNA complimentary to a sequence of a target gene that is a TRAC. In some embodiments, the engineered T cells described herein are generated using a TRAC gRNA spacer comprising the sequence set forth in SEQ ID NO: 19. In some embodiments, the engineered T cells described herein are generated using a TRAC gRNA comprising the sequence set forth in SEQ ID NO: 18. In some embodiments, the TRAC gRNA comprising the sequence set forth in SEQ ID NO: 19 targets the TRAC sequence set forth in SEQ ID NO: 26. In some embodiments, the TRAC gRNA comprising the sequence set forth in SEQ ID NO: 18 targets the TRAC sequence set forth in SEQ ID NO: 26.

In some embodiments, the engineered T cells described herein are generated using a TRAC gRNA spacer comprising the sequence set forth in SEQ ID NO: 23. In some embodiments, the engineered T cells described herein are generated using a TRAC gRNA comprising the sequence set forth in SEQ ID NO: 22. In some embodiments, the TRAC gRNA comprising the sequence set forth in SEQ ID NO: 23 targets the TRAC sequence set forth in SEQ ID NO: 26. In some embodiments, the TRAC gRNA comprising the sequence set forth in SEQ ID NO: 22 targets the TRAC sequence set forth in SEQ ID NO: 26.

In some embodiments, the engineered T cells described herein are generated using a gRNA complimentary to a sequence of a target gene that is a B2M. In some embodiments, the engineered T cells described herein are generated using a B2M gRNA spacer comprising the sequence set forth in SEQ ID NO: 21. In some embodiments, the engineered T cells described herein are generated using a B2M gRNA comprising the sequence set forth in SEQ ID NO: 20. In some embodiments, the B2M gRNA comprising the sequence set forth in SEQ ID NO: 21 targets the B2M sequence set forth in SEQ ID NO: 27. In some embodiments, the B2M gRNA comprising the sequence set forth in SEQ ID NO: 20 targets the B2M sequence set forth in SEQ ID NO: 27.

In some embodiments, the engineered T cells described herein are generated using a B2M gRNA spacer comprising the sequence set forth in SEQ ID NO: 25. In some embodiments, the engineered T cells described herein are generated using a B2M gRNA comprising the sequence set forth in SEQ ID NO: 24. In some embodiments, the B2M gRNA comprising the sequence set forth in SEQ ID NO: 25 targets the B2M sequence set forth in SEQ ID NO: 27. In some embodiments, the B2M gRNA comprising the sequence set forth in SEQ ID NO: 24 targets the B2M sequence set forth in SEQ ID NO: 27.

In some embodiments, the engineered T cells described herein are generated using a TRAC gRNA comprising the sequence set forth in SEQ ID NO: 19 and a B2M gRNA comprising the sequence set forth in SEQ ID NO: 21. In some embodiments, the engineered T cells described herein are generated using a TRAC gRNA comprising the sequence set forth in SEQ ID NO: 23 and a B2M gRNA comprising the sequence set forth in SEQ ID NO: 25.

In some embodiments, the engineered T cells described herein are generated using a TRAC gRNA comprising the sequence set forth in SEQ ID NO: 18 and a B2M gRNA comprising the sequence set forth in SEQ ID NO: 20. In some embodiments, the engineered T cells described herein are generated using a TRAC gRNA comprising the sequence set forth in SEQ ID NO: 22 and a B2M gRNA comprising the sequence set forth in SEQ ID NO: 24.

For any of the gRNA sequences provided herein, those that do not explicitly indicate modifications are meant to encompass both unmodified sequences and sequences having any suitable modifications.

In some embodiments, the engineered T cells are generated using a donor template comprising a non-homologous sequence that is a nucleic acid encoding a CAR. In some embodiments, a donor template is comprised of homology arms that correspond to sequences in a target gene that is a TRAC. In some embodiments, a 5′ homology arm (left homology arm) of the donor template comprises the sequence set forth in SEQ ID NO: 45. In some embodiments, a 3′ homology arm of the donor template comprises the sequence set forth in SEQ ID NO: 46.

In some embodiments, an exogenous promoter is an EF1a promoter comprises the sequence set forth in SEQ ID NO: 47. In some embodiments, a donor template comprises the sequence set forth in SEQ ID NO: 58. In some embodiments, a donor template comprises the sequence set forth in SEQ ID NO: 74. In some embodiments, a donor template comprises the sequence set forth in SEQ ID NO: 84. In some embodiments, a donor template comprises the sequence set forth in SEQ ID NO: 111.

In some embodiments, polynucleotides encoding gRNAs, nucleases, and donor templates are introduced into cells (e.g., T cells) using conventional viral and non-viral based gene transfer methods.

In some embodiments, a polynucleotide such as a gRNA, a sgRNA, an mRNA encoding a nuclease, or a donor template are delivered to a cell using a non-viral vector delivery system. Examples of a non-viral vector delivery system include, but are not limited to, a DNA plasmid, a DNA minicircle, a naked nucleic acid, a liposome, a ribonucleoprotein particle (RNP) or a poloxamer. In some embodiments, a method of introducing polynucleotides to a cell using a non-viral vector delivery system includes electroporation, lipofection, microinjection, biolistics, or agent-enhanced uptake.

In some embodiments, a polynucleotide such as a gRNA, a sgRNA, an mRNA encoding a nuclease, or a donor template are delivered to a cell using a viral vector delivery system. Examples of a viral vector delivery system include, but are not limited to, retroviral vectors, lentiviral vectors, adenovirus vectors, poxvirus vectors, herpesvirus vectors, and adeno-associated virus (AAV) vectors.

In some embodiments, a donor template encoding a CAR construct is delivered to a cell as one or more polynucleotides. In some embodiments, a donor template encoding a CAR construct is delivered by a viral delivery vehicle. In some embodiments, a viral delivery vehicle is an adeno-associated virus (AAV) vector.

In some embodiments, an endonuclease (e.g., Cas9) is delivered to a cell as a polypeptide. In some embodiments, an endonuclease (e.g., Cas9) is delivered to a cell separately from a genome-targeting nucleic acid (e.g., a gRNA, a sgRNA). In some embodiments, an endonuclease (e.g., Cas9) is delivered to a cell as a complex with one or more genome-targeting polynucleotides (e.g., a gRNA, a sgRNA). In some embodiments, a endonuclease or a pre-complexed endonuclease is delivered by a non-viral delivery vehicle that includes, but is not limited to, a nanoparticle, a liposome, a ribonucleoprotein, a positively charged peptide, a small molecule RNA-conjugate, an aptamer-RNA chimeras, or an RNA-fusion protein complex. In some embodiments, a method of introducing an endonuclease polypeptide or a pre-complexed endonuclease polypeptide to a cell includes electroporation, lipofection, microinjection, biolistics, or agent-enhanced uptake.

In some embodiments, a Cas9 polypeptide is pre-complexed with one or more sgRNAs to form a ribonucleoprotein particle (RNP). In some embodiments, a donor template is formulated using an AAV vector. In some embodiments, delivery to a cell of a Cas9/sgRNA RNP is performed by electroporation of the cell. In some embodiments, a donor template formulated as an AAV vector is delivered prior to electroporation. In some embodiments, a donor template formulated as an AAV vector is delivered during electroporation. In some embodiments, a donor template formulated as an AAV vector is delivered following electroporation.

In some embodiments, a gene edit performed using a CRISPR/Cas9 endonuclease results in an engineered T cell with a disrupted TRAC gene. In some embodiments, a disruption in the TRAC gene expression is created by gRNAs targeting the TRAC genomic region. In some embodiments, a disruption of a TRAC gene results in eliminated or decreased expression of the TRAC gene. In some embodiments, eliminated or decreased expression of the TRAC gene is associated with loss of function of the TCR. In some embodiments, loss of TCR function renders an engineered T cell suitable to allogeneic transplantation (i.e., minimizing the risk of inducing GvHD). In some embodiments, a disruption of a TRAC gene is created by knocking in a CAR into the TRAC gene (e.g., using an AAV vector and a donor template). In some embodiments, a disruption in the TRAC gene expression is created by gRNAs targeting the TRAC genomic region and knocking in a CAR into the CAR gene. In some embodiments, a knock-in CAR is provided by a donor template with homology arms that correspond to sequences of the TRAC surrounding the site of a DSB.

In some embodiments, a gene edit performed using a CRISPR/Cas9 endonuclease results in an engineered T cell with a disrupted B2M gene. In some embodiments, a disruption in the B2M gene expression is created by gRNAs targeting the B2M genomic region. In some embodiments, gRNAs targeting the B2M genomic region create indels in the B2M gene that disrupt or inhibit transcription and translation of an encoded gene product. In some embodiments, a disruption of a B2M gene results in eliminated or decreased expression of the B2M gene. In some embodiments, eliminated or decreased expression of the B2M gene is associated with loss of function of the MHC I complex. In some embodiments, loss of MHC I function renders an engineered T cell suitable to allogeneic transplantation (i.e., minimizing the risk of a host versus allogeneic T cell response). In some embodiments, loss of MHC I function results in increased persistence of an engineered T cell in an allogeneic recipient.

(ii) Purification of Genetically Engineered CAR-T Cells

In some embodiments, the population of engineered CAR T cells (e.g., anti-CD19, anti-CD33, anti-CD70, BCMA CAR T cells) described herein are activated and/or expanded before or after genome editing. To achieve sufficient therapeutic doses of T cell compositions, T cells are often subjected to one or more rounds of stimulation, activation, and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to genome editing. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days following genome editing. In some embodiments, T cells are activated and expanded at the same time as genome editing.

In some embodiments, the disclosure provides a method for substantially removing cells that express a detectable level of a surface protein of interest (e.g., TCR) from a population of cells comprising engineered CAR T cells. In some embodiments, substantial removal of cells of interest (e.g., TCR+ cells) occurs when less than 0.1%, less than 0.2%, less than 0.3%, less than 0.4%, less than 0.5%, less than 1%, less than 2%, less than 3%, less than 4%, less than 5% or less than 10% of the cells of interest. In some embodiments, the disclosure provides a method for substantially removing cells that express a detectable level of TCR surface protein from a population of cells comprising engineered CAR T cells.

In some embodiments, the disclosure provides a method for isolating a population of cells comprising engineered CAR-T cells, comprising: providing the population of cells wherein the engineered CAR-T cells comprise a disrupted TCR gene and a disrupted B2M gene; and removing TCR+ T cells from the population of cells, thereby isolating the population of cells comprising <1.0% TCR+ T cells. In some embodiments, the disclosure provides a method for preparing an isolated population of cells comprising engineered CAR-T cells, comprising: providing the population of cells wherein the engineered CAR-T cells comprise a disrupted TCR gene and a disrupted B2M gene; and removing TCR+ T cells from the population of cells, such that the population of cells comprises <1.0% TCR+ T cells.

In some embodiments, the disclosure provides a population of cells comprising engineered CAR T cells described herein, wherein less than 0.5% of the cells in the population express a detectable level of TCR. In some embodiments, the disclosure provides a population of cells comprising engineered CAR T cells described herein, wherein less than 0.1%, less than 0.2%, less than 0.3%, less than 0.4%, less than 0.5%, less than 1%, less than 2%, less than 3%, less than 4%, less than 5% or less than 10% of the cells in the population express a detectable level of TCR.

Removal of a subset of cells from a population can be performed using conventional cell purification methods. Non-limiting examples of cell sorting methods include fluorescence-activated cell sorting, immunomagnetic separation, chromatography, and microfluidic cell sorting. In some embodiments, TCR-expressing cells are removed from a population of cells comprising engineered T cells by immunomagnetic separation. In some embodiments, TCR-expressing cells in a population of cells comprising engineered T cells are labeled with a biotinylated antibody targeting the TCR. In some embodiments, labeled cells are removed from a population of cells comprising engineered T cells using anti-biotin magnetic beads.

(iii) Characterization of Genetically Engineered CAR T Cells

In some embodiments, the engineered T cells described herein are assessed for surface protein expression of TCR, B2M, and CAR. In some embodiments, surface protein expression is determined by flow cytometry using methods known in the art. By labeling a population of cells with an element that targets the desired cell surface marker (e.g., an antibody) and is tagged with a fluorescent molecule, flow cytometry can be used to quantify the portion of the population that is positive for the surface marker, as well as the level of surface marker expression. In some embodiments, a composition of engineered T cells is assessed by flow cytometry to determine the percentage of cells with cell-surface CAR, cell-surface TCR, and cell-surface B2M.

In some embodiments, at least 50% (e.g., at least 55%, at least 60%, at least 65%, at least 70%, or at least 75%) of the engineered T cells express a detectable level of the CAR as detected by flow cytometry.

In some embodiments, at least 50% (e.g., at least 55%, at least 60%, at least 65%, at least 70%, at least 75%, or at least 80%) of the engineered T cells express a detectable level of the CAR and do not express a detectable level of TCR surface protein or B2M surface protein as detected by flow cytometry. In some embodiments, at least 50% (e.g., at least 55%, at least 60%, at least 65%, at least 70%, at least 75%, or at least 80%) of the engineered T cells express a detectable level of the CAR and express a reduced level of TCR surface protein or B2M surface protein as detected by flow cytometry. In some embodiments, at least 50% (e.g., at least 55%, at least 60%, at least 65%, at least 70%, at least 75%, or at least 80%) of the engineered T cells express a detectable level of the CAR and do not express a detectable level of TCR surface protein or reduced level of B2M surface protein as detected by flow cytometry.

In some embodiments, engineered T cells are assessed for genomic incorporation of a CAR by digital droplet PCR (ddPCR). Digital PCR enables quantification of DNA concentration in a sample. Digital PCR is performed by fractionating a mixture of a PCR reaction (e.g., containing a sample of nucleic acid molecules and copies of a PCR probe) such that some fractions contain no PCR probe copy, while other fractions contain one or more PCR probe copies. A PCR amplification of the fractions is performed and the fractions are analyzed for a PCR reaction. A fraction containing one or more probes and one or more target DNA molecules yields a positive end-point, while a fraction containing no PCR probe yields a negative end-point. The fraction of positive reactions is then fitted to a Poisson distribution to determine the absolute copy number of target DNA molecules per given volume of the unfractionated sample (i.e., copies per microliter of sample) (see Hindson, B. et al., (2011) Anal Chem. 83:8604-8610). Digital droplet PCR is a variation of digital PCR wherein a sample of nucleic acids is fractionated into droplets using a water-oil emulsion. PCR amplification is performed on the droplets collectively, whereupon a fluidics system is used to separate the droplets and provide analysis of each individual droplet. For one skilled in the art, ddPCR is used to provide an absolute quantification of DNA in a sample, to perform a copy number variation analysis, or to assess efficiency of genomic edits. In some embodiments, genomic DNA is extracted from a composition of engineered T cells and ddPCR is used to determine an absolute quantification of CAR copies per sample composition. In some embodiments, genomic DNA is extracted from a composition of engineered T cells and ddPCR is used to assess HDR efficiency of a CAR sequence into a TRAC locus.

In some embodiments, engineered T cells are assessed for cytokine-independent growth. Engineered T cells are expected to only grow in the presence of stimulatory cytokines (e.g., IL-2, IL-7). Growth in the absence of cytokines is an indicator of tumorigenic potential. In some embodiments, engineered T cells are grown for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days in either the presence or in the absence of one or more stimulatory cytokines (e.g., IL-2, IL-7). In some embodiments, proliferation is assessed by cell count and viability using conventional methods (e.g., flow cytometry, microscopy, optical density, metabolic activity). In some embodiments, proliferation is assessed starting on day 1, day 2, day 3, day 4, day 5, day 6. In some embodiments, proliferation is assessed every 1 day, every 2 days, every 3 days, every 4 days, every 5 days, every 6 days, every 7 days, or every 8 days. In some embodiments, growth in the absence of cytokines is assessed at the end of a growth period. In some embodiments, engineered T cells with no growth in the absence of cytokines is defined as lacking tumorigenic potential. In some embodiments, no growth is defined as an expansion of the population that is less than 0.1, 0.2, 0.3, 0.4, 0.5, 0.6, 0.7, 0.8, 0.9, 1.0, 1.1, 1.2, 1.3, 1.4, or 1.5 fold between the end of the growth period relative to the beginning of the growth period. In some embodiments, engineered T cells produced using the methods described herein is defined as having no growth in the absence of cytokines when assessed at 10 days, 11 days, 12 days, 13 days, 14 days, 15 days, 16 days, 17 days, 18 days, 19 days or 20 days following culture. In some embodiments, the engineered T cells do not proliferate in the absence of cytokine stimulation, growth factor stimulation, or antigen stimulation.

D. Genetically Engineered CAR-T Cells

Genetically engineered (gene edited) CAR T cells of the present disclosure may be autologous (“self”) or non-autologous (“non-self,” e.g., allogeneic, syngeneic or xenogeneic). “Autologous” refers to cells from the same subject. “Allogeneic” refers to cells of the same species as a subject, but that differ genetically to the cells in the subject. In some embodiments, the T cells are obtained from a mammalian subject. In some embodiments, the T cells are obtained from a human subject. In some examples, the T cells are allogenic.

T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation.

In some embodiments, an isolated population of T cells is used. In some embodiments, after isolation of peripheral blood mononuclear cells (PBMC), both cytotoxic and helper T lymphocytes can be sorted into naive, memory, and effector T cell subpopulations either before or after activation, expansion, and/or genetic modification.

A specific subpopulation of T cells, expressing one or more of the following cell surface markers: TCRab, CD3, CD4, CD8, CD27 CD28, CD38 CD45RA, CD45RO, CD62L, CD127, CD122, CD95, CD197, CCR7, KLRG1, MCH-I proteins and/or MCH-II proteins, can be further isolated by positive or negative selection techniques. In some embodiments, a specific subpopulation of T cells, expressing one or more of the markers selected from the group consisting of TCRab, CD4 and/or CD8, is further isolated by positive or negative selection techniques. In some embodiments, subpopulations of T cells may be isolated by positive or negative selection prior to genetic engineering and/or post genetic engineering.

In some embodiments, an isolated population of T cells expresses one or more of the markers including, but not limited to a CD3+, CD4+, CD8+, or a combination thereof. In some embodiments, the T cells are isolated from a subject and first activated and stimulated to proliferate in vitro prior to undergoing gene editing.

In some embodiments, the amount of CD27+CD45RO− T cells within a population of donor T cells indicates the level of expression of exhaustion and senescent markers (e.g., PD1, TIM-3, LAG3, CD57) after gene editing (e.g., CRISPR-Cas9 editing). In some embodiments, a population of donor T cells comprises at least 40%, 50% or 50% CD27+CD45RO− T cells. In some embodiments, expression of exhaustion or senescent markers on donor T cells comprising at least 40%, 50% or 60% CD27+CD45RO− T cells does not significantly change after gene editing.

To achieve sufficient therapeutic doses of T cell compositions, T cells are often subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of the genome editing compositions into the T cells.

In some embodiments, T cells are activated and expanded for about 4 hours, about 6 hours, about 12 hours, about 18 hours, about 24 hours, about 36 hours, about 48 hours, about 60 hours, or about 72 hours prior to introduction of the gene editing compositions into the T cells.

In some embodiments, T cells are activated at the same time that genome editing compositions are introduced into the T cells.

E. Pharmaceutical Compositions

In some aspects, the present disclosure provides pharmaceutical compositions comprising a population of genetically engineered CAR T cells such as those disclosed herein, and a pharmaceutically acceptable carrier. For example, the pharmaceutical composition may comprise a population of the genetically engineered anti-CD19 CAR-T cells, a population of the genetically engineered anti-BCMA CAR-T cells, a population of the genetically engineered anti-CD70 CAR-T cells, or a population of the genetically engineered anti-CD33 CAR-T cells. In some aspects, the present disclosure provides pharmaceutical compositions comprising an NK cell inhibitor such as an anti-CD38 antibody (e.g., daratumumab). Such pharmaceutical compositions can be used in methods for improving a clinical outcome in human patients, which is also disclosed herein.

As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions can include a pharmaceutically acceptable salt, e.g., an acid addition salt or a base addition salt. See, e.g., Berge et al., (1977) J Pharm Sci 66:1-19.

In some embodiments, the pharmaceutical composition further comprises a pharmaceutically acceptable salt. Non-limiting examples of pharmaceutically acceptable salts include acid addition salts (formed from a free amino group of a polypeptide with an inorganic acid (e.g., hydrochloric or phosphoric acids), or an organic acid such as acetic, tartaric, mandelic, or the like). In some embodiments, the salt formed with the free carboxyl groups is derived from an inorganic base (e.g., sodium, potassium, ammonium, calcium or ferric hydroxides), or an organic base such as isopropylamine, trimethylamine, 2-ethylamino ethanol, histidine, procaine, or the like).

In some embodiments, the pharmaceutical composition disclosed herein comprises a population of the genetically engineered CAR-T cells suspended in a cryopreservation solution (e.g., CryoStor® C55). The cryopreservation solution for use in the present disclosure may also comprise adenosine, dextrose, dextran-40, lactobionic acid, sucrose, mannitol, a buffer agent such as N-)2-hydroxethyl) piperazine-N′-(2-ethanesulfonic acid) (HEPES), one or more salts (e.g., calcium chloride, magnesium chloride, potassium chloride, postassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

In some instances, a pharmaceutical composition comprising a population of genetically engineered CAR-T cells suspended in a cryopreservation solution (e.g., substantially free of serum) may be placed in storage vials.

Any of the pharmaceutical compositions disclosed herein, comprising a population of genetically engineered CAR T cells such as those disclosed herein, which optionally may be suspended in a cryopreservation solution as disclosed herein may be stored in an environment that does not substantially affect viability and bioactivity of the T cells for future use, e.g., under conditions commonly applied for storage of cells and tissues. In some examples, the pharmaceutical composition may be stored in the vapor phase of liquid nitrogen at ≤−135° C. No significant changes were observed with respect to appearance, cell count, viability, % CAR+ T cells, and % gene edit+ T cells after the cells have been stored under such conditions for a period of time.

### II. NK Cell Inhibitors

NK cells play an important role in both innate and adaptive immunity—including mediating anti-tumor and anti-viral responses. Because NK cells do not require prior sensitization or priming to mediate its cytotoxic function, they are the first line of defense against virus-infected and malignant cells that have missing or nonfunctioning MHC class I (e.g., disrupted MHC class I, or disrupted MCH Class I subunits). NK cells recognize “non-self” cells without the need for antibodies and antigen-priming. MHC class I-specific inhibitory receptors on NK cells negatively regulate NK cell function. Engagement of NK cell inhibitory receptors with their MHC class I ligand checks NK cell-mediated lysis. When MHC class I-disrupted cells fail to bind inhibitory NK receptors (e.g., KIRs), the cells become susceptible to NK cell-mediated lysis. This phenomenon is also referred to as the “missing self recognition.” See e.g., Malmberg K J et al., Immunogenetics (2017), 69:547-556; Cruz-Munoz M E et al., J. Leukoc. Biol. (2019), 105:955-971.

Therefore, engineered human CAR T cells comprising disrupted MHC class I as described herein are susceptible to NK cell-mediated lysis, thus reducing the persistence and subsequent efficacy of the engineered human CAR T cells.

Accordingly, in some embodiments the present disclosure provides NK cell inhibitors for use in combination with CAR T cell therapy comprising a population of engineered human CAR T cells as described herein.

The NK cell inhibitor to be used in the methods described herein can be a molecule that blocks, suppresses, or reduces the activity or number of NK cells, either directly or indirectly. The term “inhibitor” implies no specific mechanism of biological action whatsoever, and is deemed to expressly include and encompass all possible pharmacological, physiological, and biochemical interactions with NK cells whether direct or indirect. For the purpose of the present disclosure, it will be explicitly understood that the term “inhibitor” encompasses all the previously identified terms, titles, and functional states and characteristics whereby the NK cell itself, a biological activity of the NK cell (including but not limited to its ability to mediate cell killing), or the consequences of the biological activity, are substantially nullified, decreased, or neutralized in any meaningful degree, e.g., by at least 20%, 50%, 70%, 85%, 90%, 100%, 150%, 200%, 300%, or 500%, or by 10-fold, 20-fold, 50-fold, 100-fold, 1000-fold, or 10⁴-fold.

In some embodiments, an NK cell inhibitor reduces absolute NK cell numbers. In some embodiments, an NK cell inhibitor reduces NK cell frequency in peripheral blood mononuclear cells. In some embodiments, the NK cells are reduced by at least 10%, 20%, 30%, 40%, 50%, 60%, 70%, 80%, or 90%. In some embodiments, an NK cell inhibitor reduces the total number of NK cells in a subject receiving CAR T cell therapy compared to the total number of NK cells in a subject prior to receiving an NK cell inhibitor. In some embodiments, the NK cells are reduced to at least 20, 40, 60, 80, 100, 120, 140, 160, or 180 NK cells/μL of blood. In some embodiments, the NK cells are reduced to less than 200 NK cells/μL of blood.

In some embodiments, an NK cell inhibitor does not significantly reduce endogenous T cell numbers. In some embodiments, an NK cell inhibitor maintains endogenous T cell numbers at 85%, 90%, 95%, 100%, 105%, or 110% of T cell numbers relative to T cell numbers before NK cell inhibitor treatment. In some embodiments, an NK cell inhibitor maintains endogenous T cell numbers at approximately 1500 T cells/μL of blood. In some embodiments, an NK cell inhibitor maintains endogenous T cell number at approximately 1275, 1350, 1425, 1500, 1575, or 1650 T cells/μL of blood. In some embodiments, an NK cell inhibitor does not significantly reduce the number of engineered human CAR T cells. In some embodiments, an NK cell increases the number of engineered human CAR T cells compared to the number of engineered human CAR T cells in the absence of an NK cell inhibitor. In some embodiments, an NK cell inhibitor does not significantly activate the engineered human CAR T cells.

In some embodiments, an NK cell inhibitor reduces NK cell-mediated lysis of engineered human CAR T cells. In some embodiments, an NK cell inhibitor reduces NK cell-mediated lysis of engineered human T cells by about 10%, 20%, 30%, 40%, 50%, 60%, 70%, 80%, 90% or 100% relative to NK cell-mediated lysis of engineered human CAR T cells in the absence of an NK cell inhibitor. In some embodiments, an NK cell inhibitor reduces NK cell-mediated lysis of engineered human T cells in vivo.

In some embodiments, an NK cell inhibitor reduces an NK cell activity. In some embodiments, the disclosure provides methods for reducing NK cell activity in a subject receiving a CAR T cell therapy by administering an NK cell inhibitor. In some embodiments, an NK cell inhibitor reduces antibody-dependent cell-mediated cytotoxicity (ADCC), antibody-dependent cellular phagocytosis (ADCP), complement dependent cytotoxicity (CDC), apoptosis, or combinations thereof.

In some embodiments, an NK cell inhibitor reduces NK cell-mediated antibody-dependent cell-mediated cytotoxicity (ADCC) of engineered human CAR T cells. NK cells express Fc-receptors, e.g., FcγRIIIA and or FcγRIIC on their cell surfaces. The Fc-receptors bind the Fc portion of antibodies. Once bound, the Fc-receptors transmit activating signals through immune tyrosine-based activating motifs (ITAM), and results in downstream NK cell degranulation, cytokine secretion (e.g., IFN-γ), and cell lysis.

In some embodiments, an NK cell inhibitor reduces NK cell-mediated antibody-dependent cellular phagocytosis (ADCP) of engineered human CAR T cells. ADCP occurs when the Fc portion of an antibody engages the Fc-receptor, e.g., FcγRIIIA, FcγRIIA, or FcγRI on macrophages. The engagement of Fc receptors on macrophages triggers phagocytosis of target cells and results in macrophages engulfing and eliminating the target cells, e.g., engineered human CAR T cells.

In some embodiments, an NK cell inhibitor reduces NK cell-mediated complement dependent cytotoxicity (CDC) of engineered human CAR T cells. Antibodies bound to a cell surface, e.g., NK cell surface, trigger complement activation through the classical pathway. Complement activation induces cell lysis, phagocytosis, chemotaxis, and immune cell activation. Complement component C1 recognizes Fc portion of antibodies and becomes activated upon antibody binding. C1 activation triggers a cascade of enzyme activation, cumulating into the cleavage and activation of complement component C3 into C3a and C3b. C3b is opsonized on cell surface and triggers downstream activation of C5b-C9 components to form membrane-attack complexes (MACs) on target cell membrane, resulting in membrane disruption and cell lysis.

In some embodiments, an NK cell inhibitor reduces NK cell-mediated apoptosis of engineered human CAR T cells. Once NK cells recognize and engage target cells (e.g., engineered human T cells) through receptor binding, immunological synapses (ISs) are formed through cytoskeletal reorganization that polarizes microtubules formation, allowing transport and release of NK lytic enzymes into the target cells. Exemplary lytic enzymes include Granzyme B, perforin, FasL, TRAIL, and granulosyn. A serine protease, Granzyme B triggers apoptosis through caspase-dependent pathways by directly cleaving pro-apoptotic molecules such as caspase-8 and caspase-3. Granzyme B also induces apoptosis by cleaving the pro-apoptotic molecule, Bid, which causes cytochrome C release from mitochondria. Other than lytic degranulation, the Fas ligand (FasL) and TNR-related apoptosis-inducing ligand (TRAIL) molecules on NK cells also induce cell death. These receptors bind and activate death receptors TRAILR and Fas on target cells, and trigger a pro-apoptotic cascade involving caspases and IL1β-converting enzyme (ICE) proteases.

In addition to cytolytic functions, NK cells also exert their immunomodulatory function through the secretion of inflammatory and immunosuppressive cytokines. Upon contact with target cells, NK cells secrete Th1 cytokines, IFN-γ, TNF, GM-CSF, etc. These cytokines activate T cells, dendritic cells, macrophages, and neutrophils. NK cells additionally secrete chemokines, e.g., MIP-1α, MIP-1β, RANTES, lymphotoxin, IL-8 (CXCL8), which attracts effector cells to the activation site. In some embodiments, an NK cell inhibitor reduces an immunomodulatory function of an NK cell. In some embodiments, an NK cell inhibitor reduces secretion of inflammatory cytokines, resulting in reduced activation induced cell death of engineered human CAR T cells.

In vitro and in vivo experiments to determine NK cell activity are known in the art. Exemplary assays include cytolytic assays, ADCC assays, flow cytometry assays to determine cytokine secretion, apoptosis induction, degranulation, CDC or NK cell proliferation. See e.g., Huang M, et al., Hepatology (2013), 57:277-288; EP 2 658 871 B1; De Weers M, et al., J. Immunol. (2011) 186:1840-8; EP 1 720 907 B1; U.S. Pat. Nos. 7,829,673; 9,944,711.

A. Exemplary NK Cell Inhibitors

In some embodiments, the NK cell inhibitor is a small molecule, a monoclonal antibody or an antigen binding fragment thereof, a polypeptide, a polynucleotide, or combinations thereof.

In some embodiments, an NK cell inhibitor is a small molecule. An exemplary small molecule NK inhibitor is ruxolitinib (Jakafi®). Ruxolitinib is a Janus kinase inhibitor used in the treatment of myelofibrosis. Ruxolitinib binds and inhibits protein tyrosine kinases JAK 1 and 2. Patients treated with ruxolitinib demonstrated increased infection rates. Ruxolitinib reduces NK cell proliferation, cytokine-induced receptor expression and NK cell function—including, for example, reduced killing, reduced degranulation, reduced IFN-γ production, and reduced cytokine signaling (Schonberg et al., Blood (2014), 124(21):3169). Ruxolitinib structure and methods of preparing ruxolitinib are found, e.g., in U.S. Pat. Nos. 7,598,257, 8,415,362, 8,722,693, 8,882,481, 8,829,013, and 9,079,912. Additional exemplary small molecule immunosuppressive drugs that inhibit NK cell functions are described in Pradier A, et al., Front. Imunol. (2019), 10:556. In some embodiments, the NK cell inhibitor is ruxolitinib, cyclosporine A (CsA), tacrolimus (TAC), mycophenolic acid (MPA), mycophenolate mofetil (MMF), everolimus, or rapamycin.

In some embodiments, an NK cell inhibitor is a polypeptide. HLA-G is a non-classical class I antigen expressed in human placenta and thymic epithelial cells. Expression of the HLA-G antigen on the placenta protects the fetus from maternal immune rejection. Rouas-Freiss N, et al. Proc. Natl. Acad. Sci. (1997), 94:5249-5254. The HLA-G gene is alternatively spliced and transcribes HLA-G mRNAs encoding membrane-bound HLA-G (HLA-G1, HLA-G2, HLA-G3, and HLA-G4) and soluble HLA-G (HLA-G5, HLA-G6, and HLA-G7). HLA-G expression on cancer cells protects B cell lymphoma from NK mediated cell lysis. Similarly, transfection of HLA-G1 and HLA-G2 isoforms into K562 target cells abolished cytotoxicity mediated by NK-like YT2C2 T cell leukemia clone. Target cells transfected with extracellular HLA-G1, G2, G3, or G4 also inhibit cytotoxic activity of NK cells in a cell lysis assay. See, e.g., EP1189627, Example 3. A recombinant fusion polypeptide comprising β2M-spacer-HLA-G5 formulated into microspheres and administered intraperitoneally into mice receiving allogeneic skin transplants was able to improve graft tolerance. See, e.g., EP 2 184 297 A1. Additional HLA-G recombinant proteins have been tested as potential treatments for tissue rejection. See e.g., Favier B, et al., PLoS One (2011), 6(7):e21011; EP 2 264 067 A1. Accordingly, in some embodiments, the NK cell inhibitor is HLA-G1, HLA-G2, HLA-G3, GLA-G4, β2M-HLA-G5, HLA-G alpha 1 domain-Fc, or HLA-G alpha 1.

In some embodiments, a cell may comprise additional MHC class I gene edits. In some embodiments, a cell can be engineered to comprise an exogenous MHC class I gene. In some embodiments, a cell can be engineered to comprise an exogenous gene that encodes a functional protein. In some embodiments, a cell can be engineered to expresses an exogenous gene (e.g., at the cell surface) a detectable level (e.g., by antibody, e.g., by flow cytometry) of the protein encoded by the exogenous gene. A cell that expresses a detectable level of the exogenous protein may be referred to as a knock-in cell. For example, a cell having an MHC class I gene edit may be considered an MHC class I knock-in cell if the MHC class I molecule is detected at the cell surface using an antibody that specifically binds one or more proteins on the MHC Class I molecule.

In some embodiments, an NK cell inhibitor is a polynucleotide. In some embodiments, the polynucleotide includes, but is not limited to a small interfering RNA (siRNA), a short hairpin RNA (shRNA), or antisense oligonucleotide (ASO). In some embodiments, the polynucleotide is formulated into lipid nanoparticles (LNP) for delivery into cells. In some embodiments, the polynucleotide is conjugated for delivery to specific cell types. For example, siRNA conjugated to trivalent N-acetylgalactosamine receptor (GalNAc), for targeting liver cells. In some embodiments, the siRNA is conjugated to CpG nucleotides, which bind receptors on dendritic cells or macrophages. In some embodiments, the polynucleotide is delivered in a vector. In some embodiments, the vector is a plasmid vectors or DNA minicircles, In some embodiments, the vector is a recombinant virus vector. In some embodiments, the recombinant virus is a recombinant poxvirus, a recombination herpesvirus, a recombinant adenovirus, a recombinant lentiviral, or a recombinant vesicular stomatitis virus (VSV), and combinations thereof. In a non-limiting example, the NK cell inhibitor is a shRNA targeting the NKG2D receptor. Huang M, et al., Hepatology (2013), 57:277-288. NK mediated cytolysis is reduced when a plasmid containing shRNA targeting three murine NKG2D was injected into mice. In another embodiment, a potassium channel tetramerization domain containing 9 (KCTD9) protein is elevated in NK cells of patients with viral hepatitis. Zhang X, et al., BMC Immunol. (2018), 19:20. Injection of plasmid encoding shRNA targeting KCTD9 into a mouse hepatitis model resulted in increased survival of the mice. In some embodiments, the NK cell inhibitor is NKG2D shRNA, or KCTD9 shRNA.

In some embodiments, an NK cell inhibitor is a monoclonal antibody. Non-limiting examples of antibodies that reduce NK cell activity are disclosed in AU2005321017B2 (anti-NKG2A antibody), US20030095965A1 (bivalent antibodies to CD94/NKG2 receptors), U.S. Pat. No. 9,211,328 (antibodies to NKG2D), and U.S. Pat. No. 7,829,673 (antibodies to CD38). Accordingly, in some embodiments the NK cell inhibitor is an anti-NKG2A antibody, a bivalent antibody to CD94/NKG2 receptors, an anti-NKG2D antibody, or an anti-CD38 antibody.

B. Antibodies that Bind CD38 (Anti-CD38 Antibodies)

In some embodiments, the present disclosure provides antibodies or antigen-binding fragments thereof that specifically bind CD38 for use in the methods described herein. CD38, also known as cyclic ADP ribose hydrolase, is a 46-kDa type II transmembrane glycoprotein that synthesizes and hydrolyzes cyclic adenosine 5′-diphosphate-ribose, an intracellular calcium ion mobilizing messenger. A multifunctional protein, CD38 is also involved in receptor-mediated cell adhesion and signaling.

An antibody (interchangeably used in plural form) as used herein is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) monoclonal antibodies, but also antigen-binding fragments (such as Fab, Fab′, F(ab′)2, Fv, single chain variable fragment (scFv)), mutants thereof, fusion proteins comprising an antibody portion, humanized antibodies, chimeric antibodies, diabodies, linear antibodies, single chain antibodies, single domain antibodies (e.g., camel or llama VHH antibodies), multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies.

A typical antibody molecule comprises a heavy chain variable region (VH) and a light chain variable region (VL), which are usually involved in antigen binding. These regions/residues that are responsible for antigen-binding can be identified from amino acid sequences of the VH/VL sequences of a reference antibody (e.g., an anti-CD38 antibody as described herein) by methods known in the art. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. As used herein, a CDR may refer to the CDR defined by any method known in the art. Two antibodies having the same CDR means that the two antibodies have the same amino acid sequence of that CDR as determined by the same method. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.

An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used as provided herein can be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC).

In some embodiments, an antibody of the present disclosure is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. A humanized antibody optimally also will comprise at least a portion of an immunoglobulin constant region or domain (Fc), typically that of a human immunoglobulin. Other forms of humanized antibodies have one or more CDRs (one, two, three, four, five, and/or six) which are altered with respect to the original antibody, which are also termed one or more CDRs “derived from” one or more CDRs from the original antibody. Humanized antibodies may also involve affinity maturation.

In some embodiments, an antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, an antibody of the present disclosure specifically binds a target antigen (e.g., human CD38). An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a CD38 epitope, or is an antibody that binds this epitope with greater affinity, avidity, more readily, and/or with greater duration than it binds to other epitopes of the same antigen or a different antigen. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

Also within the scope of the present disclosure are functional variants of any of the exemplary antibodies as disclosed herein. A functional variant may contain one or more amino acid residue variations in the VH and/or VL, or in one or more of the HC CDRs and/or one or more of the VL CDRs as relative to a reference antibody, while retaining substantially similar binding and biological activities (e.g., substantially similar binding affinity, binding specificity, inhibitory activity, anti-tumor activity, or a combination thereof) as the reference antibody.

In some instances, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) A→G, S; (b) R→K, H; (c) N→Q, H; (d) D→E, N; (e) C→S, A; (f) Q→N; (g) E→D, Q; (h) G→A; (i) H→N, Q; (j) I→L, V; (k) L→I, V; (l) K→R, H; (m) M→L, I, Y; (n) F→Y, M, L; (o) P→A; (p) S→T; (q) T→S; (r) W→Y, F; (s) Y→W, F; and (t) V→I, L.

In some embodiments, the NK cell inhibitor used in the combined therapy discloses herein is an anti-CD38 antibody. CD38 is expressed on many immune cells, including lymphoid, erythroid, and myeloid cells. In addition, CD38 is highly expressed in various leukemias, myelomas and solid tumors including multiple myeloma, B-cell non-Hodgkin lymphoma (NHL), B-cell chronic lymphocytic leukemia, B-cell acute lymphoblastic leukemia (ALL), and T-cell ALL. CD38 is a marker for lymphocyte activation and is used as a prognostic marker for patients with chronic lymphocytic leukemia.

An amino acid sequence of an exemplary human CD38 protein is provided in SEQ ID NO: 59 (NCBI Reference Sequence: NP001766.2). An mRNA sequence encoding an exemplary human CD38 protein is provided in SEQ ID NO: 60 (NCBI Reference Sequence: NM_001775.4) (Homo sapiens CD38 molecule (CD38), transcript variant 1). Methods for generating antibodies that specifically bind human CD38 are known to those of ordinary skill in the art.

Anti-CD38 antibodies have been tested in various pre-clinical and clinical studies, e.g., for NK/T cell lymphoma, T-cell acute lymphoblastic leukemia, immunoglobulin. Exemplary anti-CD38 antibodies tested for anti-tumor properties include SAR650984 (also referred to as isatuximab, chimeric mAb), which is in phase I clinical trials in patients with CD38+ B-cell malignancies (Deckert J. et al., Clin. Cancer. Res. (2014): 20(17):4574-83), MOR202 (also referred to as MOR03087, fully human mAb), and TAK-079 (fully human mAb).

In some embodiments, an anti-CD38 antibody for use in the present disclosure includes SAR650984 (Isatuximab), MOR202, Ab79, Ab10, HM-025, HM-028, HM-034; as well as antibodies disclosed in U.S. Pat. Nos. 9,944,711, 7,829,673, WO2006/099875, WO 2008/047242, WO2012/092612, and EP 1 720 907 B1, herein incorporated by reference. In some embodiments, the anti-CD38 antibody disclosed herein may be a functional variant of any of the reference antibodies disclosed herein. Such a functional variant may comprise the same heavy chain and light chain complementary determining regions as the reference antibody. In some examples, the functional variant may comprise the same heavy chain variable region and the same light chain variable region as the reference antibody.

In some embodiments, the anti-CD38 antibody for use in the present disclosure is daratumumab. Daratumumab (also referred to as Darzalex®, HuMax-CD38, or IgG1-005) is a fully human IgGκ monoclonal antibody that targets CD38 and has been approved for treating multiple myeloma. It is used as a monotherapy or as a combination therapy for treating newly diagnosed or previously treated multiple myeloma patients. Daratumumab is described in U.S. Pat. No. 7,829,673 and WO2006/099875.

Daratumumab binds an epitope on CD38 that comprises two β-strands located at amino acids 233-246 and 267-280 of CD38. Experiments with CD38 mutant polypeptides show that the S274 amino acid residue is important for daratumumab binding. (van de Donk NWCJ et al., Immunol. Rev. (2016) 270:95-112). Daratumumab's binding orientation to CD38 allows for Fc-receptor mediated downstream immune processes.

Mechanisms of action attributed to Daratumumab as a lymphoma and multiple myeloma therapy includes Fc-dependent effector mechanisms such as complement-dependent cytotoxicity (CDC), natural killer (NK)-cell mediated antibody-dependent cellular cytotoxicity (ADCC) (De Weers M, et al., J. Immunol. (2011) 186:1840-8), antibody-mediated cellular phagocytosis (ADCP) (Overdijk M B et al., MAbs (2015), 7(2):311-21), and apoptosis after cross-linking (van de Donk NWCJ and Usmani S Z, Front. Immunol. (2018), 9:2134).

The full heavy chain amino acid sequence of daratumumab is set forth in SEQ ID NO: 61 and the full light chain amino acid sequence of daratumumab is set forth in SEQ ID NO: 63. The amino acid sequence of the heavy chain variable region of daratumumab is set forth in SEQ ID NO: 62 and the amino acid sequence of the light chain variable region of daratumumab is set forth in SEQ ID NO: 64. Daratumumab includes the heavy chain complementary determining regions (HCDRs) 1, 2, and 3 (SEQ ID NOs: 65, 66, and 67, respectively), and the light chain CDRs (LCDRs) 1, 2, and 3 (SEQ ID NOs. 68, 69, and 70, respectively). In some embodiments, these sequences can be used to produce a monoclonal antibody that binds CD38. For example, methods for making daratumumab are described in U.S. Pat. No. 7,829,673 (incorporated herein by reference for the purpose and subject matter referenced herein).

In some embodiments, an anti-CD38 antibody for use in the present disclosure is daratumumab, an antibody having the same functional features as daratumumab, or an antibody which binds to the same epitope as daratumumab.

In some embodiments, the anti-CD38 antibody comprises: (a) an immunoglobulin heavy chain variable region and (b) an immunoglobulin light variable region, wherein the heavy chain variable region and the light chain variable region defines a binding site (paratope) for CD38. In some embodiments, the heavy chain variable region comprises an HCDR1 comprising the amino acid sequence set forth in SEQ ID NO: 65, an HCDR2 comprising the amino acid sequence set forth in SEQ ID NO: 66; and an HCDR3 comprising the amino acid sequence in SEQ ID NO: 67. The HCDR1, HCDR2, and HCDR3 sequences are separated by the immunoglobulin framework (FR) sequences.

In some embodiments, the anti-CD38 antibody comprises: (a) an immunoglobulin light chain variable region and (b) an immunoglobulin heavy chain variable region, wherein the light chain variable region and the heavy chain variable region defines a binding site (paratope) for CD38. In some embodiments, the light chain variable region comprises an LCDR1 comprising the amino acid sequence set forth in SEQ ID NO: 68, an LCDR2 comprising the amino acid sequence set forth in SEQ ID NO: 69; and an LCDR3 comprising the amino acid sequence in SEQ ID NO: 70. The LCDR1, LCDR2, and LCDR3 sequences are separated by the immunoglobulin framework (FR) sequences.

In some embodiments, the anti-CD38 antibody comprises an immunoglobulin heavy chain variable region (VH) comprising the amino acid sequence set forth in SEQ ID NO: 62, and an immunoglobulin light chain variable region (VL). In some embodiments, the anti-CD38 antibody comprises an immunoglobulin light chain variable region (VL) comprising the amino acid sequence set forth in SEQ ID NO: 64, and an immunoglobulin heavy chain variable region (VH). In some embodiments, the anti-CD38 antibody comprises a VH comprising an amino acid sequence that is at least 70%, 75%, 70%, 85%, 90%, 95%, 96%, 97%, 98%, and 99% identical to the amino acid sequence set forth in SEQ ID NO: 62, and comprises an VL comprising an amino acid sequence that is at least 70%, 75%, 70%, 85%, 90%, 95%, 96%, 97%, 98%, and 99% identical to the amino acid sequence set forth in SEQ ID NO: 64.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

CD38 is expressed on NK cells and infusion of daratumumab results in a reduction of NK cells in peripheral blood and bone marrow. The reduction of NK cells is due to NK-cell killing via ADCC, in which NK cells mediate cytotoxic killing of neighboring NK cells. Administration of daratumumab has also been shown to decrease cell numbers of myeloid derived suppressor cells, regulatory T cells, and regulatory B cells. The elimination of regulatory immune cells results in increased T cell responses and increased T cell numbers (J Krejcik et al., Blood (2016), 128(3):384-394.

Accordingly, in some embodiments, the anti-CD38 antibody (e.g., daratumumab) reduces absolute NK cell numbers. In some embodiments, the anti-CD38 antibody reduces NK cell percentage in PBMCs. In some embodiments, the anti-CD38 antibody inhibits NK cell activity through Fc-mediated mechanisms. In other embodiments, the anti-CD38 antibody mediates the killing of NK cells through CDC. In other embodiments, the anti-CD38 antibody mediates the killing of NK cells through ADCC. In other embodiments, the anti-CD38 antibody enhances phagocytosis of NK cells. In other embodiments, the anti-CD38 antibody enhances apoptosis induction after FcγR-mediated cross-linking.

In some embodiments, the anti-CD38 antibody is daratumumab or an antibody having the same functional features as daratumumab, for example, a functional variant of daratumumab. In some examples, a functional variant comprises substantially the same VH and VL CDRs as daratumumab. For example, it may comprise only up to 8 (e.g., 8, 7, 6, 5, 4, 3, 2, or 1) amino acid residue variations in the total CDR regions of the antibody and binds the same epitope of CD38 with substantially similar affinity (e.g., having a KD value in the same order) as daratumumab. In some instances, the functional variants may have the same heavy chain CDR3 as daratumumab, and optionally the same light chain CDR3 as daratumumab. Alternatively or in addition, the functional variants may have the same heavy chain CDR2 as daratumumab. Such an anti-CD38 antibody may comprise a VH fragment having CDR amino acid residue variations in only the heavy chain CDR1 as compared with the VH of daratumumab. In some examples, the anti-CD38 antibody may further comprise a VL fragment having the same VL CDR3, and optionally same VL CDR1 or VL CDR2 as daratumumab. Alternatively or in addition, the amino acid residue variations can be conservative amino acid residue substitutions (see above disclosures).

In some embodiments, the anti-CD38 antibody may comprise heavy chain CDRs that are at least 80% (e.g., 85%, 90%, 95%, or 98%) sequence identity, individually or collectively, as compared with the VH CDRs of daratumumab. Alternatively or in addition, the anti-CD38 antibody may comprise light chain CDRs that are at least 80% (e.g., 85%, 90%, 95%, or 98%) sequence identity, individually or collectively, as compared with the V_{L} CDRs as daratumumab. As used herein, “individually” means that one CDR of an antibody shares the indicated sequence identity relative to the corresponding CDR of daratumumab. “Collectively” means that three VH or VL CDRs of an antibody in combination share the indicated sequence identity relative the corresponding three VH or VL CDRs of daratumumab.

In some embodiments, the anti-CD38 antibody binds to the same epitope bound by daratumumab on human CD38. In some embodiments, the anti-CD38 antibody competes with daratumumab for binding to human CD38.

Competition assays for determining whether an antibody binds to the same epitope as daratumumab, or competes with daratumumab for binding to CD38, are known in the art. Exemplary competition assays include immunoassays (e.g., ELISA assay, RIA assays), surface plasmon resonance, (e.g., BIAcore analysis), bio-layer interferometry, and flow cytometry.

A competition assay typically involves an immobilized antigen (e.g., CD38), a test antibody (e.g., CD38-binding antibody) and a reference antibody (e.g., daratumumab). Either one of the reference or test antibody is labeled, and the other unlabeled. In some embodiments, competitive binding is determined by the amount of a reference antibody bound to the immobilized antigen in increasing concentrations of the test antibody. Antibodies that compete with a reference antibody include antibodies that bind the same or overlapping epitopes as the reference antibody. In some embodiments, the test antibodies bind to adjacent, non-overlapping epitopes such that the proximity of the antibodies causes a steric hindrance sufficient to affect the binding of the reference antibody to the antigen.

A competition assay can be conducted in both directions to ensure that the presence of the label or steric hindrance does not interfere or inhibit binding to the epitope. For example, in the first direction, the reference antibody is labeled and the test antibody is unlabeled. In the second direction, the test antibody is labeled, and the reference antibody is unlabeled. In another embodiment, in the first direction, the reference antibody is bound to the immobilized antigen, and increasing concentrations of the test antibody are added to measure competitive binding. In the second direction, the test antibody is bound to the immobilized antigen, and increasing concentrations of the reference antibody are added to measure competitive binding.

In some embodiments, two antibodies can be determined to bind to the same epitope if essentially all amino acid mutations in the antigen that reduce or eliminate the binding of one antibody reduce or eliminate binding of the other. Two antibodies can be determined to bind to overlapping epitopes if only a subset of the mutations that reduce or eliminate the binding of one antibody reduces or eliminates the binding of the other.

In some embodiments, the heavy chain of any of the anti-CD38 antibodies as described herein (e.g., daratumumab) may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-CD38 antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

Any of the anti-CD38 antibodies, including human antibodies or humanized antibodies, can be prepared by conventional approaches, for example, hybridoma technology, antibody library screening, or recombinant technology. See, for example, Harlow and Lane, (1998) Antibodies: A Laboratory Manual, Cold Spring Harbor Laboratory, New York, WO 87/04462, Morrison et al., (1984) Proc. Nat. Acad. Sci. 81:6851, and Queen et al., Proc. Natl. Acad. Sci. USA, 86:10029-10033 (1989).

It should be understood that the described antibodies are only exemplary and that any anti-CD38 antibodies can be used in the compositions and methods disclosed herein. Methods for producing antibodies are known to those of skill in the art.

### III. Combined Therapy

In some aspects, the present disclosure provides a combined therapy involving any of the genetically engineered CAR-T cells and any of the NK cell inhibitors to improve a clinical outcome in a subject (e.g., a human patient) receiving the combined therapy. In some embodiments, the combined therapy comprises administering an effective amount of a population of genetically engineered CAR-T cells as disclosed herein to a subject who is receiving or has received an NK cell inhibitor as also disclosed herein. In other embodiments, the combined therapy comprises administering an effective amount of an NK cell inhibitor to a subject who is receiving or has received a population of genetically engineered CAR-T cells. In yet other embodiments, the combined therapy comprises administering to a subject an effective amount of a population of genetically engineered CAR-T cells and an effective amount of an NK cell inhibitor, either concurrently or sequentially.

A. Improving Clinical Outcome

As used herein, “clinical outcome” refers to one or more therapeutic effects (direct or indirect) in a subject resulting from the combined therapy that lead to an improved treatment efficacy relative to the CAR-T cell monotherapy and/or the NK cell inhibitor monotherapy. In some instances, an improved clinical outcome comprises one or more actual therapeutic benefits, for example, an increase of clinical response to the CAR-T cells (e.g., reduced disease burden, alleviation of one or more disease symptoms, and/or a prolonged survival rate). In the context of cancer therapy, therapeutic benefits may comprise reduced tumor volume, reduced tumor cell numbers, and/or increased anti-tumor responses. Alternatively, an improved clinical outcome comprises enhancement of one or more desired features and/or reduction of one or more undesired features, which ultimately lead to therapeutic benefits. For example, an improved clinical outcome may comprise an increase of persistence of the engineered human CAR-T cells in the subject, a decrease of CAR-T cell lysis, which may be induced by NK cells, a reduction of NK cells, or a combination thereof.

The clinical outcome of a treatment comprising a composition for the treatment of a medical condition can be determined by the skilled clinician. A treatment is considered “effective treatment,” if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Clinical outcome can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments, methods for increasing a clinical response in a subject receiving a CAR T cell therapy are provided, wherein the method comprises administering to the subject an effective amount of an NK cell inhibitor to a subject that has received or is receiving a CAR T cell therapy comprising a population of engineered human CAR T cells comprising a disrupted MHC class I complex.

In some embodiments, methods for increasing a clinical response in a subject receiving a CAR T cell therapy are provided, wherein the method comprises administering to the subject a CAR T cell therapy comprising a population of engineered human CAR T cells comprising a disrupted MHC class I complex, wherein the subject has received or is receiving an effective amount of an NK cell inhibitor.

In some embodiments, methods for increasing a clinical response in a subject receiving a CAR T cell therapy are provided, wherein the method comprises administering to the subject an effective amount of (a) a CAR T cell therapy comprising a population of engineered human CAR T cells comprising a disrupted MHC class I complex; and (b) an NK cell inhibitor. In some embodiments, the increase in clinical response is relative to a therapy comprising the CAR T cell therapy alone, or relative to a therapy comprising the NK cell inhibitor alone. In some embodiments, the increase in clinical response is additive or synergistic.

In some embodiments, a clinical response is a reduction in tumor volume or tumor size. In some embodiments, a clinical response is an inhibition of tumor growth. In some embodiments, a clinical response is reduction in tumor cell numbers. In some embodiments, a clinical response is the period of remission. In some embodiments, an NK cell inhibitor increases a clinical response in a subject receiving CAR T cell therapy compared to a subject receiving CAR T cell therapy in the absence of an NK cell inhibitor.

In some embodiments, the disclosure provides methods for increasing the anti-tumor response of a CAR T cell therapy in a subject by administering an NK cell inhibitor.

In some embodiments, methods for increasing anti-tumor response of a CAR T cell therapy in a subject are provided, wherein the method comprises administering to the subject a CAR T cell therapy comprising a population of engineered human CAR T cells comprising a disrupted MHC class I complex, wherein the subject has received or is receiving an effective amount of an NK cell inhibitor. In some embodiments, increased anti-tumor response is an increase in cytotoxic molecules. In some embodiments, increased anti-tumor response is an increase in Granzyme B activity in tumor cells, or an increase in tumor cell numbers or tumor cell fraction with Granzyme B expression. In some embodiments, increased anti-tumor response is an increase in secretion of IFN-γ cytokine by immune cells or by engineered human CAR T cells. In some embodiments, increased anti-tumor response is an increase in tumor cell killing mediated by immune cells or by engineered human CAR T cells.

In some embodiments, increasing clinical response of CAR T cell therapy efficacy is increased anti-tumor activity, increased persistence of engineered CAR T cells, decreased cell lysis of engineered CAR T cells, or extended remission. In some embodiments, improved clinical outcome is an increase in overall survival or progression free survival, decreased time receiving therapy, increased maximal therapy response, increased clinical benefit (e.g., stable disease, partial response, or complete response to therapy), reduced adverse events, or combinations thereof. In some embodiments, the disclosure provides methods of improving engineered human CAR T cells persistence in subjects receiving CAR T cell therapy by administering an NK cell inhibitor.

In some embodiments, methods for reducing NK cell activity in a subject receiving a CAR T cell therapy are provided, wherein the method comprises administering to the subject a CAR T cell therapy comprising a population of engineered human CAR T cells comprising a disrupted MHC class I complex, wherein the subject has received or is receiving an effective amount of an NK cell inhibitor.

In some embodiments, methods for reducing NK cell activity in a subject receiving a CAR T cell therapy are provided, wherein the method comprises administering to the subject an effective amount of an NK cell inhibitor to a subject that has received or is receiving a CAR T cell therapy comprising a population of engineered human CAR T cells comprising a disrupted MHC class I complex.

In some embodiments, methods for reducing NK cell activity in a subject are provided, wherein the method comprises administering to the subject an effective amount of (a) an NK cell inhibitor; and (b) a CAR T cell therapy comprising a population of engineered human CAR T cells comprising a disrupted MHC class I complex.

In some embodiments, an NK cell inhibitor is administered before the administration of the engineered human CAR T cells. In some embodiments, an NK cell inhibitor is administered concurrently with the administration of the engineered human CAR T cells. In some embodiments, an NK cell inhibitor is administered after the administration of the engineered human CAR T cells. In some embodiments, the engineered human CAR T cells is given in combination with an NK cell inhibitor, wherein the engineered human CAR T cells is given before, concurrent, or after the administration of the NK cell inhibitor.

In some embodiments, more than one dose of an NK cell inhibitor is administered to a subject. In some embodiments, an initial dose of an NK cell inhibitor is administered to a subject prior to, concurrently with, or after CAR T cell therapy. In some embodiments, at least one subsequent dose of an NK cell inhibitor is administered. In some embodiments, the same NK cell inhibitor is administered at the initial dose and at a subsequent dose. In some embodiments, different NK cell inhibitors are administered at the initial dose and at subsequent dose(s). In some embodiments, the subsequent dose of the NK inhibitor is administered when NK cell numbers in the subject recover to about 10%, 20%, 30%, 40%, 50%, 60%, 70%, 80%, 90%, or 100% of NK cell numbers prior to the administration of the NK cell inhibitor. In some embodiments, NK cell numbers are measured 1, 2, 3, 4, 5, 6, 7, and/or 8 weeks after administration of a NK cell inhibitor. In some embodiments, NK cell numbers are measured 3, 4, 5, 6, 7, 8, and/or 12 months after administration of a NK cell inhibitor. In some embodiments, NK cell numbers are measured 1, 2, 3, 4, 5, 6, 7, and/or 8 weeks after administration of CAR T therapy. In some embodiments, a subsequent dose of an NK cell inhibitor maintains persistence of engineered human T cells.

B. Administration of Genetically Engineered CAR-T Cells and NK Cell Inhibitors

Any of the genetically engineered CAR-T cells disclosed herein can be co-used with any of the NK cell inhibitors also disclosed herein in the combined therapy. Such genetically engineered CAR-T cells and NK cell inhibitors may be given to a subject in need of the treatment via a suitable route, for example, intravenous infusion.

In some embodiments, the engineered T cells comprise a population of cells comprising engineered human T cells comprising: (i) a TRAC gene disrupted by insertion of a nucleic acid encoding a chimeric antigen receptor (CAR) having the amino acid sequence set forth in SEQ ID NO 40; and (ii) a disrupted β2M gene. In some embodiments, the nucleic acid encoding the CAR comprises the nucleotide sequence set forth in SEQ ID NO: 39. In some embodiments, the disrupted TRAC gene comprises a deletion of the nucleotide sequence of SEQ ID NO: 28. In some embodiments, the disrupted TRAC gene comprises the nucleotide sequence set forth in SEQ ID NO: 58. In some embodiments, less than 0.5% of the engineered T cells express a detectable level of TCR surface protein. In some embodiments, less than 30% of the engineered T cells express a detectable level of β2M surface protein. In some embodiments, at least 50% the engineered T cells express a detectable level of CAR surface protein. In some embodiments, the engineered human T cells are allogeneic.

In some embodiments, the engineered T cells comprise a population of cells comprising engineered human T cells comprising: (i) a TRAC gene disrupted by insertion of a nucleic acid encoding a chimeric antigen receptor (CAR) having the amino acid sequence set forth in SEQ ID NO 76; and (ii) a disrupted β2M gene. In some embodiments, the nucleic acid encoding the CAR comprises the nucleotide sequence set forth in SEQ ID NO:75. In some embodiments, the disrupted TRAC gene comprises a deletion of the nucleotide sequence of SEQ ID NO: 28. In some embodiments, the disrupted TRAC gene comprises the sequence set forth in SEQ ID NO: 74. In some embodiments, less than 0.5% of the engineered T cells express a detectable level of TCR surface protein. In some embodiments, less than 30% of the engineered T cells express a detectable level of β2M surface protein. In some embodiments, at least 50% the engineered T cells express a detectable level of CAR surface protein. In some embodiments, the engineered human T cells are allogeneic.

In some embodiments, the engineered T cells comprise a population of cells comprising engineered human T cells comprising: (i) a TRAC gene disrupted by insertion of a nucleic acid encoding a chimeric antigen receptor (CAR) having the amino acid sequence set forth in SEQ ID NO 86; and (ii) a disrupted β2M gene. In some embodiments, the nucleic acid encoding the CAR comprises the nucleotide sequence set forth in SEQ ID NO: 85. In some embodiments, the disrupted TRAC gene comprises a deletion of the nucleotide sequence of SEQ ID NO: 28. In some embodiments, the disrupted TRAC gene comprises the sequence set forth in SEQ ID NO: 84. In some embodiments, less than 0.5% of the engineered T cells express a detectable level of TCR surface protein. In some embodiments, less than 30% of the engineered T cells express a detectable level of β2M surface protein. In some embodiments, at least 50% the engineered T cells express a detectable level of CAR surface protein. In some embodiments, the engineered human T cells are allogeneic.

In some embodiments, the engineered T cells comprise a population of cells comprising engineered human T cells comprising: (i) a TRAC gene disrupted by insertion of a nucleic acid encoding a chimeric antigen receptor (CAR) having the amino acid sequence set forth in SEQ ID NO: 115; and (ii) a disrupted β2M gene. In some embodiments, the nucleic acid encoding the CAR comprises the nucleotide sequence set forth in SEQ ID NO: 112. In some embodiments, the disrupted TRAC gene comprises a deletion of the nucleotide sequence of SEQ ID NO: 28. In some embodiments, the disrupted TRAC gene comprises the sequence set forth in SEQ ID NO: 111. In some embodiments, less than 0.5% of the engineered T cells express a detectable level of TCR surface protein. In some embodiments, less than 30% of the engineered T cells express a detectable level of β2M surface protein. In some embodiments, at least 50% the engineered T cells express a detectable level of CAR surface protein. In some embodiments, the engineered human T cells are allogeneic.

The combined therapy disclosed herein may involve any of the above specific genetically engineered CAR-T cells and an NK cell inhibitor, for example, daratumumab or a functional variant thereof.

The step of administering CAR T cell therapy may include the placement (e.g., transplantation) of cells, e.g., engineered human CAR T cells, into a subject, by a method or route that results in at least partial localization of the introduced cells at a desired site, such as tumor, such that a desired effect(s) is produced. Engineered T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. For example, in some aspects described herein, an effective amount of engineered T cells is administered via a systemic route of administration, such as an intraperitoneal or intravenous route.

A subject may be any subject for whom treatment or therapy is desired. In some embodiments, the subject is a mammal. In some embodiments, the subject is a human.

In some embodiments, an engineered human CAR T cell population and/or NK cell inhibitor being administered according to the methods described herein does not induce toxicity in the subject, e.g., the engineered human CAR T cells and/or NK cell inhibitors do not induce toxicity in non-cancer cells. In some embodiments, an engineered human CAR T cell population and/or NK cell inhibitor being administered does not trigger complement mediated lysis or does not stimulate antibody-dependent cell mediated cytotoxicity (ADCC). In some embodiments, an engineered human CAR T cell population and/or NK cell inhibitor being administered does not trigger apoptosis. In some embodiments, an engineered human CAR T cell population and/or NK cell inhibitor being administered does not trigger ADCP.

An effective amount refers to the amount of a population of engineered human CAR T cells and/or an NK cell inhibitor needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. In some embodiments, an effective amount of an NK cell inhibitor refers to the amount needed to prevent or alleviate at least one of NK cell-mediated lysis of engineered human CAR T cells or an NK cell activity. In some embodiments, an effective amount of an NK cell inhibitor refers to the amount needed to allow for persistence of engineered human CAR T cells.

It is understood that for any given case, an appropriate effective amount can be determined by one of ordinary skill in the art using routine experimentation.

In some embodiments, a subject is administered a population of cells comprising engineered T cells (e.g., engineered human CAR T cells) at a dose of about 1×10⁷, 2×10⁷, 3×10⁷, 4×10⁷, 5×10⁷, 6×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, or 1×10⁹ engineered T cells expressing a detectable level of CAR described herein. In some embodiments, a subject is administered a population of cells comprising engineered T cells (e.g., engineered human CAR T cells) at a dose of about 1×10⁷, 3×10⁷, 1×10⁸, 3×10⁸, or 1×10⁹ engineered T cells expressing a detectable level of CAR described herein. In some embodiments, a subject is administered a population of cells comprising engineered T cells (e.g., engineered human T cells) at a dose of about 3×10⁷, 1×10⁸ or 3×10⁸ engineered T cells expressing a detectable level of CAR described herein. In some embodiments, a subject is administered a population of cells comprising engineered T cells (e.g., engineered human CAR T cells) at a dose of about 1×10⁷-3×10⁸ engineered T cells expressing a detectable level of CAR described herein.

In some embodiments, a subject is administered a population of cells comprising engineered T cells (e.g., engineered human CAR T cells) at a dose of about 1×10⁷ engineered T cells expressing a detectable level of CAR described herein. In some embodiments, a subject is administered a population of cells comprising engineered T cells (e.g., engineered human CAR T cells) at a dose of about 3×10⁷ engineered T cells expressing a detectable level of CAR described herein. In some embodiments, a subject is administered a population of cells comprising engineered T cells (e.g., engineered human CAR T cells) at a dose of about 1×10⁸ engineered T cells expressing a detectable level of CAR described herein. In some embodiments, a subject is administered a population of cells comprising engineered T cells (e.g., engineered human T cells) at a dose of about 3×10⁸ engineered T cells expressing a detectable level of CAR described herein. In some embodiments, a subject is administered a population of cells comprising engineered T cells (e.g., engineered human T cells) at a dose of about 1×10⁹ engineered T cells expressing a detectable level of CAR described herein.

In some embodiments, a subject is administered cells at a dose of about 1×10⁷ engineered human CAR T cells expressing a detectable level of CAR (e.g., CD19, CD33, CD70, or BCMA antigen binding domain). In some embodiments, a subject is administered cells at a dose of about 3×10⁷ engineered human CAR T cells expressing a detectable level of CAR (e.g., CD19, CD33, CD70, or BCMA antigen binding domain). In some embodiments, a subject is administered cells at a dose of about 1×10⁸ engineered human CAR T cells expressing a detectable level of CAR (e.g., CD19, CD33, CD70, or BCMA antigen binding domain). In some embodiments, a subject is administered cells at a dose of about 3×10⁸ engineered human CAR T cells expressing a detectable level of CAR (e.g., CD19, CD33, CD70, or BCMA antigen binding domain).

In some embodiments, the cells are derived from one or more donors. In some examples described herein, the cells are expanded in culture prior to administration to a subject in need thereof.

Modes of administration for engineered human CAR T cells and/or NK cell inhibitors include injection and infusion. Injection includes, without limitation, intravenous, intrathecal, intraperitoneal, intraspinal, intracerebro spinal, and intrasternal infusion. In some embodiments, the route is intravenous.

In some embodiments, engineered human CAR T cells and/or NK cell inhibitors are administered systemically, which refers to the administration other than directly into a target site, tissue, or organ, such that it enters, instead, the subject's circulatory system and, thus, is subject to metabolism and other like processes.

C. Target Cancers

In some embodiments, provided herein are methods for treating cancer (e.g., leukemias, e.g., acute myeloid leukemia). In some embodiments, the methods comprise delivering CAR T cell therapy as described herein to a subject (e.g., a human patient) having a cancer.

Non-limiting examples of cancers that may be treated as provided herein include multiple myeloma, leukemia, and/or clear cell renal cell carcinoma (ccRCC). Non-limiting examples of leukemias that may be treated as provided herein include T cell leukemia, chronic lymphocytic leukemia (CLL), Hodgkin's lymphoma, T cell lymphoma, non-Hodgkin lymphomas (e.g., diffuse large B-cell lymphoma (DLBCL), high grade B-cell lymphoma, transformed follicular lymphoma (FL), grade 3B FL, and Richter's transformation of CLL), and acute lymphoblastic leukemia (ALL). In some embodiment, the methods comprise delivering the CAR T cells (e.g., anti-BCMA, anti-CD19, anti-CD33 and/or anti-CD70 CAR T cells) of the present disclosure to a subject having multiple myeloma, leukemia, or lymphoma. Other non-limiting examples of cancers (e.g., solid tumors) that may be treated as provided herein include pancreatic cancer, gastric cancer, ovarian cancer, cervical cancer, breast cancer, renal cancer, thyroid cancer, nasopharyngeal cancer, non-small cell lung (NSCLC), glioblastoma, and/or melanoma.

In some embodiments, the disclosure provides methods for treating a non-Hodgkin lymphoma (NHL) in a human patient by administering an intravenous dose of about 1×10⁷-3×10⁸, or of about 1×10⁷-1×10⁹ engineered human T cells expressing a detectable level of CAR described herein (e.g., anti-CD19 CAR). In some embodiments, the disclosure provides methods for treating a non-Hodgkin lymphoma (NHL) in a human patient by administering an intravenous dose of about 3×10⁷ engineered human T cells expressing a detectable level of CAR described herein (e.g., anti-CD19 CAR). In some embodiments, the disclosure provides methods for treating a non-Hodgkin lymphoma (NHL) in a human patient by administering an intravenous dose of about 1×10⁸ engineered human T cells expressing a detectable level of CAR described herein (e.g., anti-CD19 CAR). In some embodiments, the disclosure provides methods for treating a non-Hodgkin lymphoma (NHL) in a human patient by administering an intravenous dose of about 3×10⁸ engineered human T cells expressing a detectable level of CAR described herein (e.g., anti-CD19 CAR). In some embodiments, the disclosure provides methods for treating a non-Hodgkin lymphoma (NHL) in a human patient by administering an intravenous dose of about 1×10⁹ engineered human T cells expressing a detectable level of CAR described herein (e.g., anti-CD19 CAR).

In some embodiments, the disclosure provides methods for treating a non-Hodgkin lymphoma (NHL) in a human patient by intravenously administering cells at a dose of about 1×10⁷-3×10⁸, or a dose of about 1×10⁷-1×10⁹ engineered human CAR T cells expressing a detectable level of anti-CD19 CAR, in combination with any of the NK inhibitors disclosed herein such as daratumumab.

In some embodiments, the disclosure provides methods for treating multiple myeloma (MM) in a human patient by intravenously administering cells at a dose of about 2.5×10⁷-4.5×10⁸ engineered human CAR T cells expressing a detectable level of anti-BCMA CAR, in combination with daratumumab.

In some embodiments, the disclosure provides methods for treating a solid tumor such as renal cell carcinoma or a T cell or B cell malignancy in a human patient by intravenously administering cells at a dose of about 1.0×10⁷-1×10⁹ engineered human CAR T cells expressing a detectable level of anti-CD70 CAR, in combination with daratumumab.

In some aspects, the disclosure provides methods for administering the population of cells or an engineered CAR T cells described herein to a subject. In some aspects, the engineered T cells are engineered human T cells. In some aspects, the subject has cancer. In some aspects, the cancer expresses CD70, BMCA, CD19, CD33 or combinations thereof. In some aspects, the population of cells is administered to the subject in an amount effective to treat the cancer. In some aspects, the cancer is a solid tumor malignancy or a hematological malignancy. In some aspects, the solid tumor malignancy is selected from the group consisting of: ovarian tumor, pancreatic tumor, kidney tumor, lung tumor, and intestinal tumor. In some aspects, the population of cells is administered to the subject in an amount effective to reduce the volume of a tumor or tumor cell number in the subject.

In some aspects, the disclosure provides a method for treating cancer in a subject, comprising administering the population of cells or an engineered human CAR T cells described herein to a subject.

In some embodiments, an engineered human CAR T cell population being administered according to the methods described herein comprises allogeneic T cells obtained from one or more donors. Allogeneic refers to a cell, cell population, or biological samples comprising cells, obtained from one or more different donors of the same species, where the genes at one or more loci are not identical to the recipient. For example, an engineered CAR T cell population, being administered to a subject can be derived from T cells from one or more unrelated donors, or from one or more non-identical siblings. In some embodiments, syngeneic cell populations may be used, such as those obtained from genetically identical donors, (e.g., identical twins).

In some embodiments, the engineered human CAR T cells are allogeneic. In some embodiments, the engineered human CAR T cells are administered as an allogeneic transplant.

D. Reduction of CAR-T Cell Therapy Toxicity

Risk of tumor relapse with tumor-targeting CAR T cell therapy is thought to be due, in part, to limited persistence of CAR T cells in a subject following administration (Maude, S., et al. (2014) N Engl J Med. 371:1507-1517; Turtle, C. et al., (2016) J Clin Invest. 126:2123-2138). The combined therapy disclosed herein also aims at reducing toxicity associated with CAR-T cell therapy. Examples include host-versus-graft disease (HvGD) and graft-versus-host diseases (GvHD).

(i) Host-Versus-Graft Disease (HvGD)

In some embodiments, the engineered T cells described herein have a prolonged persistence due to a reduced or minimal host-versus-graft (HvG) response. A HvG response is defined as a functional and structural deterioration of an allogeneic graft (e.g., allogeneic donor T cell) due to an active immune response by the recipient. A source of a HvG response are antigens on an allogeneic donor cell that activate the immune system of the host. Allogeneic major histocompatibility antigens (e.g., termed human leukocyte antigens or HLAs in humans) expressed by a donor cell are strong inducers of a HvG response (Ingulli, E. (2010) Pediatr Nephrol., 25:61-74; Alelign, T. et al., (2018) J Immunol Research Article ID 5986740). Approximately 1-10% of T cells in a subject express a TCR that can recognize allogeneic HLA complexes. Recognition can be direct, wherein a host T cell expresses a TCR that directly recognizes an allogeneic HLA molecule present on a donor cell. Recognition can be indirect, wherein a donor cell is first internalized and processed by a host antigen presenting cell (APC), and a host T cell recognizes the allogeneic HLA molecule that is presented by the host APC in peptide form. Host T cells that recognize allogeneic antigens on donor cells and are activated will trigger a HvG response. Elimination of allogeneic antigens from a donor cell prior to transplantation can eliminate or reduce the risk of a HvG response, thereby increasing persistence following administration.

Accordingly, in some embodiments, the population of engineered human CAR T cells of the present disclosure is engineered by use of CRISPR-Cas9 gene-editing to induce a site-specific disruption in a target gene sequence that eliminates the expression of an allogeneic antigen. In some embodiments, an allogeneic antigen is a major histocompatibility antigen. In some embodiments, a major histocompatibility antigen is an MHC Class I complex. In some embodiments, the target gene sequence is found in the B2M gene that encodes a protein component of the MHC Class I complex. In some embodiments, genetic disruption eliminates or reduces the risk of host versus engineered CAR T cell response and gives increased engineered T cell persistence following administration in a recipient.

In some embodiments, persistence of engineered T cells (e.g., engineered human CAR T cells) is assessed by analyzing the presence and quantity of engineered human CAR T cells present in one or more tissue samples that are collected from a subject following engineered human CAR T cell administration. In some embodiments, persistence is defined as the longest duration of time from administration to a time wherein a detectable level of engineered T cells is present in a given tissue type (e.g., peripheral blood).

In some embodiments, persistence is defined as the continued absence of disease (e.g., complete response or partial response). Determination of the absence of disease and response to treatment are known to those of skill in the art and described herein.

Methods of appropriate tissue collection, preparation, and storage are known to one skilled in the art. In some embodiments, persistence of engineered T cells (e.g., engineered human CAR T cells) is assessed in one or more tissue samples from a group comprised of peripheral blood, cerebrospinal fluid, tumor, skin, bone, bone marrow, breast, kidney, liver, lung, lymph node, spleen, gastrointestinal tract, tonsils, thymus and prostate. In some embodiments, a quantity of engineered T cells (e.g., engineered human CAR T cells) is measured in a single type of tissue sample (e.g., peripheral blood). In some embodiments, a quantity of engineered T cells (e.g., engineered human CAR T cells) is measured in multiple tissue types (e.g., peripheral blood in addition to bone marrow and cerebrospinal fluid). By measuring quantity of engineered human CAR T cells in multiple tissue types, the distribution of engineered human CAR T cells throughout different tissues of the body can be determined. In some embodiments, a quantity of engineered T cells (e.g., engineered human CAR T cells) is measured in one or more tissue samples at a single time point following administration. In some embodiments, a quantity of engineered T cells (e.g., engineered human CAR T cells) is measured in one or more tissue samples at multiple time points following administration. In some embodiments, a quantity of engineered T cells (e.g., engineered human CAR T cells) present in one or more tissue samples at a time following administration is compared to a sample of the same tissue type collected from the patient prior to administration.

A detectable level of engineered T cells (e.g., engineered human CAR T cells) in a given tissue can be measured by known methodologies. Methods for assessing the presence or quantity of engineered T cells in a tissue of interest are known to those of skill in the art. Such methods include, but are not limited to, reverse transcription polymerase chain reaction (RT-PCR), competitive RT-PCR, real-time RT-PCR, RNase protection assay (RPA), quantitative immunofluorescence (QIF), flow cytometry, northern blotting, nucleic acid microarray using DNA, western blotting, enzyme-linked immunosorbent assay (ELISA), radioimmunoassay (RIA), tissue immunostaining, immunoprecipitation assay, complement fixation assay, fluorescence-activated cell sorting (FACS), mass spectrometry, magnetic bead-antibody immunoprecipitation, or protein chip.

As used herein, in some embodiments, persistence is the longest period from the time of administration to a time wherein a detectable level of engineered T cells (e.g., engineered human CAR T cells) is measured. In some embodiments, a detectable level of engineered T cells (e.g., engineered human CAR T cells) is defined in terms of the limit of detection of a method of analysis. The limit of detection can be defined as the lowest quantity of a component or substance that can be reliably and reproducibly measured by an analytical procedure when compared to a tissue sample expected to have no quantity of the component or substance of interest. A non-limiting exemplary method to determine a reproducible limit of detection is to measure the analytical signal for replicates of a zero calibrator relative to a blank sample (Armbruster, D. et al. (2008) Clin Biochem Rev. 29:S49-S52). A blank sample is known to be devoid of an analyte of interest. A zero calibrator is the highest dilution of a test sample of known concentration or quantity that gives analytical signal above that measured for the blank sample. By quantifying the analytical signal for at least 5, 6, 7, 8, 9, 10, 11, 12, 13, 14, 15, 16, 17, 18, 19, 20, 21, 22, 23, 24, 25, 26, 27, 28, 29, or 30 replicates of a zero calibrator, one can determine an average and standard deviation (SD) for the limit of detection of an analytical method of interest. Selection of a method with a suitable limit of detection for quantifying engineered T cells in a given tissue can be ascertained by one skilled in the art. In some embodiments, a detectable level of engineered T cells (e.g., engineered human CAR T cells) is any quantity of engineered T cells in a tissue sample that gives an analytical signal above the limit of detection for a method of analysis. In some embodiments, a detectable level of engineered T cells (e.g., engineered human CAR T cells) is any quantity of engineered T cells in a tissue sample that gives an analytical signal that is at least 2 SDs, 3 SDs, 4 SDs, 5 SDs, 6 SDs, 7 SDs, 8 SDs, 9 SDs, or 10 SDs, above the limit of detection for the method of analysis.

It is known that CAR-expressing engineered T cells can undergo expansion following administration to a recipient. Expansion is a response to antigen recognition and signal activation (Savoldo, B. et al. (2011) J Clin Invest. 121:1822; van der Stegen, S. et al. (2015) Nat Rev Drug Discov. 14:499-509). In some embodiments, following expansion, engineered T cells undergo a contraction period, wherein a portion of the engineered T cell population that are short-lived effector cells are eliminated and what remains is a portion of the engineered T cell population that are long-lived memory cells. In some embodiments, persistence is a measure of the longevity of the engineered T cell population following expansion and contraction. The duration of the expansion, contraction and persistence phases are evaluated using a pharmacokinetic profile. In some embodiments, a pharmacokinetic (PK) profile is a description of the quantity of engineered T cells (e.g., engineered human CAR T cells) measured in a given tissue over time and is readily ascertained by one skilled in the art by measuring the quantity of engineered T cells in a given tissue (e.g., peripheral blood) at multiple time points. In some embodiments, a measure of a PK profile provides a method of evaluating a subject. In some embodiments, a measure of a PK profile provides a method of evaluating or monitoring the effectiveness of engineered T cell therapy (e.g., engineered human CAR T cells) in a subject having cancer. In some embodiments, a measure of a PK profile provides a method of evaluating the persistence of engineered T cells (e.g., engineered human CAR T cells) in a subject. In some embodiments, a PK profile provides a method of evaluating the expansion of engineered T cells (e.g., engineered human CAR T cells in a subject. In some embodiments, a measure of persistence of engineered T cells (e.g., engineered human CAR T cells) in a subject is used to evaluate the effectiveness of engineered T cell therapy in a subject. In some embodiments, a measure of expansion of engineered T cells (e.g., engineered human CAR T cells) in a subject is used to evaluate the effectiveness of engineered T cell therapy in a subject.

In some embodiments, a PK profile is prepared by measuring a quantity of engineered T cells (e.g., engineered human CAR T cells) in a sample of a given tissue type (e.g., peripheral blood) collected from a recipient and repeating the assessment at different time points. In some embodiments, a baseline tissue sample is collected from a recipient no more than 1 day, 2 days, 3 days, 4 days, 5 days, 6 days, 7 days, 8 days, 9 days, 10 days, 12 days, 13 days, 14 days, or 15 days prior to administration. In some embodiments, tissue collection from a recipient is performed within 0.25-2 hours, within 1-3 hours, within 2-6 hours, within 3-11 hours, within 4-20 hours, within 5-48 hours of the time of administration of engineered T cells. In some embodiments, tissue collection from a recipient is performed on a daily basis starting on day 1, day 2, day 3, or day 4 and continuing through at least day 5, day 6, day 7, day 8, day 9, day 10, day 11, day 12, day 13, day 14, day 15, day 16, day 17, day 18, day 19, or day 20. In some embodiments, tissue collection from a recipient is performed at least 1 time, 2 times, 3 times, 4 times, 5 times, or 6 times per week for up to 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, or 16 weeks following administration of engineered T cells. In some embodiments, tissue collection from a recipient is performed at least 1 time, 2 times, 3 times, 4 times, 5 times, or 6 times per month for up to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, or 24 months following administration of engineered T cells. In some embodiments, tissue collection from a recipient is performed at least 1 time, 2 times, 3 times, 4 times, 5 times, or 6 times per year for up to 1 year, 2 years, 3 years, 4 years, 5 years, 6 year, 7 years, 8 years, 9 years, or 10 years following administration of engineered T cells.

In some embodiments, engineered T cell persistence is defined as the duration of time from administration wherein a quantity of engineered T cells (e.g., engineered human CAR T cells) is present that is at least 0.005-0.05%, 0.01-0.1%, 0.05-0.5%, 0.1-1%, 0.5%-5%, or 1-10% of the peak quantity of engineered T cells. In some embodiments, a persistence of engineered T cells is determined by comparing the quantity of engineered T cells (e.g., engineered human CAR T cells) measured in a given tissue type (e.g., peripheral blood) to the peak quantity of engineered T cells that is measured in the same tissue type. In some embodiments, a persistence of engineered T cells is determined by comparing the quantity of engineered T cells (e.g., engineered human CAR T cells) measured in a given tissue type (e.g., peripheral blood) to the peak quantity of engineered T cells that is measured in a different tissue type. In some embodiments, a persistence of engineered T cells is determined by comparing the quantity of engineered T cells (e.g., engineered human CAR T cells) measured in a given subject (e.g., peripheral blood) to the peak quantity of engineered T cells that is measured in the same subject. In some embodiments, a persistence of engineered T cells is determined by comparing the quantity of engineered T cells (e.g., engineered human CAR T cells) measured in a given subject (e.g., peripheral blood) to the peak quantity of engineered T cells that is measured in a different subject (e.g., a subject with partial response, a subject with complete response).

In some embodiments, a persistence of engineered T cells is present in one or more tissue types (e.g., peripheral blood) following administration wherein engineered T cells (e.g., engineered human T cells expressing a CAR) are administered on day 1. In some embodiments, a persistence of engineered T cells is present in one or more tissue types (e.g., peripheral blood) up to 1 day, 2 days, 3 days, 4, days, 5 days, 6 days, 7 days, 8 days, 9 days, 10 days, 11 days, 12 days, 13 days, 14 days, 15 days, 16 days, 17 days, 18 days, 19 days, 21 days, 21 days, 22 days, 23 days, 24 days, 25 days, 26 days, 27 days, 28 days, 29 days, 30 days, 31 days, 32 days, 33 days, 34 days, or 35 days following administration wherein engineered T cells (e.g., engineered human CAR T cells) are administered on day 1. In some embodiments, a persistence of engineered T cells is present in one or more tissue types (e.g. peripheral blood) up to 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 21 months, 22 months, 23 months, or 24 months following administration of engineered T cells (e.g., engineered human CAR T cells). In some embodiments, a persistence of engineered T cells is measured in one or more tissue types (e.g., peripheral blood) up to 1 year, 2 years, 3 years, 4 years, 5 years, 6 years, 7 years, 8 years, 9 years, and 10 years following administration of engineered T cells. In some embodiments, a persistence of engineered T cells that is at least 10-25 days, at least 25-50 days, at least 50-100 days, at least 100-364 days, at least one year, at least two years, at least three years, at least four years or at least five years from administration wherein engineered T cells are administered on day 1 is indicative of a response in a recipient (e.g., complete response or partial response).

In some embodiments, an area under the curve (AUC) is defined as a total area under the curve of quantity of engineered T cells (e.g., engineered human CAR T cells) measured over time in a given tissue type (e.g., peripheral blood). A method of calculating an AUC is known to one skilled in the art and is comprised of approximating an AUC by a series of trapezoids, computing the area of the trapezoids, and summing the area of the trapezoids to determine the AUC. In some embodiments, an AUC is defined for a PK profile wherein the quantity of engineered T cells (e.g., engineered human CAR T cells) is measured for a given tissue type over time. In some embodiments, an AUC is defined for a PK profile from one designated time point to another designated time point (i.e., AUC10-80 refers to the total area under a quantity-time curve depicting quantity from day 10 to day 80 following administration). In some embodiments, an AUC is determined for a preselected time period extending from time of administration (e.g., day 1) to a time ending on a day that is 10-20 days, 15-45 days, 20-70 days, 25-100 days, or 40-180 days following administration. In some embodiments, an AUC measured for a PK profile in a recipient is indicative of a response in the recipient (e.g., CR or PR). In some embodiments, an AUC measured for a PK profile in a recipient is indicative of a risk of relapse in the recipient.

(ii) Graft-Versus-Host Disease (GvHD)

Most CAR T cell therapies developed to-date use autologous CAR T cells (i.e., CAR T cells derived by genetic modification of a patient's own T cells). Use of allogeneic CAR T cells (i.e., CAR T cells derived by genetic modification of T cells from a genetically dissimilar human donor) carries a risk of inducing graft-versus-host disease (GvHD) since donor T cells express T cell receptors (TCRs) that are potentially reactive towards host tissue antigens and/or histocompatibility antigens, known as human leukocyte antigens or HLAs in humans. GvHD is a syndrome that occurs following transplant of allogeneic cells wherein immunocompetent donor cells (the graft) recognize and attack tissues in an immunocompromised allogeneic recipient (the host) (Barnes, D. et al., (1962) Ann NY Acad. Sci. 99:374-385; Billingham, R. (1966) Harvey Lect. 62:21-78). Substantial clinical evidence has emphasized the risk of GvHD due to administration of allogeneic T cell transplants, particularly in the setting of hematopoietic stem cell transplants (HSCTs).

HSCTs are administered as a part of a therapeutic regimen for patients suffering from refractory haematological malignancies such as leukemia or lymphoma. A substantial therapeutic effect of allogeneic HSCT (i.e., HSCT from an individual not genetically identical to the host patient) is the graft-versus-tumor (GVT) effect. Donor T cells administered in the HSCT transplant are a major mediator of the GVT effect in that some will recognize tumor antigens and allogeneic HLA antigens on tumor cells as foreign and eliminate the tumor cells (Kolb, H. (2008) Blood 112:4371-4383). However, transplanted donor T cells also have the potential to trigger GvHD by recognizing host HLAs as foreign and responding by causing tissue damage in organs such as the skin, gut, liver, and lungs. Removal of T cells from the HSCT preparation prior to transplantation can greatly reduce the occurrence of GvHD, but the risk of tumor relapse and graft failure is markedly increased (Apperley, J. et al. (1988) Br J Haematol. 69:239-245; Martin, P. et al., (1985) Blood 66:664-672; Patterson, J. et al. (1986) Br J Haematol. 63:221-230). Thus, transplantation of engineered allogeneic T cells (e.g., engineered human T cells expressing an anti-CD19, anti-CD33, anti-CD70, or BCMA CAR) has beneficial as well as deleterious outcomes: transplanted host-reactive T cells will facilitate beneficial GVT effects, but also cause destruction of healthy tissues that results in GvHD.

Chimeric antigen receptor (CAR) T cell therapies offer substantial improvement for the use of exogenously administered T cells to induce GVT effects. CARs provide modified T cells with high affinity recognition of tumor antigens, but without the requirement for HLA presentation of antigen. Additionally, the CAR provides costimulatory components that contribute to T cell activation upon antigen recognition (Sadelain M., et al. (2013) Cancer Discov. 3:388-398; Davila M., et al. (2012) Oncoimmunology 1:1577-1583). However, most CAR T cell products are produced from a mixed population of T cell clones that retain expression of the TCR and thus, have a risk of inducing GvHD when administered to a host that is genetically non-identical to the donor. To avoid the risk of inducing GvHD, the majority of CAR T cell therapies developed to-date use autologous T cells such that the TCR expressed by the CAR T cell is not reactive towards host antigens (Maude, S. et al. (2014) N Engl J Med. 371:1507-1517; Neelapu, S. et al. (2017) N Engl J Med. 377:2531-2544; Schuster, S. et al. (2017) N Engl J Med. 377:2545-2554). However, the production of autologous CAR T cells is costly and time-intensive, resulting in some patients experiencing disease progression or death while awaiting treatment.

In some embodiments, the present disclosure relates to an administration of a population of engineered T cells (e.g., CAR T cells) with a disrupted TCR and MHC, thereby reducing the risk of GvHD in the recipient patient. In some embodiments, CRISPR-Cas9 gene-editing components are used to introduce a site-specific disruption at a gene sequence that is associated with GvHD, such as the TCR and/or MCH. In some embodiments, the gene-sequence is selected from a component of the TCR. In some embodiments, the TCR component is a TRAC. In some embodiments, the site-specific disruption is a permanent deletion of at least a portion of the gene. In some embodiments, the site-specific disruption is a small deletion in the gene. In some embodiments, the site-specific disruption is a small insertion in the gene. In some embodiments, the site-specific disruption is an insertion of a nucleic acid encoding a CAR in the gene. In some embodiments, a site-specific disruption of the TRAC gene provides a T cell without a functional TCR. In some embodiments, a site-specific disruption of the TRAC locus reduces or eliminates the ability of the T cells to induce GvHD in an allogeneic recipient. In some embodiments, a site-specific disruption of a TRAC reduces the risk of GvHD following administration of allogeneic T cells in a subject.

In some embodiments, the present disclosure relates to administration of a population of cells comprising engineered T cells (e.g., engineered human CAR T cells) with a reduced risk of inducing GvHD in the recipient patient. In some embodiments, CRISPR-Cas9 gene-editing components are used to introduce a site-specific disruption at a TRAC locus. In some embodiments, a site-specific disruption in the TRAC locus is an insertion of a nucleic acid encoding a CAR in the gene. In some embodiments, a site-specific disruption in the TRAC locus provides a population of engineered T cells (e.g., engineered human CAR T cells) wherein at least 90%, 91%, 92%, 93%, 94%, 95%, 96%, 97%, 98%, or 100% of donor T cells lack expression of a functional TCR. In some embodiments, a site-specific disruption in the TRAC locus provides engineered T cells wherein at least 90%, 91%, 92%, 93%, 94%, 95%, 96%, 97%, 98%, or 100% of engineered T cells lack expression of a functional TCR. In some embodiments, a site-specific disruption in the TRAC locus and a purification step provides a cell population of engineered T cells (e.g., engineered human CAR T cells) wherein at least 97%, 97.5%, 98%, 98.5%, 99%, 99.5%, or 100% of engineered T cells lack expression of a functional TCR. In some embodiments, a site-specific disruption in the TRAC locus and a purification step provides engineered T cells wherein at least 97%, 97.5%, 98%, 98.5%, 99%, 99.5%, or 100% of engineered T cells lack expression of a functional TCR. In some embodiments, administration of a population of engineered T cells, wherein at least 97%, 97.5%, 98%, 98.5%, 99%, 99.5%, or 100% of engineered T cells (e.g., engineered human CAR T cells) lack expression of a functional TCR, reduces the risk of GvHD following administration to a recipient patient. In some embodiments, administration of engineered T cells, wherein at least 97%, 97.5%, 98%, 98.5%, 99%, 99.5%, or 100% of engineered T cells lack expression of a functional TCR, reduces the risk of GvHD following administration to a recipient patient.

Clinically, GvHD is divided into acute, chronic, and overlap syndrome based upon clinical manifestations and the time of incidence relative to administration of allogeneic donor cells. In some embodiments, administration of a population of cells comprising engineered T cells (e.g., engineered human CAR T cells) is associated with a reduced risk of acute GvHD (aGvHD) in a recipient. In some embodiments, administration of engineered T cells is associated with a reduced risk of acute GvHD (aGvHD) in a recipient. In some embodiments, administration of a population of cells comprising engineered T cells (e.g., engineered human T cells expressing a CAR) is associated with a reduced risk of chronic GvHD in a recipient. In some embodiments, administration engineered T cells is associated with a reduced risk of chronic GvHD in a recipient. In patients that develop aGvHD, symptoms can include a maculopapular rash; hyperbilirubinemia with jaundice due to damage to the small bile ducts, leading to cholestasis; nausea, vomiting, and anorexia; and watery or bloody diarrhea and cramping abdominal pain (Zeiser, R. et al. (2017) N Engl J Med 377:2167-2179). The severity of aGvHD is based upon clinical manifestations and is readily evaluated by one skilled in the art using widely accepted grading parameters as defined, for example, in Table 4.

In some embodiments, administration of a population of cells comprising engineered T cells (e.g., engineered human CAR T cells expressing a CAR) results in reduced or mild aGvHD. In some embodiments, reduced or mild aGvHD is below clinical grade 2, below clinical grade 1, or grade 0. In some embodiments, reduced or mild aGvHD is below clinical grade 2. In some embodiments, reduced or mild aGvHD is below clinical grade 1. In some embodiments, reduced or mild aGvHD is at clinical grade 0.

In some embodiments, administration of a population of cells comprising engineered T cells of the present disclosure (e.g., engineered human T cells expressing a CAR) results in few or no occurrence of aGvHD (e.g., no subjects experience clinically significant (e.g., grade 2-4), or symptoms of aGvHD following administration of the population of engineered T cell of the present disclosure. In some embodiments, administration of engineered T cells results in few or no occurrence of aGvHD (e.g., no subjects experience clinically significant (e.g., grade 2-4), or symptoms of aGvHD following administration of the engineered T cells. In some embodiments, administration of a population of cells comprising engineered T cells (e.g., engineered human CAR T cells) of the present disclosure is associated with a partial occurrence of aGvHD, wherein less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, or less than 18% of subjects experience clinically significant (e.g., grade 2-4) symptoms of aGvHD following administration. In some embodiments, administration of engineered T cells is associated with a partial occurrence of aGvHD, wherein less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, or less than 18% of subjects experience clinically significant (e.g., grade 2-4) symptoms of aGvHD following administration.

TABLE 4

| | | | | Upper | |
|---|---|---|---|---|---|
| | Skin (active erythema | Liver | | gastrointestinal | Lower GI (stool |
| Stage | only) | (bilirubin) | | (GI) | output/day) |
| 0 | No active (erythematous) | <2 | mg/dL | No or intermittent | <500 mL/day or <3 |
| | GvHD rash | | | nausea, vomiting or | episodes/day |
| | | | | anorexia | |
| 1 | Maculopapular rash <2% | 2-3 | mg/dL | Persistent nausea, | 500-999 mL/day or |
| | body surface area (BSA) | | | vomiting or anorexia | 3-4 episodes/day |
| 2 | Maculopapular rash 25- | 3.1-6 | mg/dL | — | 1000-1500 mL/day |
| | 50% BSA | | | | or 5-7 episodes/day |
| 3 | Generalized | 6.1-15 | mg/dL | — | >1500 mL/day or >7 |
| | maculopapular rash | | | | episodes/day |
| | (>50% BSA) | | | | |
| 4 | Generalized | >15 | mg/dL | — | Severe abdominal |
| | erythroderma (>50% | | | | pain with or without |
| | BSA) plus bullous | | | | ileus, or grossly |
| | formation and | | | | bloody stool |
| | desquamation >5% BSA | | | | (regardless of stool |
| | | | | | volume) |

### Overall Clinical Grade (Based Upon Most Severe Target Organ Involvement):

Grade 0: No stage 1-4 of any organ
Grade 1: Stage 1-2 without liver, upper GI or lower GI involvement
Grade 2: Stage 3 rash and/or stage 1 liver and/or stage 1 upper GI and/or stage 1 lower GI
Grade 3: Stage 2-3 liver and/or stage 2-3 lower GI, with stage 0-3 skin and/or stage 0-1 upper GI
Grade 4: Stage 4 skin, liver or lower GI involvement, with stage 0-1 upper GI

Steroids are used as a first-line treatment for aGvHD. However, response rates are limited, with only approximately 50% of patients responding to first-line therapy. Outcomes for patients with steroid-refractory aGvHD are dismal with long-term mortality rates approaching 90% (Westin, J. et al., (2011) Adv Hematol. Article ID 601953).

In some embodiments, administration of a population of cells comprising engineered T cells (e.g., engineered human CAR T cells) of the present disclosure is associated with no occurrence of steroid-refractory aGvHD, wherein no subjects experience clinically significant (e.g., grade 2-4) symptoms of steroid-refractory aGvHD following administration. In some embodiments, administration of a population of cells comprising engineered T cells (e.g., engineered human CAR T cells) of the disclosure is associated with a partial occurrence of steroid-refractory aGvHD, wherein less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, or less than 18% of subjects experience clinically significant (e.g., grade 2-4) symptoms of steroid-refractory aGvHD following administration.

In some embodiments, a subject is observed for up to 21 days, 22 days, 23 days, 24 days, 25 days, 26 days, 27 days, 28 days, 29 days, 30 days, 31 days, 32 days, 33 days, 34 days, 35 days, or 36 days following administration of a population of cells comprising engineered T cells (e.g., engineered human CAR T cells) for symptoms of aGvHD. In some embodiments, a subject is observed for at least 20-50 days, 25-70 days, 28-100 days following administration of a population of cells comprising engineered T cells (e.g., engineered human CAR T cells) for symptoms of aGvHD.

In some embodiments, the clinical setting of HSCT provides a benchmark for the incidence of aGvHD in response to administration of a population of cells comprising engineered T cells. The overall incidence of clinically significant (e.g., grade 2-4) aGvHD is approximately 50% following administration of allogeneic HSCT, but can vary widely depending upon factors that include but are not limited to HLA disparity between donor and recipient, donor and recipient gender, donor and recipient age, tissue source of the graft, and conditioning regimen intensity (Hatzimichael, E. (2010) Stem Cells and Cloning: Advances and Applications, 3:105-117). More specifically, the incidence of clinically significant (e.g., grade 2-4) aGvHD following administration of HSCT from an HLA-matched, sibling donor is approximately 20-35% (Remberger, M. et al., (2002) Br J Haematol. 119:751-759; Hahn, T. et al. (2008) J Clin Oncol. 26:5728-5734). The incidence rates increase for non-related or non-HLA-matched donors. In some embodiments, administration of engineered T cells (e.g., engineered human CAR T) is associated with an incidence of clinically significant (e.g., grade 2-4) aGvHD at least 20%, 30%, 40%, 50%, 60%, 70%, 80%, 90% or 100% lower than an incidence of clinically significant (e.g., grade 2-4) aGvHD induced by administration of allogeneic HSCT. In some embodiments, an HSCT is from an HLA-matched, sibling donor. In some embodiments, an HSCT is from an HLA-matched, non-relative donor. In some embodiments, an HSCT is from an HLA-mismatched, sibling donor. In some embodiments, an HSCT is from an HLA-mismatched, non-sibling donor.

E. Pre-Conditioning Regimen

In some embodiments, to prevent a host immune response to the CAR T cell therapy described herein, a pre-conditioning regimen is administered to a subject. Preconditioning a patient with one or more immunosuppressive chemotherapy drugs prior to administration of a T cell infusion can increase the effectiveness of donor T cells following transplantation (North, R. et al. (1982) J Exp Med, 155:1063-1074; Berenson, J. et al. (1975) J. Immunol. 115:234-238; Cheever, M. et al., (1980) J Immunol. 125:711-714).

Any of the combined therapy disclosed herein may further involve a pre-condition regimen. In some embodiments, a pre-conditioning regimen is administered prior to, concurrently with, or subsequent to administration of CAR T cell therapy (e.g., engineered T cells). In some embodiments, a pre-condition regimen is administered prior to, concurrently with, or subsequent to administration of an NK cell inhibitor.

In some embodiments, when the CAR T cell therapy includes a pre-conditioning regimen as described herein (e.g., lymphodepletion regimen), an NK cell inhibitor is administered concurrently with the pre-conditioning regimen (e.g., lymphodepletion regimen). In some embodiments, an NK cell inhibitor is administered before the administration of the pre-conditioning regimen (e.g., lymphodepletion regimen). In some embodiments, an NK cell inhibitor is administered after the administration of the pre-conditioning regimen (e.g., lymphodepletion regimen).

In some embodiments, a pre-conditioning regimen comprises a lymphodepletion regimen.

(i) Lymphodepletion

In some embodiments, a population of cells comprising engineered T cells (e.g., engineered human T cells) is administered to a subject (e.g., a human patient having a cancer, e.g., a non-Hodgkin lymphoma) after the subject has received a lymphodepleting regimen and/or an NK cell inhibitor.

Lymphodepletion (LD) chemotherapy and has the goal of enabling expansion and effector function of transplanted T cells following infusion. It is known that endogenous regulatory T cells and other immune cells can siphon certain cytokines (e.g., interleukin 7 (IL-7), IL-15, IL-2, IL-21) from circulation. A function of LD chemotherapy is the transient elimination of endogenous immune cells that function as ‘cellular sinks’ of stimulatory cytokines, thereby providing an increased abundance of cytokines that promote activation and expansion of donor T cells following administration (Gattinoni, L. et al., (2005) J Expt Med 202:907-912). Additionally, it is known that by reducing the quantity of host naïve T cells below a certain threshold, transplanted donor T cells will proliferate and differentiate in order to reestablish the depleted T cell population (Dummer, W. et al. (2002) J Clin Invest. 110:185-192; Muranski, P. et al. (2006) Nat Clin Pract Oncol. 3:668-681). Thus, another function of LD chemotherapy is the transient reduction of naïve T cells in a host to favor the proliferation and differentiation of transplanted donor T cells (e.g., engineered human T cells, e.g., engineered allogeneic human T cells). In some embodiments, the conditioning therapy is intended to reduce endogenous immune cells and increase serum levels of homeostatic cytokines and/or pro-immune factors present in a subject. In some embodiments, the conditioning therapy is intended to reduce endogenous lymphocytes below a threshold that favors the expansion of donor T cells (e.g., engineered human T cells, e.g., engineered allogeneic human T cells) upon administration. In some embodiments, the conditioning therapy creates a more optimal microenvironment for the donor T cells (e.g., engineered human T cells, e.g., engineered allogeneic human T cells) to proliferate once administered to a recipient. In some embodiments, the conditioning therapy creates a more optimal microenvironment for the donor T cells (e.g., engineered human T cells, e.g., engineered allogeneic human T cells) to have effector function once administered to a recipient.

In some embodiments, LD chemotherapy is comprised of at least one, two, three, or four chemotherapy drugs. In some embodiments, LD chemotherapy is comprised of administering one or more or more doses of cyclophosphamide. In some embodiments, LD chemotherapy is comprised of administering one or more or more doses of fludarabine. In some embodiments, LD chemotherapy is comprised of administering one or more doses of cyclophosphamide in combination with one or more doses of an additional chemotherapy drug (e.g., cyclophosphamide+fludarabine) as described in U.S. Pat. No. 9,855,298 that is incorporated as reference herein. In some embodiments, LD chemotherapy is combined with irradiation for the purposes of inducing leukapheresis.

In some embodiments, cyclophosphamide is administered intravenously (e.g., as an intravenous infusion). In some embodiments, an infusion of cyclophosphamide is administered at a dose of about 200 mg/m² to about 2000 mg/m², wherein m² indicates the body area of the recipient. In some embodiments, an infusion of cyclophosphamide is administered at a dose of about 300 mg/m². In some embodiments, an infusion of cyclophosphamide is administered at a dose of about 500 mg/m². In some embodiments, an infusion of cyclophosphamide is administered at a dose of about 750 mg/m².

In some embodiments, cyclophosphamide is administered as an infusion comprised of at least 1 dose, 2 doses, 3 doses, 4 doses, 5 doses, 6 doses, 7 doses, 8 doses, 9 doses, or 10 doses. In some embodiments, two or more sequential infusions of cyclophosphamide are administered with an intervening interval of no more than 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, or 32 hours. In some embodiments, two or more sequential infusions of cyclophosphamide are administered with an intervening interval of no more than 1 day, 2 days, 3 days, 4 days, or 5 days. In some embodiments, an intervening interval between sequential infusion of cyclophosphamide is the same. In some embodiments, an intervening interval between sequential infusion of cyclophosphamide is different. In some embodiments, two or more sequential infusions of cyclophosphamide are administered within an interval of 2 days, 3 days, 4 days, 4 days, 5 days, 6 days, 7 days, 8 days, 9 days, 10 days, 11 days, or 12 days.

In some embodiments, fludarabine is administered intravenously (e.g., as an intravenous infusion). In some embodiments, an infusion of fludarabine is administered at a dose of about 10 mg/m² to about 900 mg/m². In some embodiments, an infusion of fludarabine is administered at a dose of about 30 mg/m².

In some embodiments, fludarabine is administered as an infusion comprised of at least 1 dose, 2 doses, 3 doses, 4 doses, 5 doses, 6 doses, 7 doses, 8 doses, 9 doses, or 10 doses of cyclophosphamide. In some embodiments, two or more sequential infusions of fludarabine are administered with an intervening interval of no more than 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, or 32 hours. In some embodiments, two or more sequential infusions of fludarabine are administered with an intervening interval of no more than 1 day, 2 days, 3 days, 4 days, or 5 days. In some embodiments, an intervening interval between sequential infusion of fludarabine is the same. In some embodiments, an intervening interval between sequential infusion of fludarabine is different. In some embodiments, two or more sequential infusions of fludarabine are administered within an interval of 2 days, 3 days, 4 days, 4 days, 5 days, 6 days, 7 days, 8 days, 9 days, 10 days, 11 days, or 12 days.

In some embodiments, LD chemotherapy comprises a combination of chemotherapy drugs. In some embodiments, LD chemotherapy comprises a combination of cyclophosphamide and fludarabine. In some embodiments, cyclophosphamide and fludarabine are administered simultaneously. In some embodiments, cyclophosphamide and fludarabine are administered sequentially. In some embodiments, cyclophosphamide and fludarabine are administered sequentially, wherein an intervening interval between administration of fludarabine is not more than 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, or 20 hours. In some embodiments, cyclophosphamide is administered prior to administration of fludarabine, wherein an intervening interval between administration is not more than 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, or 20 hours.

In some embodiments, LD chemotherapy is administered daily for 1 day, for 2 days, for 3 days, for 4 days, for 5 days, for 6 days, for 7 days, for 8 days, for 9 days, for 10 days, for 11 days, or for 12 days. In some embodiments, LD chemotherapy is administered daily for 1-4 days. In some embodiments, LD chemotherapy is administered daily for 3 days.

In some embodiments, LD chemotherapy comprising cyclophosphamide and fludarabine is administered daily for 1 day, for 2 days, for 3 days, for 4 days, for 5 days, for 6 days, for 7 days, for 8 days, for 9 days, for 10 days, for 11 days, or for 12 days. In some embodiments, LD chemotherapy comprising cyclophosphamide and fludarabine is administered daily for 1-4 days.

In some embodiments, LD chemotherapy comprising cyclophosphamide and fludarabine is administered daily for 3 days.

In some embodiments, administration of LD chemotherapy (e.g., cyclophosphamide and fludarabine) is associated with increased serum levels of IL-7, IL-15, IL-2, IL-21, IL-10, IL-5, IL-8, MCP-1, PLGF, CRP, sICAM-1, sVCAM-1, or any combination thereof. In some embodiments, administration of LD chemotherapy (e.g., cyclophosphamide and fludarabine) is associated with decreased serum levels of perforin, MIP-1b, or any combination thereof. In some embodiments, administration of LD chemotherapy (e.g., cyclophosphamide and fludarabine) is associated with lymphopenia. In some embodiments, administration of LD chemotherapy (e.g., cyclophosphamide and fludarabine) is associated with decrease (e.g., depletion) of regulatory T cells in a subject.

In some embodiments, a subject is administered an LD chemotherapy regimen prior to administration of a population of cells comprising engineered T cells (e.g., engineered human CAR T cells). In some embodiments, the subject is administered the LD chemotherapy regimen one day, two days, three days, four days, five days, six days, seven days, eight days, nine days or ten days prior to administration of the population of cells comprising engineered T cells (e.g., engineered human CAR T cells). In some embodiments, the subject is administered the LD chemotherapy regimen at least one day, at least two days, at least three days, at least four days, at least five days, at least six days, at least seven days, at least eight days, at least nine days or at least ten days prior to administration of the population of cells comprising engineered T cells (e.g., engineered human CAR T cells).

In some embodiments, the subject is administered the LD chemotherapy regimen for one day, two days, three days, four days, five days, six days, seven days, eight days, nine days or ten days, and the population of cells comprising engineered T cells (e.g., engineered human CAR T cells) is administered at least one day, two days, three days, four days, five days, six days, seven days, eight days, nine days or ten days after completion of the LD chemotherapy regimen. In some embodiments, the subject is administered the LD chemotherapy regimen for 1-3 days, and the population of cells comprising engineered T cells (e.g., engineered human CAR T cells) is administered at least two days (e.g., 48 hours), but no more than seven days, after completion of the LD chemotherapy regimen. In some embodiments, the subject is administered the LD chemotherapy regimen for about 3 days, and the population of cells comprising engineered T cells (e.g., engineered human CAR T cells is administered at least two days (e.g., 48 hours), but no more than seven days, after completion of the LD chemotherapy regimen.

In some embodiments, a subject is administered cyclophosphamide and fludarabine prior to administration of a population of cells comprising engineered T cells (e.g., engineered human CAR T cells). In some embodiments, the subject is administered cyclophosphamide and fludarabine one day, two days, three days, four days, five days, six days, seven days, eight days, nine days or ten days prior to administration of the population of cells comprising engineered T cells (e.g., engineered human CAR T cells). In some embodiments, the subject is administered cyclophosphamide and fludarabine at least one day, at least two days, at least three days, at least four days, at least five days, at least six days, at least seven days, at least eight days, at least nine days or at least ten days prior to administration of the population of cells comprising engineered T cells (e.g., engineered human CAR T cells).

In some embodiments, the subject is administered cyclophosphamide and fludarabine for one day, two days, three days, four days, five days, six days, seven days, eight days, nine days or ten days, and the population of cells comprising engineered T cells (e.g., engineered human CAR T cells) is administered at least one day, two days, three days, four days, five days, six days, seven days, eight days, nine days or ten days after completion of cyclophosphamide and fludarabine administration. In some embodiments, the subject is administered cyclophosphamide and fludarabine for 1-3 days, and the population of cells comprising engineered T cells (e.g., engineered human CAR T cells) is administered at least two days (e.g., 48 hours), but no more than seven days, after completion of cyclophosphamide and fludarabine administration. In some embodiments, the subject is administered cyclophosphamide and fludarabine for about 3 days, and the population of cells comprising engineered T cells (e.g., engineered human CAR T cells) is administered at least two days (e.g., 48 hours), but no more than seven days, after completion of cyclophosphamide and fludarabine administration.

IV. Kit for Improving Clinical Outcome Associated with CAR-T Therapy

The present disclosure also provides kits for use of a population of genetically engineered CAR-T cells in combination with an NK cell inhibitor, both disclosed herein, to improve clinical outcome. Such kits may include one or more containers comprising any of the genetically engineered CAR-T cells disclosed herein, or a nucleic acid (e.g., an AAV vector) encoding the CAR construct. In some embodiments, the kit may further include a container comprising a pharmaceutical composition that comprises one or more lymphodepleting agents and one or more pharmaceutically acceptable carriers. Alternatively or in addition, the kit may further comprise a pharmaceutical composition that comprises any of the NK cell inhibitors and one or more pharmaceutically acceptable carriers.

In some embodiments, the kit can comprise instructions for use in any of the methods described herein. The included instructions can comprise a description of administration of the genetically engineered CAR-T cells, the lymphodepleting agents, and/or the NK cell inhibitor to achieve the intended activity in a human patient. The kit may further comprise a description of selecting a human patient suitable for treatment based on identifying whether the human patient is in need of the treatment.

The instructions relating to the use of a population of genetically engineered CAR-T cells, the lymphodepleting agents, and/or the NK cell inhibitor described herein generally include information as to dosage, dosing schedule, and route of administration for the intended treatment. The containers may be unit doses, bulk packages (e.g., multi-dose packages) or subunit doses. Instructions supplied in the kits of the disclosure are typically written instructions on a label or package insert. The label or package insert indicates that the population of genetically engineered T cells is used for treating, delaying the onset, and/or alleviating a renal cell carcinoma in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. 1985); Transcription and Translation (B. D. Hames & S. J. Higgins, eds. 1984); Animal Cell Culture (R. I. Freshney, ed. 1986); Immobilized Cells and Enzymes (IRL Press, 1986); and A practical Guide To Molecular Cloning (B. Perbal, John Wiley & Sons Inc., 1984).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1: Preparation of Engineered Human CAR T Cells

This example describes the production of allogeneic engineered human T cells that express a chimeric antigen receptor (CAR) and lack expression of the TCR gene and β2M gene. The cells may express a chimeric antigen receptor targeting a cancer antigen, e.g., CD19 and/or BCMA. Methods for making these CAR T cells have been described in US Publication No. US 2018-0325955, incorporated herein by reference.

Briefly, primary human T cells were first electroporated with Cas9 or Cas9:sgRNA ribonucleoprotein (RNP) complexes targeting TRAC (AGAGCAACAGTGCTGTGGCC (SEQ ID NO: 28) and B2M (GCTACTCTCTCTTTCTGGCC (SEQ ID NO: 29)). The DNA double stranded break at the TRAC locus was repaired by homology directed repair as described below.

### Generation of Anti-CD19 CAR T Cells

To generate anti-CD19 CAR T cells, the DNA double-stranded break at the TRAC locus was repaired by homology directed repair with a recombinant adeno-associated adenoviral vector, serotype 6 (AAV6) comprising the nucleotide sequence of SEQ ID NO: 57 containing right and left homology arms to the TRAC locus flanking a chimeric antigen receptor (CAR) cassette (SEQ ID NO: 58). The CAR comprised a single-chain variable fragment (scFv) derived from a murine antibody specific for CD19, a CD8 hinge region, and a transmembrane domain and a signaling domain comprising CD3z and CD28 signaling domains. The amino acid sequence of the CAR, and the nucleotide sequence encoding the same is set forth in SEQ ID NOs: 39 and 40, respectively. The AAV6 was delivered with Cas9:sgRNA RNPs (1 μM Cas9, 5 μM gRNA) to activated human T cells. The nucleofection mix contained the Nucleofector™ Solution, 5×10⁶ cells, 1 μM Cas9, and 5 μM gRNA (as described in Hendel et al., Nat Biotechnol. 2015; 33(9):985-989, PMID: 26121415).

Each RNP complex comprised saCas9 and one of two sgRNAs targeting SEQ ID NO:28 in TRAC or SEQ ID NO:29 in β2M. Exemplary TRAC sgRNAs include SEQ ID NOs: 18 and 22. Exemplary TRAC sgRNA spacers include SEQ ID NOs: 19 and 23. Exemplary β2M sgRNAs include SEQ ID NOs: 20 and 24. Exemplary β2M sgRNA spacers include SEQ ID NOs: 21 and 25. The following sgRNAs were used: TRAC (SEQ ID NO: 22) and β2M (SEQ ID NO: 24). The unmodified versions (or other modified versions) of the gRNAs may also be used (e.g., SEQ ID NO: 18 and SEQ ID NO: 20).

About one week post-electroporation, cells were either left untreated or treated with phorbol myristate acetate (PMA)/ionomycin overnight. The next day cells were processed for flow cytometry (see, e.g., Kalaitzidis D et al. J Clin Invest 2017; 127(4): 1405-1413) to assess TRAC and β2M expression levels at the cell surface of the edited cell population. The following primary antibodies were used (Table 5):

TABLE 5
Antibodies.

| Antibody | Clone | Fluor | Catalogue # | Dilution | For 1 |
|---|---|---|---|---|---|
| TCR | BW242/412 | PE | 130-091-236 | 1:100 | 1 μL |
| | | | (Miltenyi) | | |
| β2M | 2M2 | PE-Cy7 | 316318 (Biolegend) | 1:100 | 1 μL |

Anti-CD19 CAR expression was detected using biotinylated recombinant human CD19 (ACROBIOSYSTEMS INC; CD9-H825).

### Generation of Anti-BCMA CAR T Cells

To generate anti-BCMA CAR T cells, the double-stranded break at the TRAC locus was repaired by homology directed repair with an AAV6 comprising the nucleotide sequence of SEQ ID NO: 83 (comprising the donor template in SEQ ID NO: 84, encoding anti-BCMA CAR comprising the amino acid sequence of SEQ ID NO: 86) is delivered with Cas9:gRNA RNPs (1 μM Cas9, and 5 μM gRNA) to activated allogeneic human T cells. The following gRNAs may be used: TRAC (SEQ ID NO: 22), and β2M (SEQ ID NO: 24). The unmodified versions (or other modified versions) of the gRNAs may also be used (e.g., SEQ ID NO: 18 and SEQ ID NO: 20). About one (1) week post-electroporation, cells are processed for flow cytometry as described above for anti-CD19 CAR+ T cells, with the following difference. Anti-BCMA CAR expression was detected using biotinylated recombinant human BCMA (ACROS Cat #BC7-H82F0).

### Example 2: CD38 Expression on CAR T Cells

CD38 cell expression on CAR T cells was measured by flow cytometry. Specifically, approximately fifteen days after the electroporation step described above, anti-CD19 and anti-BCMA CAR T cells prepared as outlined in Example 1 were stained with a panel of antibodies, and CD38 expression was measured.

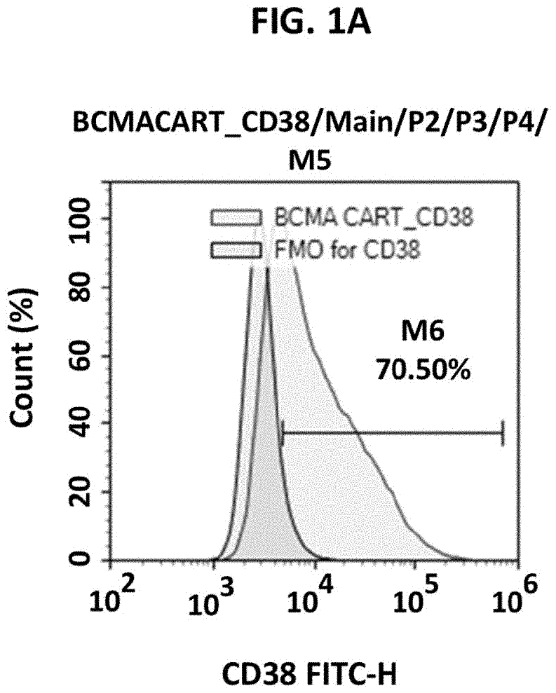

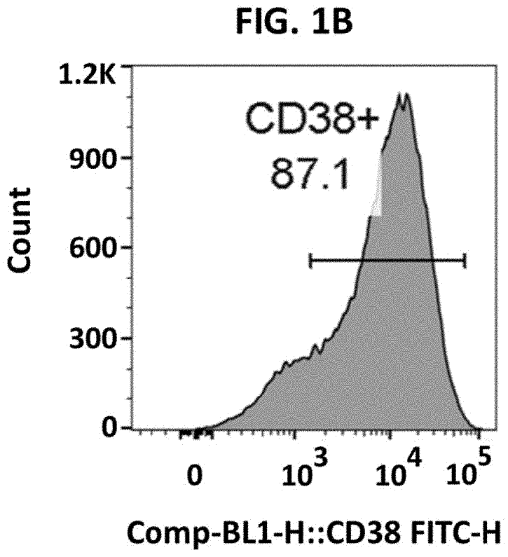

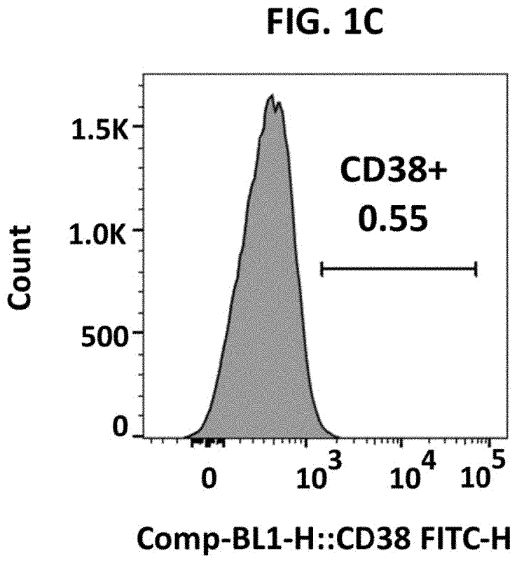

Live CAR T cells were gated by their forward scatter (FSC) and side scatter (SSC) profiles, and with a live/dead dye (cat #, L34965, ThermoFisher Scientific). The cells were then stained with a panel of antibodies: CD38 FITC (Clone HIT2, BioLegend), CD3 PE (UCHT1, Biolegend), CD4 APC/Cy7 (RPA-T4, Biolegend) and CD8 Pacific Blue (SK-1, Biolegend). CD3 T cells were then gated to measure CD38 expression. To establish gating cut-off for the CD38+ population, a fluorescent minus one (FMO) control staining was utilized (FIG. 1C). The data shows that a majority of anti-BCMA (70.5%) and anti-CD19 (87.1%) CAR T cells express CD38+.

### Example 3: CD38 Expression on NK and T Cells from Normal PBMCs

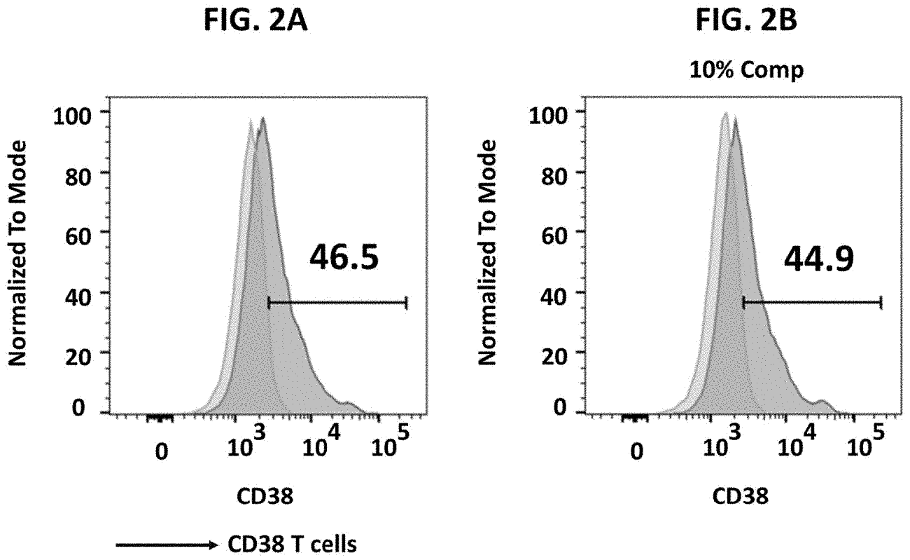

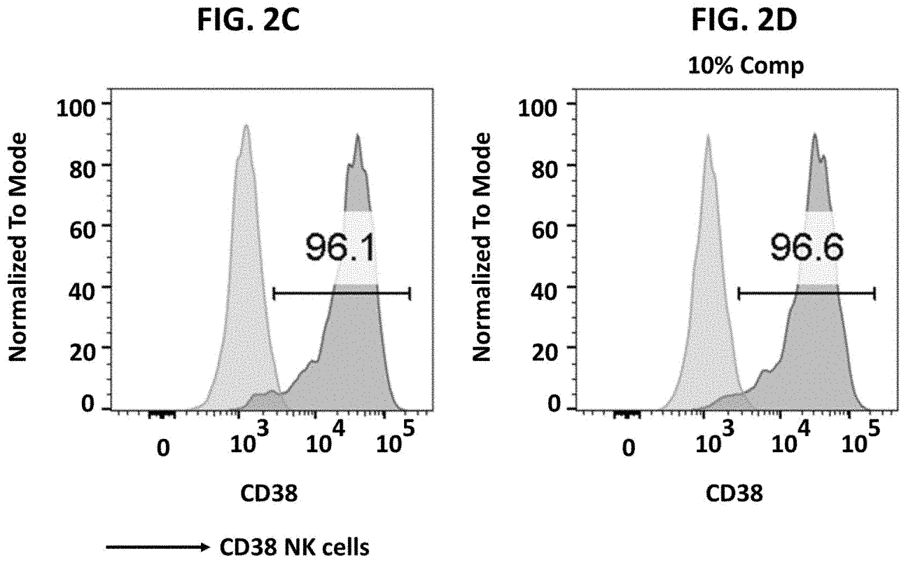

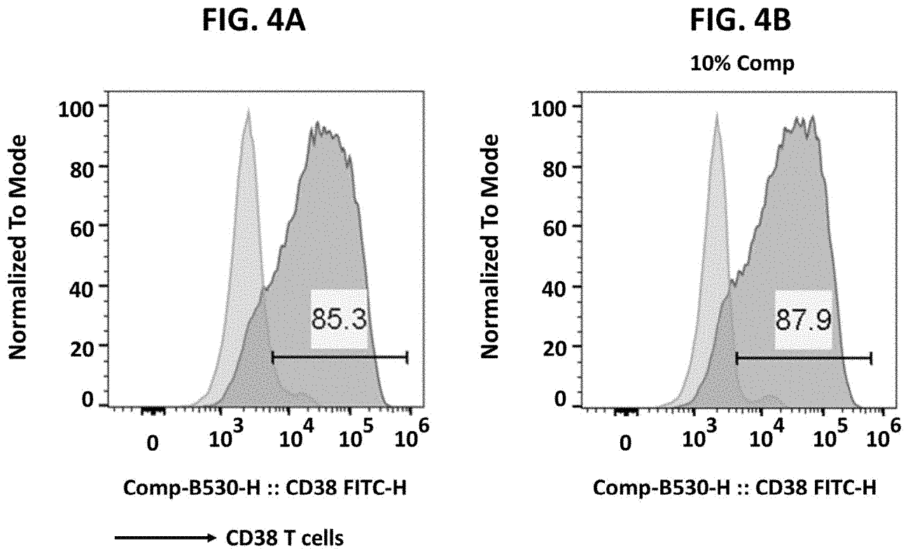

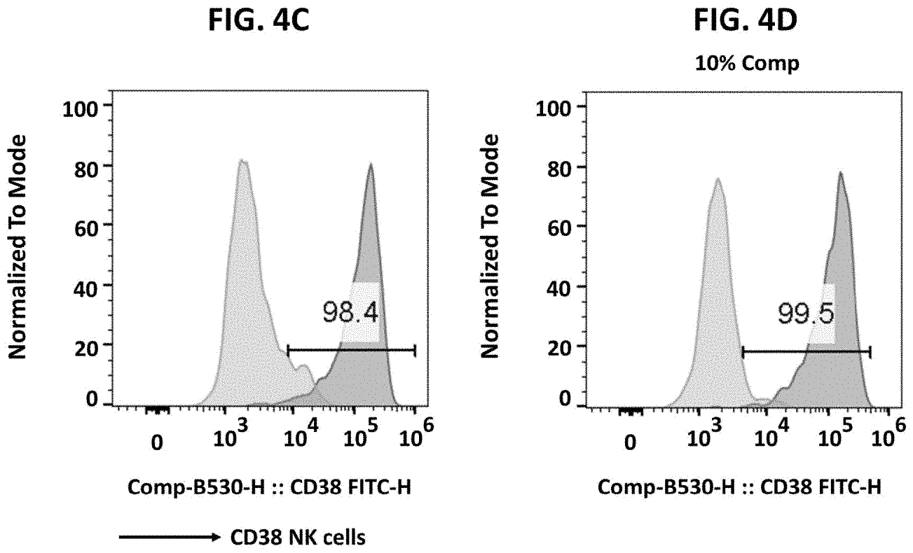

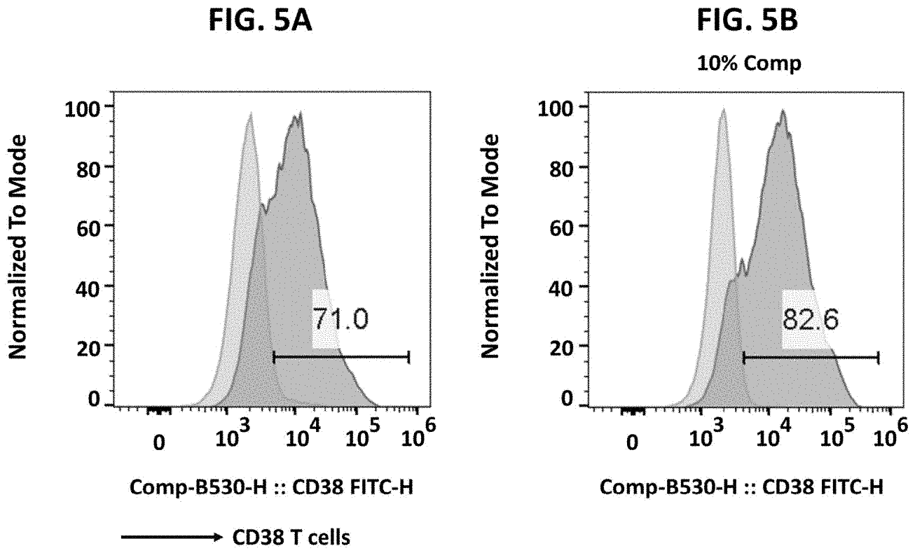

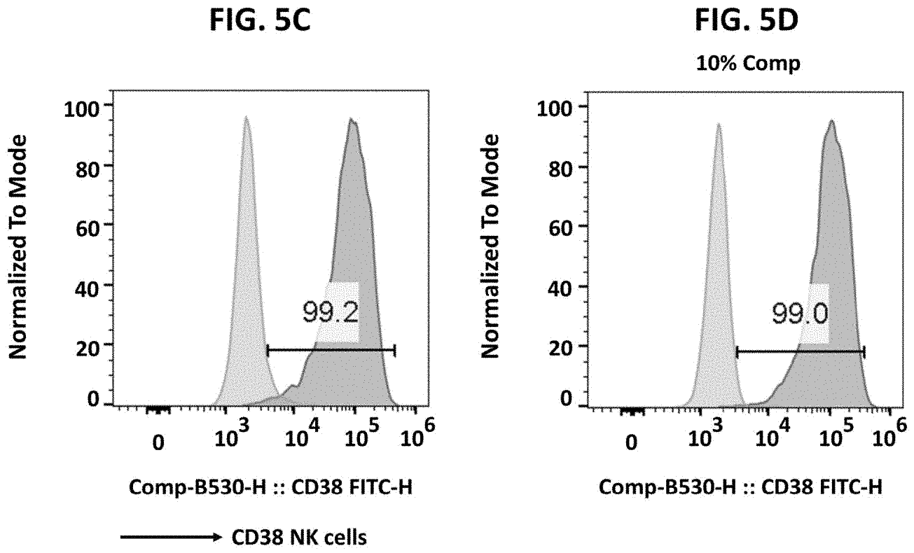

Peripheral blood mononuclear cells (PBMCs) were collected from healthy donors to assess CD38 expression in normal immune cells. PBMCs collected from two donors (Donor 3469 and Donor 3383) were cultured in media (X-vivo medium (cat #04-744, Lonza) supplemented with 5% human AB serum (cat #, HP1022HI, Valley Biomedical), IL-2 and IL7), with or without 10% complement (pooled complement serum, Innovative Research, Inc.). As complement mediated lysis is triggered by anti-CD38 antibodies, the cells were cultured in media with or without complement to evaluate the effect of complement on the CD38+ cells. Flow cytometry was used to assess CD38 expression in NK cells (CD3-, CD56+) and T cells (CD3+) at Day 0 (FIGS. 2A-2D and 3A-3D) and 72 hours (FIGS. 4A-4D and 5A-5D) of in vitro culture. The antibody panel used for flow cytometry was CD3 PE (UCHT1, BioLegend), CD38 FITC (Clone HIT2, BioLegend), CD56 APC (HCD56, BioLegend), and CD69 PECY5 (FN50, BioLegend).

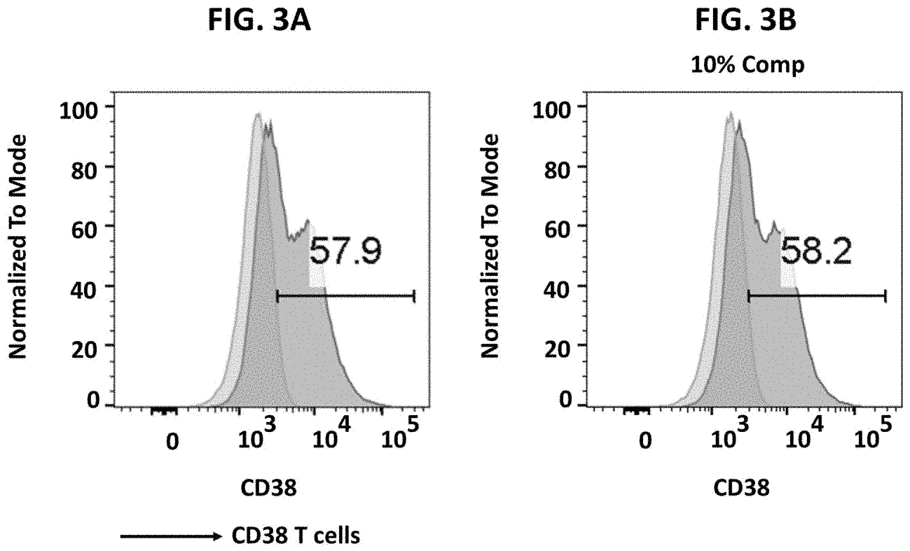

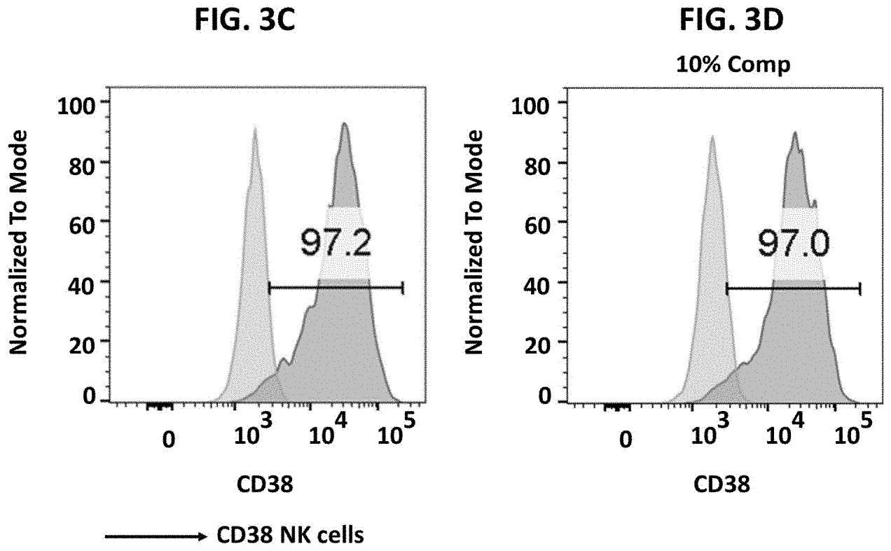

At Day 0 (1 hour after culture), the majority of NK cells and approximately half of T cells expressed CD38 on their cell surface. PBMCs from Donor 3469 showed CD38 expression in 96.1% (media alone) and 96.6% (media+10% complement) of NK cells (FIGS. 2C-2D). CD38 expression on T cells cultured in media alone or media supplemented with 10% complement was measured at 46.5% and 44.9%, respectively (FIGS. 2A-2B). Similarly, PBMCs from Donor 3383 that were cultured in media or media supplemented with 10% complement expressed CD38 in 97.2% (media alone) and 97.0% (media+10% complement) of NK cells, and in 57.9% (media alone) and 58.2% (media+complement) of T cells (FIGS. 3A-3D).

After 72 hours of culture, the majority of NK cells and T cells expressed CD38 on their cell surface. When PBMCs from Donor 3469 were cultured in media alone or media supplemented with 10% complement, CD38 expression was detected in 98.4% (media alone) and 99.5% (media+10% complement) of NK cells (FIG. 4C-4D). CD38 expression on T cells cultured in media alone or media supplemented with 10% complement was measured at 85.3% and 87.9% respectively (FIGS. 4A-4B). Similarly, PBMCs from Donor 3383 that were cultured in media or media supplemented with 10% complement expressed CD38 in 99.2% (media alone) and 99% (media+10% complement) of NK cells, and 71% (media alone) and 82.6% (media+complement) of T cells (FIGS. 5A-5D).

These data showed that the CD38 activation marker is present the majority (>90%) of NK cells and that high levels of CD38 expression are maintained when the cells are cultured in vitro, with or without complement. Although CD38 expression is relatively lower in the normal T cell population (˜ 50%), CD38 expression in CD3+ T cells increased to 71% and 85.3% after 72 hours of culture in media alone, and to 87.9% and 82.6% after 72 hours of culture in media supplemented with 10% complement. Although complement-mediated lysis has previously been reported to be elicited anti-CD38 antibodies (e.g., daratumumab), the data shows that the presence of complement does not affect cell numbers of CD38+NK or T cell numbers.

### Example 4: Daratumumab Treatment Depleted NK Cells while T Cell Numbers Remained Unaffected

Based on the expression levels of CD38 on NK and T cells, the effect of an anti-CD38 antibody, daratumumab (TAB-236, Creative Biolabs), on such cells was assessed. PBMCs from a healthy donor were cultured for 96 hours in media containing 0.01, 0.1, or 1 μg/mL of daratumumab. The effect of 10% complement on the cell cultures was also tested. Untreated cells and cells treated with 0.01, 0.1 or 1 μg/mL isotype control mAb (human IgG1k)(cat #403501, BioLegend) were used as controls. After 96 hours of culture, NK and T cell frequency and numbers were measured.

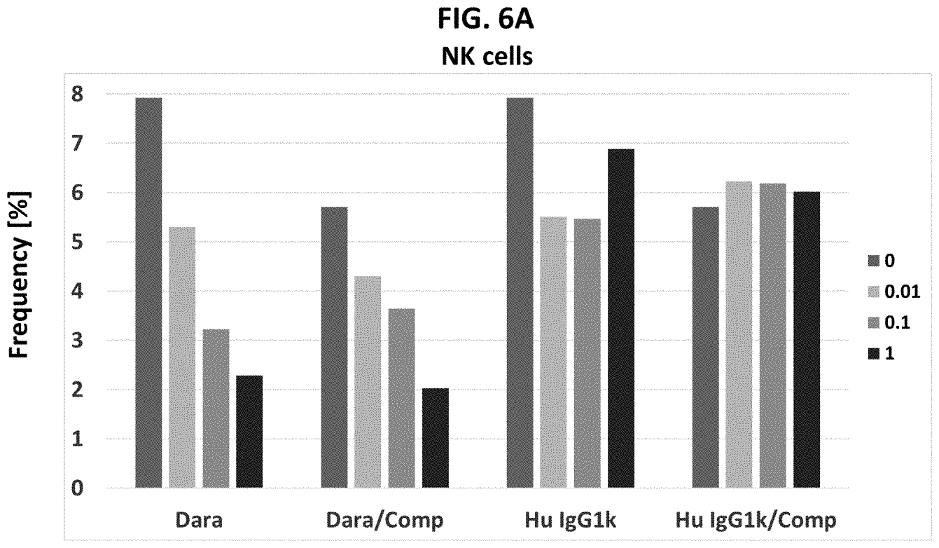

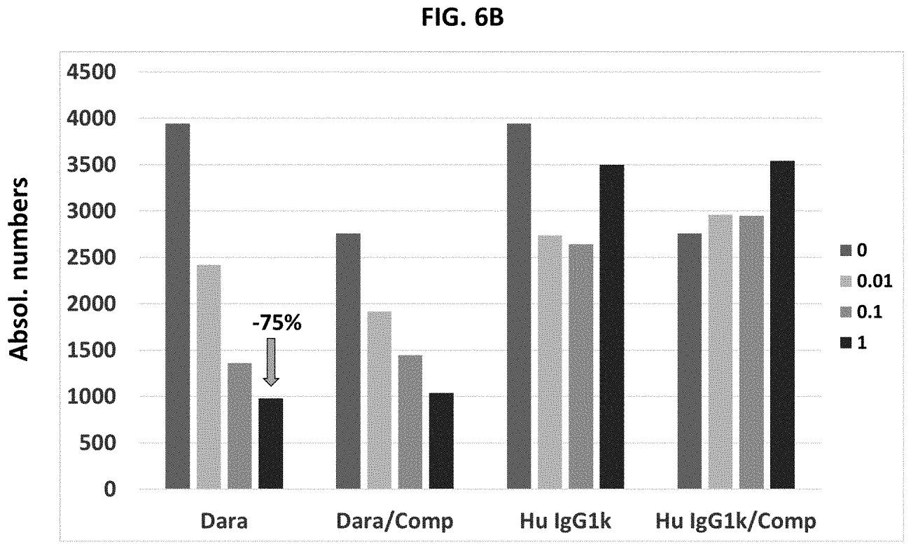

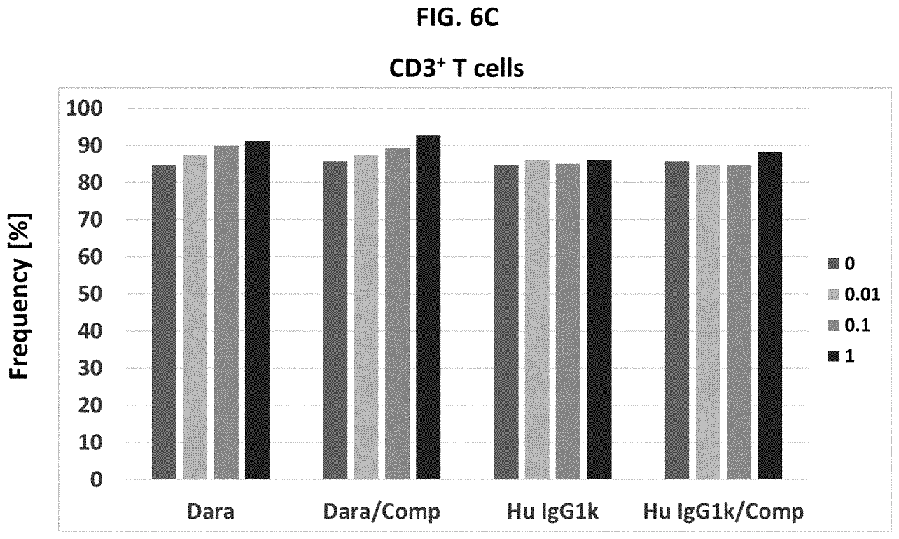

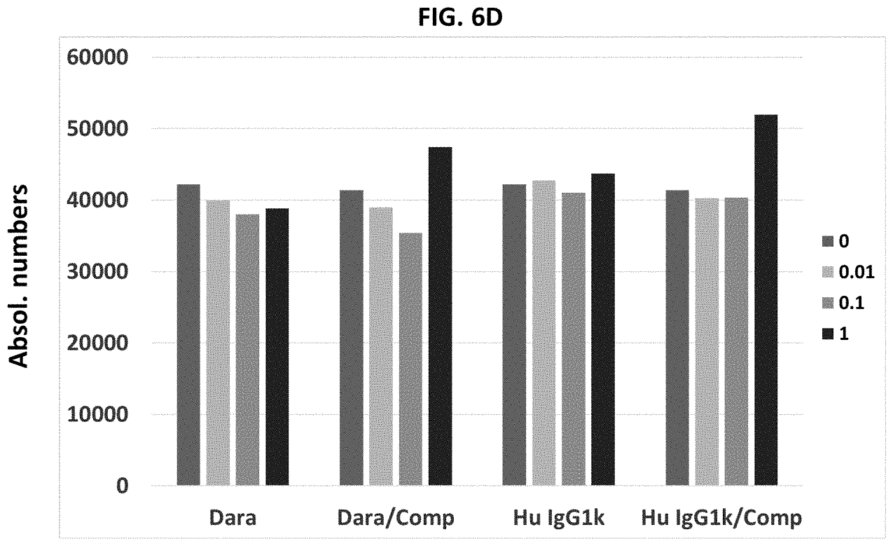

In vitro culture of daratumumab resulted in a dose-dependent decrease of NK cell frequency and numbers (FIGS. 6A-6B). At the highest dose tested, 1 μg/mL, daratumumab reduced NK cell numbers by approximately 75% after 96 hours. This effect is specific to daratumumab, as treatment with an isotype control mAb did not affect NK cell numbers. The reduction in NK cells is not complement dependent under these culture conditions, as the addition of 10% complement to the cell culture did not alter daratumumab's effect of NK cells.

In a second experiment PBMCs from a different donor, daratumumab reduced NK cell numbers ˜57% after only 72 hours (data not shown). These data demonstrate that daratumumab has similar effects on NK cells from different donor populations.

Contrary to its effect on NK cells, daratumumab did not affect T cell numbers or frequency (FIGS. 6C-6D). Although CD38 expression was detected on T cells (FIGS. 2A-2B and FIGS. 3A-3B) and in vitro culture of PBMC resulted in upregulation of CD38 surface expression in T cells (FIGS. 4A-4B and FIGS. 5A-5B), T cell numbers were surprisingly unaffected by the addition of daratumumab to the culture media.

### Example 5: Daratumumab Treatment does not Affect CAR T Numbers

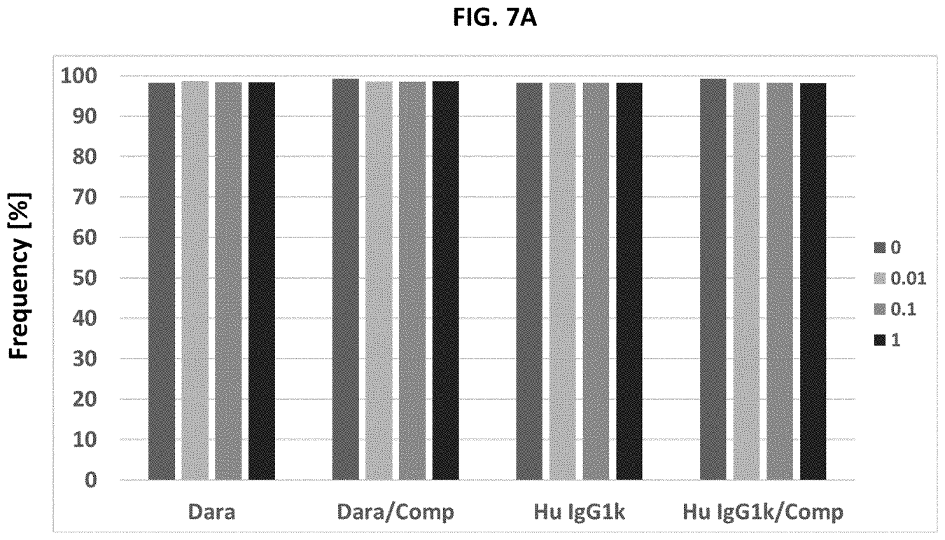

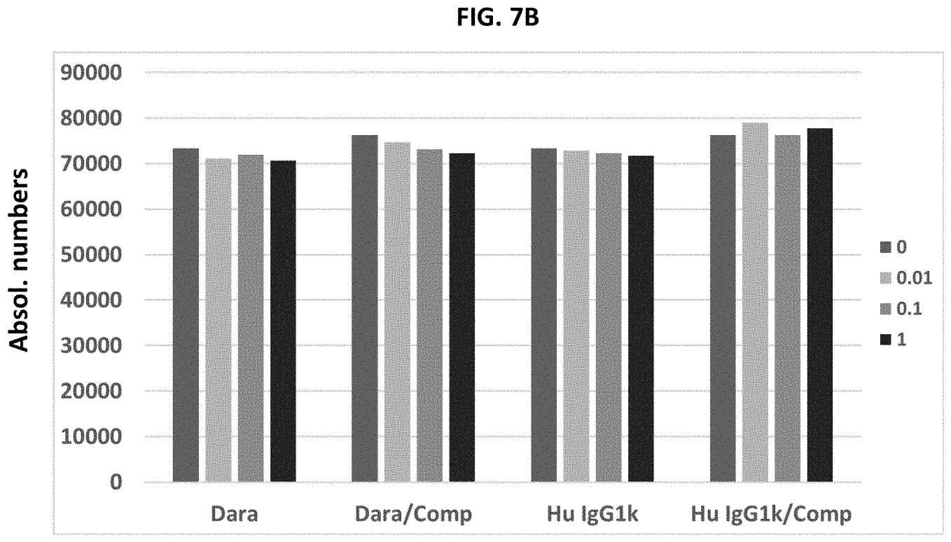

To assess whether daratumumab treatment affects CAR T cells with a disrupted β2M gene, anti-BCMA CAR T cells generated in Example 1 were treated with daratumumab with or without 10% complement. After a 72 hour culture period, anti-BCMA CAR T cell numbers and frequency were measured in a flow cytometry assay as described in Example 3 (FIGS. 7A-7B). Although a majority (70.5%) of anti-BCMA CAR T cells expressed CD38 (FIG. 1A), treatment with daratumumab, with or without 10% complement, did not affect anti-BCMA CAR T cells numbers or frequency.

### Example 6: Daratumumab Treatment does not Activate CAR T Cells

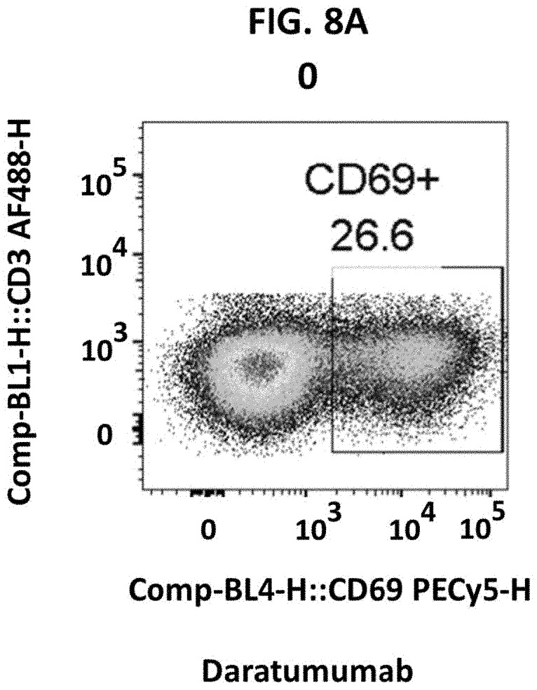

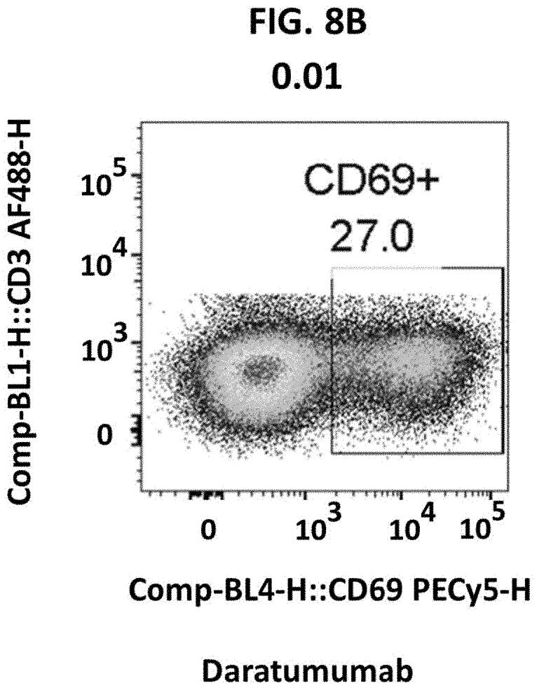

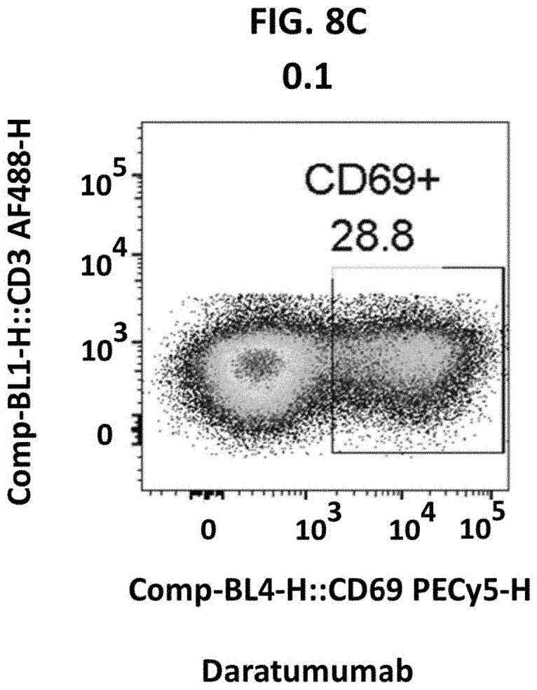

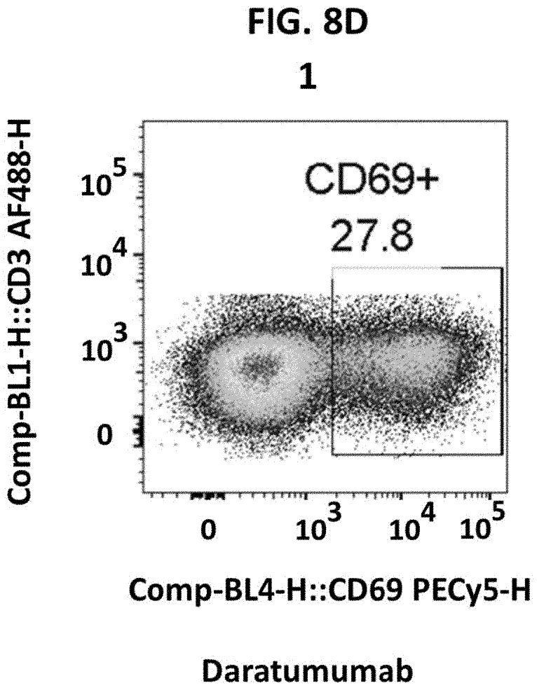

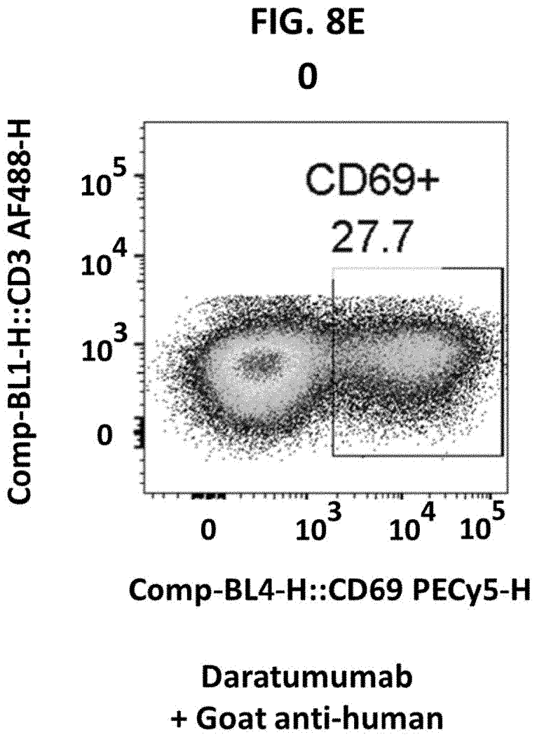

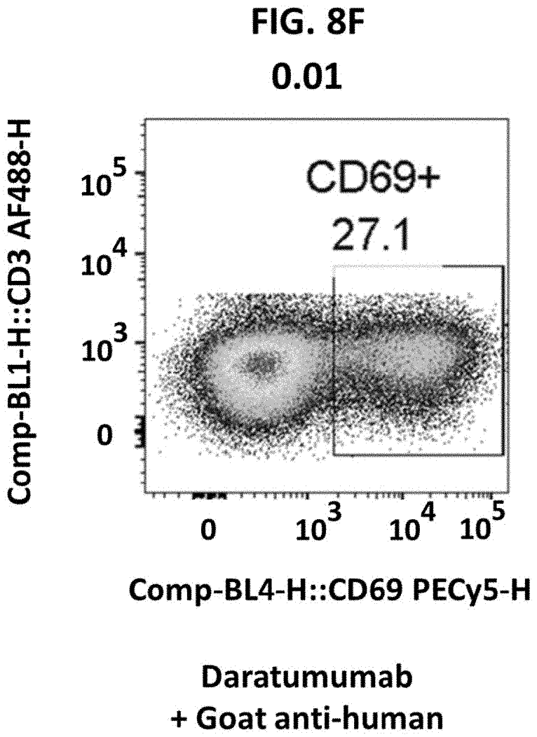

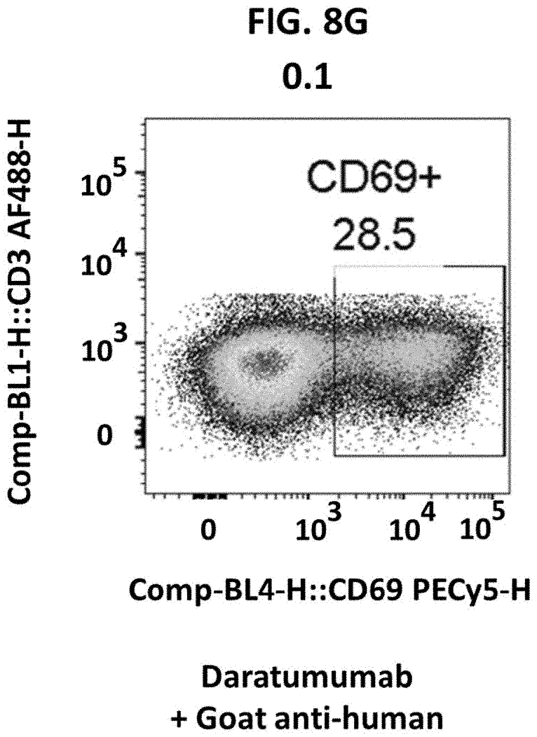

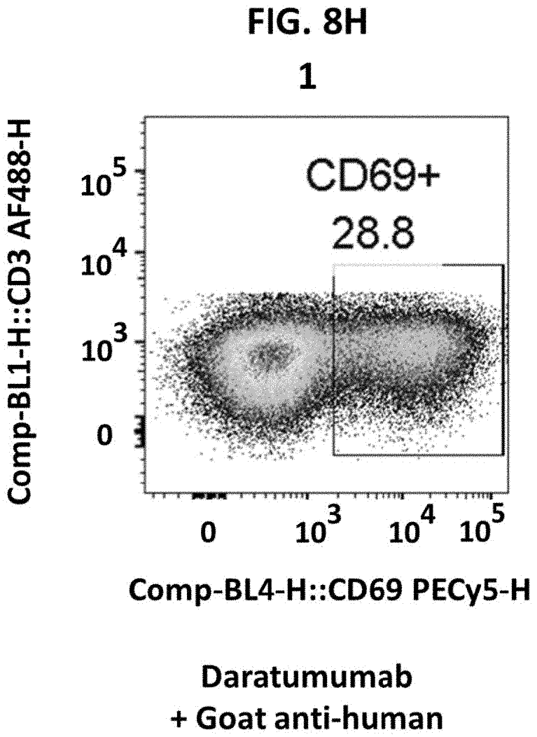

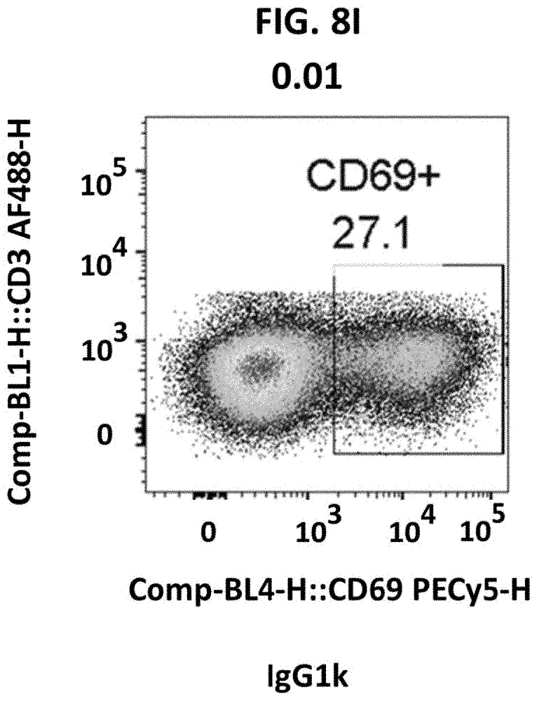

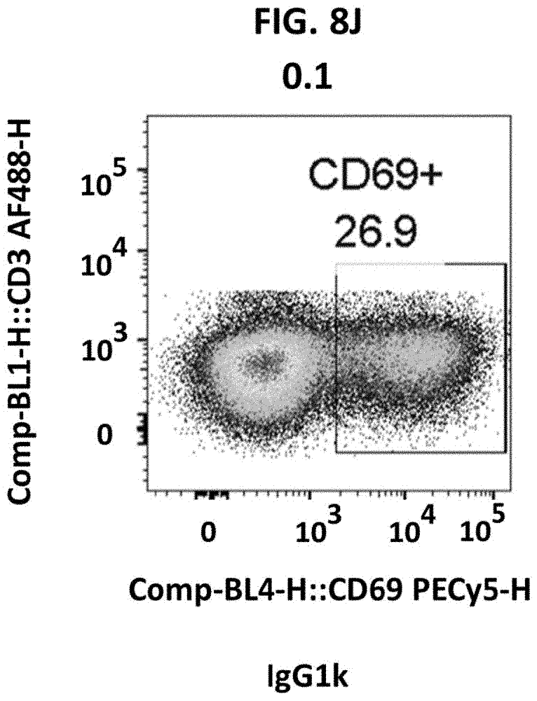

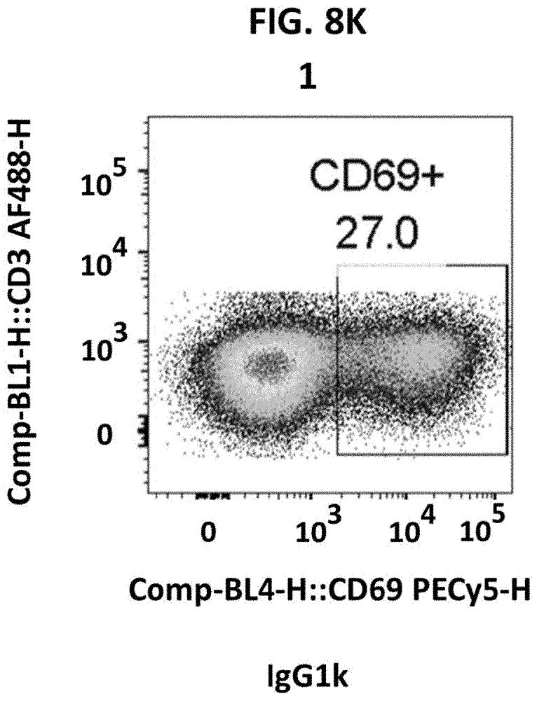

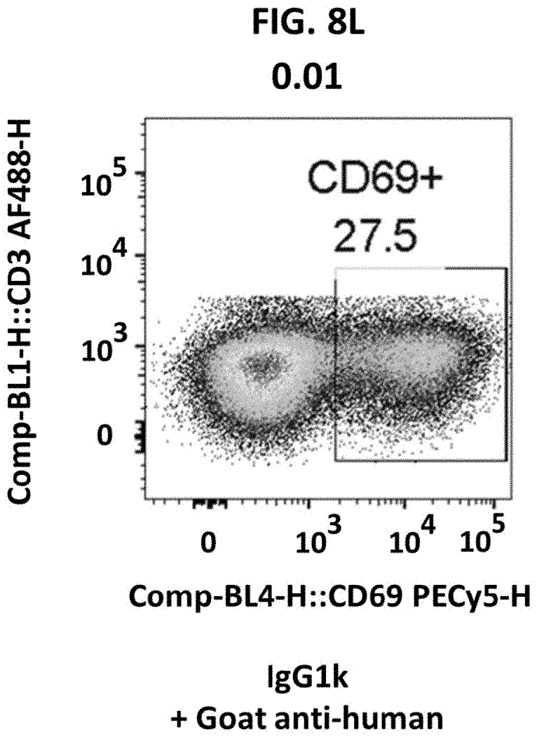

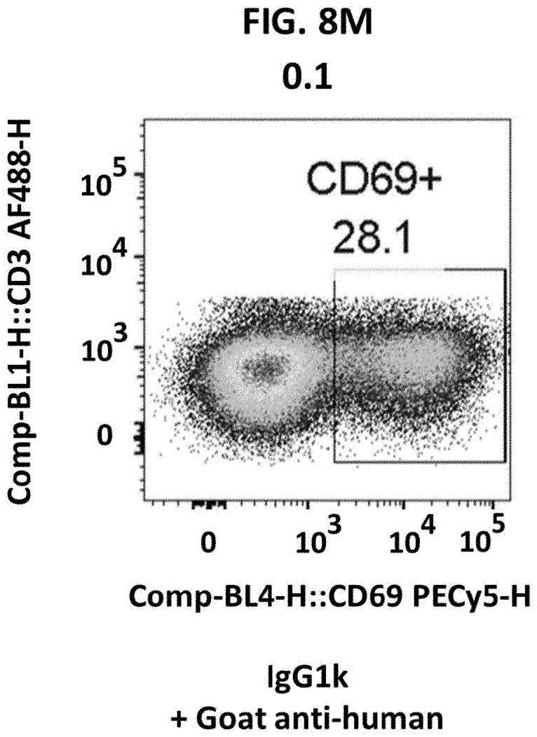

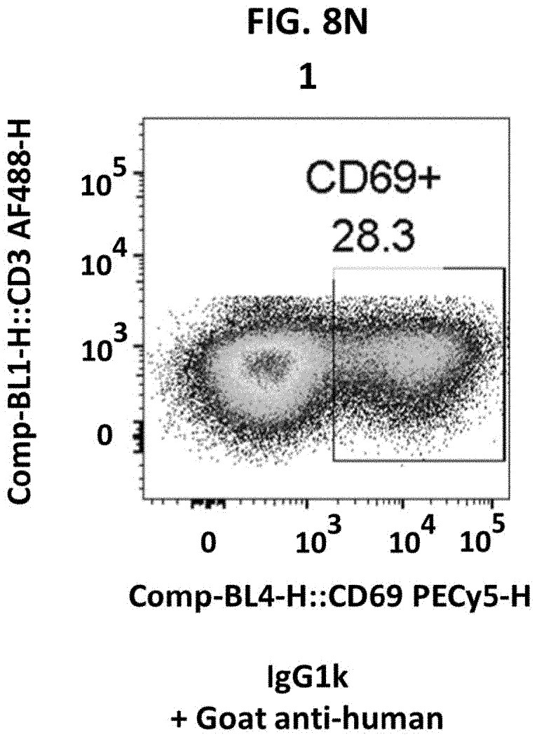

To determine whether daratumumab activates CAR T cells and causes subsequent proliferation or activation-induced cell death, anti-CD19 CAR T cells were cultured with daratumumab alone, or daratumumab with 2 μg/mL goat anti-human isotype control antibody for 24 hours. Daratumumab was used at a concentration of 0.01, 0.1, or 1 μg/mL. Untreated cells or anti-CD19 CAR T cells treated with IgG1k isotype control mAb were used as controls. Expression of the early activation marker, CD69, was assessed at the end of the 24 hour incubation period (FIGS. 8A-8N). As shown in the representative flow cytometry panels, CD69 expression was unchanged in all tested anti-CD19 CAR T cell populations, irrespective of treatment. The data thus show that despite the expression of CD38 cell surface marker (87.1%) on anti-CD19 CAR T cells (FIG. 1B), daratumumab treatment did not induce activation of the anti-CD19 CAR T cells.

### Example 7: Daratumumab Pre-Treatment Reduced NK Cell-Induced CAR T Cell Lysis

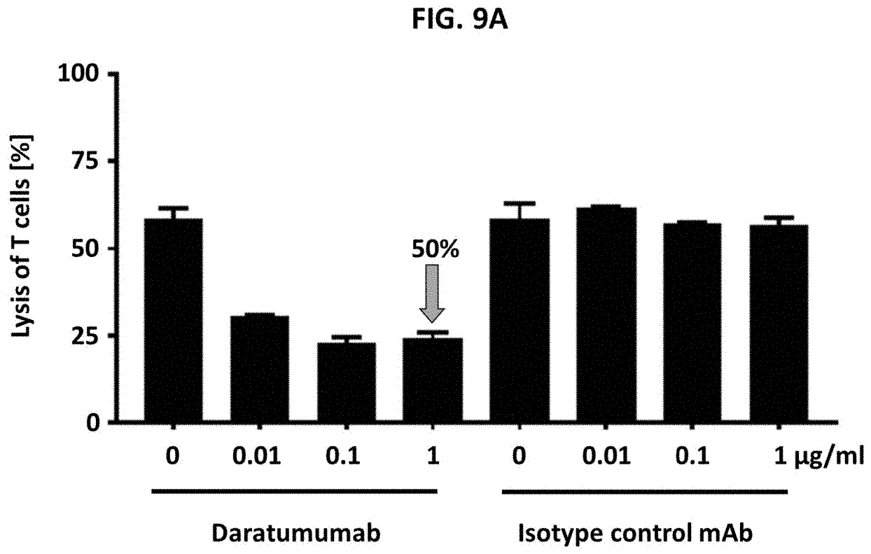

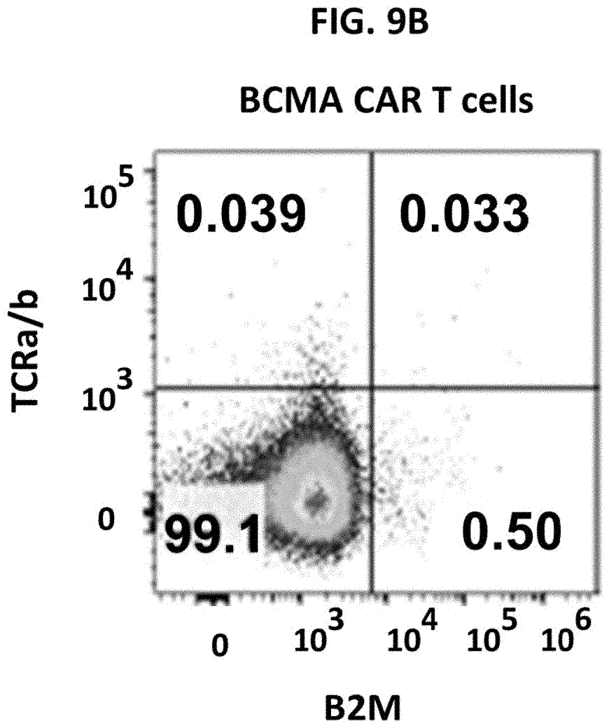

To determine if daratumumab blunts NK-cell mediated CAR T cell lysis, anti-BCMA CAR T cells were co-cultured with purified NK cells that were pre-treated for 60 hours with either daratumumab or isotype control mAb at concentrations of 0.01, 0.1, or 1 μg/mL (FIG. 9A). At the end of the 60 hour pre-treatment period, 50,000 efluor-labelled anti-BCMA CAR T cells were added to the plate containing 150,000 NK cells and Dara/isotype control, and incubated for an additional 24 hours. At the end of the 24-hour co-culture period, anti-BCMA CAR T cell lysis was measured in a cell-kill assay with DAPI.

Specifically, the anti-BCMA CAR T cells were labeled with 5 μM efluor670 (Cat #65-0840-90; ThermoFisher Scientific), washed and incubated in co-cultures with the NK cells at a 3:1 (NK:T) ratio. The co-culture was incubated 24 hr. After incubation, wells were washed and media was replaced with 150 μL of 1× FACS buffer containing a 1:500 dilution of 5 mg/mL DAPI (Molecular Probes) and 12.5 μL of CountBright beads (C36950; ThermoFisher Scientific). The cells were analyzed for cell viability by flow cytometry (i.e., viable cells being negative for DAPI staining). Pre-treatment with daratumumab resulted in a reduced anti-BCMA CAR T cell lysis in a dose-dependent manner (FIG. 9A). NK cells pretreated for 60 hours with 1 μg/mL daratumumab showed a 50% reduction in their ability to cause anti-BCMA CAR T cell lysis. This effect is daratumumab-specific, as anti-BCMA CAR T cells that were co-cultured with NK cells pretreated with isotype control mAb did not affect change in NK cell-mediated CAR T cell lysis.

### Example 8: Effect of High Concentrations of Daratumumab on NK and CAR T Cells

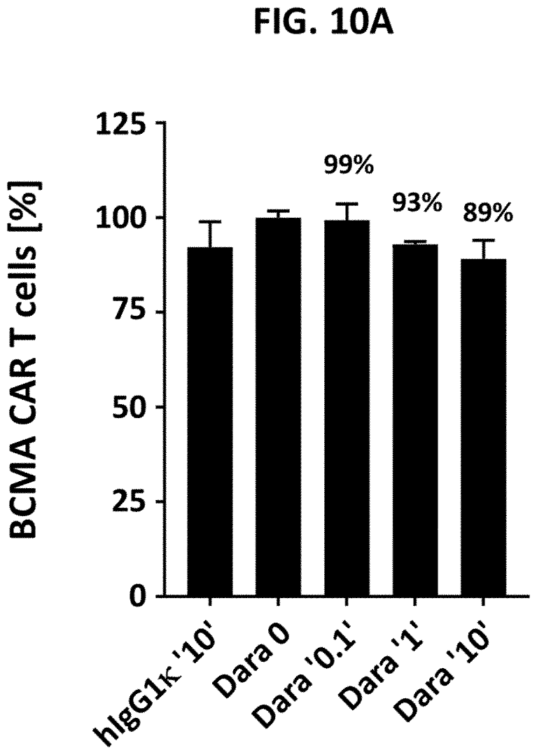

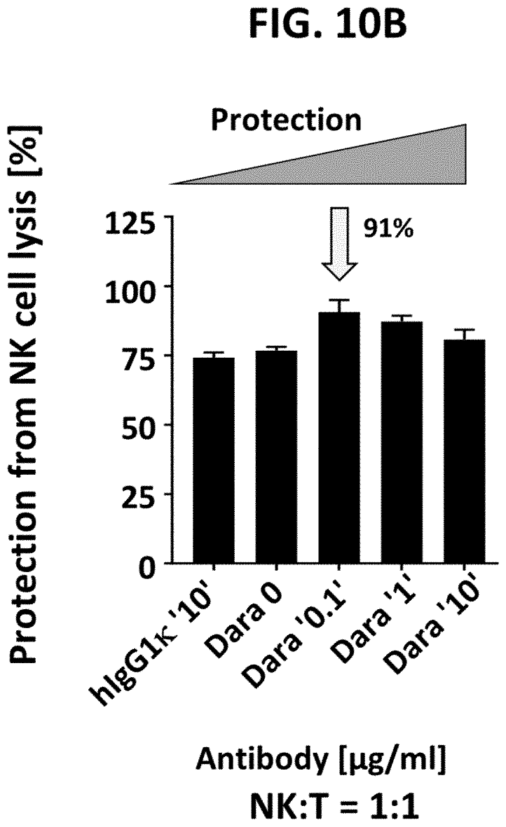

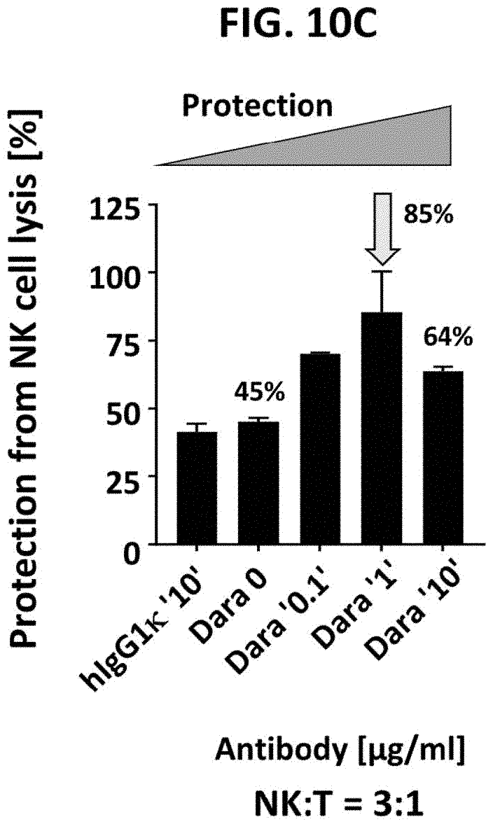

To determine if higher concentrations of daratumumab (10 μg/mL) activates CAR T cells and causes subsequent proliferation or activation-induced cell death, anti-BCMA CAR T cells deficient in B2M were cultured with daratumumab at concentrations of 0.1, 1 or 10 μg/mL for 24 hours. Untreated cells or anti-BCMA CAR T cells deficient in B2M treated with IgG1k isotype control mAb were used as controls. FIG. 10A demonstrates that increasing the concentration of daratumumab to 10 μg/mL did not significantly reduce B2M deficient CAR T cells numbers.

To determine if 10 μg/mL daratumumab blunts NK-cell mediated CAR T cell lysis, anti-BCMA CAR T cells deficient in B2M were co-cultured with purified NK cells that were pre-treated for 60 hours with either daratumumab or isotype control mAb at concentrations of 0.1, 1 or 10 μg/mL. Briefly, NK cells were plated at 50,000 or 150,000 cells per well and treated with daratumumab or the isotype control at concentrations of 0, 0.1, 1 and 10 μg/mL. After 60 hours of treatment of NK cells with daratumumab, the anti-BCMA CAR T cells were labeled with 5 μM efluor670 (Cat #65-0840-90; ThermoFisher Scientific), washed and seeded at 50,000 cells per well in co-cultures with the daratumumab-treated NK cells to make 1:1 or 3:1 (NK:T) ratio. The co-culture was incubated for further 24 hr. After incubation, wells were washed and media was replaced with 150 μL of 1× FACS buffer containing a 1:500 dilution of 5 mg/mL DAPI (Molecular Probes) and 12.5 μL of CountBright beads (C36950; ThermoFisher Scientific). The cells were analyzed for cell viability by flow cytometry (i.e., viable cells being negative for DAPI staining). Pre-treatment with daratumumab protected anti-BCMA CAR T cell from NK induced cell lysis in a dose-dependent manner (FIGS. 10B-10C). When CAR T cells were co-cultured with NK cells at a 1:1 ratio, pretreatment of the NK cells with 0.1 μg/mL daratumumab showed a maximal protective effect of 91% against anti-BCMA CAR T cell lysis (FIG. 10B). When the ratio of NK: CAR T cells increased to 3:1, daratumumab still produced a significant protective effect from NK cell lysis (85% protection) at a slightly higher dose of 1 μg/mL (FIG. 10C).

### Example 9: Effect of Increasing Doses of Daratumumab on NK and CAR T Cells

Daratumumab is prescribed in the clinic at a dose of 16 mg/kg (225 μg/mL equivalent). To determine the effect of daratumumab at high concentrations on NK and CAR T cells, anti-BCMA CAR T cells deficient in B2M were co-cultured with purified NK cells that were pre-treated for 60 hours with either human IgG1κ or daratumumab, each at concentrations of 0.01, 0.1, 1, 10, 100 or 300 μg/mL using methods as described in the previous examples. Flow cytometry was used to assess NK and CAR T cells numbers 72 hours after co-culturing with pre-treated NK cells using methods as described in the previous examples.

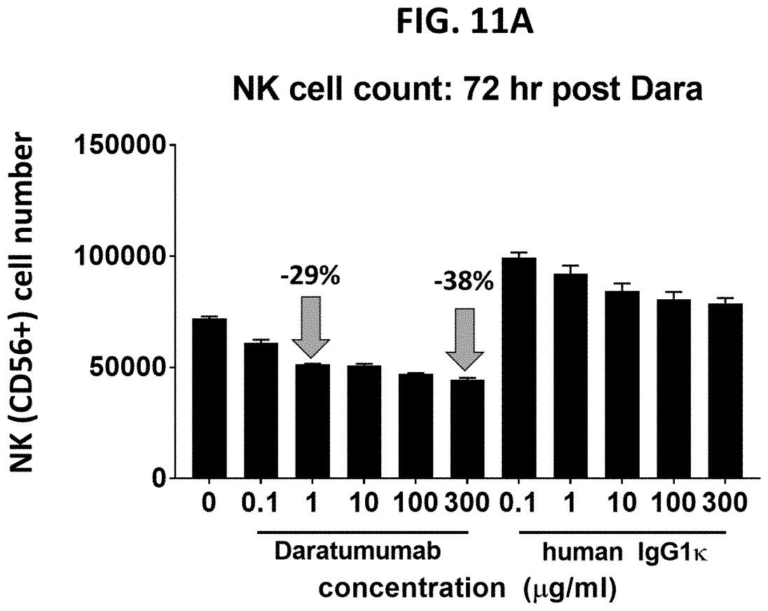

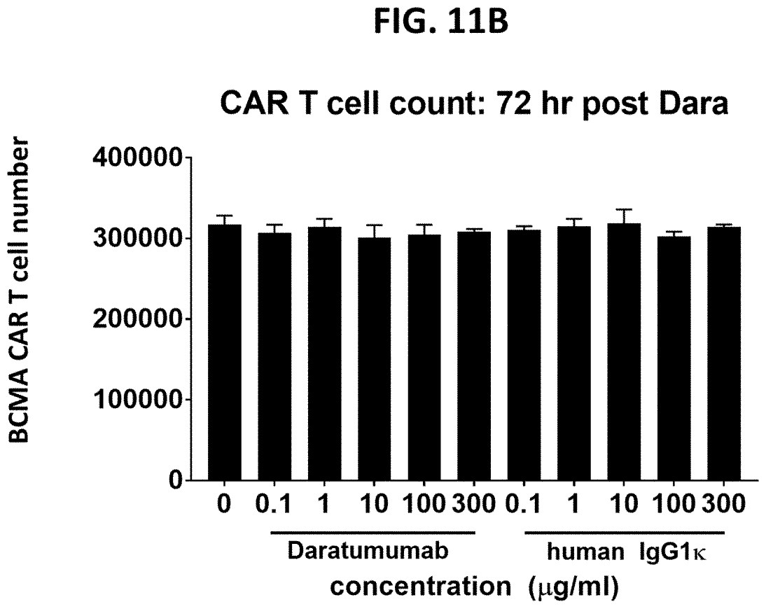

FIG. 11A demonstrates that increasing doses of daratumumab decreased NK cell number 72 hours after exposure. A 29% decrease in NK cells is seen after exposure with 1 μg/mL of daratumumab, while 300 μg/mL daratumumab results is a further 38% decrease in NK cells. In contrast, the BCMA CAR T cell numbers were unaffected by the high daratumumab concentrations (FIG. 11B).

### Example 10: In Vivo Effect of Daratumumab on NK and CAR T Cells in Murine Xenograft Model of Acute Lymphoblastic Leukemia

A disseminated mouse model was utilized to further assess the in vivo efficacy of anti-CD19 CAR T cells lacking B2M in the presence of NK cells both with and without daratumumab.

Twenty-four 5-8 week old female CIEA NOG (NOD.Cg-Prkdc^{scid}I12rg^{tm15ug}/JicTac) mice were individually housed in ventilated microisolator cages, maintained under pathogen-free conditions, 5-7 days prior to the start of the study. At the start of the study, the mice were divided into 12 treatment groups. The mice were inoculated intravenously (tail vein) to model disseminated disease. On Day 1 all mice received an intravenous injection of NALM6 tumor cells (0.5×10⁶ cells per mouse). NALM6 tumor cells used in this experiment were a human acute lymphoblastic leukemia (ALL) tumor cell line expressing GFP and luciferase. On Day 2 Groups 2-12 received an intravenous injection of NK cells, PBS, Daratumumab (DARA) and/or IgG1. PBS and IgG1 were included as negative controls. Groups 1-3 and 7-8 also received an intravenous injection of anti-CD19 CAR T cells (4×10⁶ cells per mouse) on Day 4 of the study. The anti-CD19 CAR T cells injected were prepared as described in Example 1. Groups 3, 6, 9 and 12 were negative control groups treated with IgG1 instead of Daratumumab. There were no unexpected effects of the IgG1 groups (data not shown). Details of the experimental groups are provided below in Table 6.

TABLE 6
Murine ALL Xenograft Treatment Groups.

| | NALM6 Cells | NK Cells | | DARA | IgG1 | CAR T Cells | |
|---|---|---|---|---|---|---|---|
| | 0.5 × 10⁶ | 4 × 10⁶ | | 100 | 100 | 4 × 10⁶ | |
| Group | cells/mouse | cells/mouse | PBS | μg/mouse | μg/mouse | cells/mouse | n |
| 1 | X | | | | | X | 5 |
| 2 | X | | | X | | X | 5 |
| 3 | X | | | | X | X | 5 |
| 4 | X | X | | | | | 5 |
| 5 | X | X | | X | | | 5 |
| 6 | X | X | | | X | | 5 |
| 7 | X | X | | | | X | 5 |
| 8 | X | X | | X | | X | 5 |
| 9 | X | X | | | X | X | 5 |
| 10 | X | | X | | | | 5 |
| 11 | X | | X | X | | | 5 |
| 12 | X | | X | | X | | 5 |

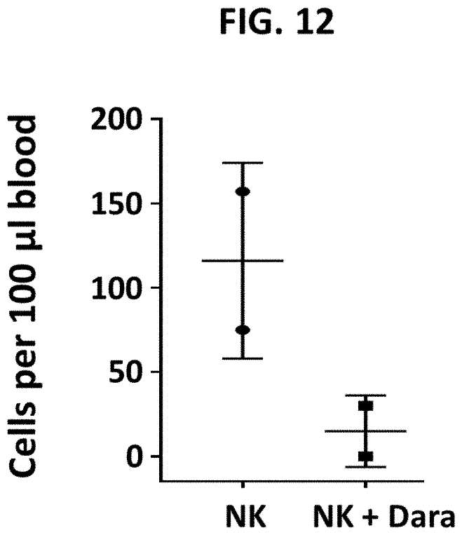

During the course of the study mice were monitored daily and body weight was measured two times weekly. Two weeks post injection, blood was collected from the mice and the number of cells was measured by flow cytometry to determine the effect of DARA on NK cells in circulation. FIG. 12 shows that DARA effectively decreased NK cell numbers the in vivo mouse models.

Disease burden was measured by bioluminescent imaging for NALM6 tumor cells marked with lentiviral vectors expressing luciferase. In brief, mice were anesthetized and luciferin administered by intraperitoneal injection. The NALM6 leukemic cells marked with luciferase metabolized luciferin and emitted light detected and quantitated using methods employed by Translations Drug Development, LLC (Scottsdale, Ariz.) and described herein. Using this method, bioluminescence (BLI; total ROI, photon/s) was measured twice weekly beginning on Day 2 of the study allowing for leukemic burden to be measured and engraftment detected.

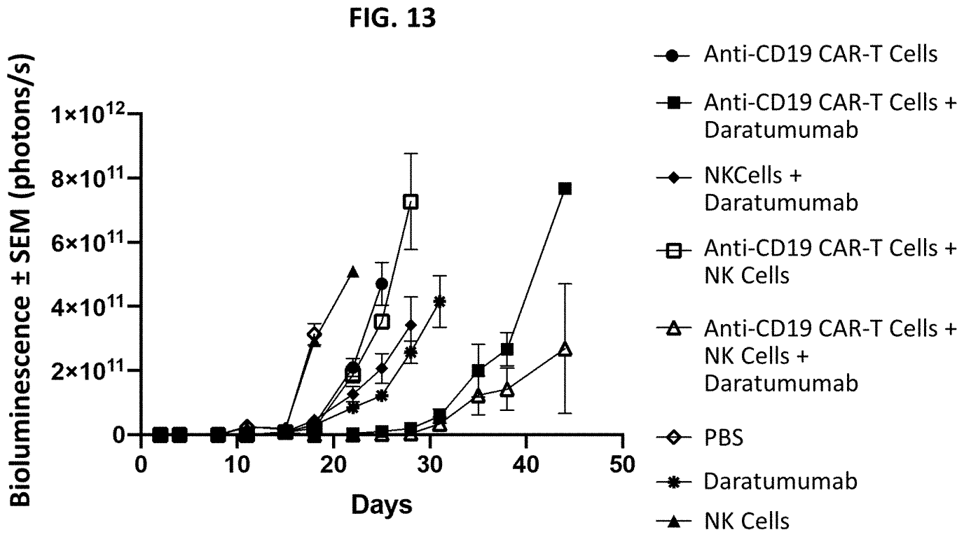

The control groups 10, 11 and 12, which did not receive NK cells, DARA or CD19 CAR T cells, showed a rapid increase in bioluminescence at 15 days and did not survive beyond 20 days (FIG. 13). Treatment with anti-CD19 CAR T cells (Group 1; FIG. 13) delayed tumor progression increased survival compared to controls. The presence of NK cells did not appear to negatively affect the efficacy of anti-CD19 CAR T cells alone. However, the addition of daratumumab dramatically increased the efficiency of anti-CD19 CAR T cells in the presence of NK cells (Group 8; FIG. 13). Unexpectedly, daratumumab alone (Group 11) and daratumumab in the presence of NK cells (Group 5) also slowed tumor growth compared to controls and had a synergistic effect when combined with CD19 CAR T cells (Group 2) (FIG. 13).

A significant endpoint (time to peri-morbidity) and the effect of T-cell engraftment were also assessed. The percentage of animal mortality and time to death were recorded for every group in the study. Mice were euthanized prior to reaching a moribund state. Mice were defined as moribund and sacrificed if one or more of the following criteria were met:
Loss of body weight of 20% or greater sustained for a period of greater than 1 week;
Tumors that inhibit normal physiological function such as eating, drinking, mobility and ability to urinate and or defecate;
Prolonged, excessive diarrhea leading to excessive weight loss (>20%); or
Persistent wheezing and respiratory distress.
   Animals were also considered moribund if there was prolonged or excessive pain or distress as defined by clinical observations such as: prostration, hunched posture, paralysis/paresis, distended abdomen, ulcerations, abscesses, seizures and/or hemorrhages. The effect of daratumumab on animal survival is provided below in Table 7 where statistical significance was determined using a Mann-Whitney test and p values were calculated compared to PBS control, (e.g., Group 10 vs. Group 1, Group 2, etc.).

TABLE 7
Effect of Daratumumab on Murine ALL Xenograft
Treatment Group Survival.

| | | Max | Median | |
|---|---|---|---|---|
| | | survival | Survival | Significance |
| Group | Treatment | (days) | (days) | (p) |
| 1 | CD19 CART | 28 | 25 | 0.0079 |
| 2 | CD19 CAR T + DARA | 44 | 42 | 0.0079 |
| 5 | NK cells + DARA | 31 | 30 | 0.0079 |
| 7 | CD19 CAR T + NK | 28 | 28 | 0.0079 |
| 8 | CD19 CAR T + NK + DARA | 44 | 42 | 0.0079 |
| 10 | PBS | 22 | 22 | 0.0079 |

### Example 11: Daratumumab Enhances the Anti-Tumor Activity of Anti-BCMA CAR-T Cells and Prolongs Survival in a Xenograft Mouse Model of Multiple Myeloma

The effect of combining daratumumab with anti-BCMA CAR-T cell treatment was tested in a subcutaneous MM.1S xenograft model in immunocompromised NOG mice (NOD.Cg-Prkdc^{scid}Il2rg^{tm15ug}/JicTac). In brief, 5 to 8-week old female NOG mice were individually housed in ventilated microisolator cages and maintained under pathogen-free conditions. The animals each received a subcutaneous inoculation in the right flank of 5×10⁶ MM.1S cells in 50% Matrigel. When the mean tumor volume reached 150 mm³ (approximately 125 to 175 mm³), the mice were randomized into groups with 5 mice per group. Tested groups included an untreated arm, daratumumab only treatment, anti-BCMA CAR-T cell only treatment (low dose or high dose), and anti-BCMA CAR-T cell (low dose or high dose) in combination with daratumumab. Anti-BCMA CAR-T cells were dosed by intravenous injection of 0.8×10⁶ (low dose) or 2.4×10⁶ (high dose) CAR+ T cells at day 0. Daratumumab was dosed IP at 15 mg/kg, twice weekly, starting 2 days prior to anti-BCMA CAR-T cell dosing.

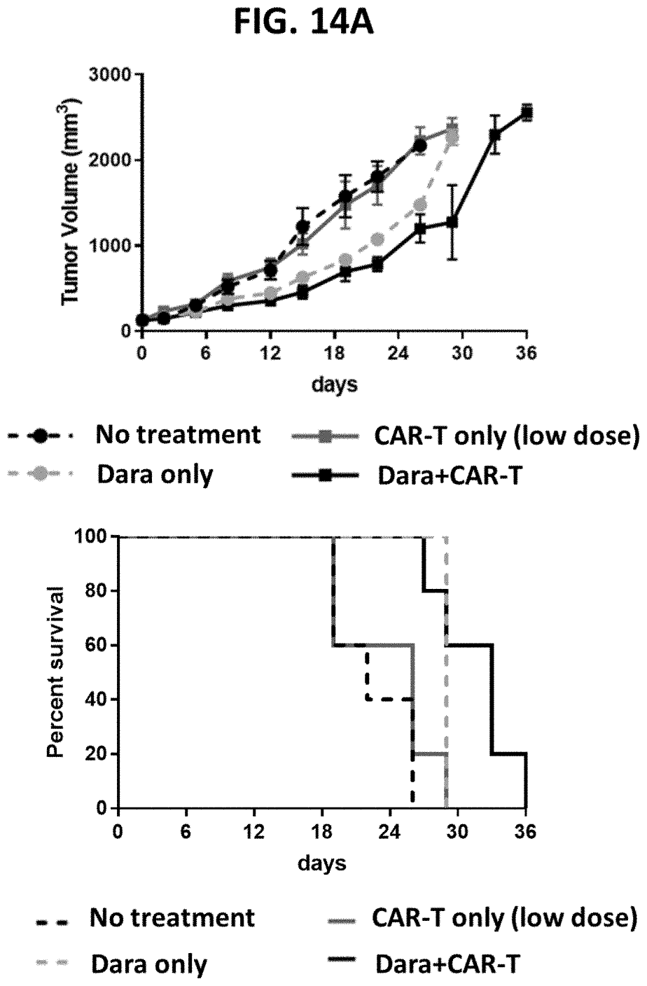

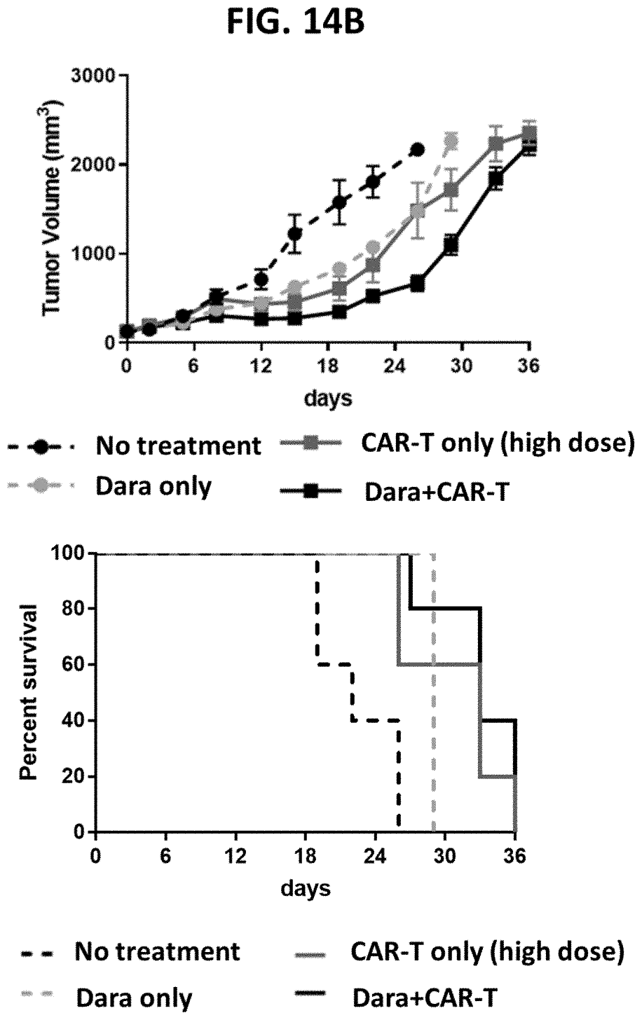

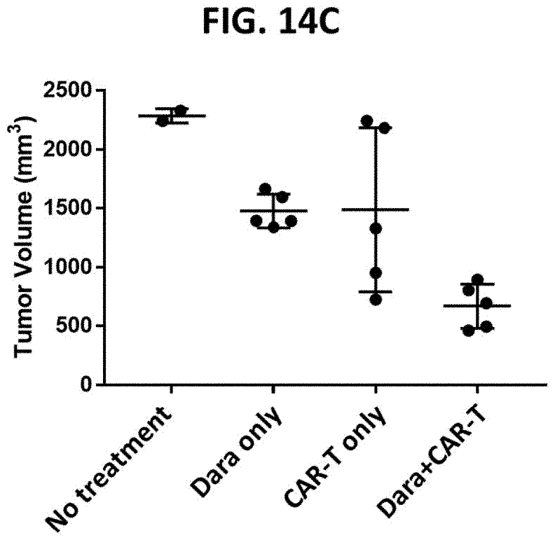

Tumor volume and body weights were measured twice weekly and individual mice were euthanized when their tumor volume reached ≥2000 mm³. In both doses of anti-BCMA CAR-T cells tested, the highest efficacy in tumor inhibition was observed in the combination arm, as compared to each single arm treatment. Additionally, prolonged survival was observed in the combination arm, in both low dose (FIG. 14A) and high dose (FIG. 14B) anti-BCMA CAR-T cell treatments, compared to either single arm treatment of daratumumab or anti-BCMA CAR-T cells. Tumor volume at day 26 is shown in FIG. 14C. Animals treated with either high dose of anti-BCMA CAR-T cells or with daratumumab only, showed mean tumor volume of ˜1500 mm³ (1486 mm³ and 1475 mm³, respectively), while mean tumor volume in the combination arm showed a mean of 668 mm³ (FIG. 14C).

In sum, these results demonstrate that the combination of anti-BCMA CAR-T cells and daratumumab showed increase efficacy in both tumor inhibition and increased survival in a mouse model of multiple myeloma compared to either anti-BCMA CAR-T cells or daratumumab alone.

SEQUENCE TABLE

| SEQ ID | | |
|---|---|---|
| NO | Description | Sequence |
| 1 | Edited TRAC gene | AAGAGCAACAAATCTGACT |
| 2 | Edited TRAC gene | AAGAGCAACAGTGCTGTGCCTGGAGCAACAAATCTGACT |
| 3 | Edited TRAC gene | AAGAGCAACAGTGCTGGAGCAACAAATCTGACT |
| 4 | Edited TRAC gene | AAGAGCAACAGTGCCTGGAGCAACAAATCTGACT |
| 5 | Edited TRAC gene | AAGAGCAACAGTGCTGACT |
| 6 | Edited TRAC gene | AAGAGCAACAGTGCTGTGGGCCTGGAGCAACAAATCTGA |
| | | CT |
| 7 | Edited TRAC gene | AAGAGCAACAGTGCTGGCCTGGAGCAACAAATCTGACT |
| 8 | Edited TRAC gene | AAGAGCAACAGTGCTGTGTGCCTGGAGCAACAAATCTGA |
| | | CT |
| 9 | Edited B2M gene | CGTGGCCTTAGCTGTGCTCGCGCTACTCTCTCTTTCTGCCT |
| | | GGAGGCTATCCAGCGTGAGTCTCTCCTACCCTCCCGCT |
| 10 | Edited B2M gene | CGTGGCCTTAGCTGTGCTCGCGCTACTCTCTCTTTCGCCTG |
| | | GAGGCTATCCAGCGTGAGTCTCTCCTACCCTCCCGCT |
| 11 | Edited B2M gene | CGTGGCCTTAGCTGTGCTCGCGCTACTCTCTCTTTCTGGA |
| | | GGCTATCCAGCGTGAGTCTCTCCTACCCTCCCGCT |
| 12 | Edited B2M gene | CGTGGCCTTAGCTGTGCTCGCGCTACTCTCTCTTTCTGGAT |
| | | AGCCTGGAGGCTATCCAGCGTGAGTCTCTCCTACCCTCCC |
| | | GCT |
| 13 | Edited B2M gene | CGTGGCCTTAGCTGTGCTCGCGCTATCCAGCGTGAGTCTC |
| | | TCCTACCCTCCCGCT |
| 14 | Edited B2M gene | CGTGGCCTTAGCTGTGCTCGCGCTACTCTCTCTTTCTGTGG |
| | | CCTGGAGGCTATCCAGCGTGAGTCTCTCCTACCCTCCCGC |
| | | T |
| 15 | sgRNA | nnnnnnnnnnnnnnnnnnnnguuuuagagcuagaaauagcaaguuaaaauaaggcu |
| | | aguccguuaucaacuugaaaaaguggcaccgagucggugcuuuu |
| 16 | sgRNA | nnnnnnnnnnnnnnnnnnnnguuuuagagcuagaaauagcaaguuaaaauaaggcu |
| | | aguccguuaucaacuugaaaaaguggcaccgagucggugc |
| 17 | sgRNA | n₍₁₇₋₃₀₎guuuuagagcuagaaauagcaaguuaaaauaaggcuaguccguuauc |
| | | aacuugaaaaaguggcaccgagucggugcu₍₁₋₈₎ |
| 18 | TRAC sgRNA | AGAGCAACAGUGCUGUGGCCguuuuagagcuagaaauagcaaguuaa |
| | unmodified | aauaaggcuaguccguuaucaacuugaaaaaguggcaccgagucggugcUUUU |
| 19 | TRAC sgRNA spacer | AGAGCAACAGUGCUGUGGCC |
| | unmodified | |
| 20 | B2M sgRNA | GCUACUCUCUCUUUCUGGCCguuuuagagcuagaaauagcaaguuaaa |
| | unmodified | auaaggcuaguccguuaucaacuugaaaaaguggcaccgagucggugcUUUU |
| 21 | B2M sgRNA spacer | GCUACUCUCUCUUUCUGGCC |
| | unmodified | |
| 22 | TRAC sgRNA | A*G*A*GCAACAGUGCUGUGGCCguuuuagagcuagaaauagcaagu |
| | modified | uaaaauaaggcuaguccguuaucaacuugaaaaaguggcaccgagucggugcU*U* |
| | *: 2′-O-methyl | U*U |
| | phosphorothioate | |
| | residue | |
| 23 | TRAC sgRNA spacer | A*G*A*GCAACAGUGCUGUGGCC |
| | modified | |
| | *: 2′-O-methyl | |
| | phosphorothioate | |
| | residue | |
| 24 | B2M sgRNA | G*C*U*ACUCUCUCUUUCUGGCCguuuuagagcuagaaauagcaagu |
| | modified | uaaaauaaggcuaguccguuaucaacuugaaaaaguggcaccgagucggugcU*U* |
| | *: 2′-O-methyl | U*U |
| | phosphorothioate | |
| | residue | |
| 25 | B2M sgRNA spacer | G*C*U*ACUCUCUCUUUCUGGCC |
| | modified | |
| | *: 2′-O-methyl | |
| | phosphorothioate | |
| | residue | |
| 26 | TRAC target | AGAGCAACAGTGCTGTGGCC (TGG) |
| | sequence with PAM | |
| 27 | B2M target sequence | GCTACTCTCTCTTTCTGGCC (TGG) |
| | with PAM | |
| 28 | exemplary TRAC | 5′-AGAGCAACAGTGCTGTGGCC-3′ |
| | target sequence | |
| 29 | exemplary B2M | 5′- GCTACTCTCTCTTTCTGGCC-3′ |
| | target sequence | |
| 30 | signal peptide | MLLLVTSLLLCELPHPAFLLIP |
| 31 | signal peptide | MALPVTALLLPLALLLHAARP |
| 32 | CD8a transmembrane | IYIWAPLAGTCGVLLLSLVITLY |
| | domain | |
| 33 | 4-1BB nucleotide | AAACGGGGCAGAAAGAAACTCCTGTATATATTCAAACAA |
| | sequence | CCATTTATGAGACCAGTACAAACTACTCAAGAGGAAGAT |
| | | GGCTGTAGCTGCCGATTTCCAGAAGAAGAAGAAGGAGGA |
| | | TGTGAACTG |
| 34 | 4-1BB amino acid | KRGRKKLLYIFKQPFMRPVQTTQEEDGCSCRFPEEEEGGCEL |
| | sequence | |
| 35 | CD28 nucleotide | TCAAAGCGGAGTAGGTTGTTGCATTCCGATTACATGAATA |
| | sequence | TGACTCCTCGCCGGCCTGGGCCGACAAGAAAACATTACC |
| | | AACCCTATGCCCCCCCACGAGACTTCGCTGCGTACAGGTC |
| | | C |
| 36 | CD28 amino acid | SKRSRLLHSDYMNMTPRRPGPTRKHYQPYAPPRDFAAYRS |
| | sequence | |
| 37 | CD3-zeta nucleotide | CGAGTGAAGTTTTCCCGAAGCGCAGACGCTCCGGCATAT |
| | sequence | CAGCAAGGACAGAATCAGCTGTATAACGAACTGAATTTG |
| | | GGACGCCGCGAGGAGTATGACGTGCTTGATAAACGCCGG |
| | | GGGAGAGACCCGGAAATGGGGGGTAAACCCCGAAGAAA |
| | | GAATCCCCAAGAAGGACTCTACAATGAACTCCAGAAGGA |
| | | TAAGATGGCGGAGGCCTACTCAGAAATAGGTATGAAGGG |
| | | CGAACGACGACGGGGAAAAGGTCACGATGGCCTCTACCA |
| | | AGGGTTGAGTACGGCAACCAAAGATACGTACGATGCACT |
| | | GCATATGCAGGCCCTGCCTCCCAGA |
| 38 | CD3-zeta amino acid | RVKFSRSADAPAYQQGQNQLYNELNLGRREEYDVLDKRRG |
| | sequence | RDPEMGGKPRRKNPQEGLYNELQKDKMAEAYSEIGMKGER |
| | | RRGKGHDGLYQGLSTATKDTYDALHMQALPPR |
| 39 | Anti-CD19 CAR | ATGCTTCTTTTGGTTACGTCTCTGTTGCTTTGCGAACTTCC |
| | FMC63-28Z | TCATCCAGCGTTCTTGCTGATCCCCGATATTCAGATGACT |
| | (FMC63-CD8[tm]- | CAGACCACCAGTAGCTTGTCTGCCTCACTGGGAGACCGA |
| | CD28[co-stimulatory | GTAACAATCTCCTGCAGGGCAAGTCAAGACATTAGCAAA |
| | domain]-CD3z) | TACCTCAATTGGTACCAGCAGAAGCCCGACGGAACGGTA |
| | | AAACTCCTCATCTATCATACGTCAAGGTTGCATTCCGGAG |
| | | TACCGTCACGATTTTCAGGTTCTGGGAGCGGAACTGACTA |
| | | TTCCTTGACTATTTCAAACCTCGAGCAGGAGGACATTGCG |
| | | ACATATTTTTGTCAACAAGGTAATACCCTCCCTTACACTT |
| | | TCGGAGGAGGAACCAAACTCGAAATTACCGGGTCCACCA |
| | | GTGGCTCTGGGAAGCCTGGCAGTGGAGAAGGTTCCACTA |
| | | AAGGCGAGGTGAAGCTCCAGGAGAGCGGCCCCGGTCTCG |
| | | TTGCCCCCAGTCAAAGCCTCTCTGTAACGTGCACAGTGAG |
| | | TGGTGTATCATTGCCTGATTATGGCGTCTCCTGGATAAGG |
| | | CAGCCCCCGCGAAAGGGTCTTGAATGGCTTGGGGTAATA |
| | | TGGGGCTCAGAGACAACGTATTATAACTCCGCTCTCAAA |
| | | AGTCGCTTGACGATAATAAAAGATAACTCCAAGAGTCAA |
| | | GTTTTCCTTAAAATGAACAGTTTGCAGACTGACGATACCG |
| | | CTATATATTATTGTGCTAAACATTATTACTACGGCGGTAG |
| | | TTACGCGATGGATTATTGGGGGCAGGGGACTTCTGTCAC |
| | | AGTCAGTAGTGCTGCTGCCTTTGTCCCGGTATTTCTCCCA |
| | | GCCAAACCGACCACGACTCCCGCCCCGCGCCCTCCGACA |
| | | CCCGCTCCCACCATCGCCTCTCAACCTCTTAGTCTTCGCC |
| | | CCGAGGCATGCCGACCCGCCGCCGGGGGTGCTGTTCATA |
| | | CGAGGGGCTTGGACTTCGCTTGTGATATTTACATTTGGGC |
| | | TCCGTTGGCGGGTACGTGCGGCGTCCTTTTGTTGTCACTC |
| | | GTTATTACTTTGTATTGTAATCACAGGAATCGCTCAAAGC |
| | | GGAGTAGGTTGTTGCATTCCGATTACATGAATATGACTCC |
| | | TCGCCGGCCTGGGCCGACAAGAAAACATTACCAACCCTA |
| | | TGCCCCCCCACGAGACTTCGCTGCGTACAGGTCCCGAGTG |
| | | AAGTTTTCCCGAAGCGCAGACGCTCCGGCATATCAGCAA |
| | | GGACAGAATCAGCTGTATAACGAACTGAATTTGGGACGC |
| | | CGCGAGGAGTATGACGTGCTTGATAAACGCCGGGGGAGA |
| | | GACCCGGAAATGGGGGGTAAACCCCGAAGAAAGAATCC |
| | | CCAAGAAGGACTCTACAATGAACTCCAGAAGGATAAGAT |
| | | GGCGGAGGCCTACTCAGAAATAGGTATGAAGGGCGAACG |
| | | ACGACGGGGAAAAGGTCACGATGGCCTCTACCAAGGGTT |
| | | GAGTACGGCAACCAAAGATACGTACGATGCACTGCATAT |
| | | GCAGGCCCTGCCTCCCAGA |
| 40 | Anti-CD19 CAR | MLLLVTSLLLCELPHPAFLLIPDIQMTQTTSSLSASLGDRVTI |
| | FMC63-28Z | SCRASQDISKYLNWYQQKPDGTVKLLIYHTSRLHSGVPSRFS |
| | (FMC63-CD8[tm]- | GSGSGTDYSLTISNLEQEDIATYFCQQGNTLPYTFGGGTKLEI |
| | CD28[co-stimulatory | TGSTSGSGKPGSGEGSTKGEVKLQESGPGLVAPSQSLSVTCT |
| | domain]-CD3z) | VSGVSLPDYGVSWIRQPPRKGLEWLGVIWGSETTYYNSALK |
| | Amino Acid | SRLTIIKDNSKSQVFLKMNSLQTDDTAIYYCAKHYYYGGSY |
| | | AMDYWGQGTSVTVSSAAAFVPVFLPAKPTTTPAPRPPTPAP |
| | | TIASQPLSLRPEACRPAAGGAVHTRGLDFACDIYIWAPLAGT |
| | | CGVLLLSLVITLYCNHRNRSKRSRLLHSDYMNMTPRRPGPT |
| | | RKHYQPYAPPRDFAAYRSRVKFSRSADAPAYQQGQNQLYN |
| | | ELNLGRREEYDVLDKRRGRDPEMGGKPRRKNPQEGLYNEL |
| | | QKDKMAEAYSEIGMKGERRRGKGHDGLYQGLSTATKDTY |
| | | DALHMQALPPR |
| 41 | Left ITR (5′ ITR) | TTGGCCACTCCCTCTCTGCGCGCTCGCTCGCTCACTGAGG |
| | | CCGGGCGACCAAAGGTCGCCCGACGCCCGGGCTTTGCCC |
| | | GGGCGGCCTCAGTGAGCGAGCGAGCGCGCAGAGAGGGA |
| | | GTGGCCAACTCCATCACTAGGGGTTCCT |
| 42 | Left ITR (5′ ITR) | CCTGCAGGCAGCTGCGCGCTCGCTCGCTCACTGAGGCCG |
| | (alternate) | CCCGGGCGTCGGGCGACCTTTGGTCGCCCGGCCTCAGTG |
| | | AGCGAGCGAGCGCGCAGAGAGGGAGTGGCCAACTCCATC |
| | | ACTAGGGGTTCCT |
| 43 | Right ITR (3′ ITR) | AGGAACCCCTAGTGATGGAGTTGGCCACTCCCTCTCTGCG |
| | | CGCTCGCTCGCTCACTGAGGCCGCCCGGGCAAAGCCCGG |
| | | GCGTCGGGCGACCTTTGGTCGCCCGGCCTCAGTGAGCGA |
| | | GCGAGCGCGCAGAGAGGGAGTGGCCAA |
| 44 | Right ITR (3′ ITR) | AGGAACCCCTAGTGATGGAGTTGGCCACTCCCTCTCTGCG |
| | (alternate) | CGCTCGCTCGCTCACTGAGGCCGGGCGACCAAAGGTCGC |
| | | CCGACGCCCGGGCTTTGCCCGGGCGGCCTCAGTGAGCGA |
| | | GCGAGCGCGCAGCTGCCTGCAGG |
| 45 | TRAC-LHA (800 bp) | GAGATGTAAGGAGCTGCTGTGACTTGCTCAAGGCCTTAT |
| | | ATCGAGTAAACGGTAGTGCTGGGGCTTAGACGCAGGTGT |
| | | TCTGATTTATAGTTCAAAACCTCTATCAATGAGAGAGCAA |
| | | TCTCCTGGTAATGTGATAGATTTCCCAACTTAATGCCAAC |
| | | ATACCATAAACCTCCCATTCTGCTAATGCCCAGCCTAAGT |
| | | TGGGGAGACCACTCCAGATTCCAAGATGTACAGTTTGCTT |
| | | TGCTGGGCCTTTTTCCCATGCCTGCCTTTACTCTGCCAGA |
| | | GTTATATTGCTGGGGTTTTGAAGAAGATCCTATTAAATAA |
| | | AAGAATAAGCAGTATTATTAAGTAGCCCTGCATTTCAGGT |
| | | TTCCTTGAGTGGCAGGCCAGGCCTGGCCGTGAACGTTCAC |
| | | TGAAATCATGGCCTCTTGGCCAAGATTGATAGCTTGTGCC |
| | | TGTCCCTGAGTCCCAGTCCATCACGAGCAGCTGGTTTCTA |
| | | AGATGCTATTTCCCGTATAAAGCATGAGACCGTGACTTGC |
| | | CAGCCCCACAGAGCCCCGCCCTTGTCCATCACTGGCATCT |
| | | GGACTCCAGCCTGGGTTGGGGCAAAGAGGGAAATGAGAT |
| | | CATGTCCTAACCCTGATCCTCTTGTCCCACAGATATCCAG |
| | | AACCCTGACCCTGCCGTGTACCAGCTGAGAGACTCTAAA |
| | | TCCAGTGACAAGTCTGTCTGCCTATTCACCGATTTTGATT |
| | | CTCAAACAAATGTGTCACAAAGTAAGGATTCTGATGTGT |
| | | ATATCACAGACAAAACTGTGCTAGACATGAGGTCTATGG |
| | | ACTTCA |
| 46 | TRAC-RHA (800 bp) | TGGAGCAACAAATCTGACTTTGCATGTGCAAACGCCTTCA |
| | | ACAACAGCATTATTCCAGAAGACACCTTCTTCCCCAGCCC |
| | | AGGTAAGGGCAGCTTTGGTGCCTTCGCAGGCTGTTTCCTT |
| | | GCTTCAGGAATGGCCAGGTTCTGCCCAGAGCTCTGGTCA |
| | | ATGATGTCTAAAACTCCTCTGATTGGTGGTCTCGGCCTTA |
| | | TCCATTGCCACCAAAACCCTCTTTTTACTAAGAAACAGTG |
| | | AGCCTTGTTCTGGCAGTCCAGAGAATGACACGGGAAAAA |
| | | AGCAGATGAAGAGAAGGTGGCAGGAGAGGGCACGTGGC |
| | | CCAGCCTCAGTCTCTCCAACTGAGTTCCTGCCTGCCTGCC |
| | | TTTGCTCAGACTGTTTGCCCCTTACTGCTCTTCTAGGCCTC |
| | | ATTCTAAGCCCCTTCTCCAAGTTGCCTCTCCTTATTTCTCC |
| | | CTGTCTGCCAAAAAATCTTTCCCAGCTCACTAAGTCAGTC |
| | | TCACGCAGTCACTCATTAACCCACCAATCACTGATTGTGC |
| | | CGGCACATGAATGCACCAGGTGTTGAAGTGGAGGAATTA |
| | | AAAAGTCAGATGAGGGGTGTGCCCAGAGGAAGCACCATT |
| | | CTAGTTGGGGGAGCCCATCTGTCAGCTGGGAAAAGTCCA |
| | | AATAACTTCAGATTGGAATGTGTTTTAACTCAGGGTTGAG |
| | | AAAACAGCTACCTTCAGGACAAAAGTCAGGGAAGGGCTC |
| | | TCTGAAGAAATGCTACTTGAAGATACCAGCCCTACCAAG |
| | | GGCAGGGAGAGGACCCTATAGAGGCCTGGGACAGGAGC |
| | | TCAATGAGAAAGG |
| 47 | EF1a | GGCTCCGGTGCCCGTCAGTGGGCAGAGCGCACATCGCCC |
| | | ACAGTCCCCGAGAAGTTGGGGGGAGGGGTCGGCAATTGA |
| | | ACCGGTGCCTAGAGAAGGTGGCGCGGGGTAAACTGGGAA |
| | | AGTGATGTCGTGTACTGGCTCCGCCTTTTTCCCGAGGGTG |
| | | GGGGAGAACCGTATATAAGTGCAGTAGTCGCCGTGAACG |
| | | TTCTTTTTCGCAACGGGTTTGCCGCCAGAACACAGGTAAG |
| | | TGCCGTGTGTGGTTCCCGCGGGCCTGGCCTCTTTACGGGT |
| | | TATGGCCCTTGCGTGCCTTGAATTACTTCCACTGGCTGCA |
| | | GTACGTGATTCTTGATCCCGAGCTTCGGGTTGGAAGTGGG |
| | | TGGGAGAGTTCGAGGCCTTGCGCTTAAGGAGCCCCTTCG |
| | | CCTCGTGCTTGAGTTGAGGCCTGGCCTGGGCGCTGGGGCC |
| | | GCCGCGTGCGAATCTGGTGGCACCTTCGCGCCTGTCTCGC |
| | | TGCTTTCGATAAGTCTCTAGCCATTTAAAATTTTTGATGA |
| | | CCTGCTGCGACGCTTTTTTTCTGGCAAGATAGTCTTGTAA |
| | | ATGCGGGCCAAGATCTGCACACTGGTATTTCGGTTTTTGG |
| | | GGCCGCGGGCGGCGACGGGGCCCGTGCGTCCCAGCGCAC |
| | | ATGTTCGGCGAGGCGGGGCCTGCGAGCGCGGCCACCGAG |
| | | AATCGGACGGGGGTAGTCTCAAGCTGGCCGGCCTGCTCT |
| | | GGTGCCTGGCCTCGCGCCGCCGTGTATCGCCCCGCCCTGG |
| | | GCGGCAAGGCTGGCCCGGTCGGCACCAGTTGCGTGAGCG |
| | | GAAAGATGGCCGCTTCCCGGCCCTGCTGCAGGGAGCTCA |
| | | AAATGGAGGACGCGGCGCTCGGGAGAGCGGGCGGGTGA |
| | | GTCACCCACACAAAGGAAAAGGGCCTTTCCGTCCTCAGC |
| | | CGTCGCTTCATGTGACTCCACGGAGTACCGGGCGCCGTCC |
| | | AGGCACCTCGATTAGTTCTCGAGCTTTTGGAGTACGTCGT |
| | | CTTTAGGTTGGGGGGAGGGGTTTTATGCGATGGAGTTTCC |
| | | CCACACTGAGTGGGTGGAGACTGAAGTTAGGCCAGCTTG |
| | | GCACTTGATGTAATTCTCCTTGGAATTTGCCCTTTTTGAGT |
| | | TTGGATCTTGGTTCATTCTCAAGCCTCAGACAGTGGTTCA |
| | | AAGTTTTTTTCTTCCATTTCAGGTGTCGTGA |
| 48 | GM-CSF signal | ATGCTTCTTTTGGTTACGTCTCTGTTGCTTTGCGAACTTCC |
| | peptide coding | TCATCCAGCGTTCTTGCTGATCCCC |
| | sequence | |
| 49 | Anti-CD19 scFv | GATATTCAGATGACTCAGACCACCAGTAGCTTGTCTGCCT |
| | coding sequence | CACTGGGAGACCGAGTAACAATCTCCTGCAGGGCAAGTC |
| | | AAGACATTAGCAAATACCTCAATTGGTACCAGCAGAAGC |
| | | CCGACGGAACGGTAAAACTCCTCATCTATCATACGTCAA |
| | | GGTTGCATTCCGGAGTACCGTCACGATTTTCAGGTTCTGG |
| | | GAGCGGAACTGACTATTCCTTGACTATTTCAAACCTCGAG |
| | | CAGGAGGACATTGCGACATATTTTTGTCAACAAGGTAAT |
| | | ACCCTCCCTTACACTTTCGGAGGAGGAACCAAACTCGAA |
| | | ATTACCGGGTCCACCAGTGGCTCTGGGAAGCCTGGCAGT |
| | | GGAGAAGGTTCCACTAAAGGCGAGGTGAAGCTCCAGGAG |
| | | AGCGGCCCCGGTCTCGTTGCCCCCAGTCAAAGCCTCTCTG |
| | | TAACGTGCACAGTGAGTGGTGTATCATTGCCTGATTATGG |
| | | CGTCTCCTGGATAAGGCAGCCCCCGCGAAAGGGTCTTGA |
| | | ATGGCTTGGGGTAATATGGGGCTCAGAGACAACGTATTA |
| | | TAACTCCGCTCTCAAAAGTCGCTTGACGATAATAAAAGA |
| | | TAACTCCAAGAGTCAAGTTTTCCTTAAAATGAACAGTTTG |
| | | CAGACTGACGATACCGCTATATATTATTGTGCTAAACATT |
| | | ATTACTACGGCGGTAGTTACGCGATGGATTATTGGGGGC |
| | | AGGGGACTTCTGTCACAGTCAGTAGT |
| 50 | CD19 scFv amino | DIQMTQTTSSLSASLGDRVTISCRASQDISKYLNWYQQKPD |
| | acid sequence | GTVKLLIYHTSRLHSGVPSRFSGSGSGTDYSLTISNLEQEDIA |
| | Linker underlined | TYFCQQGNTLPYTFGGGTKLEITGSTSGSGKPGSGEGSTKGE |
| | | VKLQESGPGLVAPSQSLSVTCTVSGVSLPDYGVSWIRQPPRK |
| | | GLEWLGVIWGSETTYYNSALKSRLTIIKDNSKSQVFLKMNS |
| | | LQTDDTAIYYCAKHYYYGGSYAMDYWGQGTSVTVSS |
| 51 | CD8a extracellular + | GCTGCTGCCTTTGTCCCGGTATTTCTCCCAGCCAAACCGA |
| | CD8a transmembrane + | CCACGACTCCCGCCCCGCGCCCTCCGACACCCGCTCCCAC |
| | 5′Linker | CATCGCCTCTCAACCTCTTAGTCTTCGCCCCGAGGCATGC |
| | (underlined) | CGACCCGCCGCCGGGGGTGCTGTTCATACGAGGGGCTTG |
| | | GACTTCGCTTGTGATATTTACATTTGGGCTCCGTTGGCGG |
| | | GTACGTGCGGCGTCCTTTTGTTGTCACTCGTTATTACTTTG |
| | | TATTGTAATCACAGGAATCGC |
| 52 | CD8a extracellular + | TTTGTCCCGGTATTTCTCCCAGCCAAACCGACCACGACTC |
| | CD8a transmembrane | CCGCCCCGCGCCCTCCGACACCCGCTCCCACCATCGCCTC |
| | (without linker) | TCAACCTCTTAGTCTTCGCCCCGAGGCATGCCGACCCGCC |
| | | GCCGGGGGTGCTGTTCATACGAGGGGCTTGGACTTCGCTT |
| | | GTGATATTTACATTTGGGCTCCGTTGGCGGGTACGTGCGG |
| | | CGTCCTTTTGTTGTCACTCGTTATTACTTTGTATTGTAATC |
| | | ACAGGAATCGC |
| 53 | CD8a extracellular + | FVPVFLPAKPTTTPAPRPPTPAPTIASQPLSLRPEACRPAAGG |
| | CD8a transmembrane | AVHTRGLDFACDIYIWAPLAGTCGVLLLSLVITLYCNHRNR |
| 54 | CD19 VH | EVKLQESGPGLVAPSQSLSVTCTVSGVSLPDYGVSWIRQPPR |
| | | KGLEWLGVIWGSETTYYNSALKSRLTIIKDNSKSQVFLKMN |
| | | SLQTDDTAIYYCAKHYYYGGSYAMDYWGQGTSVTVSS |
| 55 | CD19 VL | DIQMTQTTSSLSASLGDRVTISCRASQDISKYLNWYQQKPD |
| | | GTVKLLIYHTSRLHSGVPSRFSGSGSGTDYSLTISNLEQEDIA |
| | | TYFCQQGNTLPYTFGGGTKLEIT |
| 56 | CD19 linker | GSTSGSGKPGSGEGSTKG |
| 57 | CD19 | CCTGCAGGCAGCTGCGCGCTCGCTCGCTCACTGAGGCCG |
| | rAAV | CCCGGGCGTCGGGCGACCTTTGGTCGCCCGGCCTCAGTG |
| | | AGCGAGCGAGCGCGCAGAGAGGGAGTGGCCAACTCCATC |
| | | ACTAGGGGTTCCTGCGGCCGCACGCGTGAGATGTAAGGA |
| | | GCTGCTGTGACTTGCTCAAGGCCTTATATCGAGTAAACGG |
| | | TAGTGCTGGGGCTTAGACGCAGGTGTTCTGATTTATAGTT |
| | | CAAAACCTCTATCAATGAGAGAGCAATCTCCTGGTAATG |
| | | TGATAGATTTCCCAACTTAATGCCAACATACCATAAACCT |
| | | CCCATTCTGCTAATGCCCAGCCTAAGTTGGGGAGACCACT |
| | | CCAGATTCCAAGATGTACAGTTTGCTTTGCTGGGCCTTTT |
| | | TCCCATGCCTGCCTTTACTCTGCCAGAGTTATATTGCTGG |
| | | GGTTTTGAAGAAGATCCTATTAAATAAAAGAATAAGCAG |
| | | TATTATTAAGTAGCCCTGCATTTCAGGTTTCCTTGAGTGG |
| | | CAGGCCAGGCCTGGCCGTGAACGTTCACTGAAATCATGG |
| | | CCTCTTGGCCAAGATTGATAGCTTGTGCCTGTCCCTGAGT |
| | | CCCAGTCCATCACGAGCAGCTGGTTTCTAAGATGCTATTT |
| | | CCCGTATAAAGCATGAGACCGTGACTTGCCAGCCCCACA |
| | | GAGCCCCGCCCTTGTCCATCACTGGCATCTGGACTCCAGC |
| | | CTGGGTTGGGGCAAAGAGGGAAATGAGATCATGTCCTAA |
| | | CCCTGATCCTCTTGTCCCACAGATATCCAGAACCCTGACC |
| | | CTGCCGTGTACCAGCTGAGAGACTCTAAATCCAGTGACA |
| | | AGTCTGTCTGCCTATTCACCGATTTTGATTCTCAAACAAA |
| | | TGTGTCACAAAGTAAGGATTCTGATGTGTATATCACAGAC |
| | | AAAACTGTGCTAGACATGAGGTCTATGGACTTCAGGCTC |
| | | CGGTGCCCGTCAGTGGGCAGAGCGCACATCGCCCACAGT |
| | | CCCCGAGAAGTTGGGGGGAGGGGTCGGCAATTGAACCGG |
| | | TGCCTAGAGAAGGTGGCGCGGGGTAAACTGGGAAAGTGA |
| | | TGTCGTGTACTGGCTCCGCCTTTTTCCCGAGGGTGGGGGA |
| | | GAACCGTATATAAGTGCAGTAGTCGCCGTGAACGTTCTTT |
| | | TTCGCAACGGGTTTGCCGCCAGAACACAGGTAAGTGCCG |
| | | TGTGTGGTTCCCGCGGGCCTGGCCTCTTTACGGGTTATGG |
| | | CCCTTGCGTGCCTTGAATTACTTCCACTGGCTGCAGTACG |
| | | TGATTCTTGATCCCGAGCTTCGGGTTGGAAGTGGGTGGGA |
| | | GAGTTCGAGGCCTTGCGCTTAAGGAGCCCCTTCGCCTCGT |
| | | GCTTGAGTTGAGGCCTGGCCTGGGCGCTGGGGCCGCCGC |
| | | GTGCGAATCTGGTGGCACCTTCGCGCCTGTCTCGCTGCTT |
| | | TCGATAAGTCTCTAGCCATTTAAAATTTTTGATGACCTGC |
| | | TGCGACGCTTTTTTTCTGGCAAGATAGTCTTGTAAATGCG |
| | | GGCCAAGATCTGCACACTGGTATTTCGGTTTTTGGGGCCG |
| | | CGGGCGGCGACGGGGCCCGTGCGTCCCAGCGCACATGTT |
| | | CGGCGAGGCGGGGCCTGCGAGCGCGGCCACCGAGAATCG |
| | | GACGGGGGTAGTCTCAAGCTGGCCGGCCTGCTCTGGTGC |
| | | CTGGCCTCGCGCCGCCGTGTATCGCCCCGCCCTGGGCGGC |
| | | AAGGCTGGCCCGGTCGGCACCAGTTGCGTGAGCGGAAAG |
| | | ATGGCCGCTTCCCGGCCCTGCTGCAGGGAGCTCAAAATG |
| | | GAGGACGCGGCGCTCGGGAGAGCGGGCGGGTGAGTCAC |
| | | CCACACAAAGGAAAAGGGCCTTTCCGTCCTCAGCCGTCG |
| | | CTTCATGTGACTCCACGGAGTACCGGGCGCCGTCCAGGC |
| | | ACCTCGATTAGTTCTCGAGCTTTTGGAGTACGTCGTCTTT |
| | | AGGTTGGGGGGAGGGGTTTTATGCGATGGAGTTTCCCCA |
| | | CACTGAGTGGGTGGAGACTGAAGTTAGGCCAGCTTGGCA |
| | | CTTGATGTAATTCTCCTTGGAATTTGCCCTTTTTGAGTTTG |
| | | GATCTTGGTTCATTCTCAAGCCTCAGACAGTGGTTCAAAG |
| | | TTTTTTTCTTCCATTTCAGGTGTCGTGACCACCATGCTTCT |
| | | TTTGGTTACGTCTCTGTTGCTTTGCGAACTTCCTCATCCAG |
| | | CGTTCTTGCTGATCCCCGATATTCAGATGACTCAGACCAC |
| | | CAGTAGCTTGTCTGCCTCACTGGGAGACCGAGTAACAAT |
| | | CTCCTGCAGGGCAAGTCAAGACATTAGCAAATACCTCAA |
| | | TTGGTACCAGCAGAAGCCCGACGGAACGGTAAAACTCCT |
| | | CATCTATCATACGTCAAGGTTGCATTCCGGAGTACCGTCA |
| | | CGATTTTCAGGTTCTGGGAGCGGAACTGACTATTCCTTGA |
| | | CTATTTCAAACCTCGAGCAGGAGGACATTGCGACATATTT |
| | | TTGTCAACAAGGTAATACCCTCCCTTACACTTTCGGAGGA |
| | | GGAACCAAACTCGAAATTACCGGGTCCACCAGTGGCTCT |
| | | GGGAAGCCTGGCAGTGGAGAAGGTTCCACTAAAGGCGAG |
| | | GTGAAGCTCCAGGAGAGCGGCCCCGGTCTCGTTGCCCCC |
| | | AGTCAAAGCCTCTCTGTAACGTGCACAGTGAGTGGTGTAT |
| | | CATTGCCTGATTATGGCGTCTCCTGGATAAGGCAGCCCCC |
| | | GCGAAAGGGTCTTGAATGGCTTGGGGTAATATGGGGCTC |
| | | AGAGACAACGTATTATAACTCCGCTCTCAAAAGTCGCTTG |
| | | ACGATAATAAAAGATAACTCCAAGAGTCAAGTTTTCCTT |
| | | AAAATGAACAGTTTGCAGACTGACGATACCGCTATATAT |
| | | TATTGTGCTAAACATTATTACTACGGCGGTAGTTACGCGA |
| | | TGGATTATTGGGGGCAGGGGACTTCTGTCACAGTCAGTA |
| | | GTGCTGCTGCCTTTGTCCCGGTATTTCTCCCAGCCAAACC |
| | | GACCACGACTCCCGCCCCGCGCCCTCCGACACCCGCTCCC |
| | | ACCATCGCCTCTCAACCTCTTAGTCTTCGCCCCGAGGCAT |
| | | GCCGACCCGCCGCCGGGGGTGCTGTTCATACGAGGGGCT |
| | | TGGACTTCGCTTGTGATATTTACATTTGGGCTCCGTTGGC |
| | | GGGTACGTGCGGCGTCCTTTTGTTGTCACTCGTTATTACTT |
| | | TGTATTGTAATCACAGGAATCGCTCAAAGCGGAGTAGGT |
| | | TGTTGCATTCCGATTACATGAATATGACTCCTCGCCGGCC |
| | | TGGGCCGACAAGAAAACATTACCAACCCTATGCCCCCCC |
| | | ACGAGACTTCGCTGCGTACAGGTCCCGAGTGAAGTTTTCC |
| | | CGAAGCGCAGACGCTCCGGCATATCAGCAAGGACAGAAT |
| | | CAGCTGTATAACGAACTGAATTTGGGACGCCGCGAGGAG |
| | | TATGACGTGCTTGATAAACGCCGGGGGAGAGACCCGGAA |
| | | ATGGGGGGTAAACCCCGAAGAAAGAATCCCCAAGAAGG |
| | | ACTCTACAATGAACTCCAGAAGGATAAGATGGCGGAGGC |
| | | CTACTCAGAAATAGGTATGAAGGGCGAACGACGACGGGG |
| | | AAAAGGTCACGATGGCCTCTACCAAGGGTTGAGTACGGC |
| | | AACCAAAGATACGTACGATGCACTGCATATGCAGGCCCT |
| | | GCCTCCCAGATAATAATAAAATCGCTATCCATCGAAGAT |
| | | GGATGTGTGTTGGTTTTTTGTGTGTGGAGCAACAAATCTG |
| | | ACTTTGCATGTGCAAACGCCTTCAACAACAGCATTATTCC |
| | | AGAAGACACCTTCTTCCCCAGCCCAGGTAAGGGCAGCTT |
| | | TGGTGCCTTCGCAGGCTGTTTCCTTGCTTCAGGAATGGCC |
| | | AGGTTCTGCCCAGAGCTCTGGTCAATGATGTCTAAAACTC |
| | | CTCTGATTGGTGGTCTCGGCCTTATCCATTGCCACCAAAA |
| | | CCCTCTTTTTACTAAGAAACAGTGAGCCTTGTTCTGGCAG |
| | | TCCAGAGAATGACACGGGAAAAAAGCAGATGAAGAGAA |
| | | GGTGGCAGGAGAGGGCACGTGGCCCAGCCTCAGTCTCTC |
| | | CAACTGAGTTCCTGCCTGCCTGCCTTTGCTCAGACTGTTT |
| | | GCCCCTTACTGCTCTTCTAGGCCTCATTCTAAGCCCCTTCT |
| | | CCAAGTTGCCTCTCCTTATTTCTCCCTGTCTGCCAAAAAA |
| | | TCTTTCCCAGCTCACTAAGTCAGTCTCACGCAGTCACTCA |
| | | TTAACCCACCAATCACTGATTGTGCCGGCACATGAATGCA |
| | | CCAGGTGTTGAAGTGGAGGAATTAAAAAGTCAGATGAGG |
| | | GGTGTGCCCAGAGGAAGCACCATTCTAGTTGGGGGAGCC |
| | | CATCTGTCAGCTGGGAAAAGTCCAAATAACTTCAGATTG |
| | | GAATGTGTTTTAACTCAGGGTTGAGAAAACAGCTACCTTC |
| | | AGGACAAAAGTCAGGGAAGGGCTCTCTGAAGAAATGCTA |
| | | CTTGAAGATACCAGCCCTACCAAGGGCAGGGAGAGGACC |
| | | CTATAGAGGCCTGGGACAGGAGCTCAATGAGAAAGGTAA |
| | | CCACGTGCGGACCGAGGCTGCAGCGTCGTCCTCCCTAGG |
| | | AACCCCTAGTGATGGAGTTGGCCACTCCCTCTCTGCGCGC |
| | | TCGCTCGCTCACTGAGGCCGGGCGACCAAAGGTCGCCCG |
| | | ACGCCCGGGCTTTGCCCGGGCGGCCTCAGTGAGCGAGCG |
| | | AGCGCGCAGCTGCCTGCAGG |
| 58 | CD19 | GAGATGTAAGGAGCTGCTGTGACTTGCTCAAGGCCTTAT |
| | LHA to RHA | ATCGAGTAAACGGTAGTGCTGGGGCTTAGACGCAGGTGT |
| | | TCTGATTTATAGTTCAAAACCTCTATCAATGAGAGAGCAA |
| | | TCTCCTGGTAATGTGATAGATTTCCCAACTTAATGCCAAC |
| | | ATACCATAAACCTCCCATTCTGCTAATGCCCAGCCTAAGT |
| | | TGGGGAGACCACTCCAGATTCCAAGATGTACAGTTTGCTT |
| | | TGCTGGGCCTTTTTCCCATGCCTGCCTTTACTCTGCCAGA |
| | | GTTATATTGCTGGGGTTTTGAAGAAGATCCTATTAAATAA |
| | | AAGAATAAGCAGTATTATTAAGTAGCCCTGCATTTCAGGT |
| | | TTCCTTGAGTGGCAGGCCAGGCCTGGCCGTGAACGTTCAC |
| | | TGAAATCATGGCCTCTTGGCCAAGATTGATAGCTTGTGCC |
| | | TGTCCCTGAGTCCCAGTCCATCACGAGCAGCTGGTTTCTA |
| | | AGATGCTATTTCCCGTATAAAGCATGAGACCGTGACTTGC |
| | | CAGCCCCACAGAGCCCCGCCCTTGTCCATCACTGGCATCT |
| | | GGACTCCAGCCTGGGTTGGGGCAAAGAGGGAAATGAGAT |
| | | CATGTCCTAACCCTGATCCTCTTGTCCCACAGATATCCAG |
| | | AACCCTGACCCTGCCGTGTACCAGCTGAGAGACTCTAAA |
| | | TCCAGTGACAAGTCTGTCTGCCTATTCACCGATTTTGATT |
| | | CTCAAACAAATGTGTCACAAAGTAAGGATTCTGATGTGT |
| | | ATATCACAGACAAAACTGTGCTAGACATGAGGTCTATGG |
| | | ACTTCAGGCTCCGGTGCCCGTCAGTGGGCAGAGCGCACA |
| | | TCGCCCACAGTCCCCGAGAAGTTGGGGGGAGGGGTCGGC |
| | | AATTGAACCGGTGCCTAGAGAAGGTGGCGCGGGGTAAAC |
| | | TGGGAAAGTGATGTCGTGTACTGGCTCCGCCTTTTTCCCG |
| | | AGGGTGGGGGAGAACCGTATATAAGTGCAGTAGTCGCCG |
| | | TGAACGTTCTTTTTCGCAACGGGTTTGCCGCCAGAACACA |
| | | GGTAAGTGCCGTGTGTGGTTCCCGCGGGCCTGGCCTCTTT |
| | | ACGGGTTATGGCCCTTGCGTGCCTTGAATTACTTCCACTG |
| | | GCTGCAGTACGTGATTCTTGATCCCGAGCTTCGGGTTGGA |
| | | AGTGGGTGGGAGAGTTCGAGGCCTTGCGCTTAAGGAGCC |
| | | CCTTCGCCTCGTGCTTGAGTTGAGGCCTGGCCTGGGCGCT |
| | | GGGGCCGCCGCGTGCGAATCTGGTGGCACCTTCGCGCCT |
| | | GTCTCGCTGCTTTCGATAAGTCTCTAGCCATTTAAAATTTT |
| | | TGATGACCTGCTGCGACGCTTTTTTTCTGGCAAGATAGTC |
| | | TTGTAAATGCGGGCCAAGATCTGCACACTGGTATTTCGGT |
| | | TTTTGGGGCCGCGGGCGGCGACGGGGCCCGTGCGTCCCA |
| | | GCGCACATGTTCGGCGAGGCGGGGCCTGCGAGCGCGGCC |
| | | ACCGAGAATCGGACGGGGGTAGTCTCAAGCTGGCCGGCC |
| | | TGCTCTGGTGCCTGGCCTCGCGCCGCCGTGTATCGCCCCG |
| | | CCCTGGGCGGCAAGGCTGGCCCGGTCGGCACCAGTTGCG |
| | | TGAGCGGAAAGATGGCCGCTTCCCGGCCCTGCTGCAGGG |
| | | AGCTCAAAATGGAGGACGCGGCGCTCGGGAGAGCGGGC |
| | | GGGTGAGTCACCCACACAAAGGAAAAGGGCCTTTCCGTC |
| | | CTCAGCCGTCGCTTCATGTGACTCCACGGAGTACCGGGCG |
| | | CCGTCCAGGCACCTCGATTAGTTCTCGAGCTTTTGGAGTA |
| | | CGTCGTCTTTAGGTTGGGGGGAGGGGTTTTATGCGATGGA |
| | | GTTTCCCCACACTGAGTGGGTGGAGACTGAAGTTAGGCC |
| | | AGCTTGGCACTTGATGTAATTCTCCTTGGAATTTGCCCTTT |
| | | TTGAGTTTGGATCTTGGTTCATTCTCAAGCCTCAGACAGT |
| | | GGTTCAAAGTTTTTTTCTTCCATTTCAGGTGTCGTGACCAC |
| | | CATGCTTCTTTTGGTTACGTCTCTGTTGCTTTGCGAACTTC |
| | | CTCATCCAGCGTTCTTGCTGATCCCCGATATTCAGATGAC |
| | | TCAGACCACCAGTAGCTTGTCTGCCTCACTGGGAGACCG |
| | | AGTAACAATCTCCTGCAGGGCAAGTCAAGACATTAGCAA |
| | | ATACCTCAATTGGTACCAGCAGAAGCCCGACGGAACGGT |
| | | AAAACTCCTCATCTATCATACGTCAAGGTTGCATTCCGGA |
| | | GTACCGTCACGATTTTCAGGTTCTGGGAGCGGAACTGACT |
| | | ATTCCTTGACTATTTCAAACCTCGAGCAGGAGGACATTGC |
| | | GACATATTTTTGTCAACAAGGTAATACCCTCCCTTACACT |
| | | TTCGGAGGAGGAACCAAACTCGAAATTACCGGGTCCACC |
| | | AGTGGCTCTGGGAAGCCTGGCAGTGGAGAAGGTTCCACT |
| | | AAAGGCGAGGTGAAGCTCCAGGAGAGCGGCCCCGGTCTC |
| | | GTTGCCCCCAGTCAAAGCCTCTCTGTAACGTGCACAGTGA |
| | | GTGGTGTATCATTGCCTGATTATGGCGTCTCCTGGATAAG |
| | | GCAGCCCCCGCGAAAGGGTCTTGAATGGCTTGGGGTAAT |
| | | ATGGGGCTCAGAGACAACGTATTATAACTCCGCTCTCAA |
| | | AAGTCGCTTGACGATAATAAAAGATAACTCCAAGAGTCA |
| | | AGTTTTCCTTAAAATGAACAGTTTGCAGACTGACGATACC |
| | | GCTATATATTATTGTGCTAAACATTATTACTACGGCGGTA |
| | | GTTACGCGATGGATTATTGGGGGCAGGGGACTTCTGTCA |
| | | CAGTCAGTAGTGCTGCTGCCTTTGTCCCGGTATTTCTCCC |
| | | AGCCAAACCGACCACGACTCCCGCCCCGCGCCCTCCGAC |
| | | ACCCGCTCCCACCATCGCCTCTCAACCTCTTAGTCTTCGC |
| | | CCCGAGGCATGCCGACCCGCCGCCGGGGGTGCTGTTCAT |
| | | ACGAGGGGCTTGGACTTCGCTTGTGATATTTACATTTGGG |
| | | CTCCGTTGGCGGGTACGTGCGGCGTCCTTTTGTTGTCACT |
| | | CGTTATTACTTTGTATTGTAATCACAGGAATCGCTCAAAG |
| | | CGGAGTAGGTTGTTGCATTCCGATTACATGAATATGACTC |
| | | CTCGCCGGCCTGGGCCGACAAGAAAACATTACCAACCCT |
| | | ATGCCCCCCCACGAGACTTCGCTGCGTACAGGTCCCGAGT |
| | | GAAGTTTTCCCGAAGCGCAGACGCTCCGGCATATCAGCA |
| | | AGGACAGAATCAGCTGTATAACGAACTGAATTTGGGACG |
| | | CCGCGAGGAGTATGACGTGCTTGATAAACGCCGGGGGAG |
| | | AGACCCGGAAATGGGGGGTAAACCCCGAAGAAAGAATC |
| | | CCCAAGAAGGACTCTACAATGAACTCCAGAAGGATAAGA |
| | | TGGCGGAGGCCTACTCAGAAATAGGTATGAAGGGCGAAC |
| | | GACGACGGGGAAAAGGTCACGATGGCCTCTACCAAGGGT |
| | | TGAGTACGGCAACCAAAGATACGTACGATGCACTGCATA |
| | | TGCAGGCCCTGCCTCCCAGATAATAATAAAATCGCTATCC |
| | | ATCGAAGATGGATGTGTGTTGGTTTTTTGTGTGTGGAGCA |
| | | ACAAATCTGACTTTGCATGTGCAAACGCCTTCAACAACA |
| | | GCATTATTCCAGAAGACACCTTCTTCCCCAGCCCAGGTAA |
| | | GGGCAGCTTTGGTGCCTTCGCAGGCTGTTTCCTTGCTTCA |
| | | GGAATGGCCAGGTTCTGCCCAGAGCTCTGGTCAATGATG |
| | | TCTAAAACTCCTCTGATTGGTGGTCTCGGCCTTATCCATT |
| | | GCCACCAAAACCCTCTTTTTACTAAGAAACAGTGAGCCTT |
| | | GTTCTGGCAGTCCAGAGAATGACACGGGAAAAAAGCAGA |
| | | TGAAGAGAAGGTGGCAGGAGAGGGCACGTGGCCCAGCC |
| | | TCAGTCTCTCCAACTGAGTTCCTGCCTGCCTGCCTTTGCTC |
| | | AGACTGTTTGCCCCTTACTGCTCTTCTAGGCCTCATTCTAA |
| | | GCCCCTTCTCCAAGTTGCCTCTCCTTATTTCTCCCTGTCTG |
| | | CCAAAAAATCTTTCCCAGCTCACTAAGTCAGTCTCACGCA |
| | | GTCACTCATTAACCCACCAATCACTGATTGTGCCGGCACA |
| | | TGAATGCACCAGGTGTTGAAGTGGAGGAATTAAAAAGTC |
| | | AGATGAGGGGTGTGCCCAGAGGAAGCACCATTCTAGTTG |
| | | GGGGAGCCCATCTGTCAGCTGGGAAAAGTCCAAATAACT |
| | | TCAGATTGGAATGTGTTTTAACTCAGGGTTGAGAAAACA |
| | | GCTACCTTCAGGACAAAAGTCAGGGAAGGGCTCTCTGAA |
| | | GAAATGCTACTTGAAGATACCAGCCCTACCAAGGGCAGG |
| | | GAGAGGACCCTATAGAGGCCTGGGACAGGAGCTCAATGA |
| | | GAAAGG |
| 59 | CD38 | MANCEFSPVSGDKPCCRLSRRAQLCLGVSILVLILVVVLAV |
| | | VVPRWRQQWSGPGTTKRFPETVLARCVKYTEIHPEMRHVD |
| | | CQSVWDAFKGAFISKHPCNITEEDYQPLMKLGTQTVPCNKI |
| | | LLWSRIKDLAHQFTQVQRDMFTLEDTLLGYLADDLTWCGE |
| | | FNTSKINYQSCPDWRKDCSNNPVSVFWKTVSRRFAEAACD |
| | | VVHVMLNGSRSKIFDKNSTFGSVEVHNLQPEKVQTLEAWVI |
| | | HGGREDSRDLCQDPTIKELESIISKRNIQFSCKNIYRPDKFLQ |
| | | CVKNPEDSSCTSEI |
| 60 | CD38 mRNA | ATGGCCAACTGCGAGTTCAGCCCGGTGTCCGGGGACAAA |
| | | CCCTGCTGCCGGCTCTCTAGGAGAGCCCAACTCTGTCTTG |
| | | GCGTCAGTATCCTGGTCCTGATCCTCGTCGTGGTGCTCGC |
| | | GGTGGTCGTCCCGAGGTGGCGCCAGCAGTGGAGCGGTCC |
| | | GGGCACCACCAAGCGCTTTCCCGAGACCGTCCTGGCGCG |
| | | ATGCGTCAAGTACACTGAAATTCATCCTGAGATGAGACA |
| | | TGTAGACTGCCAAAGTGTATGGGATGCTTTCAAGGGTGC |
| | | ATTTATTTCAAAACATCCTTGCAACATTACTGAAGAAGAC |
| | | TATCAGCCACTAATGAAGTTGGGAACTCAGACCGTACCTT |
| | | GCAACAAGATTCTTCTTTGGAGCAGAATAAAAGATCTGG |
| | | CCCATCAGTTCACACAGGTCCAGCGGGACATGTTCACCCT |
| | | GGAGGACACGCTGCTAGGCTACCTTGCTGATGACCTCAC |
| | | ATGGTGTGGTGAATTCAACACTTCCAAAATAAACTATCA |
| | | ATCTTGCCCAGACTGGAGAAAGGACTGCAGCAACAACCC |
| | | TGTTTCAGTATTCTGGAAAACGGTTTCCCGCAGGTTTGCA |
| | | GAAGCTGCCTGTGATGTGGTCCATGTGATGCTCAATGGAT |
| | | CCCGCAGTAAAATCTTTGACAAAAACAGCACTTTTGGGA |
| | | GTGTGGAAGTCCATAATTTGCAACCAGAGAAGGTTCAGA |
| | | CACTAGAGGCCTGGGTGATACATGGTGGAAGAGAAGATT |
| | | CCAGAGACTTATGCCAGGATCC |
| | | CACCATAAAAGAGCTGGAATCGATTATAAGCAAAAGGAA |
| | | TATTCAATTTTCCTGCAAGAATATCTACAGACCTGACAAG |
| | | TTTCTTCAGTGTGTGAAAAATCCTGAGGATTCATCTTGCA |
| | | CATCTGAGATCTGA |
| 61 | daratumumab heavy | EVQLLESGGG LVQPGGSLRL SCAVSGFTFN |
| | chain full sequence | SFAMSWVRQAPGKGLEWVSA ISGSGGGTYY |
| | | ADSVKGRFTISRDNSKNTLY LQMNSLRAED TAVYFCAKDK |
| | | ILWFGEPVFD YWGQGTLVTV SSASTKGPSV FPLAPSSKST |
| | | SGGTAALGCL VKDYFPEPVT VSWNSGALTS |
| | | GVHTFPAVLQ SSGLYSLSSV VTVPSSSLGT QTYICNVNHK |
| | | PSNTKVDKRV EPKSCDKTHT CPPCPAPELL GGPSVFLFPP |
| | | KPKDTLMISR TPEVTCVVVD VSHEDPEVKF |
| | | NWYVDGVEVH NAKTKPREEQ |
| | | YNSTYRVVSV LTVLHQDWLN GKEYKCKVSN |
| | | KALPAPIEKT ISKAKGQPRE PQVYTLPPSR EEMTKNQVSL |
| | | TCLVKGFYPS DIAVEWESNG QPENNYKTTP PVLDSDGSFF |
| | | LYSKLTVDKS RWQQGNVFSC SVMHEALHNH |
| | | YTQKSLSLSP GK |
| 62 | daratumumab heavy | EVQLLESGGGLVQPGGSLRLSCAVSGFTFNSFAMSWVRQAP |
| | chain variable region | GKGLEWVSAISGSGGGTYYADSVKGRFTISRDNSKNTLYLQ |
| | | MNSLRAEDTAVYFCAKDKILWFGEPVFDYWGQGTLVTVSS |
| | | AS |
| 63 | daratumumab light | EIVLTQSPAT LSLSPGERAT LSCRASQSVS SYLAWYQQKP |
| | chain full sequence | QAPRLLIYD ASNRATGIPA RFSGSGSGTD FTLTISSLEP |
| | | EDFAVYYCQQ RSNWPPTFGQ GTKVEIKRTV AAPSVFIFPP |
| | | SDEQLKSGTA SVVCLLNNFY PREAKVQWKV |
| | | DNALQSGNSQ ESVTEQDSKD STYSLSSTLT LSKADYEKHK |
| | | VYACEVTHQG LSSPVTKSFN RGEC |
| 64 | daratumumab light | EIVLTQSPAT LSLSPGERAT LSCRASQSVS SYLAWYQQKP |
| | chain variable region | GQAPRLLIYD ASNRATGIPA RFSGSGSGTD FTLTISSLEP |
| | | EDFAVYYCQQ RSNWPPTFGQ GTKVEIK |
| 65 | daratumumab heavy | SFAMS |
| | chain CDR1 | |
| 66 | daratumumab heavy | AISGSGGGTY YADSVKG |
| | chain CDR2 | |
| 67 | daratumumab heavy | DKILWFGEPV FDY |
| | chain CDR3 | |
| 68 | daratumumab light | RASQSVSSYL A |
| | chain CDR1 | |
| 69 | daratumumab light | DASNRAT |
| | chain CDR2 | |
| 70 | daratumumab light | QQRSNWPPT |
| | chain CDR3 | |
| 71 | CD70 sgRNA | GCUUUGGUCCCAUUGGUCGCguuuuagagcuagaaauagcaaguuaa |
| | (E1_T7) | aauaaggcuaguccguuaucaacuugaaaaaguggcaccgagucggugcUUUU |
| 72 | CD70 sgRNA | GCCCGCAGGACGCACCCAUAguuuuagagcuagaaauagcaaguuaaa |
| | (E1_T8) | auaaggcuaguccguuaucaacuugaaaaaguggcaccgagucggugcUUUU |
| 73 | PD-1 sgRNA | CUGCAGCUUCUCCAACACAUguuuuagagcuagaaauagcaaguuaaa |
| | | auaaggcuaguccguuaucaacuugaaaaaguggcaccgagucggugcUUUU |
| 74 | CD70 | GAGATGTAAGGAGCTGCTGTGACTTGCTCAAGGCCTTAT |
| | LHA to RHA | ATCGAGTAAACGGTAGTGCTGGGGCTTAGACGCAGGTGT |
| | (CD70B scFV with | TCTGATTTATAGTTCAAAACCTCTATCAATGAGAGAGCAA |
| | 41BB) | TCTCCTGGTAATGTGATAGATTTCCCAACTTAATGCCAAC |
| | | ATACCATAAACCTCCCATTCTGCTAATGCCCAGCCTAAGT |
| | | TGGGGAGACCACTCCAGATTCCAAGATGTACAGTTTGCTT |
| | | TGCTGGGCCTTTTTCCCATGCCTGCCTTTACTCTGCCAGA |
| | | GTTATATTGCTGGGGTTTTGAAGAAGATCCTATTAAATAA |
| | | AAGAATAAGCAGTATTATTAAGTAGCCCTGCATTTCAGGT |
| | | TTCCTTGAGTGGCAGGCCAGGCCTGGCCGTGAACGTTCAC |
| | | TGAAATCATGGCCTCTTGGCCAAGATTGATAGCTTGTGCC |
| | | TGTCCCTGAGTCCCAGTCCATCACGAGCAGCTGGTTTCTA |
| | | AGATGCTATTTCCCGTATAAAGCATGAGACCGTGACTTGC |
| | | CAGCCCCACAGAGCCCCGCCCTTGTCCATCACTGGCATCT |
| | | GGACTCCAGCCTGGGTTGGGGCAAAGAGGGAAATGAGAT |
| | | CATGTCCTAACCCTGATCCTCTTGTCCCACAGATATCCAG |
| | | AACCCTGACCCTGCCGTGTACCAGCTGAGAGACTCTAAA |
| | | TCCAGTGACAAGTCTGTCTGCCTATTCACCGATTTTGATT |
| | | CTCAAACAAATGTGTCACAAAGTAAGGATTCTGATGTGT |
| | | ATATCACAGACAAAACTGTGCTAGACATGAGGTCTATGG |
| | | ACTTCAGGCTCCGGTGCCCGTCAGTGGGCAGAGCGCACA |
| | | TCGCCCACAGTCCCCGAGAAGTTGGGGGGAGGGGTCGGC |
| | | AATTGAACCGGTGCCTAGAGAAGGTGGCGCGGGGTAAAC |
| | | TGGGAAAGTGATGTCGTGTACTGGCTCCGCCTTTTTCCCG |
| | | AGGGTGGGGGAGAACCGTATATAAGTGCAGTAGTCGCCG |
| | | TGAACGTTCTTTTTCGCAACGGGTTTGCCGCCAGAACACA |
| | | GGTAAGTGCCGTGTGTGGTTCCCGCGGGCCTGGCCTCTTT |
| | | ACGGGTTATGGCCCTTGCGTGCCTTGAATTACTTCCACTG |
| | | GCTGCAGTACGTGATTCTTGATCCCGAGCTTCGGGTTGGA |
| | | AGTGGGTGGGAGAGTTCGAGGCCTTGCGCTTAAGGAGCC |
| | | CCTTCGCCTCGTGCTTGAGTTGAGGCCTGGCCTGGGCGCT |
| | | GGGGCCGCCGCGTGCGAATCTGGTGGCACCTTCGCGCCT |
| | | GTCTCGCTGCTTTCGATAAGTCTCTAGCCATTTAAAATTTT |
| | | TGATGACCTGCTGCGACGCTTTTTTTCTGGCAAGATAGTC |
| | | TTGTAAATGCGGGCCAAGATCTGCACACTGGTATTTCGGT |
| | | TTTTGGGGCCGCGGGCGGCGACGGGGCCCGTGCGTCCCA |
| | | GCGCACATGTTCGGCGAGGCGGGGCCTGCGAGCGCGGCC |
| | | ACCGAGAATCGGACGGGGGTAGTCTCAAGCTGGCCGGCC |
| | | TGCTCTGGTGCCTGGCCTCGCGCCGCCGTGTATCGCCCCG |
| | | CCCTGGGCGGCAAGGCTGGCCCGGTCGGCACCAGTTGCG |
| | | TGAGCGGAAAGATGGCCGCTTCCCGGCCCTGCTGCAGGG |
| | | AGCTCAAAATGGAGGACGCGGCGCTCGGGAGAGCGGGC |
| | | GGGTGAGTCACCCACACAAAGGAAAAGGGCCTTTCCGTC |
| | | CTCAGCCGTCGCTTCATGTGACTCCACGGAGTACCGGGCG |
| | | CCGTCCAGGCACCTCGATTAGTTCTCGAGCTTTTGGAGTA |
| | | CGTCGTCTTTAGGTTGGGGGGAGGGGTTTTATGCGATGGA |
| | | GTTTCCCCACACTGAGTGGGTGGAGACTGAAGTTAGGCC |
| | | AGCTTGGCACTTGATGTAATTCTCCTTGGAATTTGCCCTTT |
| | | TTGAGTTTGGATCTTGGTTCATTCTCAAGCCTCAGACAGT |
| | | GGTTCAAAGTTTTTTTCTTCCATTTCAGGTGTCGTGACCAC |
| | | CATGGCGCTTCCGGTGACAGCACTGCTCCTCCCCTTGGCG |
| | | CTGTTGCTCCACGCAGCAAGGCCGCAGGTCCAGTTGGTG |
| | | CAAAGCGGGGCGGAGGTGAAAAAACCCGGCGCTTCCGTG |
| | | AAGGTGTCCTGTAAGGCGTCCGGTTATACGTTCACGAACT |
| | | ACGGGATGAATTGGGTTCGCCAAGCGCCGGGGCAGGGAC |
| | | TGAAATGGATGGGGTGGATAAATACCTACACCGGCGAAC |
| | | CTACATACGCCGACGCTTTTAAAGGGCGAGTCACTATGA |
| | | CGCGCGATACCAGCATATCCACCGCATACATGGAGCTGT |
| | | CCCGACTCCGGTCAGACGACACGGCTGTCTACTATTGTGC |
| | | TCGGGACTATGGCGATTATGGCATGGACTACTGGGGTCA |
| | | GGGTACGACTGTAACAGTTAGTAGTGGTGGAGGCGGCAG |
| | | TGGCGGGGGGGGAAGCGGAGGAGGGGGTTCTGGTGACA |
| | | TAGTTATGACCCAATCCCCAGATAGTTTGGCGGTTTCTCT |
| | | GGGCGAGAGGGCAACGATTAATTGTCGCGCATCAAAGAG |
| | | CGTTTCAACGAGCGGATATTCTTTTATGCATTGGTACCAG |
| | | CAAAAACCCGGACAACCGCCGAAGCTGCTGATCTACTTG |
| | | GCTTCAAATCTTGAGTCTGGGGTGCCGGACCGATTTTCTG |
| | | GTAGTGGAAGCGGAACTGACTTTACGCTCACGATCAGTT |
| | | CACTGCAGGCTGAGGATGTAGCGGTCTATTATTGCCAGC |
| | | ACAGTAGAGAAGTCCCCTGGACCTTCGGTCAAGGCACGA |
| | | AAGTAGAAATTAAAAGTGCTGCTGCCTTTGTCCCGGTATT |
| | | TCTCCCAGCCAAACCGACCACGACTCCCGCCCCGCGCCCT |
| | | CCGACACCCGCTCCCACCATCGCCTCTCAACCTCTTAGTC |
| | | TTCGCCCCGAGGCATGCCGACCCGCCGCCGGGGGTGCTG |
| | | TTCATACGAGGGGCTTGGACTTCGCTTGTGATATTTACAT |
| | | TTGGGCTCCGTTGGCGGGTACGTGCGGCGTCCTTTTGTTG |
| | | TCACTCGTTATTACTTTGTATTGTAATCACAGGAATCGCA |
| | | AACGGGGCAGAAAGAAACTCCTGTATATATTCAAACAAC |
| | | CATTTATGAGACCAGTACAAACTACTCAAGAGGAAGATG |
| | | GCTGTAGCTGCCGATTTCCAGAAGAAGAAGAAGGAGGAT |
| | | GTGAACTGCGAGTGAAGTTTTCCCGAAGCGCAGACGCTC |
| | | CGGCATATCAGCAAGGACAGAATCAGCTGTATAACGAAC |
| | | TGAATTTGGGACGCCGCGAGGAGTATGACGTGCTTGATA |
| | | AACGCCGGGGGAGAGACCCGGAAATGGGGGGTAAACCC |
| | | CGAAGAAAGAATCCCCAAGAAGGACTCTACAATGAACTC |
| | | CAGAAGGATAAGATGGCGGAGGCCTACTCAGAAATAGGT |
| | | ATGAAGGGCGAACGACGACGGGGAAAAGGTCACGATGG |
| | | CCTCTACCAAGGGTTGAGTACGGCAACCAAAGATACGTA |
| | | CGATGCACTGCATATGCAGGCCCTGCCTCCCAGATAATA |
| | | ATAAAATCGCTATCCATCGAAGATGGATGTGTGTTGGTTT |
| | | TTTGTGTGTGGAGCAACAAATCTGACTTTGCATGTGCAAA |
| | | CGCCTTCAACAACAGCATTATTCCAGAAGACACCTTCTTC |
| | | CCCAGCCCAGGTAAGGGCAGCTTTGGTGCCTTCGCAGGC |
| | | TGTTTCCTTGCTTCAGGAATGGCCAGGTTCTGCCCAGAGC |
| | | TCTGGTCAATGATGTCTAAAACTCCTCTGATTGGTGGTCT |
| | | CGGCCTTATCCATTGCCACCAAAACCCTCTTTTTACTAAG |
| | | AAACAGTGAGCCTTGTTCTGGCAGTCCAGAGAATGACAC |
| | | GGGAAAAAAGCAGATGAAGAGAAGGTGGCAGGAGAGGG |
| | | CACGTGGCCCAGCCTCAGTCTCTCCAACTGAGTTCCTGCC |
| | | TGCCTGCCTTTGCTCAGACTGTTTGCCCCTTACTGCTCTTC |
| | | TAGGCCTCATTCTAAGCCCCTTCTCCAAGTTGCCTCTCCTT |
| | | ATTTCTCCCTGTCTGCCAAAAAATCTTTCCCAGCTCACTA |
| | | AGTCAGTCTCACGCAGTCACTCATTAACCCACCAATCACT |
| | | GATTGTGCCGGCACATGAATGCACCAGGTGTTGAAGTGG |
| | | AGGAATTAAAAAGTCAGATGAGGGGTGTGCCCAGAGGA |
| | | AGCACCATTCTAGTTGGGGGAGCCCATCTGTCAGCTGGG |
| | | AAAAGTCCAAATAACTTCAGATTGGAATGTGTTTTAACTC |
| | | AGGGTTGAGAAAACAGCTACCTTCAGGACAAAAGTCAGG |
| | | GAAGGGCTCTCTGAAGAAATGCTACTTGAAGATACCAGC |
| | | CCTACCAAGGGCAGGGAGAGGACCCTATAGAGGCCTGGG |
| | | ACAGGAGCTCAATGAGAAAGG |
| 75 | CD70 CAR | ATGGCGCTTCCGGTGACAGCACTGCTCCTCCCCTTGGCGC |
| | nucleotide sequence | TGTTGCTCCACGCAGCAAGGCCGCAGGTCCAGTTGGTGC |
| | (CD70B scFV with | AAAGCGGGGCGGAGGTGAAAAAACCCGGCGCTTCCGTGA |
| | 41BB) | AGGTGTCCTGTAAGGCGTCCGGTTATACGTTCACGAACTA |
| | | CGGGATGAATTGGGTTCGCCAAGCGCCGGGGCAGGGACT |
| | | GAAATGGATGGGGTGGATAAATACCTACACCGGCGAACC |
| | | TACATACGCCGACGCTTTTAAAGGGCGAGTCACTATGAC |
| | | GCGCGATACCAGCATATCCACCGCATACATGGAGCTGTC |
| | | CCGACTCCGGTCAGACGACACGGCTGTCTACTATTGTGCT |
| | | CGGGACTATGGCGATTATGGCATGGACTACTGGGGTCAG |
| | | GGTACGACTGTAACAGTTAGTAGTGGTGGAGGCGGCAGT |
| | | GGCGGGGGGGGAAGCGGAGGAGGGGGTTCTGGTGACAT |
| | | AGTTATGACCCAATCCCCAGATAGTTTGGCGGTTTCTCTG |
| | | GGCGAGAGGGCAACGATTAATTGTCGCGCATCAAAGAGC |
| | | GTTTCAACGAGCGGATATTCTTTTATGCATTGGTACCAGC |
| | | AAAAACCCGGACAACCGCCGAAGCTGCTGATCTACTTGG |
| | | CTTCAAATCTTGAGTCTGGGGTGCCGGACCGATTTTCTGG |
| | | TAGTGGAAGCGGAACTGACTTTACGCTCACGATCAGTTC |
| | | ACTGCAGGCTGAGGATGTAGCGGTCTATTATTGCCAGCA |
| | | CAGTAGAGAAGTCCCCTGGACCTTCGGTCAAGGCACGAA |
| | | AGTAGAAATTAAAAGTGCTGCTGCCTTTGTCCCGGTATTT |
| | | CTCCCAGCCAAACCGACCACGACTCCCGCCCCGCGCCCTC |
| | | CGACACCCGCTCCCACCATCGCCTCTCAACCTCTTAGTCT |
| | | TCGCCCCGAGGCATGCCGACCCGCCGCCGGGGGTGCTGT |
| | | TCATACGAGGGGCTTGGACTTCGCTTGTGATATTTACATT |
| | | TGGGCTCCGTTGGCGGGTACGTGCGGCGTCCTTTTGTTGT |
| | | CACTCGTTATTACTTTGTATTGTAATCACAGGAATCGCAA |
| | | ACGGGGCAGAAAGAAACTCCTGTATATATTCAAACAACC |
| | | ATTTATGAGACCAGTACAAACTACTCAAGAGGAAGATGG |
| | | CTGTAGCTGCCGATTTCCAGAAGAAGAAGAAGGAGGATG |
| | | TGAACTGCGAGTGAAGTTTTCCCGAAGCGCAGACGCTCC |
| | | GGCATATCAGCAAGGACAGAATCAGCTGTATAACGAACT |
| | | GAATTTGGGACGCCGCGAGGAGTATGACGTGCTTGATAA |
| | | ACGCCGGGGGAGAGACCCGGAAATGGGGGGTAAACCCC |
| | | GAAGAAAGAATCCCCAAGAAGGACTCTACAATGAACTCC |
| | | AGAAGGATAAGATGGCGGAGGCCTACTCAGAAATAGGTA |
| | | TGAAGGGCGAACGACGACGGGGAAAAGGTCACGATGGC |
| | | CTCTACCAAGGGTTGAGTACGGCAACCAAAGATACGTAC |
| | | GATGCACTGCATATGCAGGCCCTGCCTCCCAGATAA |
| 76 | CD70 CAR amino | MALPVTALLLPLALLLHAARPQVQLVQSGAEVKKPGASVK |
| | acid sequence | VSCKASGYTFTNYGMNWVRQAPGQGLKWMGWINTYTGEP |
| | (CD70B scFV with | TYADAFKGRVTMTRDTSISTAYMELSRLRSDDTAVYYCAR |
| | 41BB) | DYGDYGMDYWGQGTTVTVSSGGGGSGGGGSGGGGSGDIV |
| | | MTQSPDSLAVSLGERATINCRASKSVSTSGYSFMHWYQQKP |
| | | GQPPKLLIYLASNLESGVPDRFSGSGSGTDFTLTISSLQAEDV |
| | | AVYYCQHSREVPWTFGQGTKVEIKSAAAFVPVFLPAKPTTT |
| | | PAPRPPTPAPTIASQPLSLRPEACRPAAGGAVHTRGLDFACDI |
| | | YIWAPLAGTCGVLLLSLVITLYCNHRNRKRGRKKLLYIFKQ |
| | | PFMRPVQTTQEEDGCSCRFPEEEEGGCELRVKFSRSADAPA |
| | | YQQGQNQLYNELNLGRREEYDVLDKRRGRDPEMGGKPRR |
| | | KNPQEGLYNELQKDKMAEAYSEIGMKGERRRGKGHDGLY |
| | | QGLSTATKDTYDALHMQALPPR |
| 77 | CD70A | GATATAGTTATGACCCAATCACCCGATAGTCTTGCGGTAA |
| | scFv nucleotide | GCCTGGGGGAGCGAGCAACAATAAACTGTCGGGCATCAA |
| | sequence | AATCCGTCAGTACAAGCGGGTATTCATTCATGCACTGGTA |
| | | TCAACAGAAACCCGGTCAGCCACCCAAGCTCCTGATTTAT |
| | | CTTGCGTCTAATCTTGAGTCCGGCGTCCCAGACCGGTTTT |
| | | CCGGCTCCGGGAGCGGCACGGATTTTACTCTTACTATTTC |
| | | TAGCCTTCAGGCCGAAGATGTGGCGGTATACTACTGCCA |
| | | GCATTCAAGGGAAGTTCCTTGGACGTTCGGTCAGGGCAC |
| | | GAAAGTGGAAATTAAAGGCGGGGGGGGATCCGGCGGGG |
| | | GAGGGTCTGGAGGAGGTGGCAGTGGTCAGGTCCAACTGG |
| | | TGCAGTCCGGGGCAGAGGTAAAAAAACCCGGCGCGTCTG |
| | | TTAAGGTTTCATGCAAGGCCAGTGGATATACTTTCACCAA |
| | | TTACGGAATGAACTGGGTGAGGCAGGCCCCTGGTCAAGG |
| | | CCTGAAATGGATGGGATGGATAAACACGTACACCGGTGA |
| | | ACCTACCTATGCCGATGCCTTTAAGGGTCGGGTTACGATG |
| | | ACGAGAGACACCTCCATATCAACAGCCTACATGGAGCTC |
| | | AGCAGATTGAGGAGTGACGATACGGCAGTCTATTACTGT |
| | | GCAAGAGACTACGGCGATTATGGCATGGATTACTGGGGC |
| | | CAGGGCACTACAGTAACCGTTTCCAGC |
| 78 | CD70A | DIVMTQSPDSLAVSLGERATINCRASKSVSTSGYSFMHWYQ |
| | scFv amino acid | QKPGQPPKLLIYLASNLESGVPDRFSGSGSGTDFTLTISSLQA |
| | sequence | EDVAVYYCQHSREVPWTFGQGTKVEIKGGGGSGGGGSGGG |
| | (linker underlined) | GSGQVQLVQSGAEVKKPGASVKVSCKASGYTFTNYGMNW |
| | | VRQAPGQGLKWMGWINTYTGEPTYADAFKGRVTMTRDTSI |
| | | STAYMELSRLRSDDTAVYYCARDYGDYGMDYWGQGTTVT |
| | | VSS |
| 79 | CD70B | CAGGTCCAGTTGGTGCAAAGCGGGGCGGAGGTGAAAAA |
| | scFv nucleotide | ACCCGGCGCTTCCGTGAAGGTGTCCTGTAAGGCGTCCGGT |
| | sequence | TATACGTTCACGAACTACGGGATGAATTGGGTTCGCCAA |
| | | GCGCCGGGGCAGGGACTGAAATGGATGGGGTGGATAAAT |
| | | ACCTACACCGGCGAACCTACATACGCCGACGCTTTTAAA |
| | | GGGCGAGTCACTATGACGCGCGATACCAGCATATCCACC |
| | | GCATACATGGAGCTGTCCCGACTCCGGTCAGACGACACG |
| | | GCTGTCTACTATTGTGCTCGGGACTATGGCGATTATGGCA |
| | | TGGACTACTGGGGTCAGGGTACGACTGTAACAGTTAGTA |
| | | GTGGTGGAGGCGGCAGTGGCGGGGGGGGAAGCGGAGGA |
| | | GGGGGTTCTGGTGACATAGTTATGACCCAATCCCCAGAT |
| | | AGTTTGGCGGTTTCTCTGGGCGAGAGGGCAACGATTAATT |
| | | GTCGCGCATCAAAGAGCGTTTCAACGAGCGGATATTCTTT |
| | | TATGCATTGGTACCAGCAAAAACCCGGACAACCGCCGAA |
| | | GCTGCTGATCTACTTGGCTTCAAATCTTGAGTCTGGGGTG |
| | | CCGGACCGATTTTCTGGTAGTGGAAGCGGAACTGACTTTA |
| | | CGCTCACGATCAGTTCACTGCAGGCTGAGGATGTAGCGG |
| | | TCTATTATTGCCAGCACAGTAGAGAAGTCCCCTGGACCTT |
| | | CGGTCAAGGCACGAAAGTAGAAATTAAA |
| 80 | CD70B | QVQLVQSGAEVKKPGASVKVSCKASGYTFTNYGMNWVRQ |
| | scFv amino acid | APGQGLKWMGWINTYTGEPTYADAFKGRVTMTRDTSISTA |
| | sequence | YMELSRLRSDDTAVYYCARDYGDYGMDYWGQGTTVTVSS |
| | (linker underlined) | GGGGSGGGGSGGGGSGDIVMTQSPDSLAVSLGERATINCRA |
| | | SKSVSTSGYSFMHWYQQKPGQPPKLLIYLASNLESGVPDRF |
| | | SGSGSGTDFTLTISSLQAEDVAVYYCQHSREVPWTFGQGTK |
| | | VEIK |
| 81 | CD70 VH | QVQLVQSGAEVKKPGASVKVSCKASGYTFTNYGMNWVRQ |
| | | APGQGLKWMGWINTYTGEPTYADAFKGRVTMTRDTSISTA |
| | | YMELSRLRSDDTAVYYCARDYGDYGMDYWGQGTTVTVSS |
| 82 | CD70 VL | DIVMTQSPDSLAVSLGERATINCRASKSVSTSGYSFMHWYQ |
| | | QKPGQPPKLLIYLASNLESGVPDRFSGSGSGTDFTLTISSLQA |
| | | EDVAVYYCQHSREVPWTFGQGTKVEIK |
| 83 | BCMA rAAV | CCTGCAGGCAGCTGCGCGCTCGCTCGCTCACTGAGGCCG |
| | | CCCGGGCGTCGGGCGACCTTTGGTCGCCCGGCCTCAGTG |
| | | AGCGAGCGAGCGCGCAGAGAGGGAGTGGCCAACTCCATC |
| | | ACTAGGGGTTCCTGCGGCCGCACGCGTGAGATGTAAGGA |
| | | GCTGCTGTGACTTGCTCAAGGCCTTATATCGAGTAAACGG |
| | | TAGTGCTGGGGCTTAGACGCAGGTGTTCTGATTTATAGTT |
| | | CAAAACCTCTATCAATGAGAGAGCAATCTCCTGGTAATG |
| | | TGATAGATTTCCCAACTTAATGCCAACATACCATAAACCT |
| | | CCCATTCTGCTAATGCCCAGCCTAAGTTGGGGAGACCACT |
| | | CCAGATTCCAAGATGTACAGTTTGCTTTGCTGGGCCTTTT |
| | | TCCCATGCCTGCCTTTACTCTGCCAGAGTTATATTGCTGG |
| | | GGTTTTGAAGAAGATCCTATTAAATAAAAGAATAAGCAG |
| | | TATTATTAAGTAGCCCTGCATTTCAGGTTTCCTTGAGTGG |
| | | CAGGCCAGGCCTGGCCGTGAACGTTCACTGAAATCATGG |
| | | CCTCTTGGCCAAGATTGATAGCTTGTGCCTGTCCCTGAGT |
| | | CCCAGTCCATCACGAGCAGCTGGTTTCTAAGATGCTATTT |
| | | CCCGTATAAAGCATGAGACCGTGACTTGCCAGCCCCACA |
| | | GAGCCCCGCCCTTGTCCATCACTGGCATCTGGACTCCAGC |
| | | CTGGGTTGGGGCAAAGAGGGAAATGAGATCATGTCCTAA |
| | | CCCTGATCCTCTTGTCCCACAGATATCCAGAACCCTGACC |
| | | CTGCCGTGTACCAGCTGAGAGACTCTAAATCCAGTGACA |
| | | AGTCTGTCTGCCTATTCACCGATTTTGATTCTCAAACAAA |
| | | TGTGTCACAAAGTAAGGATTCTGATGTGTATATCACAGAC |
| | | AAAACTGTGCTAGACATGAGGTCTATGGACTTCAGGCTC |
| | | CGGTGCCCGTCAGTGGGCAGAGCGCACATCGCCCACAGT |
| | | CCCCGAGAAGTTGGGGGGAGGGGTCGGCAATTGAACCGG |
| | | TGCCTAGAGAAGGTGGCGCGGGGTAAACTGGGAAAGTGA |
| | | TGTCGTGTACTGGCTCCGCCTTTTTCCCGAGGGTGGGGGA |
| | | GAACCGTATATAAGTGCAGTAGTCGCCGTGAACGTTCTTT |
| | | TTCGCAACGGGTTTGCCGCCAGAACACAGGTAAGTGCCG |
| | | TGTGTGGTTCCCGCGGGCCTGGCCTCTTTACGGGTTATGG |
| | | CCCTTGCGTGCCTTGAATTACTTCCACTGGCTGCAGTACG |
| | | TGATTCTTGATCCCGAGCTTCGGGTTGGAAGTGGGTGGGA |
| | | GAGTTCGAGGCCTTGCGCTTAAGGAGCCCCTTCGCCTCGT |
| | | GCTTGAGTTGAGGCCTGGCCTGGGCGCTGGGGCCGCCGC |
| | | GTGCGAATCTGGTGGCACCTTCGCGCCTGTCTCGCTGCTT |
| | | TCGATAAGTCTCTAGCCATTTAAAATTTTTGATGACCTGC |
| | | TGCGACGCTTTTTTTCTGGCAAGATAGTCTTGTAAATGCG |
| | | GGCCAAGATCTGCACACTGGTATTTCGGTTTTTGGGGCCG |
| | | CGGGCGGCGACGGGGCCCGTGCGTCCCAGCGCACATGTT |
| | | CGGCGAGGCGGGGCCTGCGAGCGCGGCCACCGAGAATCG |
| | | GACGGGGGTAGTCTCAAGCTGGCCGGCCTGCTCTGGTGC |
| | | CTGGCCTCGCGCCGCCGTGTATCGCCCCGCCCTGGGCGGC |
| | | AAGGCTGGCCCGGTCGGCACCAGTTGCGTGAGCGGAAAG |
| | | ATGGCCGCTTCCCGGCCCTGCTGCAGGGAGCTCAAAATG |
| | | GAGGACGCGGCGCTCGGGAGAGCGGGCGGGTGAGTCAC |
| | | CCACACAAAGGAAAAGGGCCTTTCCGTCCTCAGCCGTCG |
| | | CTTCATGTGACTCCACGGAGTACCGGGCGCCGTCCAGGC |
| | | ACCTCGATTAGTTCTCGAGCTTTTGGAGTACGTCGTCTTT |
| | | AGGTTGGGGGGAGGGGTTTTATGCGATGGAGTTTCCCCA |
| | | CACTGAGTGGGTGGAGACTGAAGTTAGGCCAGCTTGGCA |
| | | CTTGATGTAATTCTCCTTGGAATTTGCCCTTTTTGAGTTTG |
| | | GATCTTGGTTCATTCTCAAGCCTCAGACAGTGGTTCAAAG |
| | | TTTTTTTCTTCCATTTCAGGTGTCGTGACCACCATGGCGCT |
| | | TCCGGTGACAGCACTGCTCCTCCCCTTGGCGCTGTTGCTC |
| | | CACGCAGCAAGGCCGCAGGTGCAGCTGGTGCAGAGCGGA |
| | | GCCGAGCTCAAGAAGCCCGGAGCCTCCGTGAAGGTGAGC |
| | | TGCAAGGCCAGCGGCAACACCCTGACCAACTACGTGATC |
| | | CACTGGGTGAGACAAGCCCCCGGCCAAAGGCTGGAGTGG |
| | | ATGGGCTACATCCTGCCCTACAACGACCTGACCAAGTAC |
| | | AGCCAGAAGTTCCAGGGCAGGGTGACCATCACCAGGGAT |
| | | AAGAGCGCCTCCACCGCCTATATGGAGCTGAGCAGCCTG |
| | | AGGAGCGAGGACACCGCTGTGTACTACTGTACAAGGTGG |
| | | GACTGGGACGGCTTCTTTGACCCCTGGGGCCAGGGCACA |
| | | ACAGTGACCGTCAGCAGCGGCGGCGGAGGCAGCGGCGG |
| | | CGGCGGCAGCGGCGGAGGCGGAAGCGAAATCGTGATGA |
| | | CCCAGAGCCCCGCCACACTGAGCGTGAGCCCTGGCGAGA |
| | | GGGCCAGCATCTCCTGCAGGGCTAGCCAAAGCCTGGTGC |
| | | ACAGCAACGGCAACACCCACCTGCACTGGTACCAGCAGA |
| | | GACCCGGACAGGCTCCCAGGCTGCTGATCTACAGCGTGA |
| | | GCAACAGGTTCTCCGAGGTGCCTGCCAGGTTTAGCGGCA |
| | | GCGGAAGCGGCACCGACTTTACCCTGACCATCAGCAGCG |
| | | TGGAGTCCGAGGACTTCGCCGTGTATTACTGCAGCCAGA |
| | | CCAGCCACATCCCTTACACCTTCGGCGGCGGCACCAAGCT |
| | | GGAGATCAAAAGTGCTGCTGCCTTTGTCCCGGTATTTCTC |
| | | CCAGCCAAACCGACCACGACTCCCGCCCCGCGCCCTCCG |
| | | ACACCCGCTCCCACCATCGCCTCTCAACCTCTTAGTCTTC |
| | | GCCCCGAGGCATGCCGACCCGCCGCCGGGGGTGCTGTTC |
| | | ATACGAGGGGCTTGGACTTCGCTTGTGATATTTACATTTG |
| | | GGCTCCGTTGGCGGGTACGTGCGGCGTCCTTTTGTTGTCA |
| | | CTCGTTATTACTTTGTATTGTAATCACAGGAATCGCAAAC |
| | | GGGGCAGAAAGAAACTCCTGTATATATTCAAACAACCAT |
| | | TTATGAGACCAGTACAAACTACTCAAGAGGAAGATGGCT |
| | | GTAGCTGCCGATTTCCAGAAGAAGAAGAAGGAGGATGTG |
| | | AACTGCGAGTGAAGTTTTCCCGAAGCGCAGACGCTCCGG |
| | | CATATCAGCAAGGACAGAATCAGCTGTATAACGAACTGA |
| | | ATTTGGGACGCCGCGAGGAGTATGACGTGCTTGATAAAC |
| | | GCCGGGGGAGAGACCCGGAAATGGGGGGTAAACCCCGA |
| | | AGAAAGAATCCCCAAGAAGGACTCTACAATGAACTCCAG |
| | | AAGGATAAGATGGCGGAGGCCTACTCAGAAATAGGTATG |
| | | AAGGGCGAACGACGACGGGGAAAAGGTCACGATGGCCT |
| | | CTACCAAGGGTTGAGTACGGCAACCAAAGATACGTACGA |
| | | TGCACTGCATATGCAGGCCCTGCCTCCCAGATAATAATAA |
| | | AATCGCTATCCATCGAAGATGGATGTGTGTTGGTTTTTTG |
| | | TGTGTGGAGCAACAAATCTGACTTTGCATGTGCAAACGC |
| | | CTTCAACAACAGCATTATTCCAGAAGACACCTTCTTCCCC |
| | | AGCCCAGGTAAGGGCAGCTTTGGTGCCTTCGCAGGCTGTT |
| | | TCCTTGCTTCAGGAATGGCCAGGTTCTGCCCAGAGCTCTG |
| | | GTCAATGATGTCTAAAACTCCTCTGATTGGTGGTCTCGGC |
| | | CTTATCCATTGCCACCAAAACCCTCTTTTTACTAAGAAAC |
| | | AGTGAGCCTTGTTCTGGCAGTCCAGAGAATGACACGGGA |
| | | AAAAAGCAGATGAAGAGAAGGTGGCAGGAGAGGGCACG |
| | | TGGCCCAGCCTCAGTCTCTCCAACTGAGTTCCTGCCTGCC |
| | | TGCCTTTGCTCAGACTGTTTGCCCCTTACTGCTCTTCTAGG |
| | | CCTCATTCTAAGCCCCTTCTCCAAGTTGCCTCTCCTTATTT |
| | | CTCCCTGTCTGCCAAAAAATCTTTCCCAGCTCACTAAGTC |
| | | AGTCTCACGCAGTCACTCATTAACCCACCAATCACTGATT |
| | | GTGCCGGCACATGAATGCACCAGGTGTTGAAGTGGAGGA |
| | | ATTAAAAAGTCAGATGAGGGGTGTGCCCAGAGGAAGCAC |
| | | CATTCTAGTTGGGGGAGCCCATCTGTCAGCTGGGAAAAG |
| | | TCCAAATAACTTCAGATTGGAATGTGTTTTAACTCAGGGT |
| | | TGAGAAAACAGCTACCTTCAGGACAAAAGTCAGGGAAGG |
| | | GCTCTCTGAAGAAATGCTACTTGAAGATACCAGCCCTACC |
| | | AAGGGCAGGGAGAGGACCCTATAGAGGCCTGGGACAGG |
| | | AGCTCAATGAGAAAGGTAACCACGTGCGGACCGAGGCTG |
| | | CAGCGTCGTCCTCCCTAGGAACCCCTAGTGATGGAGTTGG |
| | | CCACTCCCTCTCTGCGCGCTCGCTCGCTCACTGAGGCCGG |
| | | GCGACCAAAGGTCGCCCGACGCCCGGGCTTTGCCCGGGC |
| | | GGCCTCAGTGAGCGAGCGAGCGCGCAGCTGCCTGCAGG |
| 84 | BCMA | GAGATGTAAGGAGCTGCTGTGACTTGCTCAAGGCCTTAT |
| | RHA to LHA | ATCGAGTAAACGGTAGTGCTGGGGCTTAGACGCAGGTGT |
| | | TCTGATTTATAGTTCAAAACCTCTATCAATGAGAGAGCAA |
| | | TCTCCTGGTAATGTGATAGATTTCCCAACTTAATGCCAAC |
| | | ATACCATAAACCTCCCATTCTGCTAATGCCCAGCCTAAGT |
| | | TGGGGAGACCACTCCAGATTCCAAGATGTACAGTTTGCTT |
| | | TGCTGGGCCTTTTTCCCATGCCTGCCTTTACTCTGCCAGA |
| | | GTTATATTGCTGGGGTTTTGAAGAAGATCCTATTAAATAA |
| | | AAGAATAAGCAGTATTATTAAGTAGCCCTGCATTTCAGGT |
| | | TTCCTTGAGTGGCAGGCCAGGCCTGGCCGTGAACGTTCAC |
| | | TGAAATCATGGCCTCTTGGCCAAGATTGATAGCTTGTGCC |
| | | TGTCCCTGAGTCCCAGTCCATCACGAGCAGCTGGTTTCTA |
| | | AGATGCTATTTCCCGTATAAAGCATGAGACCGTGACTTGC |
| | | CAGCCCCACAGAGCCCCGCCCTTGTCCATCACTGGCATCT |
| | | GGACTCCAGCCTGGGTTGGGGCAAAGAGGGAAATGAGAT |
| | | CATGTCCTAACCCTGATCCTCTTGTCCCACAGATATCCAG |
| | | AACCCTGACCCTGCCGTGTACCAGCTGAGAGACTCTAAA |
| | | TCCAGTGACAAGTCTGTCTGCCTATTCACCGATTTTGATT |
| | | CTCAAACAAATGTGTCACAAAGTAAGGATTCTGATGTGT |
| | | ATATCACAGACAAAACTGTGCTAGACATGAGGTCTATGG |
| | | ACTTCAGGCTCCGGTGCCCGTCAGTGGGCAGAGCGCACA |
| | | TCGCCCACAGTCCCCGAGAAGTTGGGGGGAGGGGTCGGC |
| | | AATTGAACCGGTGCCTAGAGAAGGTGGCGCGGGGTAAAC |
| | | TGGGAAAGTGATGTCGTGTACTGGCTCCGCCTTTTTCCCG |
| | | AGGGTGGGGGAGAACCGTATATAAGTGCAGTAGTCGCCG |
| | | TGAACGTTCTTTTTCGCAACGGGTTTGCCGCCAGAACACA |
| | | GGTAAGTGCCGTGTGTGGTTCCCGCGGGCCTGGCCTCTTT |
| | | ACGGGTTATGGCCCTTGCGTGCCTTGAATTACTTCCACTG |
| | | GCTGCAGTACGTGATTCTTGATCCCGAGCTTCGGGTTGGA |
| | | AGTGGGTGGGAGAGTTCGAGGCCTTGCGCTTAAGGAGCC |
| | | CCTTCGCCTCGTGCTTGAGTTGAGGCCTGGCCTGGGCGCT |
| | | GGGGCCGCCGCGTGCGAATCTGGTGGCACCTTCGCGCCT |
| | | GTCTCGCTGCTTTCGATAAGTCTCTAGCCATTTAAAATTTT |
| | | TGATGACCTGCTGCGACGCTTTTTTTCTGGCAAGATAGTC |
| | | TTGTAAATGCGGGCCAAGATCTGCACACTGGTATTTCGGT |
| | | TTTTGGGGCCGCGGGCGGCGACGGGGCCCGTGCGTCCCA |
| | | GCGCACATGTTCGGCGAGGCGGGGCCTGCGAGCGCGGCC |
| | | ACCGAGAATCGGACGGGGGTAGTCTCAAGCTGGCCGGCC |
| | | TGCTCTGGTGCCTGGCCTCGCGCCGCCGTGTATCGCCCCG |
| | | CCCTGGGCGGCAAGGCTGGCCCGGTCGGCACCAGTTGCG |
| | | TGAGCGGAAAGATGGCCGCTTCCCGGCCCTGCTGCAGGG |
| | | AGCTCAAAATGGAGGACGCGGCGCTCGGGAGAGCGGGC |
| | | GGGTGAGTCACCCACACAAAGGAAAAGGGCCTTTCCGTC |
| | | CTCAGCCGTCGCTTCATGTGACTCCACGGAGTACCGGGCG |
| | | CCGTCCAGGCACCTCGATTAGTTCTCGAGCTTTTGGAGTA |
| | | CGTCGTCTTTAGGTTGGGGGGAGGGGTTTTATGCGATGGA |
| | | GTTTCCCCACACTGAGTGGGTGGAGACTGAAGTTAGGCC |
| | | AGCTTGGCACTTGATGTAATTCTCCTTGGAATTTGCCCTTT |
| | | TTGAGTTTGGATCTTGGTTCATTCTCAAGCCTCAGACAGT |
| | | GGTTCAAAGTTTTTTTCTTCCATTTCAGGTGTCGTGACCAC |
| | | CATGGCGCTTCCGGTGACAGCACTGCTCCTCCCCTTGGCG |
| | | CTGTTGCTCCACGCAGCAAGGCCGCAGGTGCAGCTGGTG |
| | | CAGAGCGGAGCCGAGCTCAAGAAGCCCGGAGCCTCCGTG |
| | | AAGGTGAGCTGCAAGGCCAGCGGCAACACCCTGACCAAC |
| | | TACGTGATCCACTGGGTGAGACAAGCCCCCGGCCAAAGG |
| | | CTGGAGTGGATGGGCTACATCCTGCCCTACAACGACCTG |
| | | ACCAAGTACAGCCAGAAGTTCCAGGGCAGGGTGACCATC |
| | | ACCAGGGATAAGAGCGCCTCCACCGCCTATATGGAGCTG |
| | | AGCAGCCTGAGGAGCGAGGACACCGCTGTGTACTACTGT |
| | | ACAAGGTGGGACTGGGACGGCTTCTTTGACCCCTGGGGC |
| | | CAGGGCACAACAGTGACCGTCAGCAGCGGCGGCGGAGG |
| | | CAGCGGCGGCGGCGGCAGCGGCGGAGGCGGAAGCGAAA |
| | | TCGTGATGACCCAGAGCCCCGCCACACTGAGCGTGAGCC |
| | | CTGGCGAGAGGGCCAGCATCTCCTGCAGGGCTAGCCAAA |
| | | GCCTGGTGCACAGCAACGGCAACACCCACCTGCACTGGT |
| | | ACCAGCAGAGACCCGGACAGGCTCCCAGGCTGCTGATCT |
| | | ACAGCGTGAGCAACAGGTTCTCCGAGGTGCCTGCCAGGT |
| | | TTAGCGGCAGCGGAAGCGGCACCGACTTTACCCTGACCA |
| | | TCAGCAGCGTGGAGTCCGAGGACTTCGCCGTGTATTACTG |
| | | CAGCCAGACCAGCCACATCCCTTACACCTTCGGCGGCGG |
| | | CACCAAGCTGGAGATCAAAAGTGCTGCTGCCTTTGTCCCG |
| | | GTATTTCTCCCAGCCAAACCGACCACGACTCCCGCCCCGC |
| | | GCCCTCCGACACCCGCTCCCACCATCGCCTCTCAACCTCT |
| | | TAGTCTTCGCCCCGAGGCATGCCGACCCGCCGCCGGGGG |
| | | TGCTGTTCATACGAGGGGCTTGGACTTCGCTTGTGATATT |
| | | TACATTTGGGCTCCGTTGGCGGGTACGTGCGGCGTCCTTT |
| | | TGTTGTCACTCGTTATTACTTTGTATTGTAATCACAGGAA |
| | | TCGCAAACGGGGCAGAAAGAAACTCCTGTATATATTCAA |
| | | ACAACCATTTATGAGACCAGTACAAACTACTCAAGAGGA |
| | | AGATGGCTGTAGCTGCCGATTTCCAGAAGAAGAAGAAGG |
| | | AGGATGTGAACTGCGAGTGAAGTTTTCCCGAAGCGCAGA |
| | | CGCTCCGGCATATCAGCAAGGACAGAATCAGCTGTATAA |
| | | CGAACTGAATTTGGGACGCCGCGAGGAGTATGACGTGCT |
| | | TGATAAACGCCGGGGGAGAGACCCGGAAATGGGGGGTA |
| | | AACCCCGAAGAAAGAATCCCCAAGAAGGACTCTACAATG |
| | | AACTCCAGAAGGATAAGATGGCGGAGGCCTACTCAGAAA |
| | | TAGGTATGAAGGGCGAACGACGACGGGGAAAAGGTCAC |
| | | GATGGCCTCTACCAAGGGTTGAGTACGGCAACCAAAGAT |
| | | ACGTACGATGCACTGCATATGCAGGCCCTGCCTCCCAGAT |
| | | AATAATAAAATCGCTATCCATCGAAGATGGATGTGTGTT |
| | | GGTTTTTTGTGTGTGGAGCAACAAATCTGACTTTGCATGT |
| | | GCAAACGCCTTCAACAACAGCATTATTCCAGAAGACACC |
| | | TTCTTCCCCAGCCCAGGTAAGGGCAGCTTTGGTGCCTTCG |
| | | CAGGCTGTTTCCTTGCTTCAGGAATGGCCAGGTTCTGCCC |
| | | AGAGCTCTGGTCAATGATGTCTAAAACTCCTCTGATTGGT |
| | | GGTCTCGGCCTTATCCATTGCCACCAAAACCCTCTTTTTA |
| | | CTAAGAAACAGTGAGCCTTGTTCTGGCAGTCCAGAGAAT |
| | | GACACGGGAAAAAAGCAGATGAAGAGAAGGTGGCAGGA |
| | | GAGGGCACGTGGCCCAGCCTCAGTCTCTCCAACTGAGTTC |
| | | CTGCCTGCCTGCCTTTGCTCAGACTGTTTGCCCCTTACTGC |
| | | TCTTCTAGGCCTCATTCTAAGCCCCTTCTCCAAGTTGCCTC |
| | | TCCTTATTTCTCCCTGTCTGCCAAAAAATCTTTCCCAGCTC |
| | | ACTAAGTCAGTCTCACGCAGTCACTCATTAACCCACCAAT |
| | | CACTGATTGTGCCGGCACATGAATGCACCAGGTGTTGAA |
| | | GTGGAGGAATTAAAAAGTCAGATGAGGGGTGTGCCCAGA |
| | | GGAAGCACCATTCTAGTTGGGGGAGCCCATCTGTCAGCT |
| | | GGGAAAAGTCCAAATAACTTCAGATTGGAATGTGTTTTA |
| | | ACTCAGGGTTGAGAAAACAGCTACCTTCAGGACAAAAGT |
| | | CAGGGAAGGGCTCTCTGAAGAAATGCTACTTGAAGATAC |
| | | CAGCCCTACCAAGGGCAGGGAGAGGACCCTATAGAGGCC |
| | | TGGGACAGGAGCTCAATGAGAAAGG |
| 85 | BCMA CAR | ATGGCGCTTCCGGTGACAGCACTGCTCCTCCCCTTGGCGC |
| | nucleotide sequence | TGTTGCTCCACGCAGCAAGGCCGCAGGTGCAGCTGGTGC |
| | | AGAGCGGAGCCGAGCTCAAGAAGCCCGGAGCCTCCGTGA |
| | | AGGTGAGCTGCAAGGCCAGCGGCAACACCCTGACCAACT |
| | | ACGTGATCCACTGGGTGAGACAAGCCCCCGGCCAAAGGC |
| | | TGGAGTGGATGGGCTACATCCTGCCCTACAACGACCTGA |
| | | CCAAGTACAGCCAGAAGTTCCAGGGCAGGGTGACCATCA |
| | | CCAGGGATAAGAGCGCCTCCACCGCCTATATGGAGCTGA |
| | | GCAGCCTGAGGAGCGAGGACACCGCTGTGTACTACTGTA |
| | | CAAGGTGGGACTGGGACGGCTTCTTTGACCCCTGGGGCC |
| | | AGGGCACAACAGTGACCGTCAGCAGCGGCGGCGGAGGC |
| | | AGCGGCGGCGGCGGCAGCGGCGGAGGCGGAAGCGAAAT |
| | | CGTGATGACCCAGAGCCCCGCCACACTGAGCGTGAGCCC |
| | | TGGCGAGAGGGCCAGCATCTCCTGCAGGGCTAGCCAAAG |
| | | CCTGGTGCACAGCAACGGCAACACCCACCTGCACTGGTA |
| | | CCAGCAGAGACCCGGACAGGCTCCCAGGCTGCTGATCTA |
| | | CAGCGTGAGCAACAGGTTCTCCGAGGTGCCTGCCAGGTT |
| | | TAGCGGCAGCGGAAGCGGCACCGACTTTACCCTGACCAT |
| | | CAGCAGCGTGGAGTCCGAGGACTTCGCCGTGTATTACTG |
| | | CAGCCAGACCAGCCACATCCCTTACACCTTCGGCGGCGG |
| | | CACCAAGCTGGAGATCAAAAGTGCTGCTGCCTTTGTCCCG |
| | | GTATTTCTCCCAGCCAAACCGACCACGACTCCCGCCCCGC |
| | | GCCCTCCGACACCCGCTCCCACCATCGCCTCTCAACCTCT |
| | | TAGTCTTCGCCCCGAGGCATGCCGACCCGCCGCCGGGGG |
| | | TGCTGTTCATACGAGGGGCTTGGACTTCGCTTGTGATATT |
| | | TACATTTGGGCTCCGTTGGCGGGTACGTGCGGCGTCCTTT |
| | | TGTTGTCACTCGTTATTACTTTGTATTGTAATCACAGGAA |
| | | TCGCAAACGGGGCAGAAAGAAACTCCTGTATATATTCAA |
| | | ACAACCATTTATGAGACCAGTACAAACTACTCAAGAGGA |
| | | AGATGGCTGTAGCTGCCGATTTCCAGAAGAAGAAGAAGG |
| | | AGGATGTGAACTGCGAGTGAAGTTTTCCCGAAGCGCAGA |
| | | CGCTCCGGCATATCAGCAAGGACAGAATCAGCTGTATAA |
| | | CGAACTGAATTTGGGACGCCGCGAGGAGTATGACGTGCT |
| | | TGATAAACGCCGGGGGAGAGACCCGGAAATGGGGGGTA |
| | | AACCCCGAAGAAAGAATCCCCAAGAAGGACTCTACAATG |
| | | AACTCCAGAAGGATAAGATGGCGGAGGCCTACTCAGAAA |
| | | TAGGTATGAAGGGCGAACGACGACGGGGAAAAGGTCAC |
| | | GATGGCCTCTACCAAGGGTTGAGTACGGCAACCAAAGAT |
| | | ACGTACGATGCACTGCATATGCAGGCCCTGCCTCCCAGA |
| 86 | BCMA CAR amino | MALPVTALLLPLALLLHAARPQVQLVQSGAELKKPGASVK |
| | acid sequence | VSCKASGNTLTNYVIHWVRQAPGQRLEWMGYILPYNDLTK |
| | | YSQKFQGRVTITRDKSASTAYMELSSLRSEDTAVYYCTRWD |
| | | WDGFFDPWGQGTTVTVSSGGGGSGGGGSGGGGSEIVMTQS |
| | | PATLSVSPGERASISCRASQSLVHSNGNTHLHWYQQRPGQA |
| | | PRLLIYSVSNRFSEVPARFSGSGSGTDFTLTISSVESEDFAVY |
| | | YCSQTSHIPYTFGGGTKLEIKSAAAFVPVFLPAKPTTTPAPRP |
| | | PTPAPTIASQPLSLRPEACRPAAGGAVHTRGLDFACDIYIWA |
| | | PLAGTCGVLLLSLVITLYCNHRNRKRGRKKLLYIFKQPFMRP |
| | | VQTTQEEDGCSCRFPEEEEGGCELRVKFSRSADAPAYQQGQ |
| | | NQLYNELNLGRREEYDVLDKRRGRDPEMGGKPRRKNPQEG |
| | | LYNELQKDKMAEAYSEIGMKGERRRGKGHDGLYQGLSTAT |
| | | KDTYDALHMQALPPR |
| 87 | BCMA | CAGGTGCAGCTGGTGCAGAGCGGAGCCGAGCTCAAGAAG |
| | scFv nucleotide | CCCGGAGCCTCCGTGAAGGTGAGCTGCAAGGCCAGCGGC |
| | sequence | AACACCCTGACCAACTACGTGATCCACTGGGTGAGACAA |
| | | GCCCCCGGCCAAAGGCTGGAGTGGATGGGCTACATCCTG |
| | | CCCTACAACGACCTGACCAAGTACAGCCAGAAGTTCCAG |
| | | GGCAGGGTGACCATCACCAGGGATAAGAGCGCCTCCACC |
| | | GCCTATATGGAGCTGAGCAGCCTGAGGAGCGAGGACACC |
| | | GCTGTGTACTACTGTACAAGGTGGGACTGGGACGGCTTCT |
| | | TTGACCCCTGGGGCCAGGGCACAACAGTGACCGTCAGCA |
| | | GCGGCGGCGGAGGCAGCGGCGGCGGCGGCAGCGGCGGA |
| | | GGCGGAAGCGAAATCGTGATGACCCAGAGCCCCGCCACA |
| | | CTGAGCGTGAGCCCTGGCGAGAGGGCCAGCATCTCCTGC |
| | | AGGGCTAGCCAAAGCCTGGTGCACAGCAACGGCAACACC |
| | | CACCTGCACTGGTACCAGCAGAGACCCGGACAGGCTCCC |
| | | AGGCTGCTGATCTACAGCGTGAGCAACAGGTTCTCCGAG |
| | | GTGCCTGCCAGGTTTAGCGGCAGCGGAAGCGGCACCGAC |
| | | TTTACCCTGACCATCAGCAGCGTGGAGTCCGAGGACTTCG |
| | | CCGTGTATTACTGCAGCCAGACCAGCCACATCCCTTACAC |
| | | CTTCGGCGGCGGCACCAAGCTGGAGATCAAA |
| 88 | BCMA | QVQLVQSGAELKKPGASVKVSCKASGNTLTNYVIHWVRQA |
| | scFv amino acid | PGQRLEWMGYILPYNDLTKYSQKFQGRVTITRDKSASTAY |
| | sequence | MELSSLRSEDTAVYYCTRWDWDGFFDPWGQGTTVTVSSGG |
| | (linker underlined) | GGSGGGGSGGGGSEIVMTQSPATLSVSPGERASISCRASQSL |
| | | VHSNGNTHLHWYQQRPGQAPRLLIYSVSNRFSEVPARFSGS |
| | | GSGTDFTLTISSVESEDFAVYYCSQTSHIPYTFGGGTKLEIK |
| 89 | BCMA VH | QVQLVQSGAELKKPGASVKVSCKASGNTLTNYVIHWVRQA |
| | | PGQRLEWMGYILPYNDLTKYSQKFQGRVTITRDKSASTAY |
| | | MELSSLRSEDTAVYYCTRWDWDGFFDPWGQGTTVTVSS |
| 90 | BCMA VL | EIVMTQSPATLSVSPGERASISCRASQSLVHSNGNTHLHWYQ |
| | | QRPGQAPRLLIYSVSNRFSEVPARFSGSGSGTDFTLTISSVES |
| | | EDFAVYYCSQTSHIPYTFGGGTKLEIK |
| 91 | CD70 VL CDR1 | RASKSVSTSGYSFMH |
| | (Kabat) | |
| 92 | CD70 VL CDR1 | SKSVSTSGYSF |
| | (Chothia) | |
| 93 | CD70 VL CDR2 | LASNLES |
| | (Kabat) | |
| N/A | CD70 VL CDR2 | LAS |
| | (Chothia) | |
| 95 | CD70 VL CDR3 | QHSREVPWT |
| | (Kabat) | |
| 96 | CD70 VL CDR3 | SREVPW |
| | (Chothia) | |
| 97 | CD70 VH CDR1 | NYGMN |
| | (Kabat) | |
| 98 | CD70 VH CDR1 | GYTFTNYGMN |
| | (Chothia) | |
| 99 | CD70 VH CDR2 | WINTYTGEPTYADAFKG |
| | (Kabat) | |
| 100 | CD70 VH CDR2 | NTYTGE |
| | (Chothia) | |
| 101 | CD70 VH CDR3 | DYGDYGMDY |
| | (Kabat) | |
| 102 | CD70 VH CDR3 | CARDYGDYGMDYWG |
| | (Chothia) | |
| 103 | BCMA VL CDR1 | RASQSLVHSNGNTHLH |
| | (Kabat & Chothia) | |
| 104 | BCMA VL CDR2 | SVSNRFS |
| | (Kabat & Chothia) | |
| 105 | BCMA VL CDR3 | SQTSHIPYT |
| | (Kabat) | |
| 105 | BCMA VL CDR3 | SQTSHIPYT |
| | (Chothia) | |
| 106 | BCMA VH CDR1 | NYVIH |
| | (Kabat) | |
| 107 | BCMA VH CDR1 | GNTLTNY |
| | (Chothia) | |
| 108 | BCMA VH CDR2 | YILPYNDLTKYSQKFQG |
| | (Kabat) | |
| 109 | BCMA VH CDR2 | LPYNDL |
| | (Chothia) | |
| 110 | BCMA VH CDR3 | WDWDGFFDP |
| | (Kabat) | |
| 110 | BCMA VH CDR3 | WDWDGFFDP |
| | (Chothia) | |
| 111 | Anti-CD33 CAR | GAGATGTAAGGAGCTGCTGTGACTTGCTCAAGGCCTTAT |
| | Donor | ATCGAGTAAACGGTAGTGCTGGGGCTTAGACGCAGGTGT |
| | LHA to RHA | TCTGATTTATAGTTCAAAACCTCTATCAATGAGAGAGCA |
| | 41BB costim. | ATCTCCTGGTAATGTGATAGATTTCCCAACTTAATGCCAA |
| | | CATACCATAAACCTCCCATTCTGCTAATGCCCAGCCTAAG |
| | | TTGGGGAGACCACTCCAGATTCCAAGATGTACAGTTTGC |
| | | TTTGCTGGGCCTTTTTCCCATGCCTGCCTTTACTCTGCCAG |
| | | AGTTATATTGCTGGGGTTTTGAAGAAGATCCTATTAAATA |
| | | AAAGAATAAGCAGTATTATTAAGTAGCCCTGCATTTCAG |
| | | GTTTCCTTGAGTGGCAGGCCAGGCCTGGCCGTGAACGTT |
| | | CACTGAAATCATGGCCTCTTGGCCAAGATTGATAGCTTGT |
| | | GCCTGTCCCTGAGTCCCAGTCCATCACGAGCAGCTGGTTT |
| | | CTAAGATGCTATTTCCCGTATAAAGCATGAGACCGTGAC |
| | | TTGCCAGCCCCACAGAGCCCCGCCCTTGTCCATCACTGGC |
| | | ATCTGGACTCCAGCCTGGGTTGGGGCAAAGAGGGAAATG |
| | | AGATCATGTCCTAACCCTGATCCTCTTGTCCCACAGATAT |
| | | CCAGAACCCTGACCCTGCCGTGTACCAGCTGAGAGACTC |
| | | TAAATCCAGTGACAAGTCTGTCTGCCTATTCACCGATTTT |
| | | GATTCTCAAACAAATGTGTCACAAAGTAAGGATTCTGAT |
| | | GTGTATATCACAGACAAAACTGTGCTAGACATGAGGTCT |
| | | ATGGACTTCAggctccggtgcccgtcagtgggcagagcgcacatcgcccacagtcc |
| | | ccgagaagttggggggaggggtcggcaattgaaccggtgcctagagaaggtggcgcgggg |
| | | taaactgggaaagtgatgtcgtgtactggctccgcctttttcccgagggtgggggagaaccgta |
| | | tataagtgcagtagtcgccgtgaacgttctttttcgcaacgggtttgccgccagaacacaggtaa |
| | | gtgccgtgtgtggttcccgcgggcctggcctctttacgggttatggcccttgcgtgccttgaatta |
| | | cttccactggctgcagtacgtgattcttgatcccgagcttcgggttggaagtgggtgggagagtt |
| | | cgaggccttgcgcttaaggagccccttcgcctcgtgcttgagttgaggcctggcctgggcgct |
| | | ggggccgccgcgtgcgaatctggtggcaccttcgcgcctgtctcgctgctttcgataagtctct |
| | | agccatttaaaatttttgatgacctgctgcgacgctttttttctggcaagatagtcttgtaaatgcgg |
| | | gccaagatctgcacactggtatttcggtttttggggccgcgggcggcgacggggcccgtgcgt |
| | | cccagcgcacatgttcggcgaggcggggcctgcgagcgcggccaccgagaatcggacgg |
| | | gggtagtctcaagctggccggcctgctctggtgcctggcctcgcgccgccgtgtatcgccccg |
| | | ccctgggcggcaaggctggcccggtcggcaccagttgcgtgagcggaaagatggccgcttc |
| | | ccggccctgctgcagggagctcaaaatggaggacgcggcgctcgggagagcgggcgggt |
| | | gagtcacccacacaaaggaaaagggcctttccgtcctcagccgtcgcttcatgtgactccacg |
| | | gagtaccgggcgccgtccaggcacctcgattagttctcgagcttttggagtacgtcgtctttagg |
| | | ttggggggaggggattatgcgatggagtttccccacactgagtgggtggagactgaagttagg |
| | | ccagcttggcacttgatgtaattctccttggaatttgccctttttgagtttggatcttggttcattctca |
| | | agcctcagacagtggttcaaagtttttttcttccatttcaggtgtcgtgaCCACCATGGC |
| | | GCTTCCGGTGACAGCACTGCTCCTCCCCTTGGCGCTGTTG |
| | | CTCCACGCAGCAAGGCCGGAAATCGTCCTCACACAATCC |
| | | CCGGGGAGCCTCGCAGTCAGTCCTGGGGAACGAGTCACT |
| | | ATGAGCTGCAAATCCAGTCAGAGTGTTTTTTTCTCAAGTA |
| | | GCCAGAAGAACTACCTCGCATGGTACCAACAAATACCGG |
| | | GGCAATCTCCCCGCTTGCTTATATACTGGGCAAGTACCCG |
| | | CGAATCCGGCGTACCGGATCGATTCACGGGATCTGGGTC |
| | | AGGTACTGATTTCACTTTGACTATCAGCTCTGTTCAGCCT |
| | | GAAGATTTGGCAATTTACTACTGTCACCAATACTTGAGTA |
| | | GCCGAACTTTCGGCCAGGGCACGAAGCTCGAAATCAAGG |
| | | GCGGAGGGGGAGGTTCTGGTGGGGGCGGTTCTGGCGGTG |
| | | GAGGAAGCCAAGTACAGTTGCAACAGCCAGGGGCGGAG |
| | | GTCGTAAAACCTGGGGCGTCTGTCAAGATGAGCTGTAAA |
| | | GCAAGTGGATACACCTTCACCTCCTACTATATACATTGGA |
| | | TTAAGCAAACTCCGGGTCAGGGGCTGGAATGGGTTGGCG |
| | | TTATATACCCCGGGAACGATGATATATCATACAACCAAA |
| | | AATTTCAAGGCAAGGCGACTCTGACTGCCGATAAGAGTA |
| | | GCACAACAGCTTACATGCAGCTTTCTTCCCTGACCAGCGA |
| | | AGATTCAGCAGTTTACTACTGCGCTCGGGAAGTGCGCCT |
| | | GCGATACTTTGATGTCTGGGGTCAAGGAACTACAGTTAC |
| | | TGTATCAAGCAGTGCTGCTGCCTTTGTCCCGGTATTTCTC |
| | | CCAGCCAAACCGACCACGACTCCCGCCCCGCGCCCTCCG |
| | | ACACCCGCTCCCACCATCGCCTCTCAACCTCTTAGTCTTC |
| | | GCCCCGAGGCATGCCGACCCGCCGCCGGGGGTGCTGTTC |
| | | ATACGAGGGGCTTGGACTTCGCTTGTGATATTTACATTTG |
| | | GGCTCCGTTGGCGGGTACGTGCGGCGTCCTTTTGTTGTCA |
| | | CTCGTTATTACTTTGTATTGTAATCACAGGAATCGCAAAC |
| | | GGGGCAGAAAGAAACTCCTGTATATATTCAAACAACCAT |
| | | TTATGAGACCAGTACAAACTACTCAAGAGGAAGATGGCT |
| | | GTAGCTGCCGATTTCCAGAAGAAGAAGAAGGAGGATGTG |
| | | AACTGCGAGTGAAGTTTTCCCGAAGCGCAGACGCTCCGG |
| | | CATATCAGCAAGGACAGAATCAGCTGTATAACGAACTGA |
| | | ATTTGGGACGCCGCGAGGAGTATGACGTGCTTGATAAAC |
| | | GCCGGGGGAGAGACCCGGAAATGGGGGGTAAACCCCGA |
| | | AGAAAGAATCCCCAAGAAGGACTCTACAATGAACTCCAG |
| | | AAGGATAAGATGGCGGAGGCCTACTCAGAAATAGGTATG |
| | | AAGGGCGAACGACGACGGGGAAAAGGTCACGATGGCCT |
| | | CTACCAAGGGTTGAGTACGGCAACCAAAGATACGTACGA |
| | | TGCACTGCATATGCAGGCCCTGCCTCCCAGATAATAATA |
| | | AAATCGCTATCCATCGAAGATGGATGTGTGTTGGTTTTTT |
| | | GTGTGTGGAGCAACAAATCTGACTTTGCATGTGCAAACG |
| | | CCTTCAACAACAGCATTATTCCAGAAGACACCTTCTTCCC |
| | | CAGCCCAGGTAAGGGCAGCTTTGGTGCCTTCGCAGGCTG |
| | | TTTCCTTGCTTCAGGAATGGCCAGGTTCTGCCCAGAGCTC |
| | | TGGTCAATGATGTCTAAAACTCCTCTGATTGGTGGTCTCG |
| | | GCCTTATCCATTGCCACCAAAACCCTCTTTTTACTAAGAA |
| | | ACAGTGAGCCTTGTTCTGGCAGTCCAGAGAATGACACGG |
| | | GAAAAAAGCAGATGAAGAGAAGGTGGCAGGAGAGGGCA |
| | | CGTGGCCCAGCCTCAGTCTCTCCAACTGAGTTCCTGCCTG |
| | | CCTGCCTTTGCTCAGACTGTTTGCCCCTTACTGCTCTTCTA |
| | | GGCCTCATTCTAAGCCCCTTCTCCAAGTTGCCTCTCCTTA |
| | | TTTCTCCCTGTCTGCCAAAAAATCTTTCCCAGCTCACTAA |
| | | GTCAGTCTCACGCAGTCACTCATTAACCCACCAATCACTG |
| | | ATTGTGCCGGCACATGAATGCACCAGGTGTTGAAGTGGA |
| | | GGAATTAAAAAGTCAGATGAGGGGTGTGCCCAGAGGAA |
| | | GCACCATTCTAGTTGGGGGAGCCCATCTGTCAGCTGGGA |
| | | AAAGTCCAAATAACTTCAGATTGGAATGTGTTTTAACTCA |
| | | GGGTTGAGAAAACAGCTACCTTCAGGACAAAAGTCAGGG |
| | | AAGGGCTCTCTGAAGAAATGCTACTTGAAGATACCAGCC |
| | | CTACCAAGGGCAGGGAGAGGACCCTATAGAGGCCTGGG |
| | | ACAGGAGCTCAATGAGAAAGG |
| 112 | Anti-CD33 CAR | CCACCATGGCGCTTCCGGTGACAGCACTGCTCCTCCCCTT |
| | 41BB costim | GGCGCTGTTGCTCCACGCAGCAAGGCCGGAAATCGTCCT |
| | | CACACAATCCCCGGGGAGCCTCGCAGTCAGTCCTGGGGA |
| | | ACGAGTCACTATGAGCTGCAAATCCAGTCAGAGTGTTTTT |
| | | TTCTCAAGTAGCCAGAAGAACTACCTCGCATGGTACCAA |
| | | CAAATACCGGGGCAATCTCCCCGCTTGCTTATATACTGGG |
| | | CAAGTACCCGCGAATCCGGCGTACCGGATCGATTCACGG |
| | | GATCTGGGTCAGGTACTGATTTCACTTTGACTATCAGCTC |
| | | TGTTCAGCCTGAAGATTTGGCAATTTACTACTGTCACCAA |
| | | TACTTGAGTAGCCGAACTTTCGGCCAGGGCACGAAGCTC |
| | | GAAATCAAGGGCGGAGGGGGAGGTTCTGGTGGGGGCGG |
| | | TTCTGGCGGTGGAGGAAGCCAAGTACAGTTGCAACAGCC |
| | | AGGGGCGGAGGTCGTAAAACCTGGGGCGTCTGTCAAGAT |
| | | GAGCTGTAAAGCAAGTGGATACACCTTCACCTCCTACTA |
| | | TATACATTGGATTAAGCAAACTCCGGGTCAGGGGCTGGA |
| | | ATGGGTTGGCGTTATATACCCCGGGAACGATGATATATC |
| | | ATACAACCAAAAATTTCAAGGCAAGGCGACTCTGACTGC |
| | | CGATAAGAGTAGCACAACAGCTTACATGCAGCTTTCTTC |
| | | CCTGACCAGCGAAGATTCAGCAGTTTACTACTGCGCTCG |
| | | GGAAGTGCGCCTGCGATACTTTGATGTCTGGGGTCAAGG |
| | | AACTACAGTTACTGTATCAAGCAGTGCTGCTGCCTTTGTC |
| | | CCGGTATTTCTCCCAGCCAAACCGACCACGACTCCCGCC |
| | | CCGCGCCCTCCGACACCCGCTCCCACCATCGCCTCTCAAC |
| | | CTCTTAGTCTTCGCCCCGAGGCATGCCGACCCGCCGCCG |
| | | GGGGTGCTGTTCATACGAGGGGCTTGGACTTCGCTTGTG |
| | | ATATTTACATTTGGGCTCCGTTGGCGGGTACGTGCGGCGT |
| | | CCTTTTGTTGTCACTCGTTATTACTTTGTATTGTAATCACA |
| | | GGAATCGCAAACGGGGCAGAAAGAAACTCCTGTATATAT |
| | | TCAAACAACCATTTATGAGACCAGTACAAACTACTCAAG |
| | | AGGAAGATGGCTGTAGCTGCCGATTTCCAGAAGAAGAAG |
| | | AAGGAGGATGTGAACTGCGAGTGAAGTTTTCCCGAAGCG |
| | | CAGACGCTCCGGCATATCAGCAAGGACAGAATCAGCTGT |
| | | ATAACGAACTGAATTTGGGACGCCGCGAGGAGTATGACG |
| | | TGCTTGATAAACGCCGGGGGAGAGACCCGGAAATGGGG |
| | | GGTAAACCCCGAAGAAAGAATCCCCAAGAAGGACTCTAC |
| | | AATGAACTCCAGAAGGATAAGATGGCGGAGGCCTACTCA |
| | | GAAATAGGTATGAAGGGCGAACGACGACGGGGAAAAGG |
| | | TCACGATGGCCTCTACCAAGGGTTGAGTACGGCAACCAA |
| | | AGATACGTACGATGCACTGCATATGCAGGCCCTGCCTCC |
| | | CAGATAATAATAAAATCGCTATCCATCGAAGATGGATGT |
| | | GTGTTGGTTTTTTGTGTG |
| 113 | Anti-CD33 | EIVLTQSPGSLAVSPGERVTMSCKSSQSVFFSSSQKNYLAW |
| | scFv | YQQIPGQSPRLLIYWASTRESGVPDRFTGSGSGTDFTLTISSV |
| | Linker underlined | QPEDLAIYYCHQYLSSRTFGQGTKLEIKGGGGGSGGGGSGG |
| | | GGSQVQLQQPGAEVVKPGASVKMSCKASGYTFTSYYIHWI |
| | | KQTPGQGLEWVGVIYPGNDDISYNQKFQGKATLTADKSST |
| | | TAYMQLSSLTSEDSAVYYCAREVRLRYFDVWGQGTTVTVS |
| | | S |
| 114 | Anti-CD33 | GAAATCGTCCTCACACAATCCCCGGGGAGCCTCGCAGTC |
| | scFv | AGTCCTGGGGAACGAGTCACTATGAGCTGCAAATCCAGT |
| | | CAGAGTGTTTTTTTCTCAAGTAGCCAGAAGAACTACCTCG |
| | | CATGGTACCAACAAATACCGGGGCAATCTCCCCGCTTGC |
| | | TTATATACTGGGCAAGTACCCGCGAATCCGGCGTACCGG |
| | | ATCGATTCACGGGATCTGGGTCAGGTACTGATTTCACTTT |
| | | GACTATCAGCTCTGTTCAGCCTGAAGATTTGGCAATTTAC |
| | | TACTGTCACCAATACTTGAGTAGCCGAACTTTCGGCCAG |
| | | GGCACGAAGCTCGAAATCAAGGGCGGAGGGGGAGGTTC |
| | | TGGTGGGGGCGGTTCTGGCGGTGGAGGAAGCCAAGTACA |
| | | GTTGCAACAGCCAGGGGCGGAGGTCGTAAAACCTGGGGC |
| | | GTCTGTCAAGATGAGCTGTAAAGCAAGTGGATACACCTT |
| | | CACCTCCTACTATATACATTGGATTAAGCAAACTCCGGGT |
| | | CAGGGGCTGGAATGGGTTGGCGTTATATACCCCGGGAAC |
| | | GATGATATATCATACAACCAAAAATTTCAAGGCAAGGCG |
| | | ACTCTGACTGCCGATAAGAGTAGCACAACAGCTTACATG |
| | | CAGCTTTCTTCCCTGACCAGCGAAGATTCAGCAGTTTACT |
| | | ACTGCGCTCGGGAAGTGCGCCTGCGATACTTTGATGTCTG |
| | | GGGTCAAGGAACTACAGTTACTGTATCAAGC |
| 115 | Anti-CD33 | MALPVTALLLPLALLLHAARPEIVLTQSPGSLAVSPGERVTM |
| | CAR | SCKSSQSVFFSSSQKNYLAWYQQIPGQSPRLLIYWASTRESG |
| | 41BB costim. | VPDRFTGSGSGTDFTLTISSVQPEDLAIYYCHQYLSSRTFGQ |
| | | GTKLEIKGGGGGSGGGGSGGGGSQVQLQQPGAEVVKPGAS |
| | | VKMSCKASGYTFTSYYIHWIKQTPGQGLEWVGVIYPGNDDI |
| | | SYNQKFQGKATLTADKSSTTAYMQLSSLTSEDSAVYYCARE |
| | | VRLRYFDVWGQGTTVTVSSSAAAFVPVFLPAKPTTTPAPRP |
| | | PTPAPTIASQPLSLRPEACRPAAGGAVHTRGLDFACDIYIWA |
| | | PLAGTCGVLLLSLVITLYCNHRNRKRGRKKLLYIFKQPFMRP |
| | | VQTTQEEDGCSCRFPEEEEGGCELRVKFSRSADAPAYQQGQ |
| | | NQLYNELNLGRREEYDVLDKRRGRDPEMGGKPRRKNPQEG |
| | | LYNELQKDKMAEAYSEIGMKGERRRGKGHDGLYQGLSTAT |
| | | KDTYDALHMQALPPR |
| 116 | anti-CD33 antibody | QVQLQQPGAEVVKPGASVKMSCKASGYTFTSYYIHWIKQT |
| | VH | PGQGLEWVGVIYPGNDDISYNQKFQGKATLTADKSSTTAY |
| | | MQLSSLTSEDSAVYYCAREVRLRYFDVWGQGTTVTVSS |
| 117 | anti-CD33 antibody | EIVLTQSPGSLAVSPGERVTMSCKSSQSVFFSSSQKNYLAWY |
| | VL | QQIPGQSPRLLIYWASTRESGVPDRFTGSGSGTDFTLTISSVQ |
| | | PEDLAIYYCHQYLSSRTFGQGTKLEIK |
| 118 | anti-CD33 antibody | SYYIH |
| | VH CDR1 (Kabat) | |
| 119 | anti-CD33 antibody | VIYPGNDDISYNQKFQG |
| | VH CDR2 (Kabat) | |
| 120 | anti-CD33 antibody | EVRLRYFDV |
| | VH CDR3 (Kabat) | |
| 121 | anti-CD33 antibody | KSSQSVFFSSSQKNYLA |
| | VL CDR1 (Kabat & | |
| | Chothia) | |
| 122 | anti-CD33 antibody | WASTRES |
| | VL CDR2 (Kabat & | |
| | Chothia | |
| 123 | anti-CD33 antibody | HQYLSSRT |
| | VL CDR3 (Kabat & | |
| | Chothia) | |
| 124 | anti-CD19 VL CDR1 | RASQDISKYLN |
| | (Kabat) | |
| 125 | anti-CD19 VL CDR2 | HTSRLHS |
| | (Kabat) | |
| 126 | anti-CD19 VL CDR3 | QQGNTLPYT |
| | (Kabat) | |
| 127 | anti-CD19 VH | DYGVS |
| | CDR1 (Kabat) | |
| 128 | anti-CD19 VH | VIWGSETTYYNSALKS |
| | CDR2 (Kabat) | |
| 129 | anti-CD19 VH | HYYYGGSYAMDY |
| | CDR3 (Kabat) | |
| 124 | anti-CD19 VL CDR1 | RASQDISKYLN |
| | (Chothia) | |
| 125 | anti-CD19 VL CDR2 | HTSRLHS |
| | (Chothia) | |
| 126 | anti-CD19 VL CDR3 | QQGNTLPYT |
| | (Chothia) | |
| 130 | anti-CD19 VH | GVSLPDY |
| | CDR1 (Chothia) | |
| 131 | anti-CD19 VH | WGSET |
| | CDR2 (Chothia) | |
| 129 | anti-CD19 VH | HYYYGGSYAMDY |
| | CDR3 (Chothia) | |
| 132 | anti-CD33 VH | GYTFTSY |
| | CDR1 (Chothia) | |
| 94 | anti-CD33 VH | YPGNDD |
| | CDR2 (Chothia) | |
| 120 | anti-CD33 VH | EVRLRYFDV |
| | CDR3 (Chothia) | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

ClaAs used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for treating cancer, the method comprising administering to a human cancer patient:
(a) an effective amount of an NK cell inhibitor; and
(b) an effective amount of a population of engineered human T cells expressing a CAR (CAR T cells), wherein the engineered human CAR T cells comprise disrupted MHC class I, and wherein the CAR comprises an ectodomain that comprises an antigen-binding fragment, which binds a tumor antigen.

2. The method of claim 1, wherein the engineered human CAR T cells comprise a disrupted beta-2-microglobulin (β2M) gene.

3. The method of claim 1, wherein the engineered human CAR T cells comprise:
(i) a disrupted T cell receptor alpha chain constant region (TRAC) gene;
(ii) a disrupted β2M gene; and
(iii) a nucleic acid encoding the CAR.

4. The method of claim 3, wherein the nucleic acid encoding the CAR is inserted in the disrupted TRAC gene.

5. The method of claim 1, wherein the tumor antigen is CD19, CD33, CD70 or BCMA.

6. The method of claim 1, wherein the NK cell inhibitor is an antibody that specifically binds CD38.

7. The method of claim 6, wherein the antibody is daratumumab, SAR650984, or MOR202, or an antigen-binding fragment thereof.

8. The method of claim 1, wherein the NK cell inhibitor is administered prior to administration of the population of engineered human CAR T cells.

9. The method of claim 1, wherein the method further comprises a pre-conditioning regimen prior to administration of the population of engineered human CAR T cells.

10. The method of claim 9, wherein the pre-conditioning regimen comprises a lymphodepletion regimen.

11. The method of claim 10, wherein the population of engineered human CAR T cells is administered at least 48 hours after the lymphodepletion regimen, and/or no more than seven days after the lymphodepletion regimen.

12. The method of claim 1, wherein step (a) comprises one or more doses of the NK cell inhibitor.

13. The method of claim 3, wherein the NK cell inhibitor is an anti-CD38 antibody.

14. The method of claim 13, wherein the anti-CD38 antibody comprises the same heavy chain and light chain complementary determining regions as daratumumab.

15. The method of claim 14, wherein the anti-CD38 antibody comprises the same heavy chain variable region and the same light chain variable region as daratumumab.

16. The method of claim 15, wherein the NK cell inhibitor is daratumumab and each dose of daratumumab is 1 to 24 mg/kg.

17. The method of claim 15, wherein each dose of daratumumab is 16 mg/kg, which is administered to the human cancer patient as a single dose infusion or as a split dose infusion on two consecutive days, each of which is 8 mg/kg.

18. The method of 10, wherein the human cancer patient is administered a dose of about 1×10⁷-3×10⁸ engineered human CAR T cells expressing a detectable level of the CAR at least 48 hours but no more than seven days after the lymphodepletion therapy.

19. The method of claim 1, wherein the human cancer patient has a CD19⁺ cancer, a CD70⁺ cancer, a BCMA⁺ cancer, or a CD33⁺ cancer.

20. The method of claim 19, wherein the human cancer patient has multiple myeloma, leukemia, or renal cell carcinoma.


SEQUENCE LISTING (132 sequences)

SEQ ID NO: 1 - DNA, 19 bp, Artificial Sequence
  Synthetic polynucleotide
aagagcaacaaatctgact

SEQ ID NO: 2 - DNA, 39 bp, Artificial Sequence
  Synthetic polynucleotide
aagagcaacagtgctgtgcctggagcaacaaatctgact

SEQ ID NO: 3 - DNA, 33 bp, Artificial Sequence
  Synthetic polynucleotide
aagagcaacagtgctggagcaacaaatctgact

SEQ ID NO: 4 - DNA, 34 bp, Artificial Sequence
  Synthetic polynucleotide
aagagcaacagtgcctggagcaacaaatctgact

SEQ ID NO: 5 - DNA, 19 bp, Artificial Sequence
  Synthetic polynucleotide
aagagcaacagtgctgact

SEQ ID NO: 6 - DNA, 41 bp, Artificial Sequence
  Synthetic polynucleotide
aagagcaacagtgctgtgggcctggagcaacaaatctgact

SEQ ID NO: 7 - DNA, 38 bp, Artificial Sequence
  Synthetic polynucleotide
aagagcaacagtgctggcctggagcaacaaatctgact

SEQ ID NO: 8 - DNA, 41 bp, Artificial Sequence
  Synthetic polynucleotide
aagagcaacagtgctgtgtgcctggagcaacaaatctgact

SEQ ID NO: 9 - DNA, 79 bp, Artificial Sequence
  Synthetic polynucleotide
cgtggccttagctgtgctcgcgctactctctctttctgcctggaggctatccagcgtgagtctctcctaccctcccgct

SEQ ID NO: 10 - DNA, 78 bp, Artificial Sequence
  Synthetic polynucleotide
cgtggccttagctgtgctcgcgctactctctctttcgcctggaggctatccagcgtgagtctctcctaccctcccgct

SEQ ID NO: 11 - DNA, 75 bp, Artificial Sequence
  Synthetic polynucleotide
cgtggccttagctgtgctcgcgctactctctctttctggaggctatccagcgtgagtctctcctaccctcccgct

SEQ ID NO: 12 - DNA, 84 bp, Artificial Sequence
  Synthetic polynucleotide
cgtggccttagctgtgctcgcgctactctctctttctggatagcctggaggctatccagcgtgagtctctcctaccctcccgct

SEQ ID NO: 13 - DNA, 55 bp, Artificial Sequence
  Synthetic polynucleotide
cgtggccttagctgtgctcgcgctatccagcgtgagtctctcctaccctcccgct

SEQ ID NO: 14 - DNA, 82 bp, Artificial Sequence
  Synthetic polynucleotide
cgtggccttagctgtgctcgcgctactctctctttctgtggcctggaggctatccagcgtgagtctctcctaccctcccgct

SEQ ID NO: 15 - RNA, 100 nt, Artificial Sequence
  Synthetic polynucleotide
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 16 - RNA, 96 nt, Artificial Sequence
  Synthetic polynucleotide
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 17 - RNA, 114 nt, Artificial Sequence
  Synthetic polynucleotide
  misc_feature (1)..(17): n is a, c, g, or u
  misc_feature (18)..(30): May be absent
  misc_feature (108)..(114): May be absent
nnnnnnnnnnnnnnnnnnnnnnnnnnnnnnguuuuagagcuagaaauagcaaguuaaaauaaggcuaguccguuaucaacuugaaaaaguggcaccgagucggugcuuuuuuuu

SEQ ID NO: 18 - RNA, 100 nt, Artificial Sequence
  Synthetic polynucleotide
agagcaacagugcuguggccguuuuagagcuagaaauagcaaguuaaaauaaggcuaguccguuaucaacuugaaaaaguggcaccgagucggugcuuuu

SEQ ID NO: 19 - RNA, 20 nt, Artificial Sequence
  Synthetic polynucleotide
agagcaacagugcuguggcc

SEQ ID NO: 20 - RNA, 100 nt, Artificial Sequence
  Synthetic polynucleotide
gcuacucucucuuucuggccguuuuagagcuagaaauagcaaguuaaaauaaggcuaguccguuaucaacuugaaaaaguggcaccgagucggugcuuuu

SEQ ID NO: 21 - RNA, 20 nt, Artificial Sequence
  Synthetic polynucleotide
gcuacucucucuuucuggcc

SEQ ID NO: 22 - RNA, 100 nt, Artificial Sequence; identical to SEQ ID NO: 18
  Synthetic polynucleotide
  misc_feature (1)..(4): Modified by 2′-O′methyl phosphorothioate linkage
  misc_feature (97)..(100): Modified by 2′-O′methyl phosphorothioate linkage

SEQ ID NO: 23 - RNA, 20 nt, Artificial Sequence
  Synthetic polynucleotide
  misc_feature (1)..(4): Modified by 2′-O′methyl phosphorothioate linkage
agagcaacagugcuguggcc

SEQ ID NO: 24 - RNA, 100 nt, Artificial Sequence; identical to SEQ ID NO: 20
  Synthetic polynucleotide
  misc_feature (1)..(4): Modified by 2′-O′methyl phosphorothioate linkage
  misc_feature (97)..(100): Modified by 2′-O′methyl phosphorothioate linkage

SEQ ID NO: 25 - RNA, 20 nt, Artificial Sequence
  Synthetic polynucleotide
  misc_feature (1)..(4): Modified by 2′-O′methyl phosphorothioate linkage
gcuacucucucuuucuggcc

SEQ ID NO: 26 - DNA, 23 bp, Artificial Sequence
  Synthetic polynucleotide
agagcaacagtgctgtggcctgg

SEQ ID NO: 27 - DNA, 23 bp, Artificial Sequence
  Synthetic polynucleotide
gctactctctctttctggcctgg

SEQ ID NO: 28 - DNA, 20 bp, Artificial Sequence
  Synthetic polynucleotide
agagcaacagtgctgtggcc

SEQ ID NO: 29 - DNA, 20 bp, Artificial Sequence
  Synthetic polynucleotide
gctactctctctttctggcc

SEQ ID NO: 30 - protein, 22 aa, Artificial Sequence
  Synthetic polypeptide
MLLLVTSLLLCELPHPAFLLIP

SEQ ID NO: 31 - protein, 21 aa, Artificial Sequence
  Synthetic polypeptide
MALPVTALLLPLALLLHAARP

SEQ ID NO: 32 - protein, 23 aa, Artificial Sequence
  Synthetic polypeptide
IYIWAPLAGTCGVLLLSLVITLY

SEQ ID NO: 33 - protein, 126 aa, Artificial Sequence
  Synthetic polypeptide
AAACGGGGCAGAAAGAAACTCCTGTATATATTCAAACAACCATTTATGAGACCAGTACAAACTACTCAAGAGGAAGATGGCTGTAGCTGCCGATTTCCAGAAGAAGAAGAAGGAGGATGTGAACTG

SEQ ID NO: 34 - protein, 42 aa, Artificial Sequence
  Synthetic polypeptide
KRGRKKLLYIFKQPFMRPVQTTQEEDGCSCRFPEEEEGGCEL

SEQ ID NO: 35 - protein, 120 aa, Artificial Sequence
  Synthetic polypeptide
TCAAAGCGGAGTAGGTTGTTGCATTCCGATTACATGAATATGACTCCTCGCCGGCCTGGGCCGACAAGAAAACATTACCAACCCTATGCCCCCCCACGAGACTTCGCTGCGTACAGGTCC

SEQ ID NO: 36 - protein, 40 aa, Artificial Sequence
  Synthetic polypeptide
SKRSRLLHSDYMNMTPRRPGPTRKHYQPYAPPRDFAAYRS

SEQ ID NO: 37 - protein, 336 aa, Artificial Sequence
  Synthetic polypeptide
CGAGTGAAGTTTTCCCGAAGCGCAGACGCTCCGGCATATCAGCAAGGACAGAATCAGCTGTATAACGAACTGAATTTGGGACGCCGCGAGGAGTATGACGTGCTTGATAAACGCCGGGGGAGAGACCCGGAAATGGGGGGTAAACCCCGAAGAAAGAATCCCCAAGAAGGACTCTACAATGAACTCCAGAAGGATAAGATGGCGGAGGCCTACTCAGAAATAGGTATGAAGGGCGAACGACGACGGGGAAAAGGTCACGATGGCCTCTACCAAGGGTTGAGTACGGCAACCAAAGATACGTACGATGCACTGCATATGCAGGCCCTGCCTCCCAGA

SEQ ID NO: 38 - protein, 112 aa, Artificial Sequence
  Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - DNA, 1518 bp, Artificial Sequence
  Synthetic polynucleotide
atgcttcttttggttacgtctctgttgctttgcgaacttcctcatccagcgttcttgctgatccccgatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 40 - protein, 506 aa, Artificial Sequence
  Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - DNA, 145 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 42 - DNA, 130 bp, Artificial Sequence
  Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 43 - DNA, 145 bp, Artificial Sequence
  Synthetic polynucleotide
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 44 - DNA, 141 bp, Artificial Sequence
  Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 45 - DNA, 800 bp, Artificial Sequence
  Synthetic polynucleotide
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 46 - DNA, 804 bp, Artificial Sequence
  Synthetic polynucleotide
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 47 - DNA, 1178 bp, Artificial Sequence
  Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 48 - DNA, 66 bp, Artificial Sequence
  Synthetic polynucleotide
atgcttcttttggttacgtctctgttgctttgcgaacttcctcatccagcgttcttgctgatcccc

SEQ ID NO: 49 - DNA, 735 bp, Artificial Sequence
  Synthetic polynucleotide
gatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagt

SEQ ID NO: 50 - protein, 245 aa, Artificial Sequence
  Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 51 - DNA, 261 bp, Artificial Sequence
  Synthetic polynucleotide
gctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 52 - DNA, 252 bp, Artificial Sequence
  Synthetic polynucleotide
tttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgc

SEQ ID NO: 53 - protein, 84 aa, Artificial Sequence
  Synthetic polypeptide
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 54 - protein, 120 aa, Artificial Sequence
  Synthetic polypeptide
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 55 - protein, 107 aa, Artificial Sequence
  Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 56 - protein, 18 aa, Artificial Sequence
  Synthetic polypeptide
GSTSGSGKPGSGEGSTKG

SEQ ID NO: 57 - DNA, 4682 bp, Artificial Sequence
  Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatgcttcttttggttacgtctctgttgctttgcgaacttcctcatccagcgttcttgctgatccccgatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 58 - DNA, 4358 bp, Artificial Sequence
  Synthetic polynucleotide
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatgcttcttttggttacgtctctgttgctttgcgaacttcctcatccagcgttcttgctgatccccgatattcagatgactcagaccaccagtagcttgtctgcctcactgggagaccgagtaacaatctcctgcagggcaagtcaagacattagcaaatacctcaattggtaccagcagaagcccgacggaacggtaaaactcctcatctatcatacgtcaaggttgcattccggagtaccgtcacgattttcaggttctgggagcggaactgactattccttgactatttcaaacctcgagcaggaggacattgcgacatatttttgtcaacaaggtaataccctcccttacactttcggaggaggaaccaaactcgaaattaccgggtccaccagtggctctgggaagcctggcagtggagaaggttccactaaaggcgaggtgaagctccaggagagcggccccggtctcgttgcccccagtcaaagcctctctgtaacgtgcacagtgagtggtgtatcattgcctgattatggcgtctcctggataaggcagcccccgcgaaagggtcttgaatggcttggggtaatatggggctcagagacaacgtattataactccgctctcaaaagtcgcttgacgataataaaagataactccaagagtcaagttttccttaaaatgaacagtttgcagactgacgataccgctatatattattgtgctaaacattattactacggcggtagttacgcgatggattattgggggcaggggacttctgtcacagtcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 59 - protein, 300 aa, Artificial Sequence
  Synthetic polypeptide
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 60 - DNA, 903 bp, Artificial Sequence
  Synthetic polynucleotide
atggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctga

SEQ ID NO: 61 - protein, 452 aa, Artificial Sequence
  Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 124 aa, Artificial Sequence
  Synthetic polypeptide
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSAS

SEQ ID NO: 63 - protein, 213 aa, Artificial Sequence
  Synthetic polypeptide
EIVLTQSPATLSLSPGERATLSCRASQSVSSYLAWYQQKPQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 107 aa, Artificial Sequence [sequence omitted]

SEQ ID NO: 65 - protein, 5 aa, Artificial Sequence
  Synthetic polypeptide
SFAMS

SEQ ID NO: 66 - protein, 17 aa, Artificial Sequence
  Synthetic polypeptide
AISGSGGGTYYADSVKG

SEQ ID NO: 67 - protein, 13 aa, Artificial Sequence
  Synthetic polypeptide
DKILWFGEPVFDY

SEQ ID NO: 68 - protein, 11 aa, Artificial Sequence
  Synthetic polypeptide
RASQSVSSYLA

SEQ ID NO: 69 - protein, 7 aa, Artificial Sequence
  Synthetic polypeptide
DASNRAT

SEQ ID NO: 70 - protein, 9 aa, Artificial Sequence
  Synthetic polypeptide
QQRSNWPPT

SEQ ID NO: 71 - RNA, 100 nt, Artificial Sequence
  Synthetic polynucleotide
gcuuuggucccauuggucgcguuuuagagcuagaaauagcaaguuaaaauaaggcuaguccguuaucaacuugaaaaaguggcaccgagucggugcuuuu

SEQ ID NO: 72 - RNA, 100 nt, Artificial Sequence
  Synthetic polynucleotide
gcccgcaggacgcacccauaguuuuagagcuagaaauagcaaguuaaaauaaggcuaguccguuaucaacuugaaaaaguggcaccgagucggugcuuuu

SEQ ID NO: 73 - RNA, 100 nt, Artificial Sequence
  Synthetic polynucleotide
cugcagcuucuccaacacauguuuuagagcuagaaauagcaaguuaaaauaaggcuaguccguuaucaacuugaaaaaguggcaccgagucggugcuuuu

SEQ ID NO: 74 - DNA, 4364 bp, Artificial Sequence
  Synthetic polynucleotide
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 75 - DNA, 1527 bp, Artificial Sequence
  Synthetic polynucleotide
atggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataa

SEQ ID NO: 76 - protein, 508 aa, Artificial Sequence
  Synthetic polypeptide
MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - DNA, 735 bp, Artificial Sequence
  Synthetic polynucleotide
gatatagttatgacccaatcacccgatagtcttgcggtaagcctgggggagcgagcaacaataaactgtcgggcatcaaaatccgtcagtacaagcgggtattcattcatgcactggtatcaacagaaacccggtcagccacccaagctcctgatttatcttgcgtctaatcttgagtccggcgtcccagaccggttttccggctccgggagcggcacggattttactcttactatttctagccttcaggccgaagatgtggcggtatactactgccagcattcaagggaagttccttggacgttcggtcagggcacgaaagtggaaattaaaggcggggggggatccggcgggggagggtctggaggaggtggcagtggtcaggtccaactggtgcagtccggggcagaggtaaaaaaacccggcgcgtctgttaaggtttcatgcaaggccagtggatatactttcaccaattacggaatgaactgggtgaggcaggcccctggtcaaggcctgaaatggatgggatggataaacacgtacaccggtgaacctacctatgccgatgcctttaagggtcgggttacgatgacgagagacacctccatatcaacagcctacatggagctcagcagattgaggagtgacgatacggcagtctattactgtgcaagagactacggcgattatggcatggattactggggccagggcactacagtaaccgtttccagc

SEQ ID NO: 78 - protein, 245 aa, Artificial Sequence
  Synthetic polypeptide
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKGGGGSGGGGSGGGGSGQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 79 - DNA, 735 bp, Artificial Sequence
  Synthetic polynucleotide
caggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaa

SEQ ID NO: 80 - protein, 245 aa, Artificial Sequence
  Synthetic polypeptide
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 81 - protein, 118 aa, Artificial Sequence
  Synthetic polypeptide
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 82 - protein, 111 aa, Artificial Sequence
  Synthetic polypeptide
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 83 - DNA, 4688 bp, Artificial Sequence
  Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 84 - DNA, 4364 bp, Artificial Sequence
  Synthetic polynucleotide
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 85 - DNA, 1524 bp, Artificial Sequence
  Synthetic polynucleotide
atggcgcttccggtgacagcactgctcctccccttggcgctgttgctccacgcagcaaggccgcaggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 86 - protein, 508 aa, Artificial Sequence
  Synthetic polypeptide
MALPVTALLLPLALLLHAARPQVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - DNA, 735 bp, Artificial Sequence
  Synthetic polynucleotide
caggtgcagctggtgcagagcggagccgagctcaagaagcccggagcctccgtgaaggtgagctgcaaggccagcggcaacaccctgaccaactacgtgatccactgggtgagacaagcccccggccaaaggctggagtggatgggctacatcctgccctacaacgacctgaccaagtacagccagaagttccagggcagggtgaccatcaccagggataagagcgcctccaccgcctatatggagctgagcagcctgaggagcgaggacaccgctgtgtactactgtacaaggtgggactgggacggcttctttgacccctggggccagggcacaacagtgaccgtcagcagcggcggcggaggcagcggcggcggcggcagcggcggaggcggaagcgaaatcgtgatgacccagagccccgccacactgagcgtgagccctggcgagagggccagcatctcctgcagggctagccaaagcctggtgcacagcaacggcaacacccacctgcactggtaccagcagagacccggacaggctcccaggctgctgatctacagcgtgagcaacaggttctccgaggtgcctgccaggtttagcggcagcggaagcggcaccgactttaccctgaccatcagcagcgtggagtccgaggacttcgccgtgtattactgcagccagaccagccacatcccttacaccttcggcggcggcaccaagctggagatcaaa

SEQ ID NO: 88 - protein, 245 aa, Artificial Sequence
  Synthetic polypeptide
QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSSGGGGSGGGGSGGGGSEIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIK

SEQ ID NO: 89 - protein, 118 aa, Artificial Sequence
  Synthetic polypeptide
QVQLVQSGAELKKPGASVKVSCKASGNTLTNYVIHWVRQAPGQRLEWMGYILPYNDLTKYSQKFQGRVTITRDKSASTAYMELSSLRSEDTAVYYCTRWDWDGFFDPWGQGTTVTVSS

SEQ ID NO: 90 - protein, 112 aa, Artificial Sequence
  Synthetic polypeptide
EIVMTQSPATLSVSPGERASISCRASQSLVHSNGNTHLHWYQQRPGQAPRLLIYSVSNRFSEVPARFSGSGSGTDFTLTISSVESEDFAVYYCSQTSHIPYTFGGGTKLEIK

SEQ ID NO: 91 - protein, 15 aa, Artificial Sequence
  Synthetic polypeptide
RASKSVSTSGYSFMH

SEQ ID NO: 92 - protein, 11 aa, Artificial Sequence
  Synthetic polypeptide
SKSVSTSGYSF

SEQ ID NO: 93 - protein, 7 aa, Artificial Sequence
  Synthetic polypeptide
LASNLES

SEQ ID NO: 94 - protein, 6 aa, Artificial Sequence
  Synthetic polypeptide
YPGNDD

SEQ ID NO: 95 - protein, 9 aa, Artificial Sequence
  Synthetic polypeptide
QHSREVPWT

SEQ ID NO: 96 - protein, 6 aa, Artificial Sequence
  Synthetic polypeptide
SREVPW

SEQ ID NO: 97 - protein, 5 aa, Artificial Sequence
  Synthetic polypeptide
NYGMN

SEQ ID NO: 98 - protein, 10 aa, Artificial Sequence
  Synthetic polypeptide
GYTFTNYGMN

SEQ ID NO: 99 - protein, 17 aa, Artificial Sequence
  Synthetic polypeptide
WINTYTGEPTYADAFKG

SEQ ID NO: 100 - protein, 6 aa, Artificial Sequence
  Synthetic polypeptide
NTYTGE

SEQ ID NO: 101 - protein, 9 aa, Artificial Sequence
  Synthetic polypeptide
DYGDYGMDY

SEQ ID NO: 102 - protein, 14 aa, Artificial Sequence
  Synthetic polypeptide
CARDYGDYGMDYWG

SEQ ID NO: 103 - protein, 16 aa, Artificial Sequence
  Synthetic polypeptide
RASQSLVHSNGNTHLH

SEQ ID NO: 104 - protein, 7 aa, Artificial Sequence
  Synthetic polypeptide
SVSNRFS

SEQ ID NO: 105 - protein, 9 aa, Artificial Sequence
  Synthetic polypeptide
SQTSHIPYT

SEQ ID NO: 106 - protein, 5 aa, Artificial Sequence
  Synthetic polypeptide
NYVIH

SEQ ID NO: 107 - protein, 7 aa, Artificial Sequence
  Synthetic polypeptide
GNTLTNY

SEQ ID NO: 108 - protein, 17 aa, Artificial Sequence
  Synthetic polypeptide
YILPYNDLTKYSQKFQG

SEQ ID NO: 109 - protein, 6 aa, Artificial Sequence
  Synthetic polypeptide
LPYNDL

SEQ ID NO: 110 - protein, 9 aa, Artificial Sequence
  Synthetic polypeptide
WDWDGFFDP

SEQ ID NO: 111 - DNA, 4367 bp, Artificial Sequence
  Synthetic polynucleotide
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccggaaatcgtcctcacacaatccccggggagcctcgcagtcagtcctggggaacgagtcactatgagctgcaaatccagtcagagtgtttttttctcaagtagccagaagaactacctcgcatggtaccaacaaataccggggcaatctccccgcttgcttatatactgggcaagtacccgcgaatccggcgtaccggatcgattcacgggatctgggtcaggtactgatttcactttgactatcagctctgttcagcctgaagatttggcaatttactactgtcaccaatacttgagtagccgaactttcggccagggcacgaagctcgaaatcaagggcggagggggaggttctggtgggggcggttctggcggtggaggaagccaagtacagttgcaacagccaggggcggaggtcgtaaaacctggggcgtctgtcaagatgagctgtaaagcaagtggatacaccttcacctcctactatatacattggattaagcaaactccgggtcaggggctggaatgggttggcgttatataccccgggaacgatgatatatcatacaaccaaaaatttcaaggcaaggcgactctgactgccgataagagtagcacaacagcttacatgcagctttcttccctgaccagcgaagattcagcagtttactactgcgctcgggaagtgcgcctgcgatactttgatgtctggggtcaaggaactacagttactgtatcaagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 112 - DNA, 1585 bp, Artificial Sequence
  Synthetic polynucleotide
ccaccatggcgcttccggtgacagcactgctcctccccttggcgctgttgctccacgcagcaaggccggaaatcgtcctcacacaatccccggggagcctcgcagtcagtcctggggaacgagtcactatgagctgcaaatccagtcagagtgtttttttctcaagtagccagaagaactacctcgcatggtaccaacaaataccggggcaatctccccgcttgcttatatactgggcaagtacccgcgaatccggcgtaccggatcgattcacgggatctgggtcaggtactgatttcactttgactatcagctctgttcagcctgaagatttggcaatttactactgtcaccaatacttgagtagccgaactttcggccagggcacgaagctcgaaatcaagggcggagggggaggttctggtgggggcggttctggcggtggaggaagccaagtacagttgcaacagccaggggcggaggtcgtaaaacctggggcgtctgtcaagatgagctgtaaagcaagtggatacaccttcacctcctactatatacattggattaagcaaactccgggtcaggggctggaatgggttggcgttatataccccgggaacgatgatatatcatacaaccaaaaatttcaaggcaaggcgactctgactgccgataagagtagcacaacagcttacatgcagctttcttccctgaccagcgaagattcagcagtttactactgcgctcgggaagtgcgcctgcgatactttgatgtctggggtcaaggaactacagttactgtatcaagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtg

SEQ ID NO: 113 - protein, 246 aa, Artificial Sequence
  Synthetic polypeptide
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKGGGGGSGGGGSGGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 114 - DNA, 738 bp, Artificial Sequence
  Synthetic polynucleotide
gaaatcgtcctcacacaatccccggggagcctcgcagtcagtcctggggaacgagtcactatgagctgcaaatccagtcagagtgtttttttctcaagtagccagaagaactacctcgcatggtaccaacaaataccggggcaatctccccgcttgcttatatactgggcaagtacccgcgaatccggcgtaccggatcgattcacgggatctgggtcaggtactgatttcactttgactatcagctctgttcagcctgaagatttggcaatttactactgtcaccaatacttgagtagccgaactttcggccagggcacgaagctcgaaatcaagggcggagggggaggttctggtgggggcggttctggcggtggaggaagccaagtacagttgcaacagccaggggcggaggtcgtaaaacctggggcgtctgtcaagatgagctgtaaagcaagtggatacaccttcacctcctactatatacattggattaagcaaactccgggtcaggggctggaatgggttggcgttatataccccgggaacgatgatatatcatacaaccaaaaatttcaaggcaaggcgactctgactgccgataagagtagcacaacagcttacatgcagctttcttccctgaccagcgaagattcagcagtttactactgcgctcgggaagtgcgcctgcgatactttgatgtctggggtcaaggaactacagttactgtatcaagc

SEQ ID NO: 115 - protein, 509 aa, Artificial Sequence
  Synthetic polypeptide
MALPVTALLLPLALLLHAARPEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKGGGGGSGGGGSGGGGSQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - protein, 118 aa, Artificial Sequence
  Synthetic polypeptide
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 117 - protein, 112 aa, Artificial Sequence
  Synthetic polypeptide
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIK

SEQ ID NO: 118 - protein, 5 aa, Artificial Sequence
  Synthetic polypeptide
SYYIH

SEQ ID NO: 119 - protein, 17 aa, Artificial Sequence
  Synthetic polypeptide
VIYPGNDDISYNQKFQG

SEQ ID NO: 120 - protein, 9 aa, Artificial Sequence
  Synthetic polypeptide
EVRLRYFDV

SEQ ID NO: 121 - protein, 17 aa, Artificial Sequence
  Synthetic polypeptide
KSSQSVFFSSSQKNYLA

SEQ ID NO: 122 - protein, 7 aa, Artificial Sequence
  Synthetic polypeptide
WASTRES

SEQ ID NO: 123 - protein, 8 aa, Artificial Sequence
  Synthetic polypeptide
HQYLSSRT

SEQ ID NO: 124 - protein, 11 aa, Artificial Sequence
  Synthetic polypeptide
RASQDISKYLN

SEQ ID NO: 125 - protein, 7 aa, Artificial Sequence
  Synthetic polypeptide
HTSRLHS

SEQ ID NO: 126 - protein, 9 aa, Artificial Sequence
  Synthetic polypeptide
QQGNTLPYT

SEQ ID NO: 127 - protein, 5 aa, Artificial Sequence
  Synthetic polypeptide
DYGVS

SEQ ID NO: 128 - protein, 16 aa, Artificial Sequence
  Synthetic polypeptide
VIWGSETTYYNSALKS

SEQ ID NO: 129 - protein, 12 aa, Artificial Sequence
  Synthetic polypeptide
HYYYGGSYAMDY

SEQ ID NO: 130 - protein, 7 aa, Artificial Sequence
  Synthetic polypeptide
GVSLPDY

SEQ ID NO: 131 - protein, 5 aa, Artificial Sequence
  Synthetic polypeptide
WGSET

SEQ ID NO: 132 - protein, 7 aa, Artificial Sequence
  Synthetic polypeptide
GYTFTSY